(19) United States
(12) Patent Application Publication
(10) Pub. No.: US 2026/0190602 A1
(43) Pub. Date: Jul. 2, 2026

(54) ORGANIC ELECTROLUMINESCENT MATERIAL AND DEVICE

(71) Applicant: BEIJING SUMMER SPROUT TECHNOLOGY CO., LTD., Beijing (CN)

(72) Inventors: Feng Li, Beijing (CN); Xuan Zhang, Beijing (CN); Yang Wang, Beijing (CN); Chi Yuen Raymond Kwong, Beijing (CN); Chuanjun Xia, Beijing (CN)

(73) Assignee: BEIJING SUMMER SPROUT TECHNOLOGY CO., LTD., Beijing (CN)

(21) Appl. No.: 19/434,806

(22) Filed: Dec. 29, 2025

(30) Foreign Application Priority Data
Dec. 31, 2024 (CN) 202411993838.5

(51) Int. Cl. H10K 50/11; C07B 59/00; C07D 403/10; C09K 11/06; H10K 85/30; H10K 85/40; H10K 85/60; H10K 101/00

(52) U.S. Cl. CPC H10K 50/11; C07B 59/004; C07D 403/10; C09K 11/06; H10K 85/342; H10K 85/346; H10K 85/40; H10K 85/622; H10K 85/626; H10K 85/631; H10K 85/654; H10K 85/656; H10K 85/6572; H10K 85/6574; H10K 85/6576; C07B 2200/05; C09K 2211/1029; H10K 2101/90

(57) ABSTRACT

Provided are an organic electroluminescent material and device. The organic electroluminescent material is a compound having a structure of Formula 1. The compound may be used as a host material, an electron transporting material, or a hole blocking material in an organic electroluminescent device and can greatly improve device performance, for example, improve device efficiency and achieve an unexpectedly significant improvement in device lifetime. Further provided are an organic electroluminescent device comprising the organic electroluminescent material and a compound composition comprising the organic electroluminescent material.

### CROSS-REFERENCE TO RELATED APPLICATION(S)

This application claims priority to Chinese Patent Application No. 202411993838.5 filed on Dec. 31, 2024, the disclosure of which is incorporated herein by reference in its entirety.

### TECHNICAL FIELD

The present disclosure relates to compounds for organic electronic devices such as organic light-emitting devices. More particularly, the present disclosure relates to a compound having a structure of Formula 1, an organic electroluminescent device comprising the compound, and a compound composition comprising the compound.

### BACKGROUND

Organic electronic devices include, but are not limited to, the following types: organic light-emitting diodes (OLEDs), organic field-effect transistors (O-FETs), organic light-emitting transistors (OLETs), organic photovoltaic devices (OPVs), dye-sensitized solar cells (DSSCs), organic optical detectors, organic photoreceptors, organic field-quench devices (OFQDs), light-emitting electrochemical cells (LECs), organic laser diodes and organic plasmon emitting devices.

In 1987, Tang and Van Slyke of Eastman Kodak reported a bilayer organic electroluminescent device, which includes an arylamine hole transporting layer and a tris-8-hydroxyquinolato-aluminum layer as the electron and emitting layer (Applied Physics Letters, 1987, 51 (12): 913-915). Once a bias is applied to the device, green light was emitted from the device. This device laid the foundation for the development of modem organic light-emitting diodes (OLEDs). State-of-the-art OLEDs may include multiple layers such as charge injection and transporting layers, charge and exciton blocking layers, and one or multiple emissive layers between the cathode and anode. Since the OLED is a self-emitting solid state device, it offers tremendous potential for display and lighting applications. In addition, the inherent properties of organic materials, such as their flexibility, may make them well suited for particular applications such as fabrication on flexible substrates.

The OLED can be categorized as three different types according to its emitting mechanism. The OLED invented by Tang and van Slyke is a fluorescent OLED. It only utilizes singlet emission. The triplets generated in the device are wasted through nonradiative decay channels. Therefore, the internal quantum efficiency (IQE) of the fluorescent OLED is only 25%. This limitation hindered the commercialization of OLED. In 1997, Forrest and Thompson reported phosphorescent OLED, which uses triplet emission from heavy metal containing complexes as the emitter. As a result, both singlet and triplets can be harvested, achieving 100% IQE. The discovery and development of phosphorescent OLED contributed directly to the commercialization of active-matrix OLED (AMOLED) due to its high efficiency. Recently, Adachi achieved high efficiency through thermally activated delayed fluorescence (TADF) of organic compounds. These emitters have small singlet-triplet gap that makes the transition from triplet back to singlet possible. In the TADF device, the triplet excitons can go through reverse intersystem crossing to generate singlet excitons, resulting in high IQE.

OLEDs can also be classified as small molecule and polymer OLEDs according to the forms of the materials used. A small molecule refers to any organic or organometallic material that is not a polymer. The molecular weight of the small molecule can be large as long as it has well defined structure. Dendrimers with well-defined structures are considered as small molecules. Polymer OLEDs include conjugated polymers and non-conjugated polymers with pendant emitting groups. Small molecule OLED can become the polymer OLED if post polymerization occurred during the fabrication process.

There are various methods for OLED fabrication. Small molecule OLEDs are generally fabricated by vacuum thermal evaporation. Polymer OLEDs are fabricated by solution process such as spin-coating, inkjet printing, and slit printing. If the material can be dissolved or dispersed in a solvent, the small molecule OLED can also be produced by solution process.

The emitting color of the OLED can be achieved by emitter structural design. An OLED may include one emitting layer or a plurality of emitting layers to achieve desired spectrum. In the case of green, yellow, and red OLEDs, phosphorescent emitters have successfully reached commercialization. Blue phosphorescent device still suffers from non-saturated blue color, short device lifetime, and high operating voltage. Commercial full-color OLED displays normally adopt a hybrid strategy, using fluorescent blue and phosphorescent yellow, or red and green. At present, efficiency roll-off of phosphorescent OLEDs at high brightness remains a problem. In addition, it is desirable to have more saturated emitting color, higher efficiency, and longer device lifetime.

Triazine-based organic semiconductor materials are widely applied to OLEDs due to their superior photoelectric performance, redox performance, and stability, etc.

CN118475572A has disclosed an organic compound having a structure of

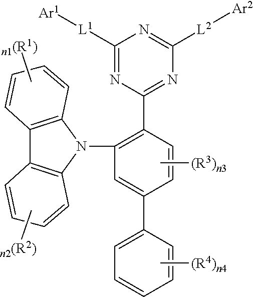

and an organic light-emitting device comprising the compound, wherein Ar¹ and Ar² are independently substituted or unsubstituted C6 to C30 heteroaryl and substituted or unsubstituted C2 to C30 heterocyclic, respectively; L¹ and L² are each independently a single bond, substituted or unsubstituted C6 to C30 arylene, or substituted or unsubstituted C2 to C30 heteroarylene; R¹ and R² are each independently hydrogen, deuterium, or substituted or unsubstituted C6 to C30 heteroaryl; and R³ and R⁴ are each independently hydrogen or deuterium. Further, this application has disclosed the following compounds among specific structures:

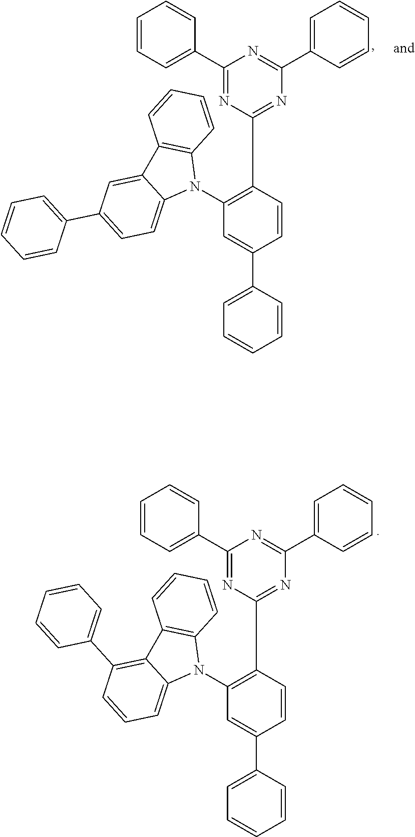 [SMILES: c1ccc(-c2ccc(-c3nc(-c4ccccc4)nc(-c4ccccc4)n3)c(-n3c4ccccc4c4cc(-c5ccccc5)ccc43)c2)cc1 ; c1ccc(-c2ccc(-c3nc(-c4ccccc4)nc(-c4ccccc4)n3)c(-n3c4ccccc4c4c(-c5ccccc5)cccc43)c2)cc1]

However, this application has neither specifically disclosed compounds with the substituent R¹ or R² on carbazole being selected from triphenylenyl nor particularly taught excellent effects of the compounds with carbazole substituted with triphenylene and similar structures and their effects on device performance compared with compounds with carbazole unsubstituted or substituted with other substituents.

CN107667102A has disclosed a light-emitting material having a structure represented by

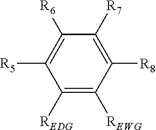

and a device comprising the same, wherein R_{EDG} is an electron-donating group, R_{EWG} is an electron-withdrawing group, and R₅ to R₈ are each independently selected from hydrogen, deuterium, halogen, substituted or unsubstituted C4 to C6 aryl, or substituted or unsubstituted C1 to C3 alkyl. Further, this application has disclosed the following compounds among specific structures:

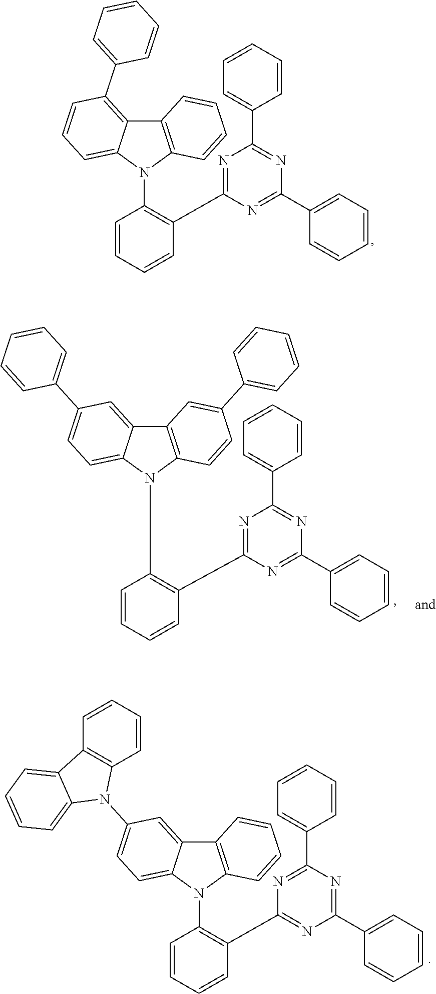 [SMILES: c1ccc(-c2nc(-c3ccccc3)nc(-c3ccccc3-n3c4ccccc4c4c(-c5ccccc5)cccc43)n2)cc1 ; c1ccc(-c2ccc3c(c2)c2cc(-c4ccccc4)ccc2n3-c2ccccc2-c2nc(-c3ccccc3)nc(-c3ccccc3)n2)cc1 ; c1ccc(-c2nc(-c3ccccc3)nc(-c3ccccc3-n3c4ccccc4c4cc(-n5c6ccccc6c6ccccc65)ccc43)n2)cc1]

Similarly, this application has neither disclosed compounds with carbazole substituted with triphenylenyl nor particularly taught unique advantages of the compounds with carbazole further substituted with triphenylene and similar structures and their effects on device performance.

Currently reported triazine-based organic semiconductor materials have certain limitations in terms of carrier transporting ability and lifetime of optoelectronic devices. Therefore, the application potential of such materials is worthy of further research and development.

### SUMMARY

The present disclosure aims to provide a series of compounds each having a structure of Formula 1 to solve at least part of the preceding problems. The compounds may be applied to organic electroluminescent devices and can greatly improve device performance, for example, improve device efficiency and, in particular, achieve an unexpectedly significant improvement in device lifetime.

According to an embodiment of the present disclosure, disclosed is a compound having a structure of Formula 1:

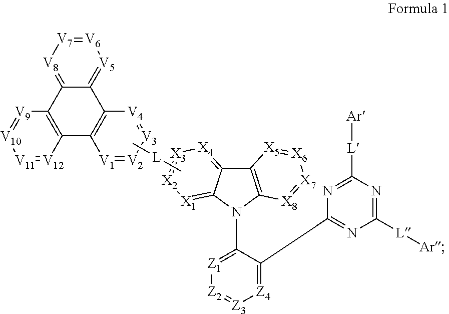

wherein
V₁ to V₄ are, at each occurrence identically or differently, selected from C, CRᵥ, or N, and one of V₁ to V₄ is selected from C and joined to L; V₅ to V₁₂ are, at each occurrence identically or differently, selected from CRᵥ or N;
X₁ to X₄ are, at each occurrence identically or differently, selected from C, CRₓ, or N, and one of X₁ to X₄ is selected from C and joined to L;
X₅ to X₅ are, at each occurrence identically or differently, selected from CRₓ or N;
Z₁ to Z₄ are, at each occurrence identically or differently, selected from CR_{z} or N;
L, L′, and L″ are, at each occurrence identically or differently, selected from a single bond, substituted or unsubstituted alkylene having 1 to 30 carbon atoms, substituted or unsubstituted cycloalkylene having 3 to 30 carbon atoms, substituted or unsubstituted arylene having 6 to 30 carbon atoms, substituted or unsubstituted heteroarylene having 3 to 30 carbon atoms, or a combination thereof;
Ar′ and Ar″ are, at each occurrence identically or differently, selected from substituted or unsubstituted aryl having 6 to 30 carbon atoms, substituted or unsubstituted heteroaryl having 3 to 30 carbon atoms, or a combination thereof;
Rᵥ and R_{z} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
Rₓ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, an isocyano group, a hydroxyl group, a sulfanyl group, a sulfinyl group, a sulfonyl group, a phosphino group, and combinations thereof;
adjacent substituents Rᵥ can be optionally joined to form a ring;
adjacent substituents Rₓ can be optionally joined to form a ring; and
adjacent substituents R_{z} can be optionally joined to form a ring.

According to an embodiment of the present disclosure, further disclosed is an organic electroluminescent device comprising an anode, a cathode, and an organic layer disposed between the anode and the cathode, wherein the organic layer comprises the compound of Formula 1 according to the preceding embodiment.

According to another embodiment of the present disclosure, further disclosed is a compound composition comprising the compound of Formula 1 according to the preceding embodiment.

The present disclosure discloses a series of compounds each having a structure of Formula 1. The compounds may be used as host materials, electron transporting materials, or hole blocking materials in organic electroluminescent devices and can greatly improve device performance, for example, improve device efficiency and achieve an unexpectedly significant improvement in device lifetime.

### BRIEF DESCRIPTION OF DRAWINGS

FIG. 1 is a schematic diagram of an organic light-emitting device that may comprise a compound and a compound composition disclosed herein.

FIG. 2 is a schematic diagram of another organic light-emitting device that may comprise a compound and a compound composition disclosed herein.

### DETAILED DESCRIPTION

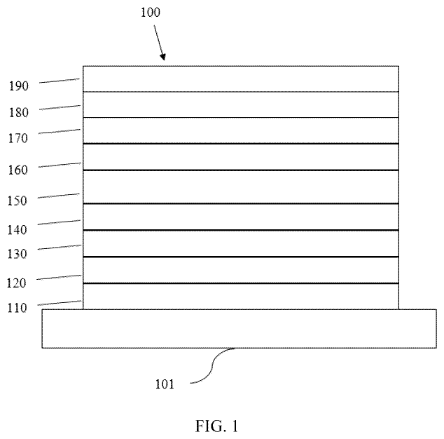

OLEDs can be fabricated on various types of substrates such as glass, plastic, and metal foil. FIG. 1 schematically shows an organic light-emitting device 100 without limitation. The figures are not necessarily drawn to scale. Some of the layers in the figures can also be omitted as needed. Device 100 may include a substrate 101, an anode 110, a hole injection layer 120, a hole transport layer 130, an electron blocking layer 140, an emissive layer 150, a hole blocking layer 160, an electron transport layer 170, an electron injection layer 180 and a cathode 190. Device 100 may be fabricated by depositing the layers described in order. The properties and functions of these various layers, as well as example materials, are described in more detail in U.S. Pat. No. 7,279,704 at cols. 6-10, the contents of which are incorporated by reference herein in its entirety.

More examples for each of these layers are available. For example, a flexible and transparent substrate-anode combination is disclosed in U.S. Pat. No. 5,844,363, which is incorporated by reference herein in its entirety. An example of a p-doped hole transport layer is m-MTDATA doped with F4-TCNQ at a molar ratio of 50:1, as disclosed in U.S. Patent Application Publication No. 2003/0230980, which is incorporated by reference herein in its entirety. Examples of host materials are disclosed in U.S. Pat. No. 6,303,238 to Thompson et al., which is incorporated by reference herein in its entirety. An example of an n-doped electron transport layer is BPhen doped with Li at a molar ratio of 1:1, as disclosed in U.S. Patent Application Publication No. 2003/0230980, which is incorporated by reference herein in its entirety. U.S. Pat. Nos. 5,703,436 and 5,707,745, which are incorporated by reference herein in their entireties, disclose examples of cathodes including composite cathodes having a thin layer of metal such as Mg:Ag with an overlying transparent, electrically-conductive, sputter-deposited ITO layer. The theory and use of blocking layers are described in more detail in U.S. Pat. No. 6,097,147 and U.S. Patent Application Publication No. 2003/0230980, which are incorporated by reference herein in their entireties. Examples of injection layers are provided in U.S. Patent Application Publication No. 2004/0174116, which is incorporated by reference herein in its entirety. A description of protective layers may be found in U.S. Patent Application Publication No. 2004/0174116, which is incorporated by reference herein in its entirety.

The layered structure described above is provided by way of non-limiting examples. Functional OLEDs may be achieved by combining the various layers described in different ways, or layers may be omitted entirely. It may also include other layers not specifically described. Within each layer, a single material or a mixture of multiple materials can be used to achieve optimum performance. Any functional layer may include several sublayers. For example, the emissive layer may have two layers of different emitting materials to achieve desired emission spectrum.

In one embodiment, an OLED may be described as having an “organic layer” disposed between a cathode and an anode. This organic layer may include a single layer or multiple layers.

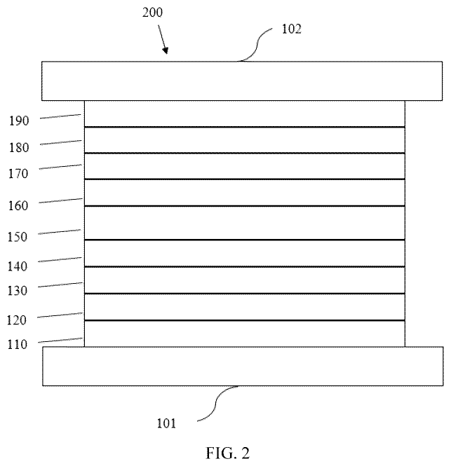

An OLED can be encapsulated by a barrier layer. FIG. 2 schematically shows an organic light emitting device 200 without limitation. FIG. 2 differs from FIG. 1 in that the organic light emitting device include a barrier layer 102, which is above the cathode 190, to protect it from harmful species from the environment such as moisture and oxygen. Any material that can provide the barrier function can be used as the barrier layer such as glass or organic-inorganic hybrid layers. The barrier layer should be placed directly or indirectly outside of the OLED device. Multilayer thin film encapsulation was described in U.S. Pat. No. 7,968,146, which is incorporated by reference herein in its entirety.

Devices fabricated in accordance with embodiments of the present disclosure can be incorporated into a wide variety of consumer products that have one or more of the electronic component modules (or units) incorporated therein. Some examples of such consumer products include flat panel displays, monitors, medical monitors, televisions, billboards, lights for interior or exterior illumination and/or signaling, heads-up displays, fully or partially transparent displays, flexible displays, smart phones, tablets, phablets, wearable devices, smart watches, laptop computers, digital cameras, camcorders, viewfinders, micro-displays, 3-D displays, vehicles displays, and vehicle tail lights.

The materials and structures described herein may be used in other organic electronic devices listed above.

As used herein, “top” means furthest away from the substrate, while “bottom” means closest to the substrate. Where a first layer is described as “disposed over” a second layer, the first layer is disposed further away from the substrate. There may be other layers between the first and second layers, unless it is specified that the first layer is “in contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

It is believed that the internal quantum efficiency (IQE) of fluorescent OLEDs can exceed the 25% spin statistics limit through delayed fluorescence. As used herein, there are two types of delayed fluorescence, i.e. P-type delayed fluorescence and E-type delayed fluorescence. P-type delayed fluorescence is generated from triplet-triplet annihilation (TTA).

On the other hand, E-type delayed fluorescence does not rely on the collision of two triplets, but rather on the transition between the triplet states and the singlet excited states. Compounds that are capable of generating E-type delayed fluorescence are required to have very small singlet-triplet gaps to convert between energy states. Thermal energy can activate the transition from the triplet state back to the singlet state. This type of delayed fluorescence is also known as thermally activated delayed fluorescence (TADF). A distinctive feature of TADF is that the delayed component increases as temperature rises. If the reverse intersystem crossing (RISC) rate is fast enough to minimize the non-radiative decay from the triplet state, the fraction of back populated singlet excited states can potentially reach 75%. The total singlet fraction can be 100%, far exceeding 25% of the spin statistics limit for electrically generated excitons.

E-type delayed fluorescence characteristics can be found in an exciplex system or in a single compound. Without being bound by theory, it is believed that E-type delayed fluorescence requires the luminescent material to have a small singlet-triplet energy gap (ΔE_{S-T}). Organic, non-metal containing, donor-acceptor luminescent materials may be able to achieve this. The emission in these materials is generally characterized as a donor-acceptor charge-transfer (CT) type emission. The spatial separation of the HOMO and LUMO in these donor-acceptor type compounds generally results in small ΔE_{S-T}. These states may involve CT states. Generally, donor-acceptor luminescent materials are constructed by connecting an electron donor moiety such as amino- or carbazole-derivatives and an electron acceptor moiety such as N-containing six-membered aromatic rings.

### Definition of Terms of Substituents

Halogen or halide—as used herein includes fluorine, chlorine, bromine, and iodine.

Alkyl—as used herein includes both straight and branched chain alkyl groups. Alkyl may be alkyl having 1 to 20 carbon atoms, preferably alkyl having 1 to 12 carbon atoms, and more preferably alkyl having 1 to 6 carbon atoms. Examples of alkyl groups include a methyl group, an ethyl group, a propyl group, an isopropyl group, an n-butyl group, an s-butyl group, an isobutyl group, a t-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, a neopentyl group, a 1-methylpentyl group, a 2-methylpentyl group, a 1-pentylhexyl group, a 1-butylpentyl group, a 1-heptyloctyl group, and a 3-methylpentyl group. Of the above, preferred are a methyl group, an ethyl group, a propyl group, an isopropyl group, a n-butyl group, an s-butyl group, an isobutyl group, a t-butyl group, an n-pentyl group, a neopentyl group, and an n-hexyl group.

Additionally, the alkyl group may be optionally substituted.

Cycloalkyl—as used herein includes cyclic alkyl groups. The cycloalkyl groups may be those having 3 to 20 ring carbon atoms, preferably those having 4 to 10 carbon atoms. Examples of cycloalkyl include cyclobutyl, cyclopentyl, cyclohexyl, 4-methylcyclohexyl, 4,4-dimethylcylcohexyl, 1-adamantyl, 2-adamantyl, 1-norbornyl, 2-norbomyl, and the like. Of the above, preferred are cyclopentyl, cyclohexyl, 4-methylcyclohexyl, and 4,4-dimethylcylcohexyl. Additionally, the cycloalkyl group may be optionally substituted.

Heteroalkyl—as used herein, includes a group formed by replacing one or more carbons in an alkyl chain with a hetero-atom(s) selected from the group consisting of a nitrogen atom, an oxygen atom, a sulfur atom, a selenium atom, a phosphorus atom, a silicon atom, a germanium atom, and a boron atom. Heteroalkyl may be those having 1 to 20 carbon atoms, preferably those having 1 to 10 carbon atoms, and more preferably those having 1 to 6 carbon atoms. Examples of heteroalkyl include methoxymethyl, ethoxymethyl, ethoxyethyl, methylthiomethyl, ethylthiomethyl, ethylthioethyl, methoxymethoxymethyl, ethoxymethoxymethyl, ethoxyethoxyethyl, hydroxymethyl, hydroxyethyl, hydroxypropyl, mercaptomethyl, mercaptoethyl, mercaptopropyl, aminomethyl, aminoethyl, aminopropyl, dimethylaminomethyl, trimethylgermanylmethyl, trimethylgermanylethyl, trimethylgermanylisopropyl, dimethylethylgermanylmethyl, dimethylisopropylgermanylmethyl, tert-butylmethylgermanylmethyl, triethylgermanylmethyl, triethylgermanylethyl, triisopropylgermanylmethyl, triisopropylgermanylethyl, trimethylsilylmethyl, trimethylsilylethyl, and trimethylsilylisopropyl, triisopropylsilylmethyl, triisopropylsilylethyl. Additionally, the heteroalkyl group may be optionally substituted.

Alkenyl—as used herein includes straight chain, branched chain, and cyclic alkene groups. Alkenyl may be those having 2 to 20 carbon atoms, preferably those having 2 to 10 carbon atoms. Examples of alkenyl include vinyl, 1-propenyl group, 1-butenyl, 2-butenyl, 3-butenyl, 1,3-butandienyl, 1-methylvinyl, styryl, 2,2-diphenylvinyl, 1,2-diphenylvinyl, 1-methylallyl, 1,1-dimethylallyl, 2-methylallyl, 1-phenylallyl, 2-phenylallyl, 3-phenylallyl, 3,3-diphenylallyl, 1,2-dimethylallyl, 1-phenyl-1-butenyl, 3-phenyl-1-butenyl, cyclopentenyl, cyclopentadienyl, cyclohexenyl, cycloheptenyl, cycloheptatrienyl, cyclooctenyl, cyclooctatetraenyl, and norbomenyl. Additionally, the alkenyl group may be optionally substituted.

Alkynyl—as used herein includes straight chain alkynyl groups. Alkynyl may be those having 2 to 20 carbon atoms, preferably those having 2 to 10 carbon atoms. Examples of alkynyl groups include ethynyl, propynyl, propargyl, 1-butynyl, 2-butynyl, 3-butynyl, 1-pentynyl, 2-pentynyl, 3,3-dimethyl-1-butynyl, 3-ethyl-3-methyl-1-pentynyl, 3,3-diisopropyl-1-pentynyl, phenylethynyl, phenylpropynyl, etc. Of the above, preferred are ethynyl, propynyl, propargyl, 1-butynyl, 2-butynyl, 3-butynyl, 1-pentynyl, and phenylethynyl. Additionally, the alkynyl group may be optionally substituted.

Aryl or an aromatic group—as used herein includes non-condensed and condensed systems. Aryl may be those having 6 to 30 carbon atoms, preferably those having 6 to 20 carbon atoms, and more preferably those having 6 to 12 carbon atoms. Examples of aryl groups include phenyl, biphenyl, terphenyl, triphenylene, tetraphenylene, naphthalene, anthracene, phenalene, phenanthrene, fluorene, pyrene, chrysene, perylene, and azulene, preferably phenyl, biphenyl, terphenyl, triphenylene, fluorene, and naphthalene. Examples of non-condensed aryl groups include phenyl, biphenyl-2-yl, biphenyl-3-yl, biphenyl-4-yl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-(2-phenylpropyl)phenyl, 4′-methylbiphenylyl, 4″-t-butyl-p-terphenyl-4-yl, o-cumenyl, m-cumenyl, p-cumenyl, 2,3-xylyl, 3,4-xylyl, 2,5-xylyl, mesityl, and m-quarterphenyl. Additionally, the aryl group may be optionally substituted.

Heterocyclic groups—as used herein include non-aromatic cyclic groups. Non-aromatic heterocyclic groups include saturated heterocyclic groups having 3 to 20 ring atoms and unsaturated non-aromatic heterocyclic groups having 3 to 20 ring atoms, where at least one ring atom is selected from the group consisting of a nitrogen atom, an oxygen atom, a sulfur atom, a selenium atom, a silicon atom, a phosphorus atom, a germanium atom, and a boron atom. Preferred non-aromatic heterocyclic groups are those having 3 to 7 ring atoms, each of which includes at least one hetero-atom such as nitrogen, oxygen, silicon, or sulfur. Examples of non-aromatic heterocyclic groups include oxiranyl, oxetanyl, tetrahydrofuranyl, tetrahydropyranyl, dioxolanyl, dioxanyl, aziridinyl, dihydropyrrolyl, tetrahydropyrrolyl, piperidinyl, oxazolidinyl, morpholinyl, piperazinyl, oxepinyl, thiepinyl, azepinyl, and tetrahydrosilolyl. Additionally, the heterocyclic group may be optionally substituted.

Heteroaryl—as used herein, includes non-condensed and condensed hetero-aromatic groups having 1 to 5 hetero-atoms, where at least one hetero-atom is selected from the group consisting of a nitrogen atom, an oxygen atom, a sulfur atom, a selenium atom, a silicon atom, a phosphorus atom, a germanium atom, and a boron atom. A hetero-aromatic group is also referred to as heteroaryl. Heteroaryl may be those having 3 to 30 carbon atoms, preferably those having 3 to 20 carbon atoms, and more preferably those having 3 to 12 carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridoindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Alkoxy—as used herein, is represented by —O-alkyl, —O-cycloalkyl, —O-heteroalkyl, or —O-heterocyclic group. Examples and preferred examples of alkyl, cycloalkyl, heteroalkyl, and heterocyclic groups are the same as those described above. Alkoxy groups may be those having 1 to 20 carbon atoms, preferably those having 1 to 6 carbon atoms. Examples of alkoxy groups include methoxy, ethoxy, propoxy, butoxy, pentyloxy, hexyloxy, cyclopropyloxy, cyclobutyloxy, cyclopentyloxy, cyclohexyloxy, tetrahydrofuranyloxy, tetrahydropyranyloxy, methoxypropyloxy, ethoxyethyloxy, methoxymethyloxy, and ethoxymethyloxy. Additionally, the alkoxy group may be optionally substituted.

Aryloxy—as used herein, is represented by —O-aryl or —O-heteroaryl. Examples and preferred examples of aryl and heteroaryl are the same as those described above. Aryloxy groups may be those having 6 to 30 carbon atoms, preferably those having 6 to 20 carbon atoms. Examples of aryloxy groups include phenoxy and biphenyloxy. Additionally, the aryloxy group may be optionally substituted.

Arylalkyl—as used herein, contemplates alkyl substituted with an aryl group. Arylalkyl may be those having 7 to 30 carbon atoms, preferably those having 7 to 20 carbon atoms, and more preferably those having 7 to 13 carbon atoms. Examples of arylalkyl groups include benzyl, 1-phenylethyl, 2-phenylethyl, 1-phenylisopropyl, 2-phenylisopropyl, phenyl-t-butyl, alpha-naphthylmethyl, 1-alpha-naphthylethyl, 2-alpha-naphthylethyl, 1-alpha-naphthylisopropyl, 2-alpha-naphthylisopropyl, beta-naphthylmethyl, 1-beta-naphthylethyl, 2-beta-naphthylethyl, 1-beta-naphthylisopropyl, 2-beta-naphthylisopropyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, and 1-chloro-2-phenylisopropyl. Of the above, preferred are benzyl, p-cyanobenzyl, m-cyanobenzyl, o-cyanobenzyl, 1-phenylethyl, 2-phenylethyl, 1-phenylisopropyl, and 2-phenylisopropyl. Additionally, the arylalkyl group may be optionally substituted.

Alkylsilyl—as used herein, contemplates a silyl group substituted with an alkyl group. Alkylsilyl groups may be those having 3 to 20 carbon atoms, preferably those having 3 to 10 carbon atoms. Examples of alkylsilyl groups include trimethylsilyl, triethylsilyl, methyldiethylsilyl, ethyldimethylsilyl, tripropylsilyl, tributylsilyl, triisopropylsilyl, methyldiisopropylsilyl, dimethylisopropylsilyl, tri-t-butylsilyl, triisobutylsilyl, dimethyl t-butylsilyl, and methyldi-t-butylsilyl. Additionally, the alkylsilyl group may be optionally substituted.

Arylsilyl—as used herein, contemplates a silyl group substituted with an aryl group. Arylsilyl groups may be those having 6 to 30 carbon atoms, preferably those having 8 to 20 carbon atoms. Examples of arylsilyl groups include triphenylsilyl, phenyldibiphenylylsilyl, diphenylbiphenylsilyl, phenyldiethylsilyl, diphenylethylsilyl, phenyldimethylsilyl, diphenylmethylsilyl, phenyldiisopropylsilyl, diphenylisopropylsilyl, diphenylbutylsilyl, diphenylisobutylsilyl, diphenyl t-butylsilyl. Additionally, the arylsilyl group may be optionally substituted.

Alkylgermanyl—as used herein contemplates germanyl substituted with an alkyl group. The alkylgermanyl may be those having 3 to 20 carbon atoms, preferably those having 3 to 10 carbon atoms. Examples of alkylgermanyl include trimethylgermanyl, triethylgermanyl, methyldiethylgermanyl, ethyldimethylgermanyl, tripropylgermanyl, tributylgermanyl, triisopropylgermanyl, methyldiisopropylgermanyl, dimethylisopropylgermanyl, tri-t-butylgermanyl, triisobutylgermanyl, dimethyl-t-butylgermanyl, and methyldi-t-butylgermanyl. Additionally, the alkylgermanyl may be optionally substituted.

Arylgermanyl—as used herein contemplates a germanyl substituted with at least one aryl group or heteroaryl group. Arylgermanyl may be those having 6 to 30 carbon atoms, preferably those having 8 to 20 carbon atoms. Examples of arylgermanyl include triphenylgermanyl, phenyldibiphenylylgermanyl, diphenylbiphenylgermanyl, phenyldiethylgermanyl, diphenylethylgermanyl, phenyldimethylgermanyl, diphenylmethylgermanyl, phenyldiisopropylgermanyl, diphenylisopropylgermanyl, diphenylbutylgermanyl, diphenylisobutylgermanyl, and diphenyl-t-butylgermanyl. Additionally, the arylgermanyl may be optionally substituted.

The term “aza” in azadibenzofuran, azadibenzothiophene, etc. means that one or more of C—H groups in the respective aromatic fragment are replaced by a nitrogen atom. For example, azatriphenylene encompasses dibenzo[f,h]quinoxaline, dibenzo[f,h]quinoline and other analogs with two or more nitrogens in the ring system. One of ordinary skill in the art can readily envision other nitrogen analogs of the aza-derivatives described above, and all such analogs are intended to be encompassed by the terms as set forth herein.

In the present disclosure, unless otherwise defined, when any term of the group consisting of substituted alkyl, substituted cycloalkyl, substituted heteroalkyl, substituted heterocyclic group, substituted arylalkyl, substituted alkoxy, substituted aryloxy, substituted alkenyl, substituted alkynyl, substituted aryl, substituted heteroaryl, substituted alkylsilyl, substituted arylsilyl, substituted alkylgermanyl, substituted arylgermanyl, substituted amino, substituted acyl, substituted carbonyl, a substituted carboxylic acid group, a substituted ester group, substituted sulfinyl, substituted sulfonyl, and substituted phosphino is used, it means that any group of alkyl, cycloalkyl, heteroalkyl, heterocyclic group, arylalkyl, alkoxy, aryloxy, alkenyl, alkynyl, aryl, heteroaryl, alkylsilyl, arylsilyl, amino, acyl, carbonyl, a carboxylic acid group, an ester group, sulfinyl, sulfonyl, and phosphino may be substituted with one or more moieties selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted heteroaryl having 3 to 30 carbon atoms, unsubstituted alkylsilyl having 3 to 20 carbon atoms, unsubstituted arylsilyl having 6 to 20 carbon atoms, unsubstituted alkylgermanyl having 3 to 20 carbon atoms, unsubstituted arylgermanyl having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or an attached fragment are considered to be equivalent.

In the compounds mentioned in the present disclosure, hydrogen atoms may be partially or fully replaced by deuterium. Other atoms such as carbon and nitrogen can also be replaced by their other stable isotopes. The replacement by other stable isotopes in the compounds may be preferred due to its enhancements of device efficiency and stability.

In the compounds mentioned in the present disclosure, multiple substitutions refer to a range that includes a di-substitution, up to the maximum available substitution. When substitution in the compounds mentioned in the present disclosure represents multiple substitution (including di-, tri-, and tetra-substitutions, etc.), that means the substituent may exist at a plurality of available substitution positions on its linking structure, the substituents present at a plurality of available substitution positions may be the same structure or different structures.

In the compounds mentioned in the present disclosure, adjacent substituents in the compounds cannot be joined to form a ring unless otherwise explicitly defined, for example, adjacent substituents can be optionally joined to form a ring. In the compounds mentioned in the present disclosure, the expression that adjacent substituents can be optionally joined to form a ring includes a case where adjacent substituents may be joined to form a ring and a case where adjacent substituents are not joined to form a ring. When adjacent substituents can be optionally joined to form a ring, the ring formed may be monocyclic or polycyclic (including spirocyclic, endocyclic, fusedcyclic, and etc.), as well as alicyclic, heteroalicyclic, aromatic, or heteroaromatic. In such expression, adjacent substituents may refer to substituents bonded to the same atom, substituents bonded to carbon atoms which are directly bonded to each other, or substituents bonded to carbon atoms which are more distant from each other. Preferably, adjacent substituents refer to substituents bonded to the same carbon atom and substituents bonded to carbon atoms which are directly bonded to each other.

The expression that adjacent substituents can be optionally joined to form a ring is also intended to mean that two substituents bonded to the same carbon atom are joined to each other via a chemical bond to form a ring, which can be exemplified by the following formula:

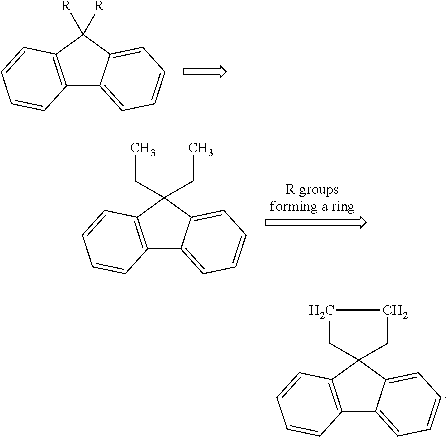

The expression that adjacent substituents can be optionally joined to form a ring is also intended to mean that two substituents bonded to carbon atoms which are directly bonded to each other are joined to each other via a chemical bond to form a ring, which can be exemplified by the following formula:

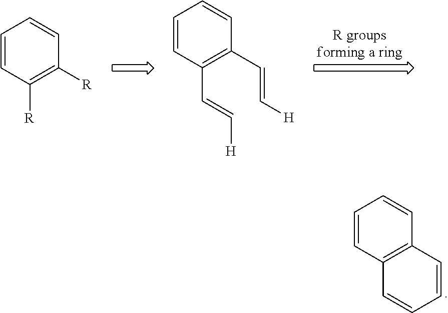

The expression that adjacent substituents can be optionally joined to form a ring is also intended to mean that two substituents bonded to a further distant carbon atom are joined to each other via a chemical bond to form a ring, which can be exemplified by the following formula:

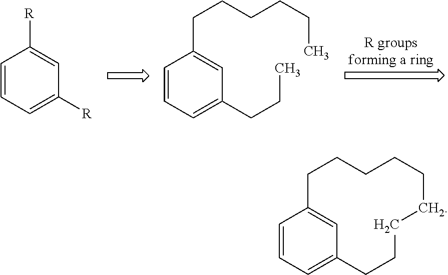

Furthermore, the expression that adjacent substituents can be optionally joined to form a ring is also intended to mean that, in the case where one of the two substituents bonded to carbon atoms which are directly bonded to each other represents hydrogen, the second substituent is bonded at a position at which the hydrogen atom is bonded, thereby forming a ring. This is exemplified by the following formula:

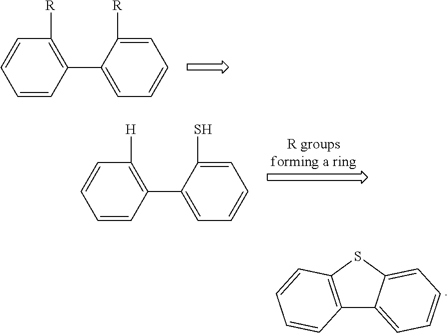

According to an embodiment of the present disclosure, disclosed is a compound having a structure of Formula 1:

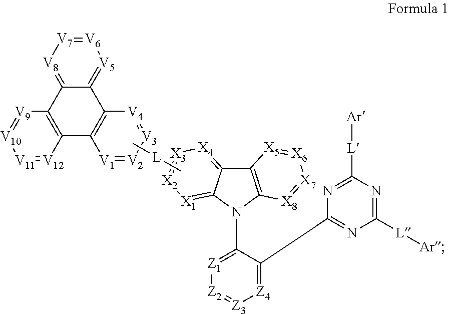

wherein
V₁ to V₄ are, at each occurrence identically or differently, selected from C, CRᵥ, or N, and one of V₁ to V₄ is selected from C and joined to L;
V₅ to V₁₂ are, at each occurrence identically or differently, selected from CRᵥ or N;
X₁ to X₄ are, at each occurrence identically or differently, selected from C, CRₓ, or N, and one of X₁ to X₄ is selected from C and joined to L;
X₅ to X₈ are, at each occurrence identically or differently, selected from CRₓ or N;
Z₁ to Z₄ are, at each occurrence identically or differently, selected from CR_{z} or N;
L, L′, and L″ are, at each occurrence identically or differently, selected from a single bond, substituted or unsubstituted alkylene having 1 to 30 carbon atoms, substituted or unsubstituted cycloalkylene having 3 to 30 carbon atoms, substituted or unsubstituted arylene having 6 to 30 carbon atoms, substituted or unsubstituted heteroarylene having 3 to 30 carbon atoms, or a combination thereof;
Ar′ and Ar″ are, at each occurrence identically or differently, selected from substituted or unsubstituted aryl having 6 to 30 carbon atoms, substituted or unsubstituted heteroaryl having 3 to 30 carbon atoms, or a combination thereof;
Rᵥ and R_{z} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
Rₓ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, an isocyano group, a hydroxyl group, a sulfanyl group, a sulfinyl group, a sulfonyl group, a phosphino group, and combinations thereof;
adjacent substituents Rᵥ can be optionally joined to form a ring;
adjacent substituents Rₓ can be optionally joined to form a ring; and
adjacent substituents R_{z} can be optionally joined to form a ring.

In the present disclosure, the expression that “adjacent substituents Rᵥ can be optionally joined to form a ring” is intended to mean that one or more of groups of substituents formed by any two adjacent substituents Rᵥ in V₁ to V₁₂ can be optionally joined to form a ring. For example, the groups of substituents include, but are not limited to, two adjacent substituents Rᵥ in V₁ and V₂, two adjacent substituents Rᵥ in V₂ and V₃, two adjacent substituents Rᵥ in V₃ and V₄, two adjacent substituents Rᵥ in V₄ and Vs, two adjacent substituents Rᵥ in V₅ and V₆, two adjacent substituents Rᵥ in V₆ and V₇, two adjacent substituents Rᵥ in V₇ and V₈, two adjacent substituents Rᵥ in V₅ and V₉, two adjacent substituents Rᵥ in V₉ and V₁₀, two adjacent substituents Rᵥ in V₁₀ and V₁₁, and two adjacent substituents Rᵥ in V₁₁ and V₁₂. Obviously, it is also possible that any adjacent substituents are not joined to form a ring.

In the present disclosure, the expression that “adjacent substituents Rₓ can be optionally joined to form a ring” is intended to mean that one or more of groups of substituents formed by any two adjacent substituents Rₓ in X₁ to X₅ can be joined to form a ring. For example, the groups of substituents include, but are not limited to, two adjacent substituents Rₓ in X₁ and X₂, two adjacent substituents Rₓ in X₂ and X₃, two adjacent substituents Rₓ in X₃ and X₄, two adjacent substituents Rₓ in X₄ and X₅, two adjacent substituents Rₓ in X₅ and X₆, two adjacent substituents Rₓ in X₆ and X₇, and two adjacent substituents Rₓ in X₇ and X₅. Obviously, it is also possible that any adjacent substituents are not joined to form a ring.

In the present disclosure, the expression that “adjacent substituents R_{z} can be optionally joined to form a ring” is intended to mean that one or more of groups of substituents formed by any two adjacent substituents R_{z} in Z₁ to Z₄ can be joined to form a ring. For example, the groups of substituents include, but are not limited to, two adjacent substituents R_{z} in Z₁ and Z₂, two adjacent substituents R_{z} in Z₂ and Z₃, and two adjacent substituents R_{z} in Z₃ and Z₄. Obviously, it is also possible that any adjacent substituents R_{z} are not joined to form a ring.

In the present disclosure, one or more groups of substituents of adjacent substituents Rᵥ, adjacent substituents Rₓ, or adjacent substituents R_{z} can be joined to form a carbocyclic ring (which may be aromatic or non-aromatic) or a heterocyclic ring (which may be aromatic or non-aromatic), more preferably form a carbocyclic or heterocyclic ring having 6 to 24 ring atoms, more preferably form a carbocyclic or heterocyclic ring having 6 to 12 ring atoms, and still more preferably form a benzene ring. Obviously, it is also possible that any adjacent substituents are not joined to form a ring. In the present disclosure, substituents that are not explicitly described as being joinable to form a ring are not joined to form a ring. For example, Rₓ and Rᵥ, Rₓ and R_{z}, or Rₓ and a substituent on L are not joined to form a ring.

According to an embodiment of the present disclosure, the compound does not comprise a substituted or unsubstituted indolocarbazole group.

According to an embodiment of the present disclosure, V₂ is selected from C and joined to L in Formula 1.

According to an embodiment of the present disclosure, X₃ or X₄ is selected from C and joined to L in Formula 1.

According to an embodiment of the present disclosure, V₂ is selected from C and joined to L in Formula 1, and X₄ is selected from C and joined to L in Formula 1.

According to an embodiment of the present disclosure, at least one of Z₁ to Z₄ is selected from CR_{z}, and the R_{z} is, at each occurrence identically or differently, selected from substituted or unsubstituted aryl having 6 to 30 carbon atoms, substituted or unsubstituted heteroaryl having 3 to 30 carbon atoms, or a combination thereof.

According to an embodiment of the present disclosure, at least one of Z₁ to Z₄ is selected from CR_{z}, and the R_{z} is, at each occurrence identically or differently, selected from substituted or unsubstituted aryl having 6 to 20 carbon atoms, substituted or unsubstituted heteroaryl having 3 to 20 carbon atoms, or a combination thereof.

According to an embodiment of the present disclosure, Z₂ or Z₃ is selected from CR_{z}, and the R_{z} is, at each occurrence identically or differently, selected from substituted or unsubstituted aryl having 6 to 30 carbon atoms, substituted or unsubstituted heteroaryl having 3 to 30 carbon atoms, or a combination thereof.

According to an embodiment of the present disclosure, at least one of Z₁ to Z₄ is selected from CR_{z}, and the R_{z} is, at each occurrence identically or differently, selected from the group consisting of: substituted or unsubstituted phenyl, substituted or unsubstituted naphthyl, substituted or unsubstituted phenanthryl, substituted or unsubstituted biphenyl, substituted or unsubstituted terphenyl, substituted or unsubstituted triphenylenyl, substituted or unsubstituted fluorenyl, substituted or unsubstituted pyridyl, substituted or unsubstituted carbazolyl, substituted or unsubstituted dibenzofuryl, substituted or unsubstituted dibenzothienyl, and combinations thereof.

According to an embodiment of the present disclosure, the compound has a structure represented by Formula 1-1 or Formula 1-2:

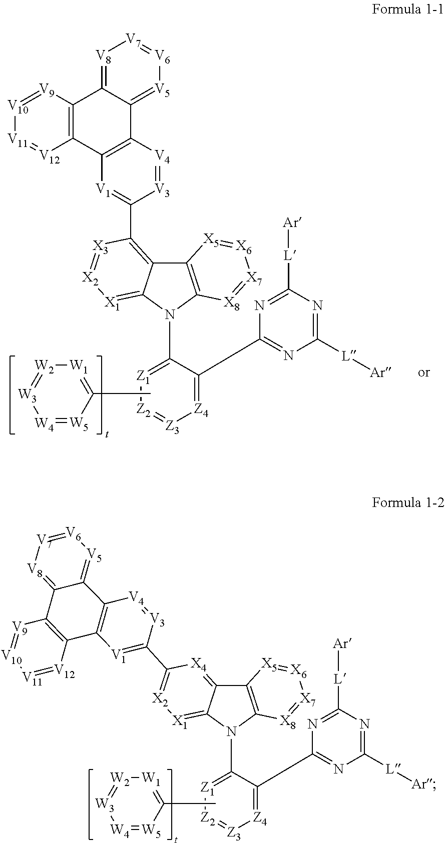

wherein
V₁ and V₃ to V₁₂ are, at each occurrence identically or differently, selected from CRᵥ or N;
X₁ to X₈ are, at each occurrence identically or differently, selected from CRₓ or N;
Z₁ to Z₄ are, at each occurrence identically or differently, selected from C, CR_{z}, or N;
W₁ to W₅ are, at each occurrence identically or differently, selected from CR_{w} or N; t is selected from 0, 1, 2, 3, or 4;
L′ and L″ are, at each occurrence identically or differently, selected from a single bond, substituted or unsubstituted alkylene having 1 to 30 carbon atoms, substituted or unsubstituted cycloalkylene having 3 to 30 carbon atoms, substituted or unsubstituted arylene having 6 to 30 carbon atoms, substituted or unsubstituted heteroarylene having 3 to 30 carbon atoms, or a combination thereof;
Ar′ and Ar″ are, at each occurrence identically or differently, selected from substituted or unsubstituted aryl having 6 to 30 carbon atoms, substituted or unsubstituted heteroaryl having 3 to 30 carbon atoms, or a combination thereof;
R_{w}, Rᵥ, and R_{z} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
Rₓ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, an isocyano group, a hydroxyl group, a sulfanyl group, a sulfinyl group, a sulfonyl group, a phosphino group, and combinations thereof;
adjacent substituents Rᵥ can be optionally joined to form a ring;
adjacent substituents Rₓ can be optionally joined to form a ring; and
adjacent substituents R_{w} can be optionally joined to form a ring.

In this embodiment, t represents the number of the structure

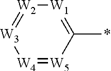

on the six-membered ring on which Z₁ to Z₄ are located in Formula 1-1 or Formula 1-2, and “*” represents a position where the structure is joined to the six-membered ring in Formula 1-1 or Formula 1-2. For example, when t is selected from 0, it indicates that the six-membered ring in Formula 1-1 or Formula 1-2 has no substitution of the structure; when t is selected from 1, it indicates that one of Z₁ to Z₄ is selected from C and joined to the structure

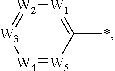

wherein “*” indicates the position of substitution; when t is selected from 2, it indicates that two of Z₁ to Z₄ are selected from C and joined to the structure

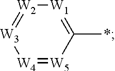

the same is true for other cases.

In the present disclosure, the expression that “adjacent substituents Rᵥ can be optionally joined to form a ring” is intended to mean that one or more of groups of substituents formed by any two adjacent substituents Rᵥ in V₁ and V₃ to V₁₂ can be optionally joined to form a ring. Obviously, it is also possible that any adjacent substituents are not joined to form a ring.

In the present disclosure, the expression that “adjacent substituents Rₓ can be optionally joined to form a ring” is intended to mean that one or more of groups of substituents formed by any two adjacent substituents Rₓ in X₁ to X₅ can be joined to form a ring. Obviously, it is also possible that any adjacent substituents are not joined to form a ring.

In the present disclosure, the expression that “adjacent substituents R_{w} can be optionally joined to form a ring” is intended to mean that one or more of groups of substituents formed by any two adjacent substituents R_{w} in W₁ to W₅ can be joined to form a ring. For example, the groups of substituents include, but are not limited to, two adjacent substituents R_{w} in W₁ and W₂, two adjacent substituents R_{w} in W₂ and W₃, two adjacent substituents R_{w} in W₃ and W₄, and two adjacent substituents R_{w} in W₄ and W₅. Obviously, it is also possible that any adjacent substituents are not joined to form a ring.

According to an embodiment of the present disclosure, V₁ to V₄ are, at each occurrence identically or differently, selected from C or CRᵥ.

According to an embodiment of the present disclosure, t is selected from 0, and Z₁ to Z₄ are, at each occurrence identically or differently, selected from CR_{z} or N; or t is selected from 1, Z₂ or is selected from C and joined to

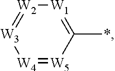

and Z₁ and Z₄ are, at each occurrence identically or differently, selected from CR_{z} or N.

According to an embodiment of the present disclosure, V₅ to V₁₂ are, at each occurrence identically or differently, selected from CRᵥ.

According to an embodiment of the present disclosure, X₁ to X₄ are, at each occurrence identically or differently, selected from C or CRₓ.

According to an embodiment of the present disclosure, X₅ to X₅ are, at each occurrence identically or differently, selected from CRₓ.

According to an embodiment of the present disclosure, Z₁ to Z₄ are, at each occurrence identically or differently, selected from CR_{z}.

According to an embodiment of the present disclosure, W₁ to W₅ are, at each occurrence identically or differently, selected from CR_{w}.

According to an embodiment of the present disclosure, Ar′ and Ar″ are, at each occurrence identically or differently, selected from substituted or unsubstituted aryl having 6 to 20 carbon atoms, substituted or unsubstituted heteroaryl having 3 to 20 carbon atoms, or a combination thereof.

According to an embodiment of the present disclosure, Ar′ and Ar″ are, at each occurrence identically or differently, selected from substituted or unsubstituted aryl having 6 to 20 carbon atoms.

According to an embodiment of the present disclosure, Ar′ and Ar″ are, at each occurrence identically or differently, selected from substituted or unsubstituted aryl having 6 to 18 carbon atoms, substituted or unsubstituted heteroaryl having 3 to 18 carbon atoms, or a combination thereof.

According to an embodiment of the present disclosure, Ar′ and Ar″ are, at each occurrence identically or differently, selected from substituted or unsubstituted aryl having 6 to 12 carbon atoms, substituted or unsubstituted heteroaryl having 3 to 20 carbon atoms, or a combination thereof; and the substituted aryl or the substituted heteroaryl refers to that aryl or heteroaryl may be substituted with one or more groups selected from the group consisting of: deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 12 carbon atoms, unsubstituted heteroaryl having 6 to 30 carbon atoms, unsubstituted alkylsilyl having 3 to 20 carbon atoms, unsubstituted arylsilyl having 6 to 20 carbon atoms, unsubstituted alkylgermanyl having 3 to 20 carbon atoms, unsubstituted arylgermanyl having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

According to an embodiment of the present disclosure, Ar′ and Ar″ are, at each occurrence identically or differently, selected from the group consisting of: substituted or unsubstituted phenyl, substituted or unsubstituted naphthyl, substituted or unsubstituted phenanthryl, substituted or unsubstituted biphenyl, substituted or unsubstituted terphenyl, substituted or unsubstituted triphenylenyl, substituted or unsubstituted fluorenyl, substituted or unsubstituted pyridyl, substituted or unsubstituted pyrimidinyl, substituted or unsubstituted carbazolyl, substituted or unsubstituted dibenzofuryl, substituted or unsubstituted dibenzothienyl, and combinations thereof.

According to an embodiment of the present disclosure, L, L′, and L″ are, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms, or a combination thereof.

According to an embodiment of the present disclosure, L, L′, and L″ are, at each occurrence identically or differently, selected from a single bond, substituted or unsubstituted phenylene, substituted or unsubstituted biphenylylene, substituted or unsubstituted naphthylene, substituted or unsubstituted pyridylene, or a combination thereof.

According to an embodiment of the present disclosure, L, L′, and L″ are, at each occurrence identically or differently, selected from a single bond or phenylene.

According to an embodiment of the present disclosure, R_{w}, R_{y}, and R_{z} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 20 carbon atoms, substituted or unsubstituted heteroaryl having 3 to 20 carbon atoms, cyano, and combinations thereof.

According to an embodiment of the present disclosure, R_{w}, Rᵥ, and R_{z} are, at each occurrence identically or differently, selected from the group consisting of: hydrogen, deuterium, fluorine, substituted or unsubstituted phenyl, substituted or unsubstituted naphthyl, substituted or unsubstituted biphenyl, substituted or unsubstituted terphenyl, substituted or unsubstituted triphenylenyl, substituted or unsubstituted fluorenyl, substituted or unsubstituted pyridyl, substituted or unsubstituted pyrimidinyl, substituted or unsubstituted carbazolyl, substituted or unsubstituted dibenzofuryl, substituted or unsubstituted dibenzothienyl, cyano, and combinations thereof.

According to an embodiment of the present disclosure, Rₓ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 20 carbon atoms, substituted or unsubstituted heteroaryl having 3 to 20 carbon atoms, and combinations thereof.

According to an embodiment of the present disclosure, Rₓ is, at each occurrence identically or differently, selected from the group consisting of: hydrogen, deuterium, fluorine, substituted or unsubstituted phenyl, substituted or unsubstituted naphthyl, substituted or unsubstituted biphenyl, substituted or unsubstituted terphenyl, substituted or unsubstituted triphenylenyl, substituted or unsubstituted fluorenyl, substituted or unsubstituted pyridyl, substituted or unsubstituted carbazolyl, substituted or unsubstituted dibenzofuryl, substituted or unsubstituted dibenzothienyl, and combinations thereof.

According to an embodiment of the present disclosure, the compound is selected from the group consisting of Compound A-1 to Compound A-882, wherein for specific structures of Compound A-1 to Compound A-882 are shown in claim 9.

According to an embodiment of the present disclosure, hydrogens in structures of Compound A-1 to Compound A-882 can be partially or fully substituted with deuterium.

According to an embodiment of the present disclosure, further disclosed is an organic electroluminescent device comprising:
an anode,
a cathode, and
an organic layer disposed between the anode and the cathode, wherein the organic layer comprises a compound having a structure of Formula 1, and the compound having the structure of Formula 1 is as shown in any one of the preceding embodiments.

According to an embodiment of the present disclosure, the organic layer is an emissive layer, and the compound is a host compound.

According to an embodiment of the present disclosure, the organic layer is an electron transporting layer, and the compound is an electron transporting compound.

According to an embodiment of the present disclosure, the organic layer is a hole blocking layer, and the compound is a hole blocking compound.

According to an embodiment of the present disclosure, the organic layer is an emissive layer, the compound is a host compound, the emissive layer further comprises a second host compound, and the second host compound comprises at least one chemical group selected from the group consisting of: benzene, pyridine, pyrimidine, triazine, carbazole, azacarbazole, indolocarbazole, dibenzothiophene, aza-dibenzothiophene, dibenzofuran, azadibenzofuran, dibenzoselenophene, triphenylene, azatriphenylene, fluorene, silafluorene, naphthalene, quinoline, isoquinoline, quinazoline, quinoxaline, phenanthrene, azaphenanthrene, and combinations thereof.

According to an embodiment of the present disclosure, the second host compound comprises at least one chemical group selected from the group consisting of: benzene, carbazole, indolocarbazole, dibenzofuran, dibenzothiophene, and combinations thereof.

According to an embodiment of the present disclosure, the second host compound has a structure represented by Formula 2 or Formula 3:

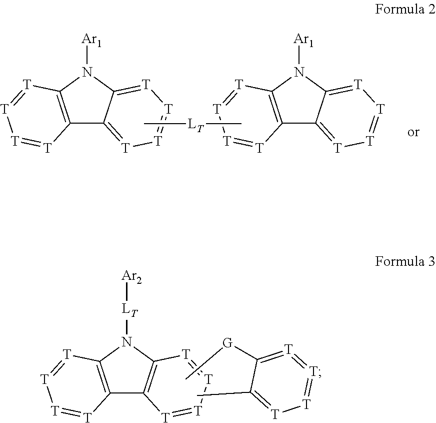

wherein
T is, at each occurrence identically or differently, selected from C, CRₜ, or N;
G is, at each occurrence identically or differently, selected from C(R_{g})₂, NR_{g}, O, or S; when multiple R_{g} are present at the same time, the multiple R_{g} are the same or different;
L_{T} is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms, or a combination thereof;
Rₜ and R_{g} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
Ar₁ and Ar₂ are, at each occurrence identically or differently, selected from substituted or unsubstituted aryl having 6 to 30 carbon atoms, substituted or unsubstituted heteroaryl having 3 to 30 carbon atoms, or a combination thereof; and
adjacent substituents Rₜ, R_{g} can be optionally joined to form a ring.

In the present disclosure, the expression that “adjacent substituents Rₜ, R_{g} can be optionally joined to form a ring” is intended to mean that any one or more of groups of adjacent substituents, such as two substituents Rₜ, two substituents R_{g}, and substituents Rₜ and R_{g}, can be joined to form a ring. Obviously, it is also possible that none of these substituents are joined to form a ring.

According to an embodiment of the present disclosure, the second host compound has a structure represented by one of Formula 2-a to Formula 2-j and Formula 3-a to Formula 3-f:

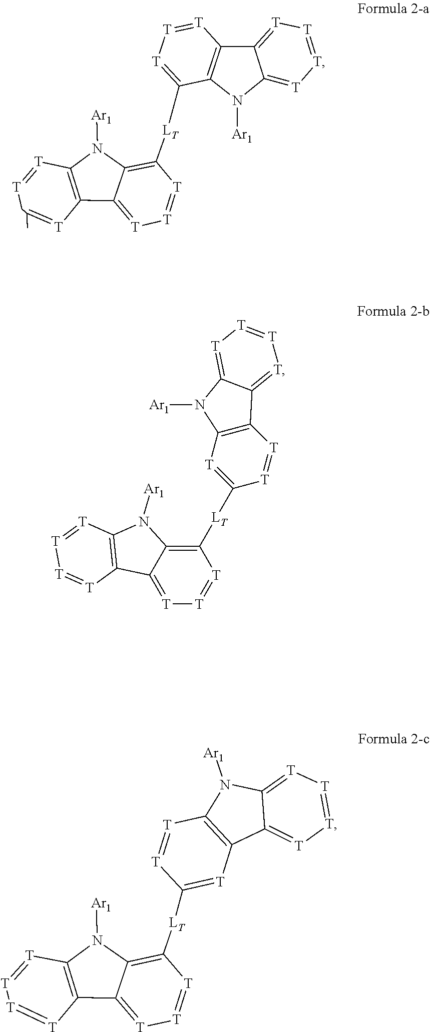

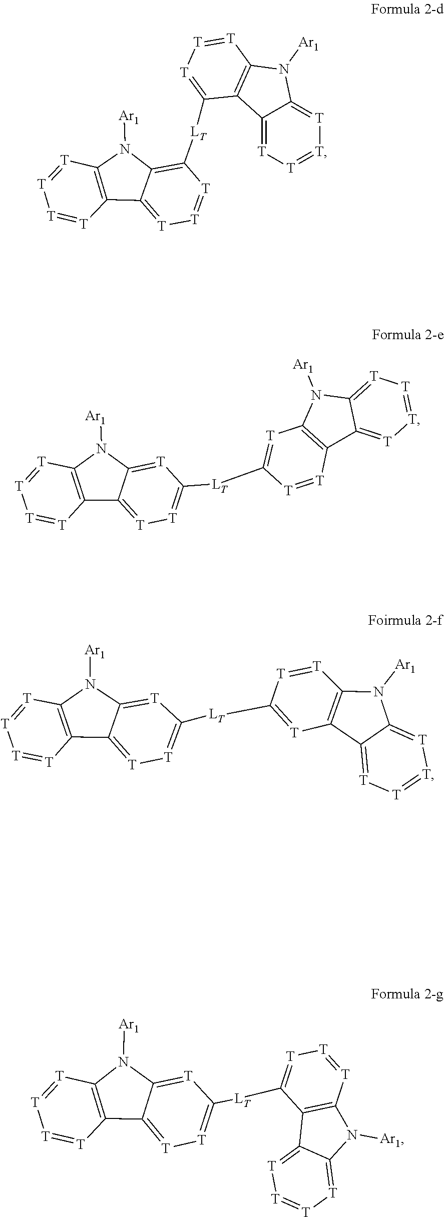

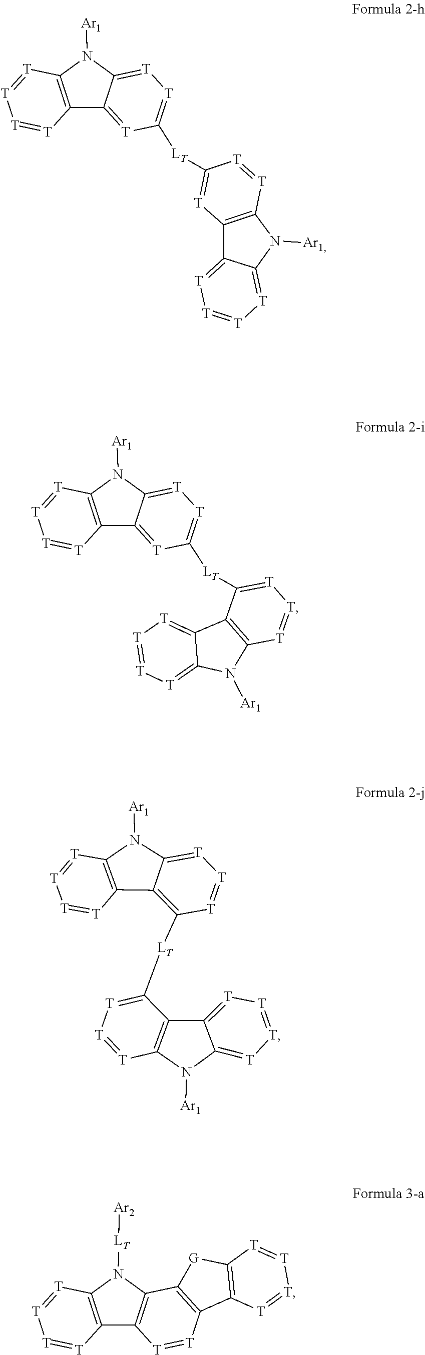

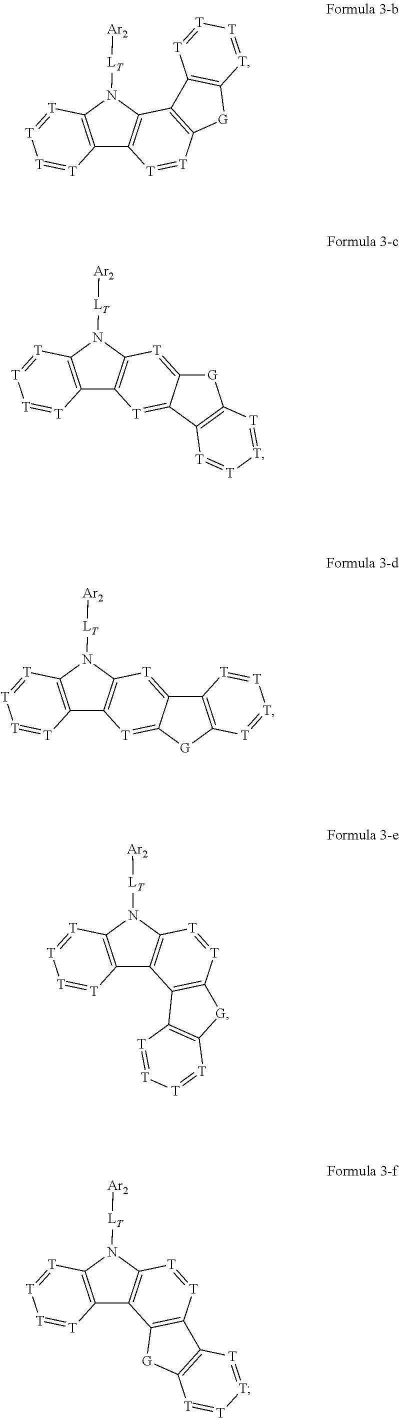

wherein
T is, at each occurrence identically or differently, selected from CRₜ or N;
G is, at each occurrence identically or differently, selected from C(R_{g})₂, NR_{g}, O, or S; when multiple R_{g} are present at the same time, the multiple R_{g} are the same or different;
L_{T} is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms, or a combination thereof;
Ar₁ and Ar₂ are, at each occurrence identically or differently, selected from substituted or unsubstituted aryl having 6 to 30 carbon atoms, substituted or unsubstituted heteroaryl having 3 to 30 carbon atoms, or a combination thereof;
Rₜ and R_{g} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents Rₜ, R_{g} can be optionally joined to form a ring.

According to an embodiment of the present disclosure, in Formula 2-a to Formula 2-j and Formula 3-a to Formula 3-f, T is, at each occurrence identically or differently, selected from CRₜ.

According to an embodiment of the present disclosure, in Formula 2-a to Formula 2-j and Formula 3-a to Formula 3-f, T is, at each occurrence identically or differently, selected from CRₜ or N, and at least one T is selected from N, for example, one T is selected from N or two T are selected from N.

According to an embodiment of the present disclosure, the second host compound is selected from the group consisting of Compound PH-1 to Compound PH-115:

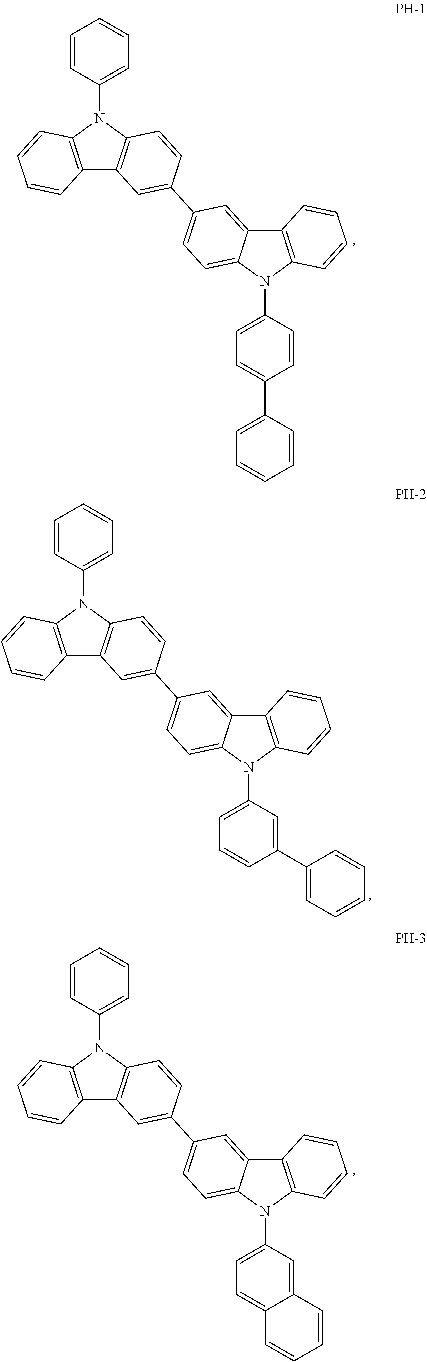 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4ccc5ccccc5c4)ccc32)cc1]

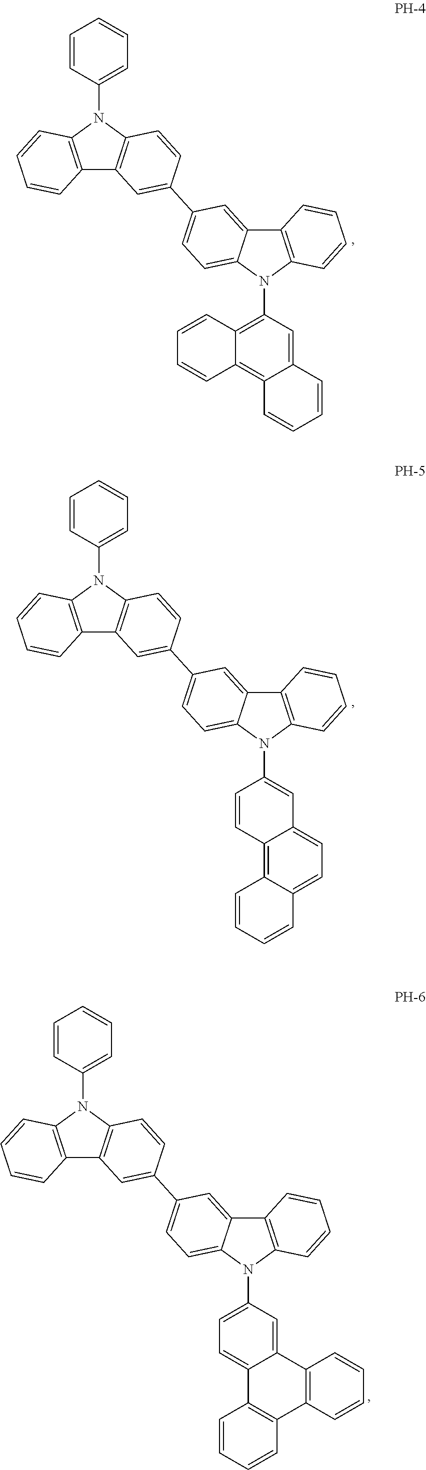 [SMILES: c1ccc(-n2c3ccccc3c3cc(-c4ccc5c(c4)c4ccccc4n5-c4cc5ccccc5c5ccccc45)ccc32)cc1 ; c1ccc(-n2c3ccccc3c3cc(-c4ccc5c(c4)c4ccccc4n5-c4ccc5c(ccc6ccccc65)c4)ccc32)cc1 ; c1ccc(-n2c3ccccc3c3cc(-c4ccc5c(c4)c4ccccc4n5-c4ccc5c6ccccc6c6ccccc6c5c4)ccc32)cc1]

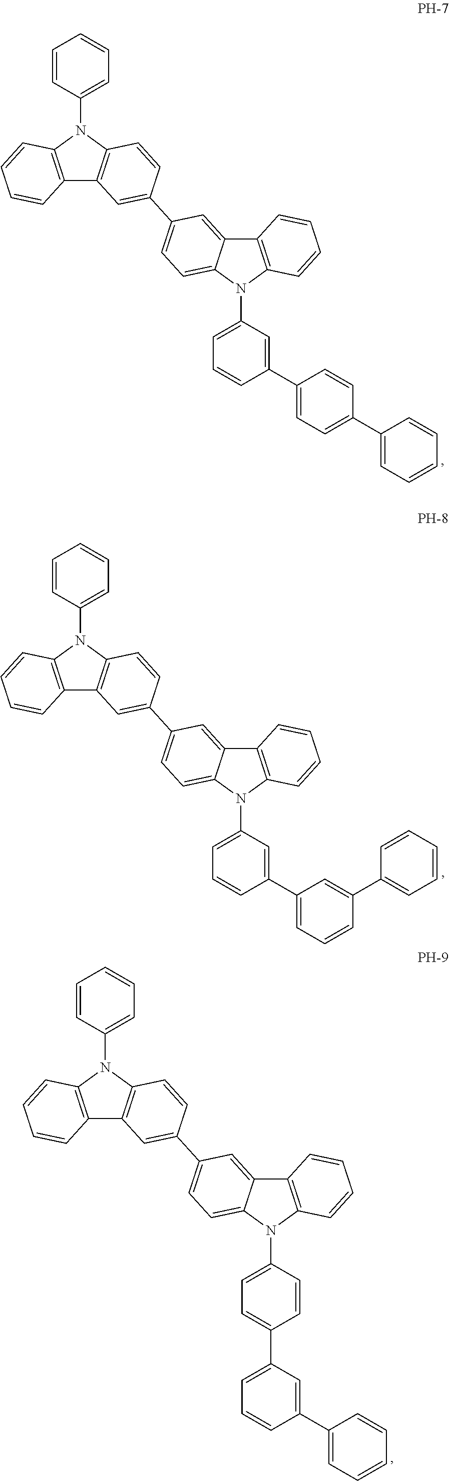 [SMILES: c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2cccc(-c3cccc(-n4c5ccccc5c5cc(-c6ccc7c(c6)c6ccccc6n7-c6ccccc6)ccc54)c3)c2)cc1 ; c1ccc(-c2cccc(-c3ccc(-n4c5ccccc5c5cc(-c6ccc7c(c6)c6ccccc6n7-c6ccccc6)ccc54)cc3)c2)cc1]

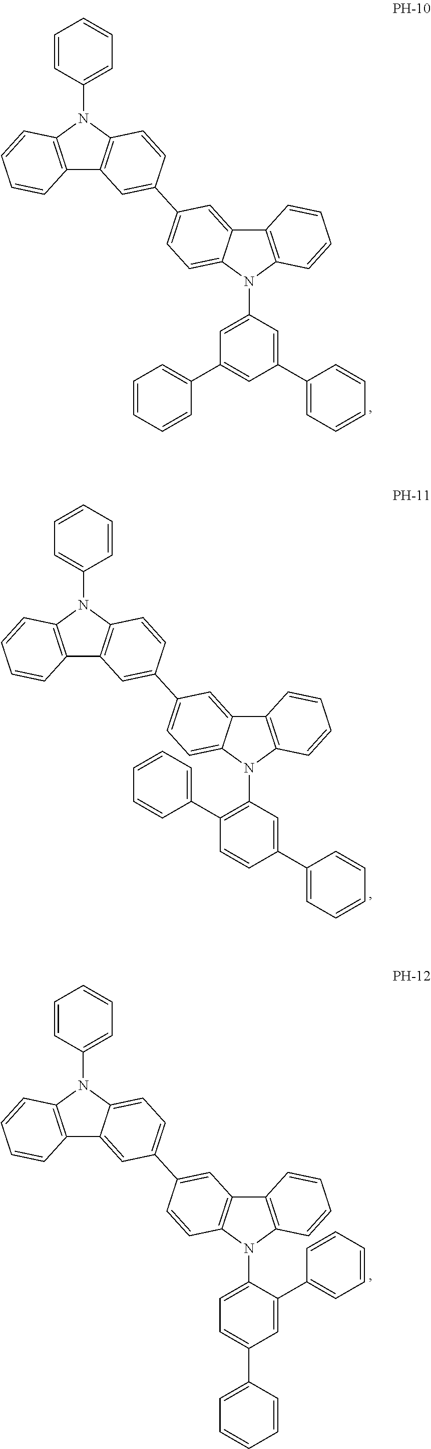 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2ccc(-c3ccccc3)c(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)c(-c3ccccc3)c2)cc1]

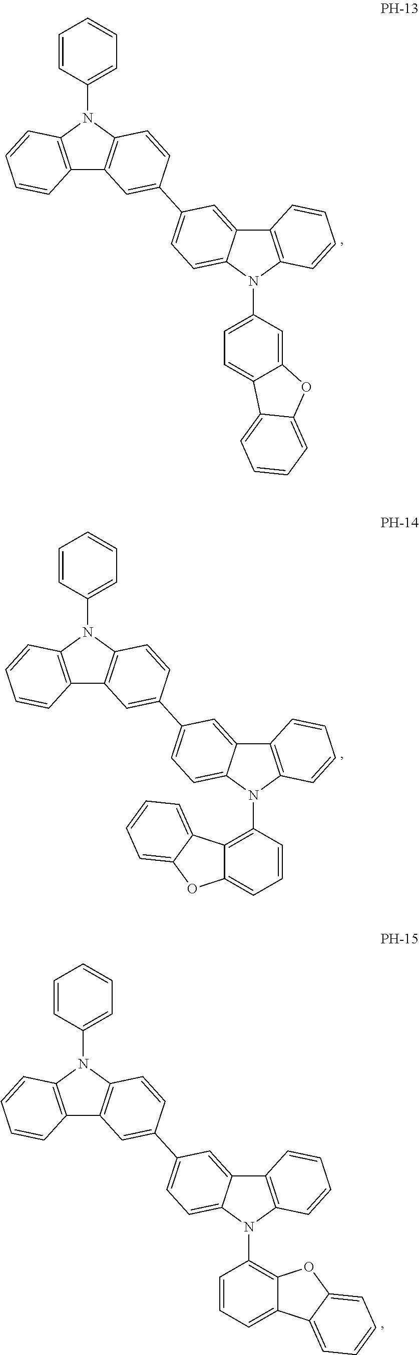 [SMILES: c1ccc(-n2c3ccccc3c3cc(-c4ccc5c(c4)c4ccccc4n5-c4ccc5c(c4)oc4ccccc45)ccc32)cc1 ; c1ccc(-n2c3ccccc3c3cc(-c4ccc5c(c4)c4ccccc4n5-c4cccc5oc6ccccc6c45)ccc32)cc1 ; c1ccc(-n2c3ccccc3c3cc(-c4ccc5c(c4)c4ccccc4n5-c4cccc5c4oc4ccccc45)ccc32)cc1]

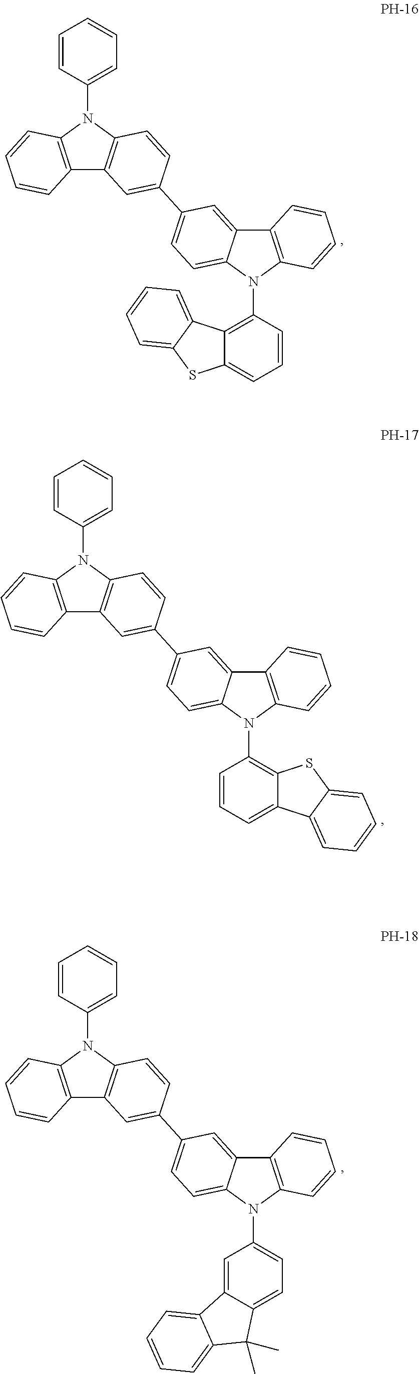 [SMILES: c1ccc(-n2c3ccccc3c3cc(-c4ccc5c(c4)c4ccccc4n5-c4cccc5sc6ccccc6c45)ccc32)cc1 ; c1ccc(-n2c3ccccc3c3cc(-c4ccc5c(c4)c4ccccc4n5-c4cccc5c4sc4ccccc45)ccc32)cc1 ; CC1(C)c2ccccc2-c2cc(-n3c4ccccc4c4cc(-c5ccc6c(c5)c5ccccc5n6-c5ccccc5)ccc43)ccc21]

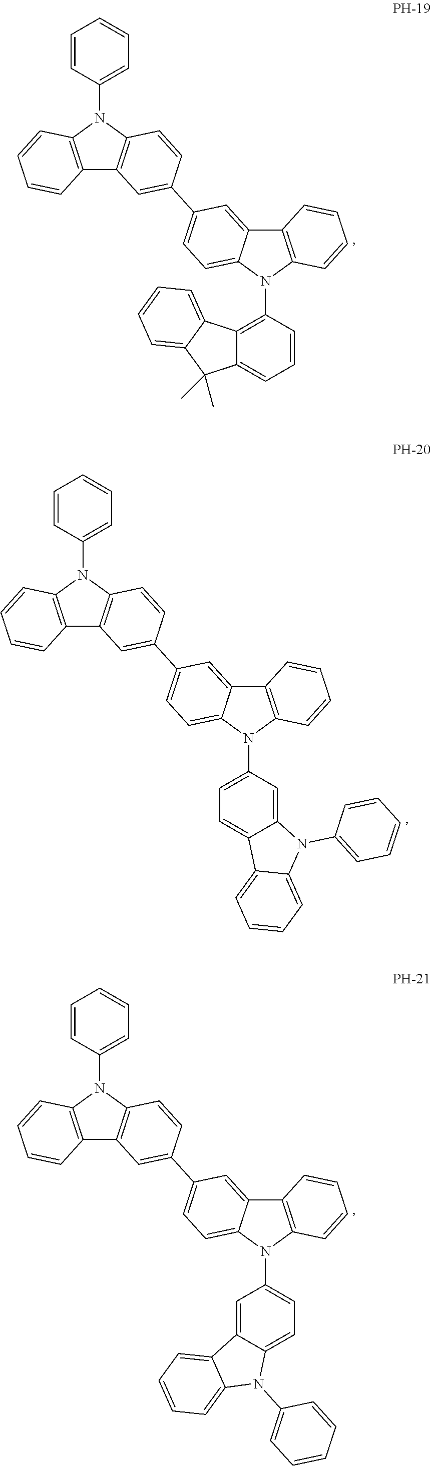 [SMILES: CC1(C)c2ccccc2-c2c(-n3c4ccccc4c4cc(-c5ccc6c(c5)c5ccccc5n6-c5ccccc5)ccc43)cccc21 ; c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-n2c3ccccc3c3cc(-c4ccc5c(c4)c4ccccc4n5-c4ccc5c(c4)c4ccccc4n5-c4ccccc4)ccc32)cc1]

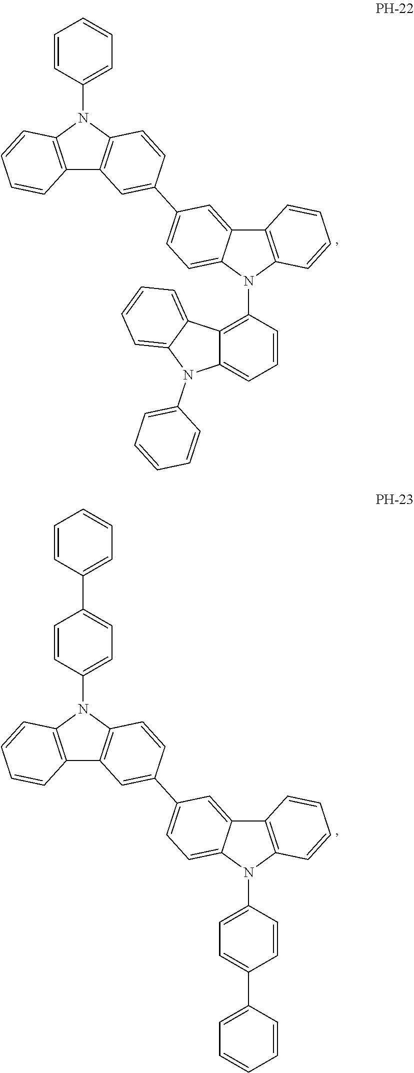 [SMILES: c1ccc(-n2c3ccccc3c3cc(-c4ccc5c(c4)c4ccccc4n5-c4cccc5c4c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

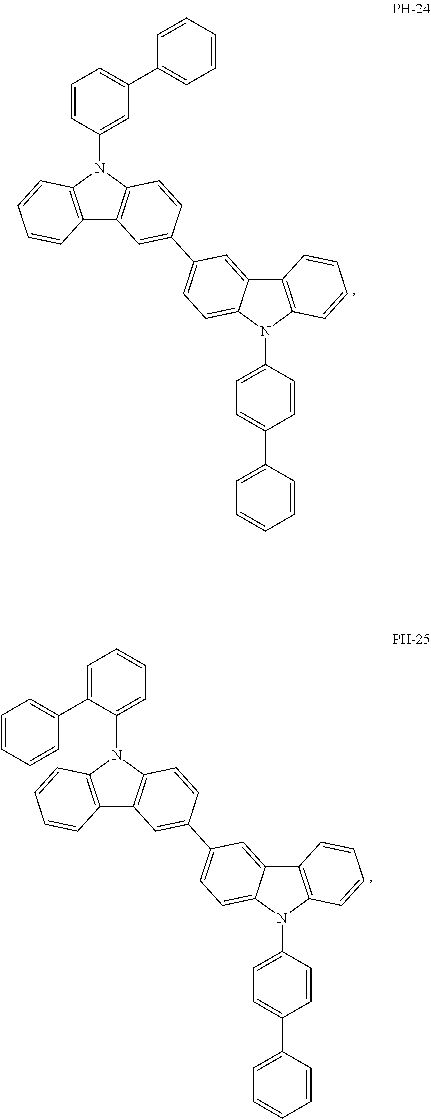 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5-c5ccccc5)ccc43)cc2)cc1]

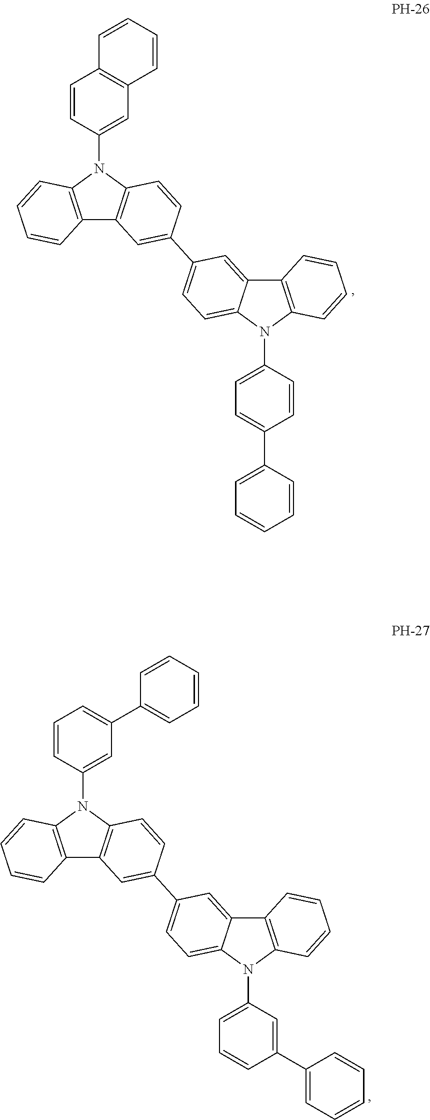 [SMILES: c1ccc(-c2ccc(-n3c4ccccc4c4cc(-c5ccc6c(c5)c5ccccc5n6-c5ccc6ccccc6c5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

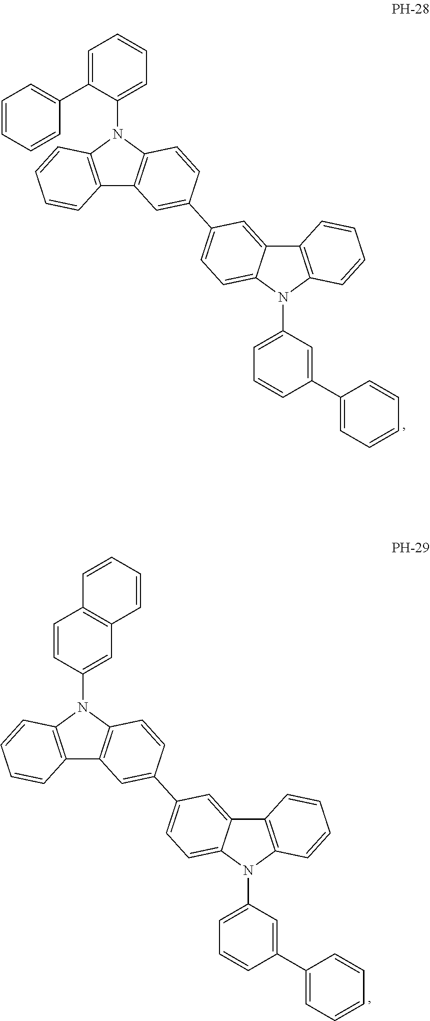 [SMILES: c1ccc(-c2cccc(-n3c4ccccc4c4cc(-c5ccc6c(c5)c5ccccc5n6-c5ccccc5-c5ccccc5)ccc43)c2)cc1 ; c1ccc(-c2cccc(-n3c4ccccc4c4cc(-c5ccc6c(c5)c5ccccc5n6-c5ccc6ccccc6c5)ccc43)c2)cc1]

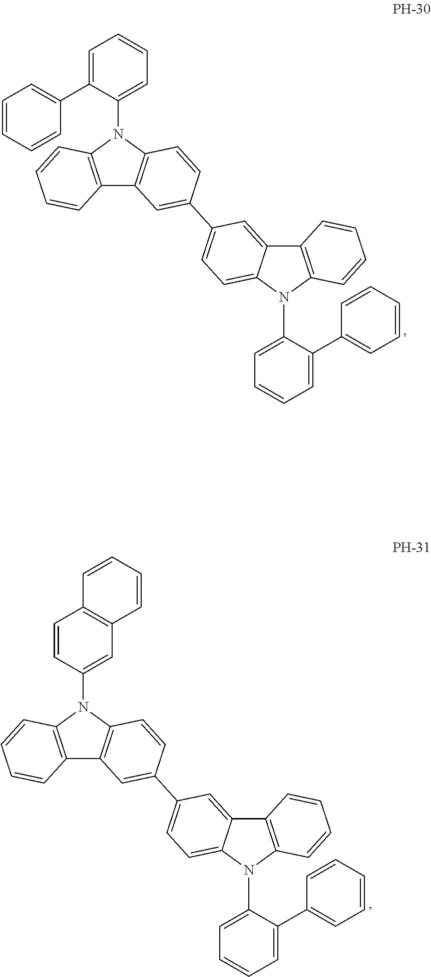 [SMILES: c1ccc(-c2ccccc2-n2c3ccccc3c3cc(-c4ccc5c(c4)c4ccccc4n5-c4ccccc4-c4ccccc4)ccc32)cc1 ; c1ccc(-c2ccccc2-n2c3ccccc3c3cc(-c4ccc5c(c4)c4ccccc4n5-c4ccc5ccccc5c4)ccc32)cc1]

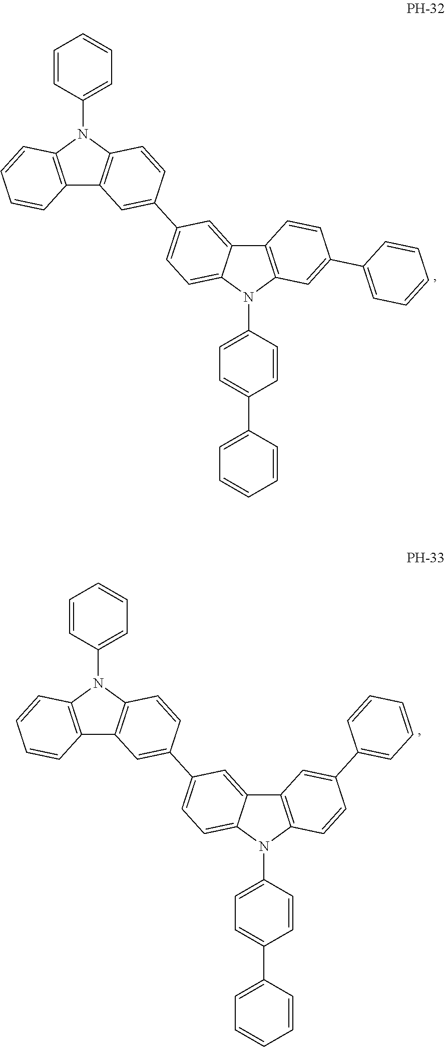 [SMILES: c1ccc(-c2ccc(-n3c4ccc(-c5ccc6c(c5)c5ccccc5n6-c5ccccc5)cc4c4ccc(-c5ccccc5)cc43)cc2)cc1 ; c1ccc(-c2ccc(-n3c4ccc(-c5ccccc5)cc4c4cc(-c5ccc6c(c5)c5ccccc5n6-c5ccccc5)ccc43)cc2)cc1]

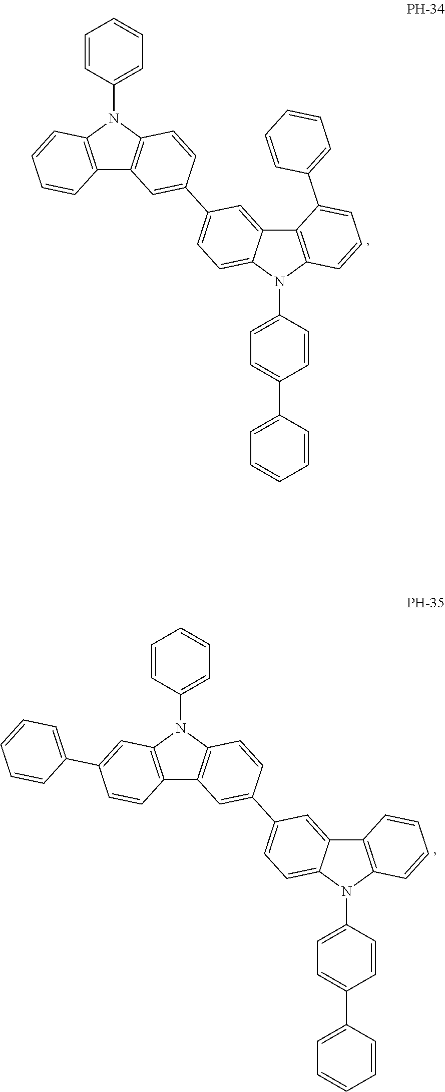 [SMILES: c1ccc(-c2ccc(-n3c4ccc(-c5ccc6c(c5)c5ccccc5n6-c5ccccc5)cc4c4c(-c5ccccc5)cccc43)cc2)cc1 ; c1ccc(-c2ccc(-n3c4ccccc4c4cc(-c5ccc6c(c5)c5ccc(-c7ccccc7)cc5n6-c5ccccc5)ccc43)cc2)cc1]

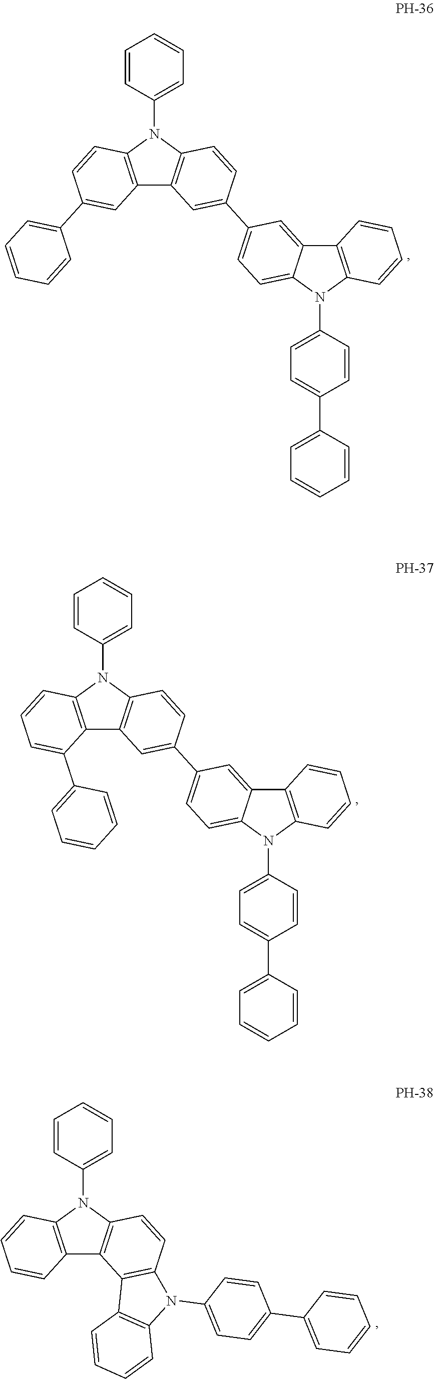 [SMILES: c1ccc(-c2ccc(-n3c4ccccc4c4cc(-c5ccc6c(c5)c5cc(-c7ccccc7)ccc5n6-c5ccccc5)ccc43)cc2)cc1 ; c1ccc(-c2ccc(-n3c4ccccc4c4cc(-c5ccc6c(c5)c5c(-c7ccccc7)cccc5n6-c5ccccc5)ccc43)cc2)cc1 ; c1ccc(-c2ccc(-n3c4ccccc4c4c5c6ccccc6n(-c6ccccc6)c5ccc43)cc2)cc1]

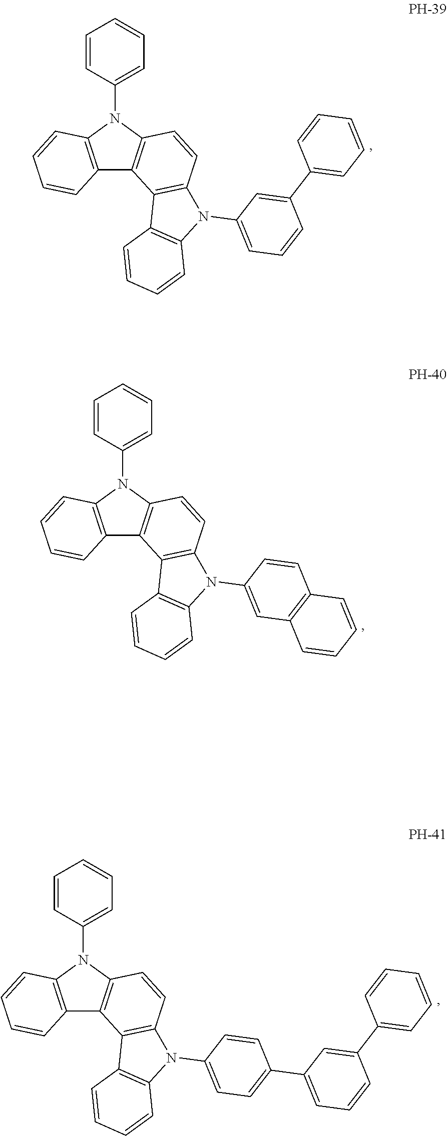 [SMILES: c1ccc(-c2cccc(-n3c4ccccc4c4c5c6ccccc6n(-c6ccccc6)c5ccc43)c2)cc1 ; c1ccc(-n2c3ccccc3c3c4c5ccccc5n(-c5ccc6ccccc6c5)c4ccc32)cc1 ; c1ccc(-c2cccc(-c3ccc(-n4c5ccccc5c5c6c7ccccc7n(-c7ccccc7)c6ccc54)cc3)c2)cc1]

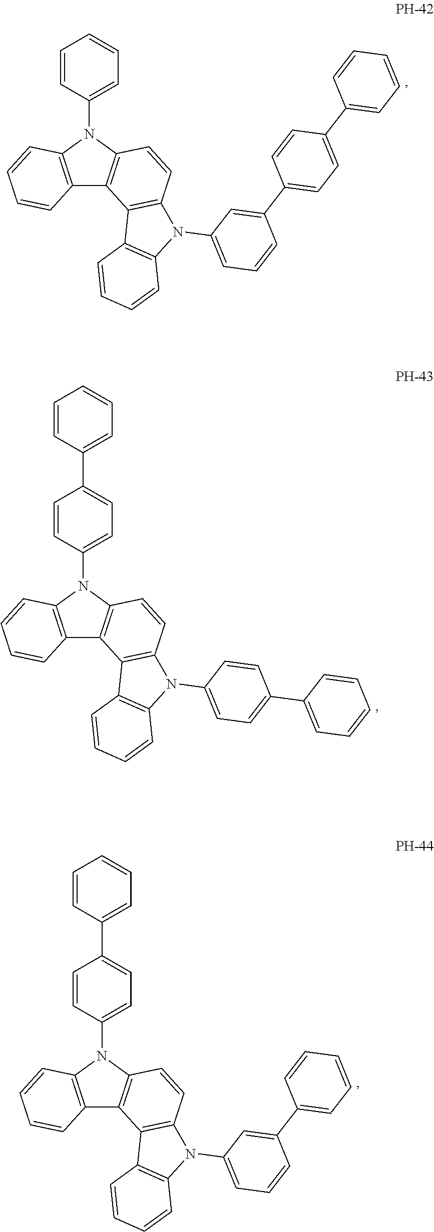 [SMILES: c1ccc(-c2ccc(-c3cccc(-n4c5ccccc5c5c6c7ccccc7n(-c7ccccc7)c6ccc54)c3)cc2)cc1 ; c1ccc(-c2ccc(-n3c4ccccc4c4c5c6ccccc6n(-c6ccc(-c7ccccc7)cc6)c5ccc43)cc2)cc1 ; c1ccc(-c2ccc(-n3c4ccccc4c4c5c6ccccc6n(-c6cccc(-c7ccccc7)c6)c5ccc43)cc2)cc1]

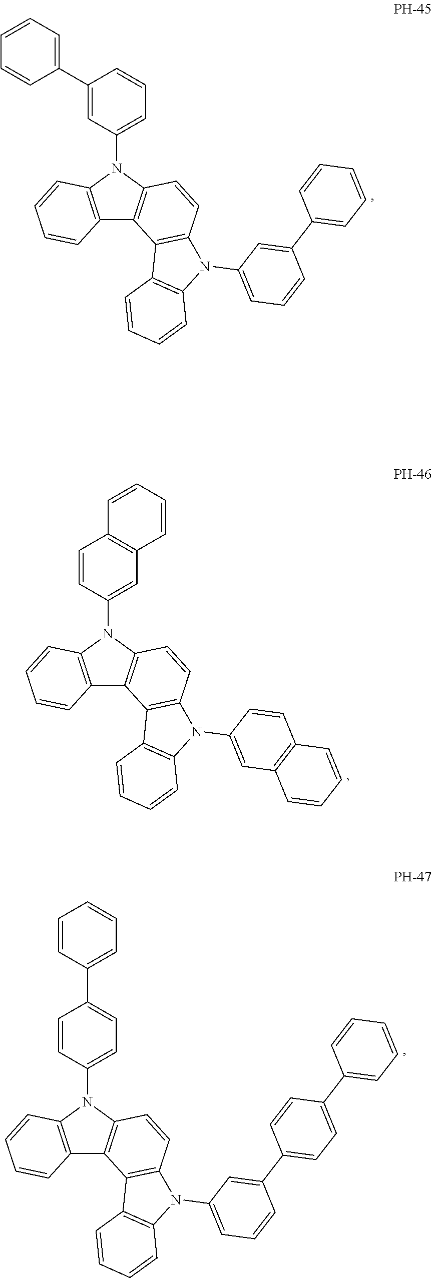 [SMILES: c1ccc(-c2cccc(-n3c4ccccc4c4c5c6ccccc6n(-c6cccc(-c7ccccc7)c6)c5ccc43)c2)cc1 ; c1ccc2cc(-n3c4ccccc4c4c5c6ccccc6n(-c6ccc7ccccc7c6)c5ccc43)ccc2c1 ; c1ccc(-c2ccc(-c3cccc(-n4c5ccccc5c5c6c7ccccc7n(-c7ccc(-c8ccccc8)cc7)c6ccc54)c3)cc2)cc1]

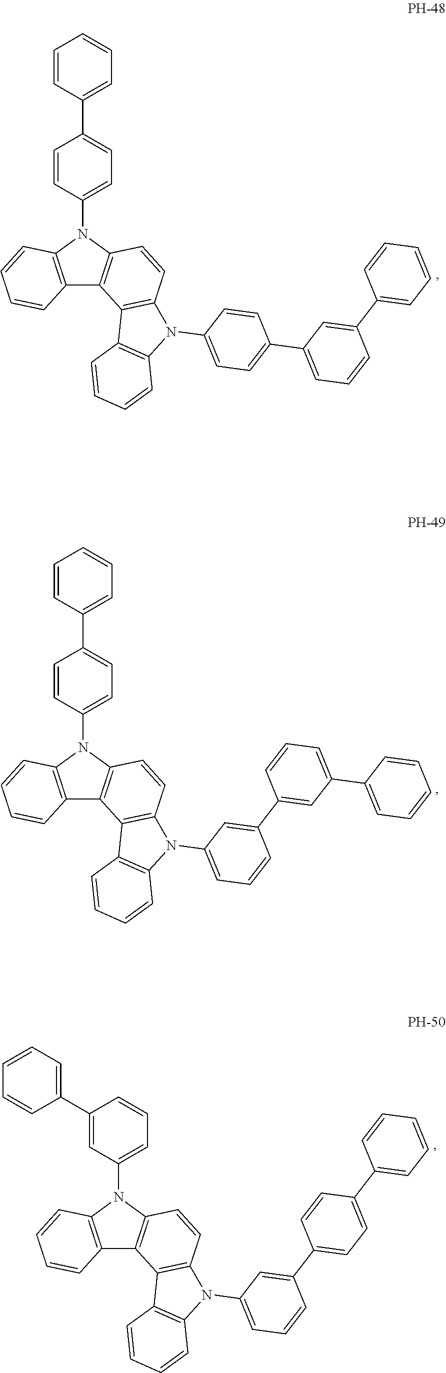 [SMILES: c1ccc(-c2ccc(-n3c4ccccc4c4c5c6ccccc6n(-c6ccc(-c7cccc(-c8ccccc8)c7)cc6)c5ccc43)cc2)cc1 ; c1ccc(-c2ccc(-n3c4ccccc4c4c5c6ccccc6n(-c6cccc(-c7cccc(-c8ccccc8)c7)c6)c5ccc43)cc2)cc1 ; c1ccc(-c2ccc(-c3cccc(-n4c5ccccc5c5c6c7ccccc7n(-c7cccc(-c8ccccc8)c7)c6ccc54)c3)cc2)cc1]

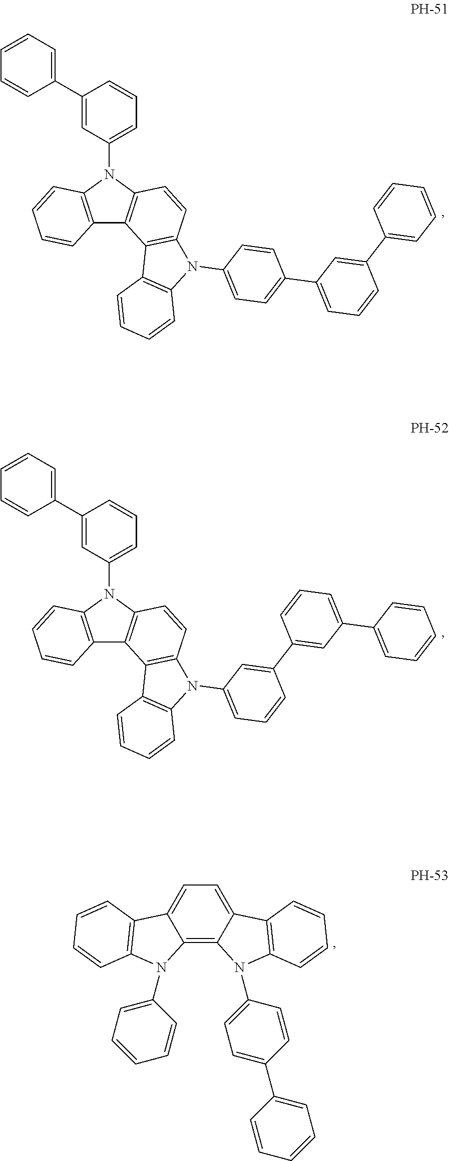 [SMILES: c1ccc(-c2cccc(-c3ccc(-n4c5ccccc5c5c6c7ccccc7n(-c7cccc(-c8ccccc8)c7)c6ccc54)cc3)c2)cc1 ; c1ccc(-c2cccc(-c3cccc(-n4c5ccccc5c5c6c7ccccc7n(-c7cccc(-c8ccccc8)c7)c6ccc54)c3)c2)cc1 ; c1ccc(-c2ccc(-n3c4ccccc4c4ccc5c6ccccc6n(-c6ccccc6)c5c43)cc2)cc1]

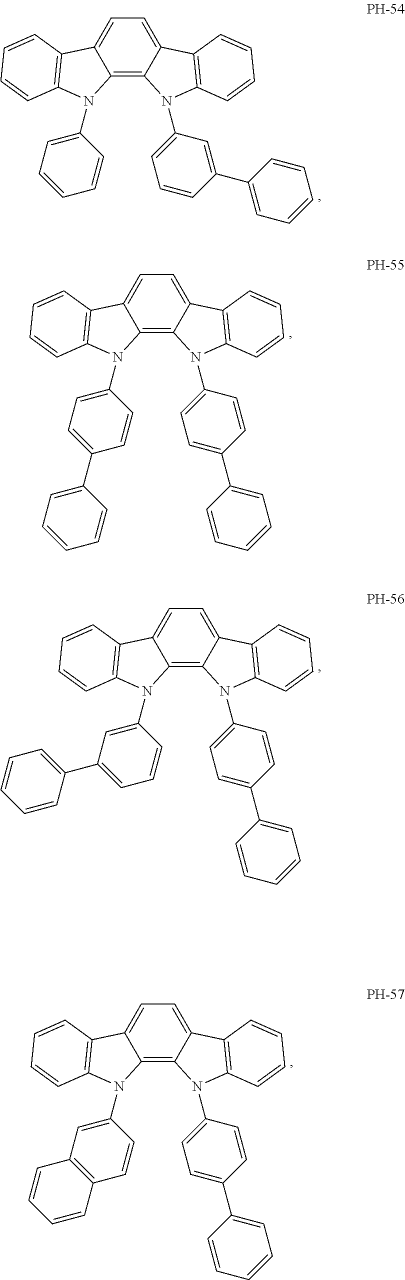 [SMILES: c1ccc(-c2cccc(-n3c4ccccc4c4ccc5c6ccccc6n(-c6ccccc6)c5c43)c2)cc1 ; c1ccc(-c2ccc(-n3c4ccccc4c4ccc5c6ccccc6n(-c6ccc(-c7ccccc7)cc6)c5c43)cc2)cc1 ; c1ccc(-c2ccc(-n3c4ccccc4c4ccc5c6ccccc6n(-c6cccc(-c7ccccc7)c6)c5c43)cc2)cc1 ; c1ccc(-c2ccc(-n3c4ccccc4c4ccc5c6ccccc6n(-c6ccc7ccccc7c6)c5c43)cc2)cc1]

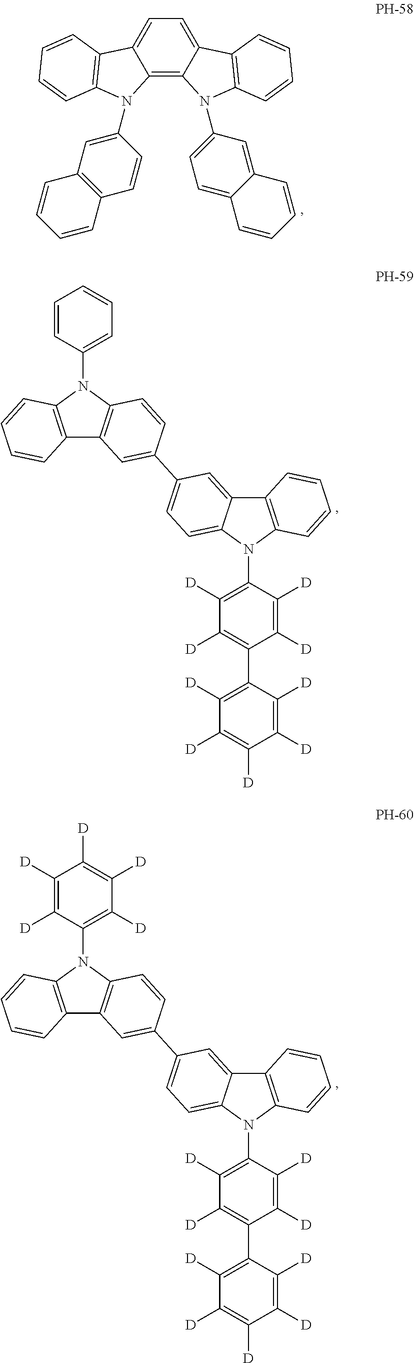 [SMILES: c1ccc2cc(-n3c4ccccc4c4ccc5c6ccccc6n(-c6ccc7ccccc7c6)c5c43)ccc2c1 ; [2H]c1c([2H])c([2H])c(-c2c([2H])c([2H])c(-n3c4ccccc4c4cc(-c5ccc6c(c5)c5ccccc5n6-c5ccccc5)ccc43)c([2H])c2[2H])c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c([2H])c([2H])c5[2H])ccc43)c([2H])c2[2H])c([2H])c1[2H]]

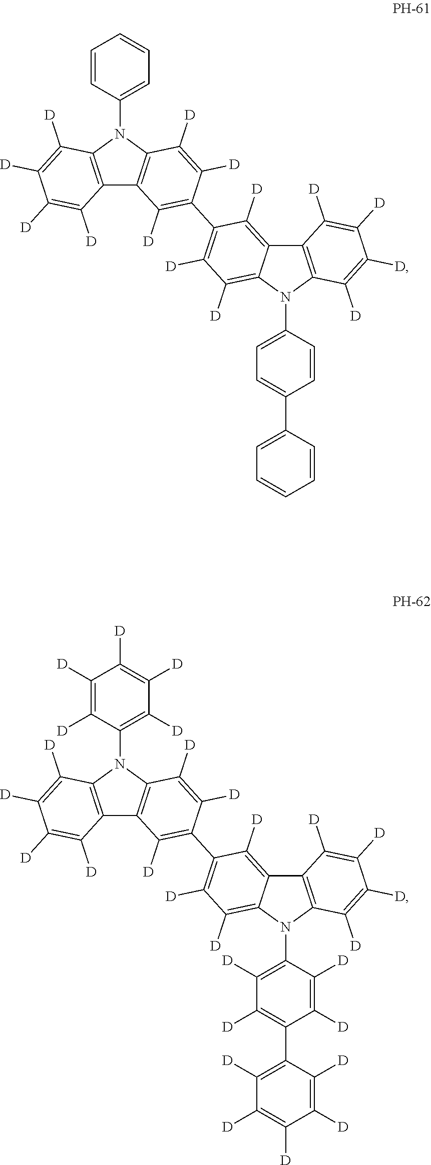

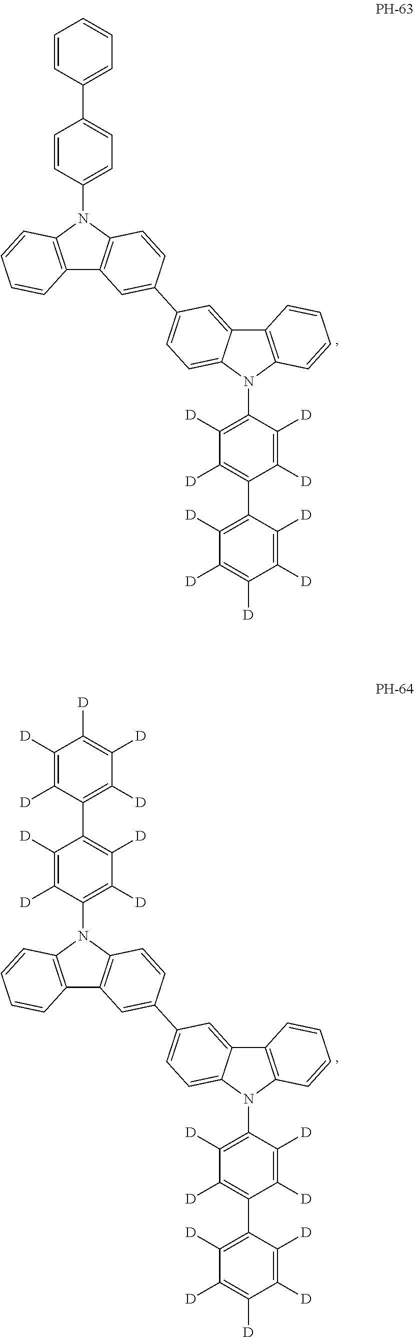 [SMILES: [2H]c1c([2H])c([2H])c(-c2c([2H])c([2H])c(-n3c4ccccc4c4cc(-c5ccc6c(c5)c5ccccc5n6-c5ccc(-c6ccccc6)cc5)ccc43)c([2H])c2[2H])c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c([2H])c6[2H])c([2H])c5[2H])ccc43)c([2H])c2[2H])c([2H])c1[2H]]

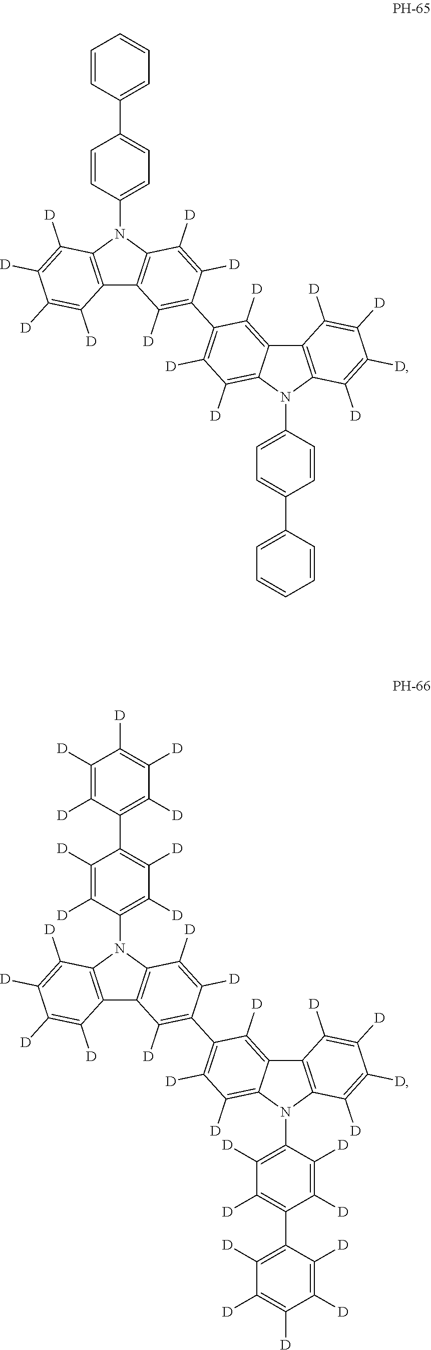

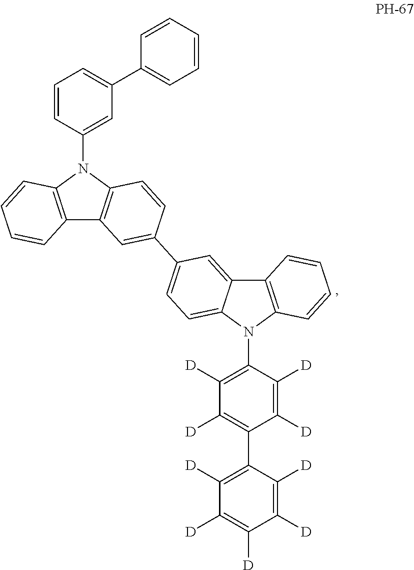 [SMILES: [2H]c1c([2H])c([2H])c(-c2c([2H])c([2H])c(-n3c4ccccc4c4cc(-c5ccc6c(c5)c5ccccc5n6-c5cccc(-c6ccccc6)c5)ccc43)c([2H])c2[2H])c([2H])c1[2H]]

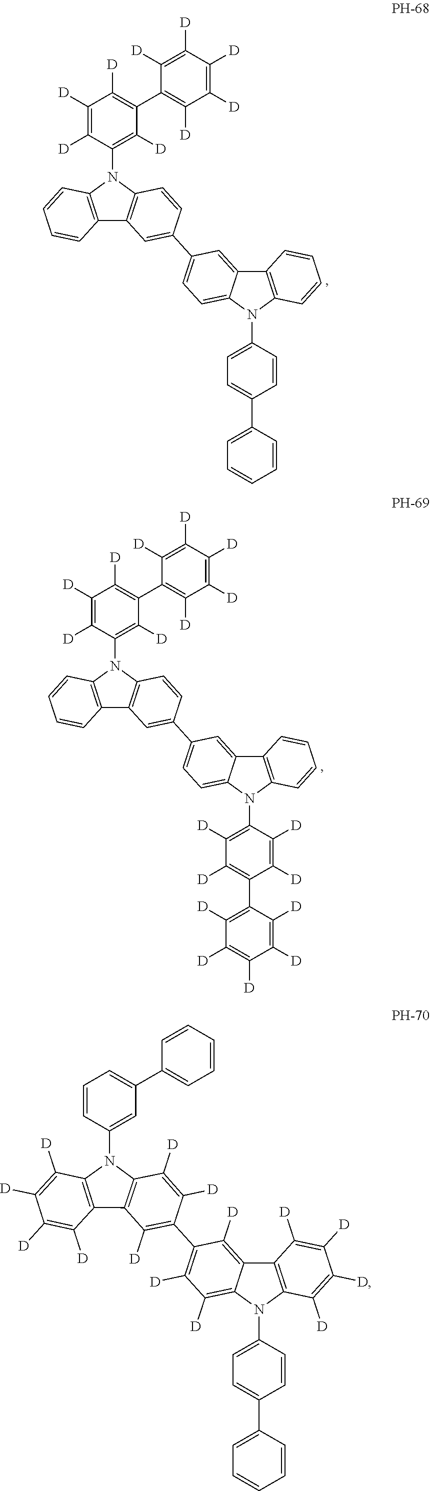

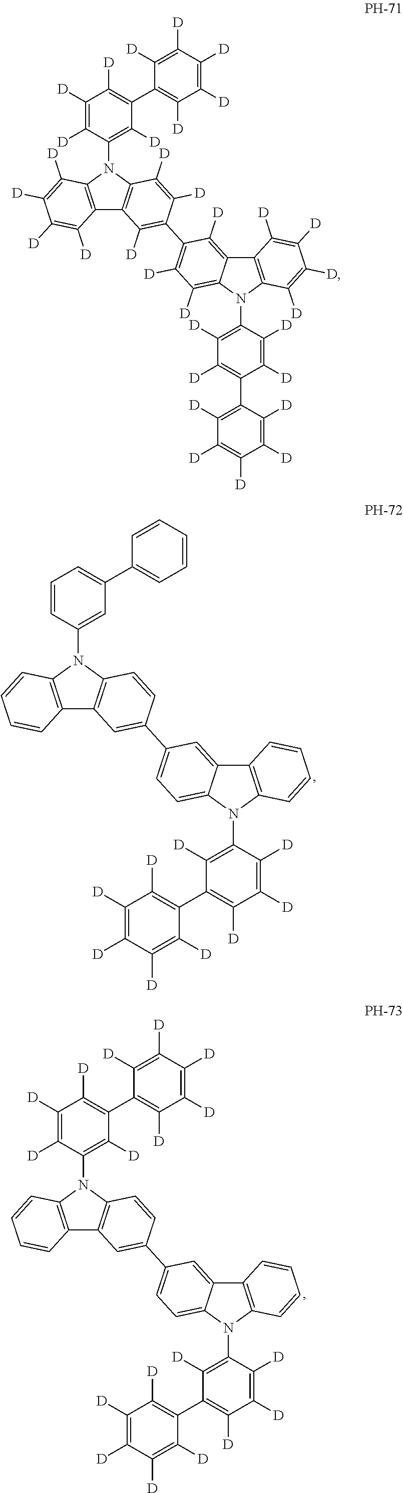

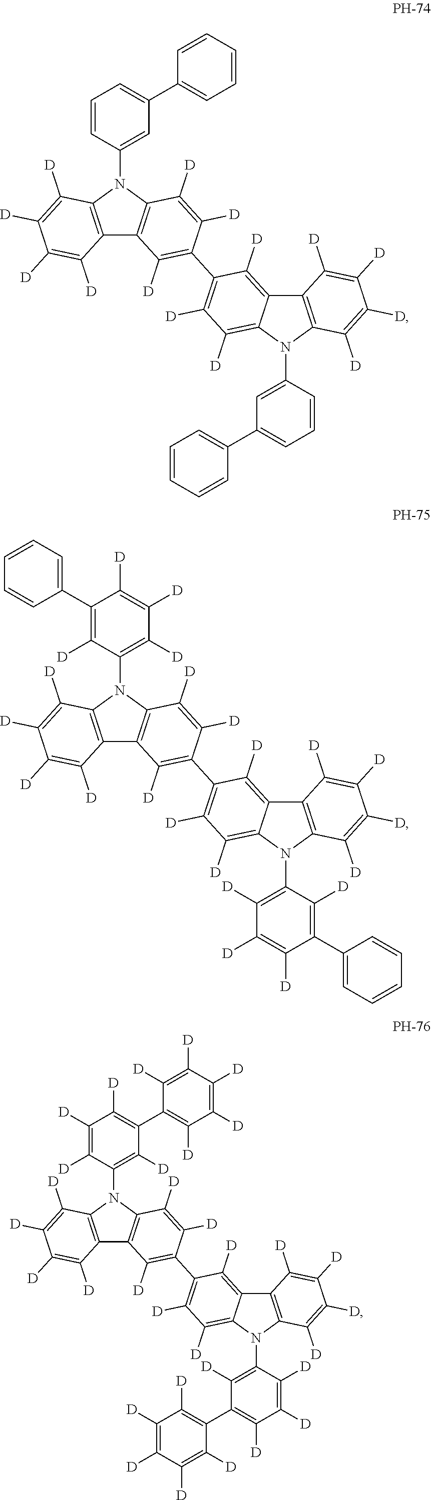

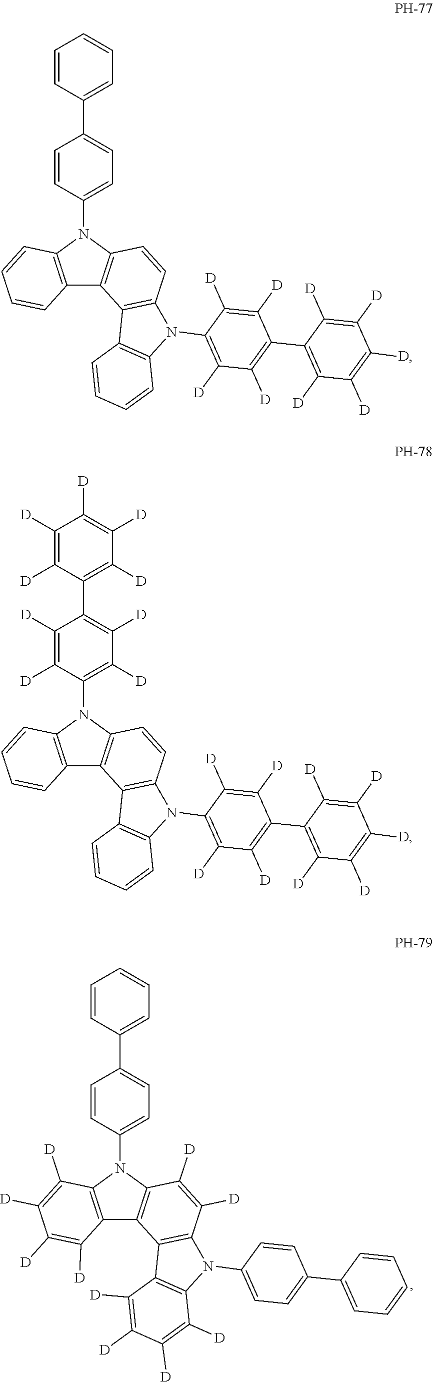

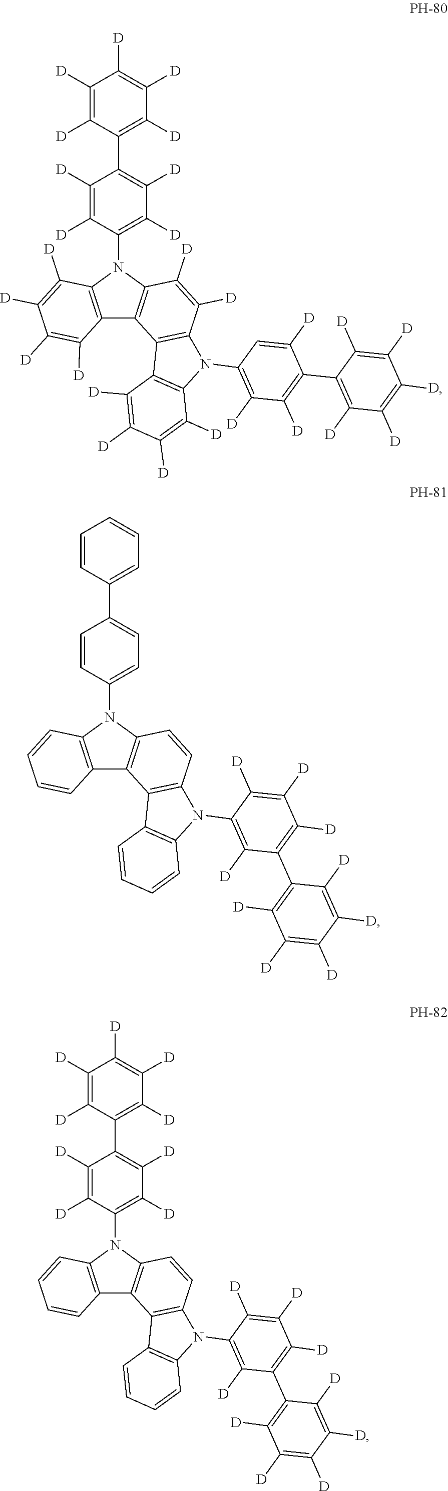

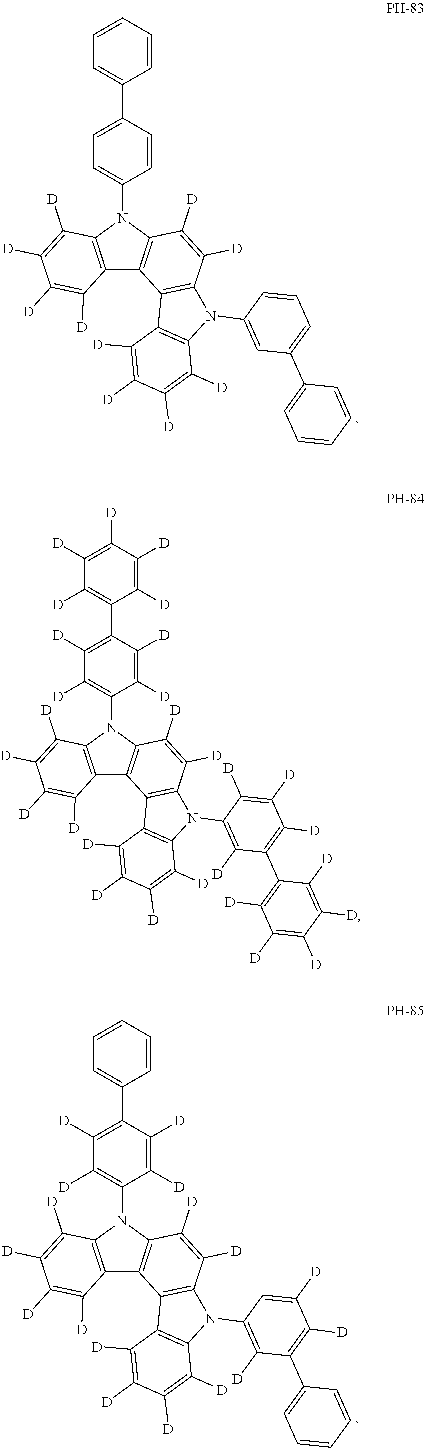

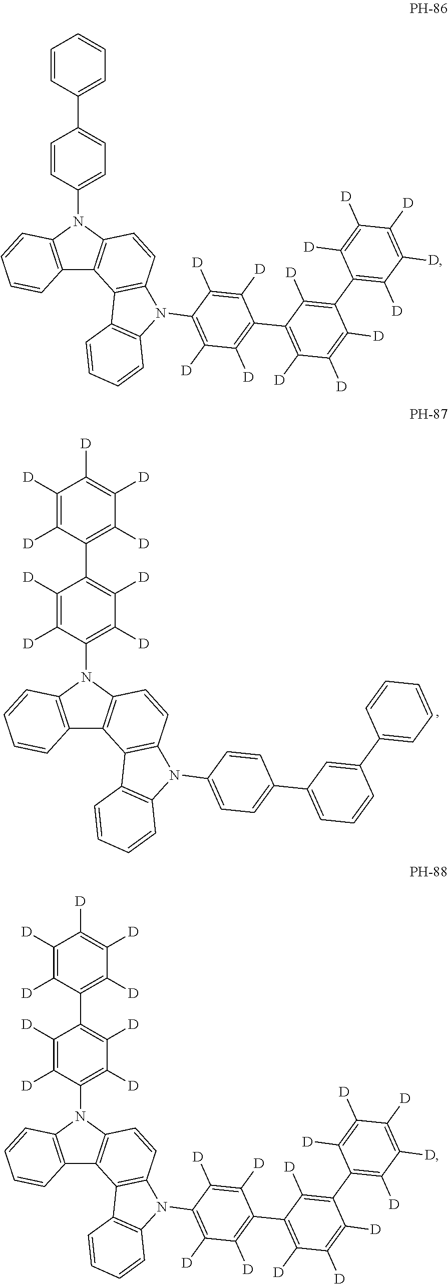

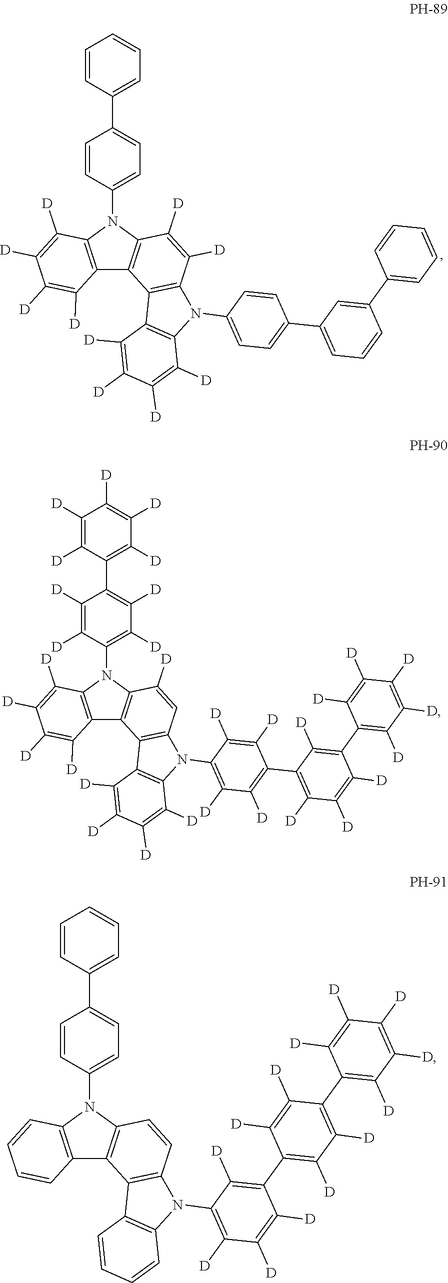

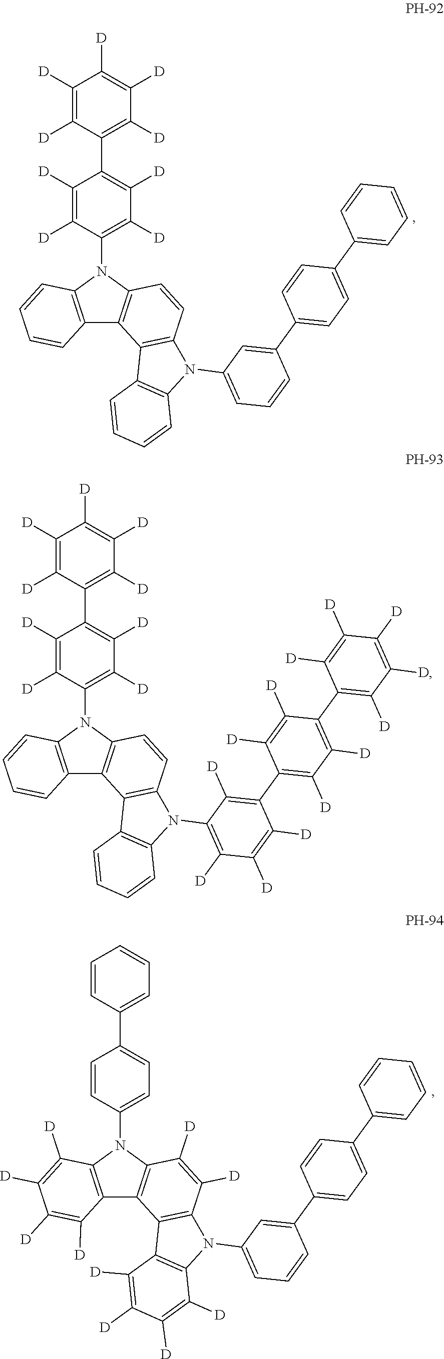

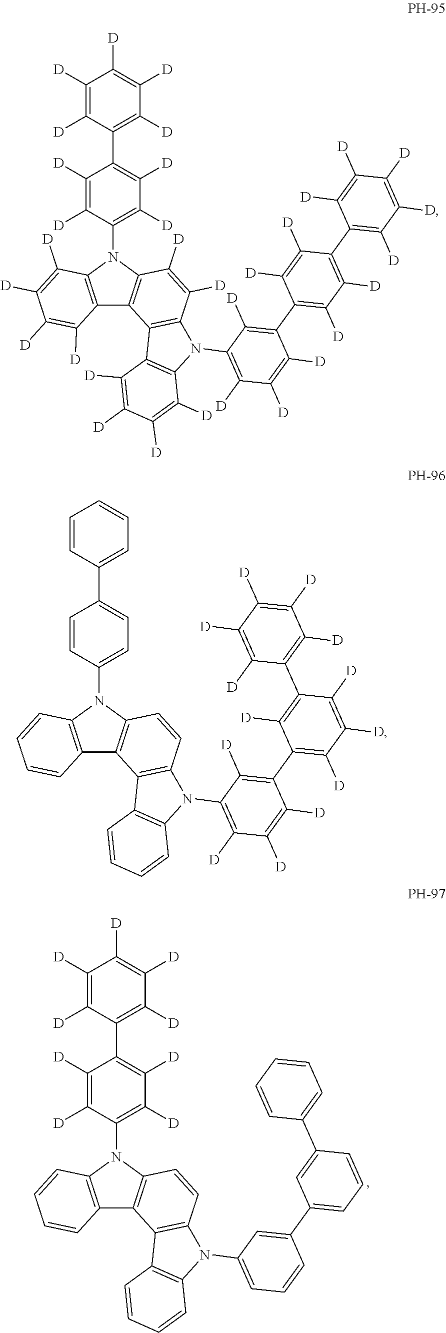

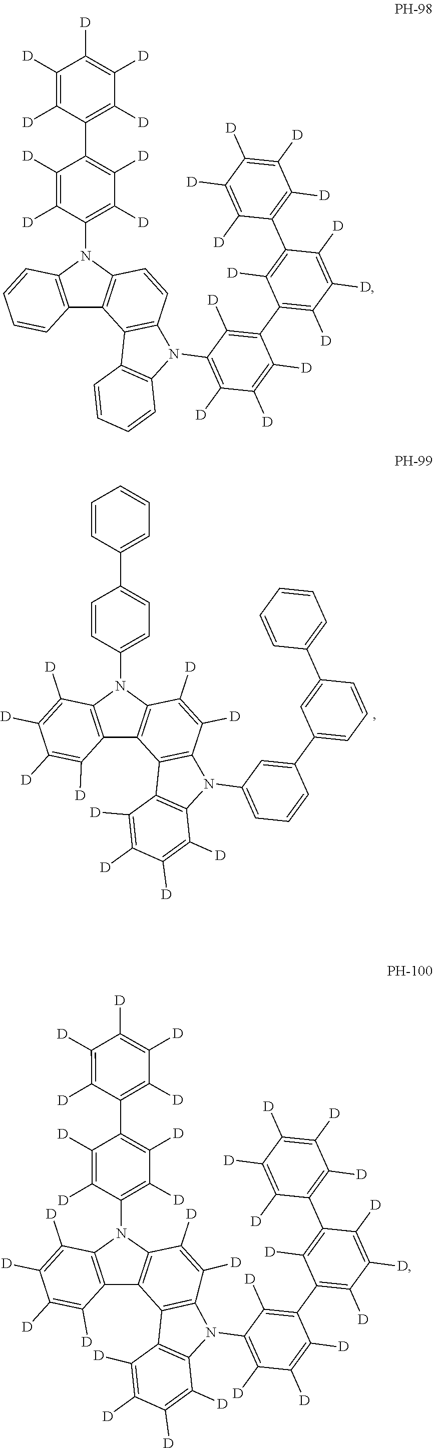

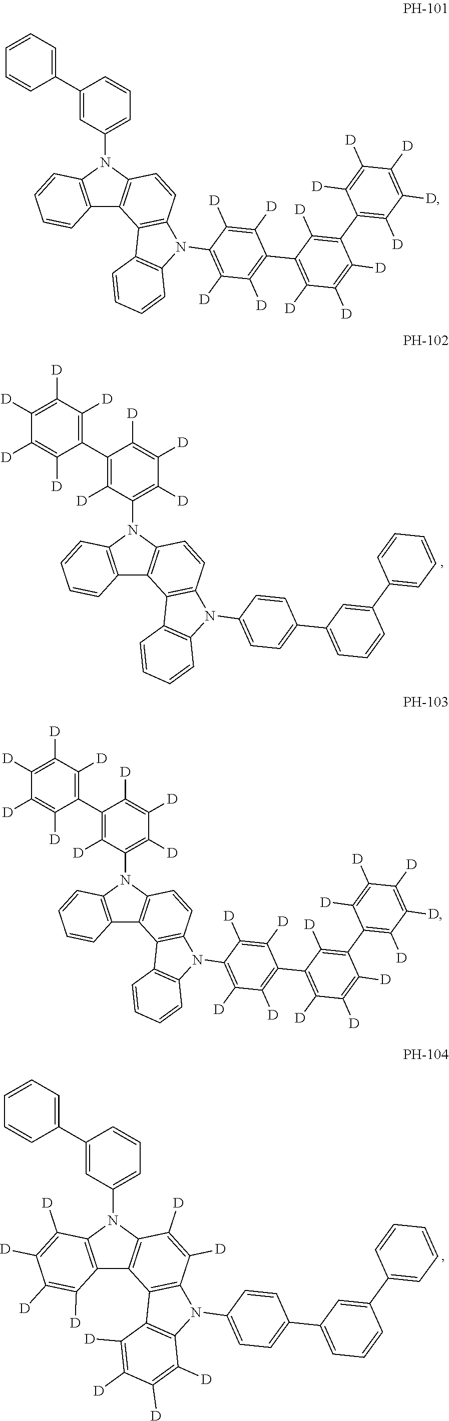

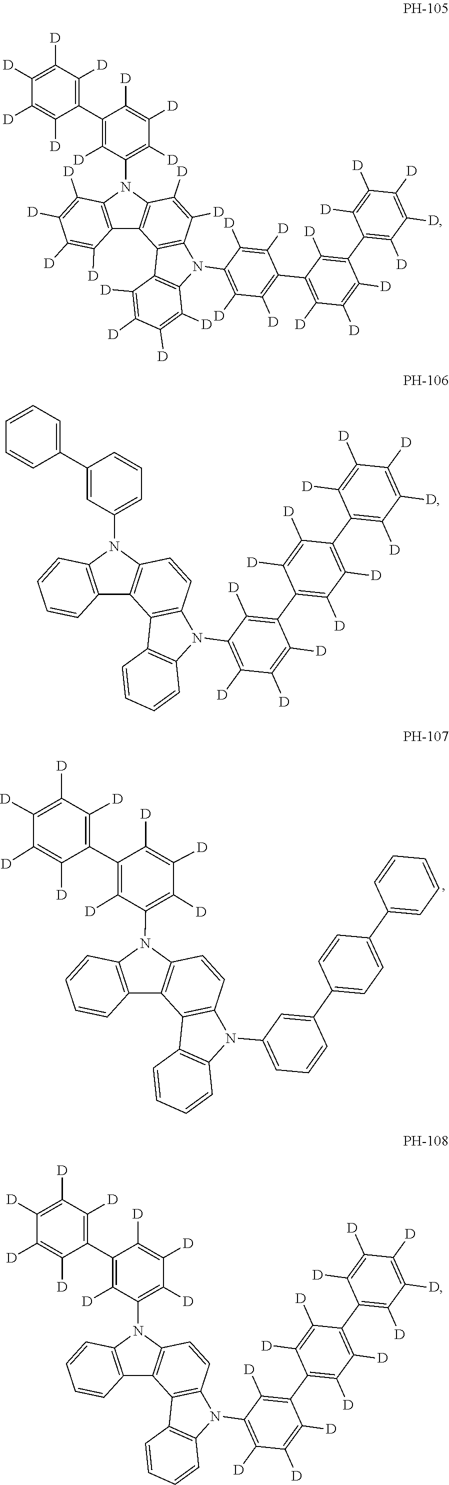

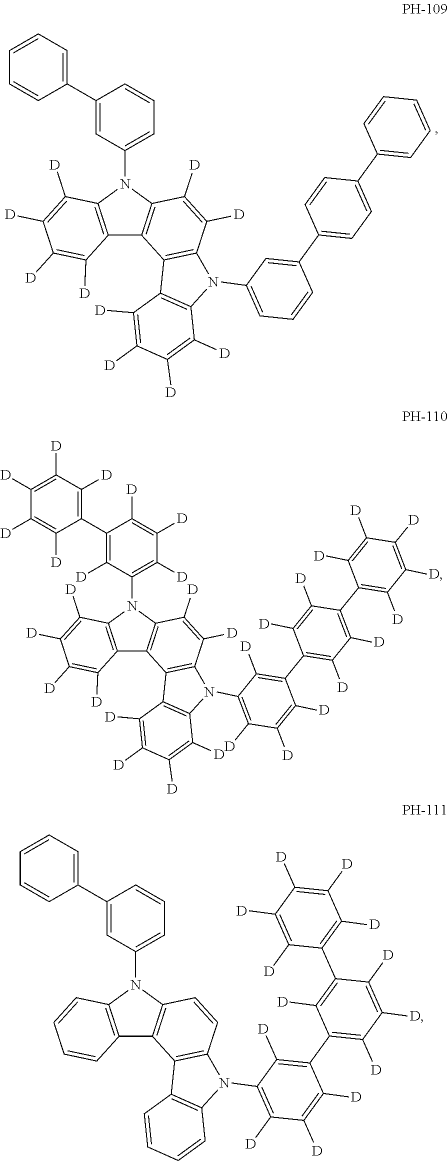

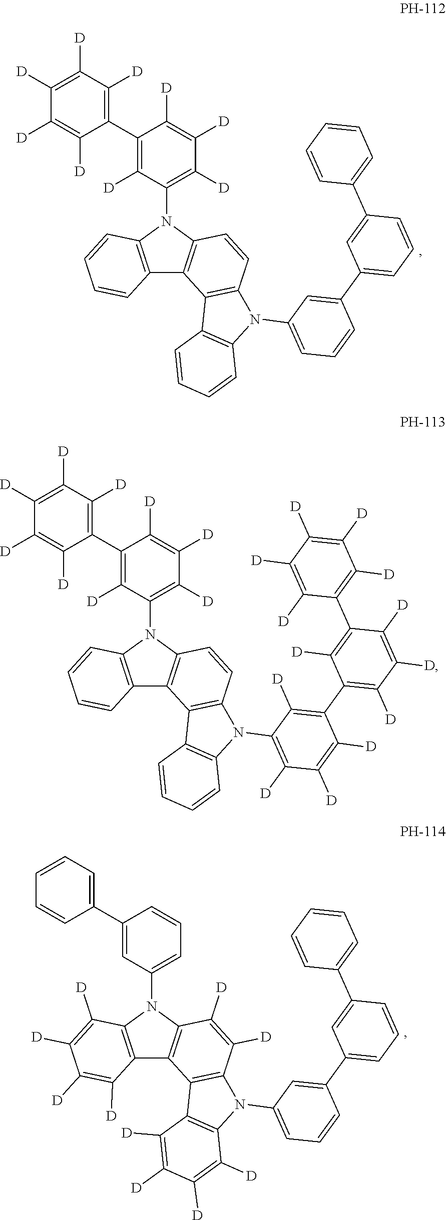

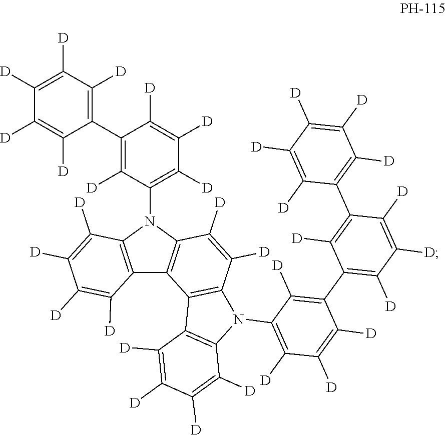

wherein hydrogens in Compound PH-1 to Compound PH-115 can be partially or fully substituted with deuterium.

According to an embodiment of the present disclosure, the organic layer is an emissive layer, and the compound is a host compound; the emissive layer further comprises at least one metal complex, and the at least one metal complex has a general formula of M(Lₐ)ₘ(L_{b})ₙ(L_{c})_{q};
the metal M is selected from a metal with a relative atomic mass greater than 40;
Lₐ, L_{b}, and L_{c} are a first ligand, a second ligand, and a third ligand coordinated to the metal M, respectively; Lₐ, L_{b}, and L_{c} may be the same or different;
Lₐ, L_{b}, and L_{c} can be optionally joined to form a multidentate ligand;
m is 1, 2, or 3, n is 0, 1, or 2, q is 0, 1, or 2, and m+n+q is equal to the oxidation state of the metal M; when m is greater than or equal to 2, multiple La may be the same or different; when n is 2, two L_{b} may be the same or different; when q is 2, two L_{c} may be the same or different;
the ligand Lₐ has a structure represented by Formula 4:

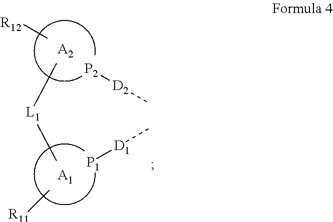

the ring A₁ and the ring A₂ are, at each occurrence identically or differently, selected from a substituted or unsubstituted aromatic ring having 5 to 30 ring atoms, a substituted or unsubstituted heteroaromatic ring having 5 to 30 ring atoms, or a combination thereof;
P₁ and P₂ are, at each occurrence identically or differently, selected from C or N;
D₁ and D₂ are, at each occurrence identically or differently, selected from a single bond, O, or S;
L₁ is selected from the group consisting of: a single bond, BR′, CR′R′, NR′, O, SiR′R′, PR′, S, GeR′R′, Se, substituted or unsubstituted vinylene, ethynylene, substituted or unsubstituted arylene having 6 to 30 carbon atoms, substituted or unsubstituted heteroarylene having 5 to 30 carbon atoms, and combinations thereof; when two R′ are present at the same time, the two R′ are the same or different;
R₁₁ and R₁₂ represent, at each occurrence identically or differently, mono-substitution, multiple substitutions, or non-substitution;
R₁₁, R₁₂, and R′ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R₁₁, R₁₂, R′ can be optionally joined to form a ring; and
the ligands L_{b} and L_{c} are, at each occurrence identically or differently, selected from a monoanionic bidentate ligand.

In this embodiment, the expression that “adjacent substituents R₁₁, R₁₂, R′ can be optionally joined to form a ring” is intended to mean that any one or more of groups of adjacent substituents, such as two substituents R₁₁, two substituents R₁₂, two substituents R′, and substituents R₁₁ and R₁₂, can be joined to form a ring. Obviously, it is also possible that none of these substituents are joined to form a ring.

According to an embodiment of the present disclosure, the ligands L_{b} and L_{c} are, at each occurrence identically or differently, selected from the group consisting of the following structures:

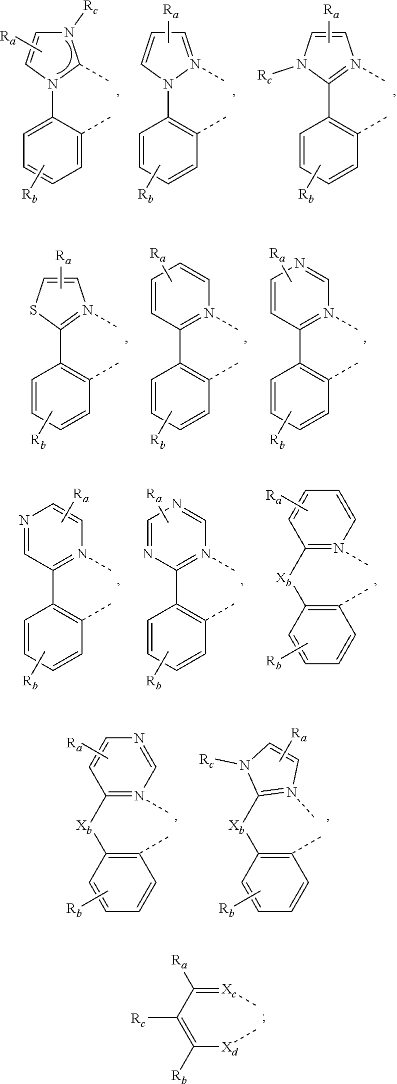

wherein
Rₐ and R_{b} represent, at each occurrence identically or differently, mono-substitution, multiple substitutions, or non-substitution;
X_{b} is, at each occurrence identically or differently, selected from the group consisting of: O, S, Se, NR_{N1}, and CR_{C1}R_{C2};
X_{c} and X_{d} are, at each occurrence identically or differently, selected from the group consisting of: O, S, Se, and NR_{N2};
Rₐ, R_{b}, R_{c}, R_{N1}, R_{N2}, R_{C1}, and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents Rₐ, R_{b}, R_{c}, R_{N1}, R_{N2}, R_{C1}, and R_{C2} can be optionally joined to form a ring.

In this embodiment, the expression that “adjacent substituents Rₐ, R_{b}, RC, R_{N1}, R_{N2}, R_{C1}, and R_{C2} can be optionally joined to form a ring” is intended to mean that any one or more of groups of adjacent substituents, such as two substituents Rₐ, two substituents R_{b}, substituents Rₐ and R_{b}, two substituents R_{c}, substituents Rₐ and R_{c}, substituents R_{b} and R_{c}, substituents Rₐ and R_{N1}, substituents R_{b} and R_{N1}, substituents Rₐ and R_{C1}, substituents Rₐ and R_{C2}, substituents R_{b} and R_{C1}, substituents R_{b} and R_{C2}, substituents Rₐ and R_{N2}, substituents R_{b} and R_{N2}, and substituents R_{C1} and R_{C2}, can be joined to form a ring. For example, adjacent substituents Rₐ, R_{b} in

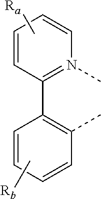

can be optionally joined to form a ring, which can form one or more of the following structures including, but not limited to,

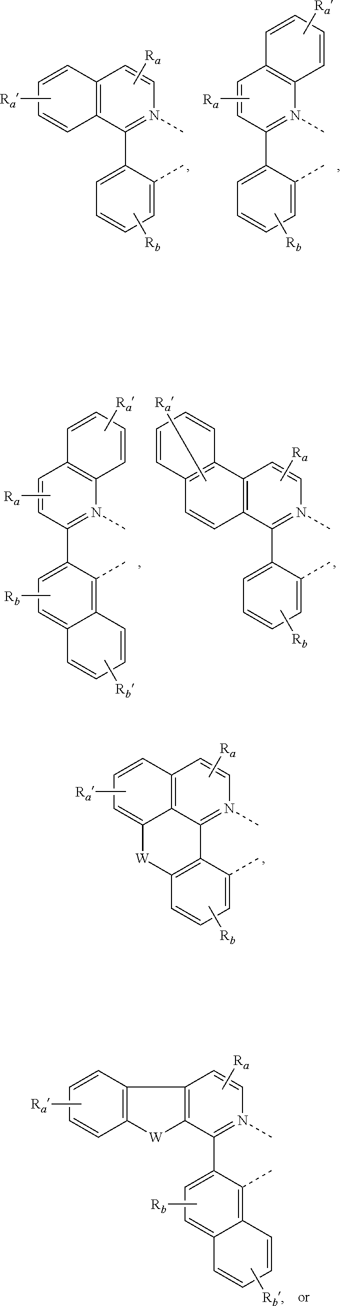

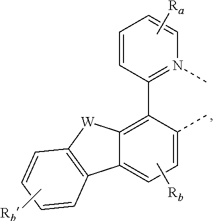

wherein W is selected from O, S, Se, NR′_{w}, or CR′_{w}R′_{w}, and R′_{w}, Rₐ′, and R_{b}′ are defined the same as Rₐ. Obviously, it is also possible that none of these substituents are joined to form a ring.

According to an embodiment of the present disclosure, the metal M is, at each occurrence identically or differently, selected from the group consisting of Cu, Ag, Au, Ru, Rh, Pd, Os, Ir, and Pt.

According to an embodiment of the present disclosure, the metal M is, at each occurrence identically or differently, selected from Pt or Ir.

According to an embodiment of the present disclosure, the metal complex has a general formula of M(Lₐ₁)ⱼ(L_{b1})ₖ, wherein the metal M is selected from a metal with a relative atomic mass greater than 40;
Lₐ₁ and L_{b1} are a first ligand and a second ligand coordinated to M, respectively; Lₐ₁ and L_{b1} can be optionally joined to form a multidentate ligand;
j is 1, 2, or 3, k is 0, 1, or 2, and j+k is equal to the oxidation state of M; when j is greater than or equal to 2, multiple Lₐ₁ may be the same or different; when k is 2, two L_{b1} may be the same or different;
Lₐ₁ has a structure represented by Formula 4-1:

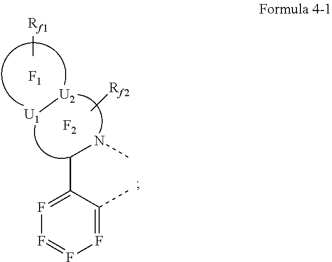

wherein
the ring F₁ is selected from a five-membered unsaturated carbocyclic ring, a benzene ring, a five-membered heteroaromatic ring, or a six-membered heteroaromatic ring;
the ring F₂ is selected from a five-membered heteroaromatic ring or a six-membered heteroaromatic ring;
the ring F₁ and the ring F₂ are fused via U₁ and U₂; U₁ and U₂ are, at each occurrence identically or differently, selected from C or N;
R_{f1} and R_{f2} represent, at each occurrence identically or differently, mono-substitution, multiple substitutions, or non-substitution;
F is, at each occurrence identically or differently, selected from CR_{f} or N;
R_{f1}, R_{f2}, and R_{f} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R_{f1}, R_{f2}, R_{f} can be optionally joined to form a ring;
the ligand L_{b1} has a structure represented by Formula 4-2:

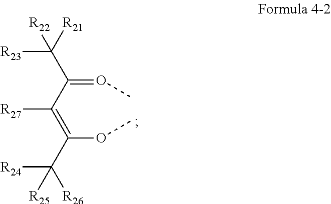

wherein R₂₁ to R₂₇ are each independently selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

In the present disclosure, the expression that “adjacent substituents R_{f1}, R_{f2}, R_{f} can be optionally joined to form a ring” is intended to mean that in the presence of substituents R_{f1}, R_{f2}, and R_{f}, any one or more of groups of adjacent substituents, such as adjacent substituents R_{f1}, adjacent substituents R_{f}, adjacent substituents R_{f}, adjacent substituents R_{f1} and R_{f2}, adjacent substituents R_{f1} and R_{f}, and adjacent substituents R_{f2} and R_{f}, can be joined to form a ring. Obviously, in the presence of substituents R_{f1}, R_{f2}, and R_{f}, it is also possible of none of these groups of substituents are joined to form a ring.

According to an embodiment of the present disclosure, in Formula 4-2, at least one of R₂₁ to R₂₃ is selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, or a combination thereof; and/or at least one of R₂₄ to R₂₆ is selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, or a combination thereof.

According to an embodiment of the present disclosure, in Formula 4-2, at least two of R₂₁ to R₂₃ are selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, or a combination thereof; and/or at least two of R₂₄ to R₂₆ are selected from substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, or a combination thereof.

According to an embodiment of the present disclosure, in Formula 4-2, at least two of R₂₁ to R₂₃ are, at each occurrence identically or differently, selected from substituted or unsubstituted alkyl having 2 to 20 carbon atoms, substituted or unsubstituted cycloalkyl having 3 to 20 ring carbon atoms, substituted or unsubstituted heteroalkyl having 2 to 20 carbon atoms, or a combination thereof; and/or at least two of R₂₄ to R₂₆ are, at each occurrence identically or differently, selected from substituted or unsubstituted alkyl having 2 to 20 carbon atoms, substituted or unsubstituted cycloalkyl having 3 to 20 ring carbon atoms, substituted or unsubstituted heteroalkyl having 2 to 20 carbon atoms, or a combination thereof.

According to an embodiment of the present disclosure, the metal complex has a general formula of Ir(Lₐ₁)₂(L_{b1}).

According to an embodiment of the present disclosure, the metal complex is an Ir complex and comprises a ligand Lₐ₁, and the Lₐ₁ has a structure represented by Formula 4-1 and comprises at least one structural unit selected from the group consisting of an aromatic ring formed by fusing a six-membered ring to a six-membered ring, a heteroaromatic ring formed by fusing a six-membered ring to a six-membered ring, an aromatic ring formed by fusing a six-membered ring to a five-membered ring, and a heteroaromatic ring formed by fusing a six-membered ring to a five-membered ring.

According to an embodiment of the present disclosure, the metal complex is an Ir complex and comprises a ligand Lₐ₁, and the Lₐ₁ has a structure represented by Formula 4-1 and comprises at least one structural unit selected from the group consisting of naphthalene, phenanthrene, quinoline, isoquinoline, and azaphenanthrene.

According to an embodiment of the present disclosure, the metal complex has a structure represented by Formula 4-3:

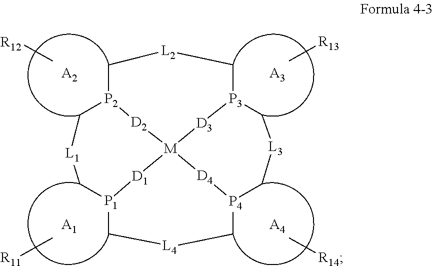

wherein
the metal M is, at each occurrence identically or differently, selected from a metal with a relative molecular mass greater than 40;
the rings A₁ to A₄ are, at each occurrence identically or differently, selected from a substituted or unsubstituted aromatic ring having 6 to 30 ring atoms, a substituted or unsubstituted heteroaromatic ring having 5 to 30 ring atoms, or a combination thereof;
L₁ to L₄ are, at each occurrence identically or differently, selected from the group consisting of: a single bond, BR′, CR′R′, NR′, O, SiR′R′, PR′, S, GeR′R′, Se, substituted or unsubstituted vinylene, ethynylene, substituted or unsubstituted arylene having 6 to 30 carbon atoms, substituted or unsubstituted heteroarylene having 5 to 30 carbon atoms, and combinations thereof; when two R′ are present at the same time, the two R′ are the same or different;
P₁ to P₄ are, at each occurrence identically or differently, selected from C or N;
D₁ to D₄ are, at each occurrence identically or differently, selected from a single bond, O, or S;
R₁₁ to R₁₄ represent, at each occurrence identically or differently, mono-substitution, multiple substitutions, or non-substitution;
R₁₁ to R₁₄ and R′ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents R₁₁ to R₁₄, R′ can be optionally joined to form a ring.

In this embodiment, the expression that “adjacent substituents R₁₁ to R₁₄, R′ can be optionally joined to form a ring” is intended to mean that any one or more of groups of adjacent substituents, such as adjacent substituents R₁₁, adjacent substituents R₁₂, adjacent substituents R₁₃, adjacent substituents R₁₄, and adjacent substituents R′, can be joined to form a ring. Obviously, it is also possible that none of these substituents are joined to form a ring.

According to an embodiment of the present disclosure, the metal complex has a general formula of Ir(Lₐ)ₘ(L_{b})₃₋ₘ and a structure represented by Formula 4-4:

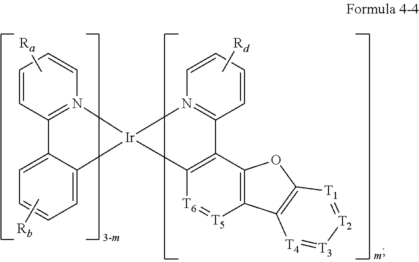

wherein
m is 0, 1, 2, or 3; when m is 2 or 3, multiple Lₐ are the same or different; when m is 0 or 1, multiple L_{b} are the same or different;
T₁ to T₆ are, at each occurrence identically or differently, selected from CR₁ or N;
Rₐ, R_{b}, and R_{d} represent, at each occurrence identically or differently, mono-substitution, multiple substitutions, or non-substitution;
Rₐ, R_{b}, R_{d}, and R₁ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents Rₐ, R_{b} can be optionally joined to form a ring; and
adjacent substituents R_{d}, R₁ can be optionally joined to form a ring.

In this embodiment, the expression that “adjacent substituents Rₐ, R_{b} can be optionally joined to form a ring” is intended to mean that any one or more of groups of adjacent substituents, such as two adjacent substituents Rₐ, two adjacent substituents R_{b}, and adjacent substituents Rₐ and R_{b}, can be joined to form a ring. Obviously, it is also possible that none of these substituents are joined to form a ring.

In this embodiment, the expression that “adjacent substituents R_{d}, R_{T} can be optionally joined to form a ring” is intended to mean that any one or more of groups of adjacent substituents, such as two adjacent substituents R_{T} and two adjacent substituents R_{d}, can be joined to form a ring. Obviously, it is also possible that none of these substituents are joined to form a ring.

According to an embodiment of the present disclosure, at least one of T₁ to T₆ is selected from CR₁, and the R₁ is selected from substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, or substituted or unsubstituted heteroaryl having 3 to 30 carbon atoms.

According to an embodiment of the present disclosure, at least one of T₁ to T₆ is selected from CR₁, and the R₁ is fluorine or cyano.

According to an embodiment of the present disclosure, at least one of T₁ to T₄ is selected from CR₁, and the R₁ is fluorine or cyano.

According to an embodiment of the present disclosure, at least two of T₁ to T₆ are selected from CR₁, one R_{T} is fluorine or cyano, and another R_{T} is selected from substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, or substituted or unsubstituted heteroaryl having 3 to 30 carbon atoms.

According to an embodiment of the present disclosure, T₁ to T₆ are, at each occurrence identically or differently, selected from CR₁ or N, and at least one of T₁ to T₆ is selected from N, for example, one of T₁ to T₆ is selected from N or two of T₁ to T₆ are selected from N.

According to an embodiment of the present disclosure, the metal complex is selected from the group consisting of Compound GD1 to Compound GD77:

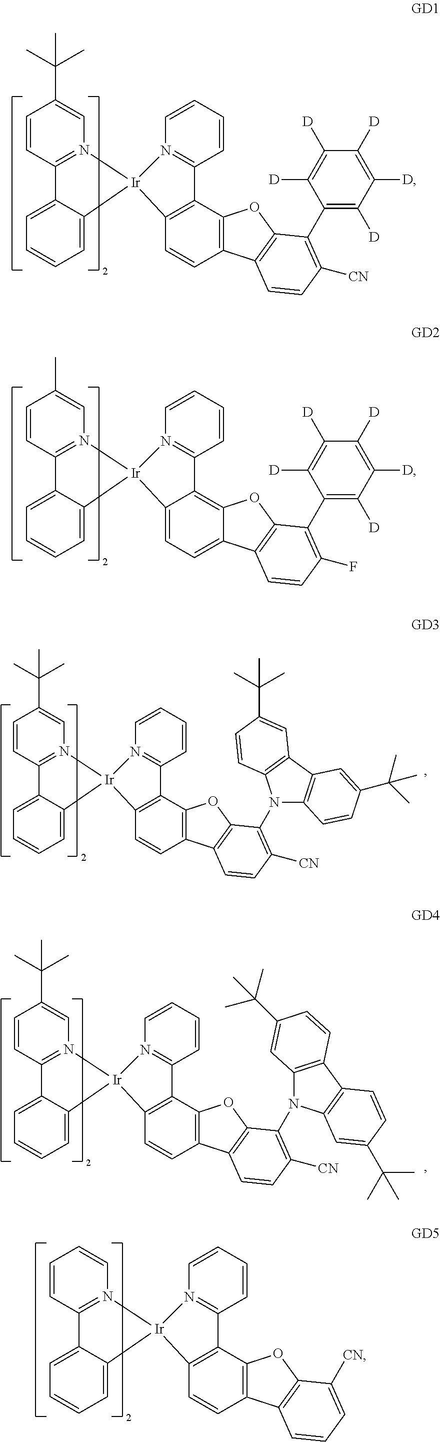

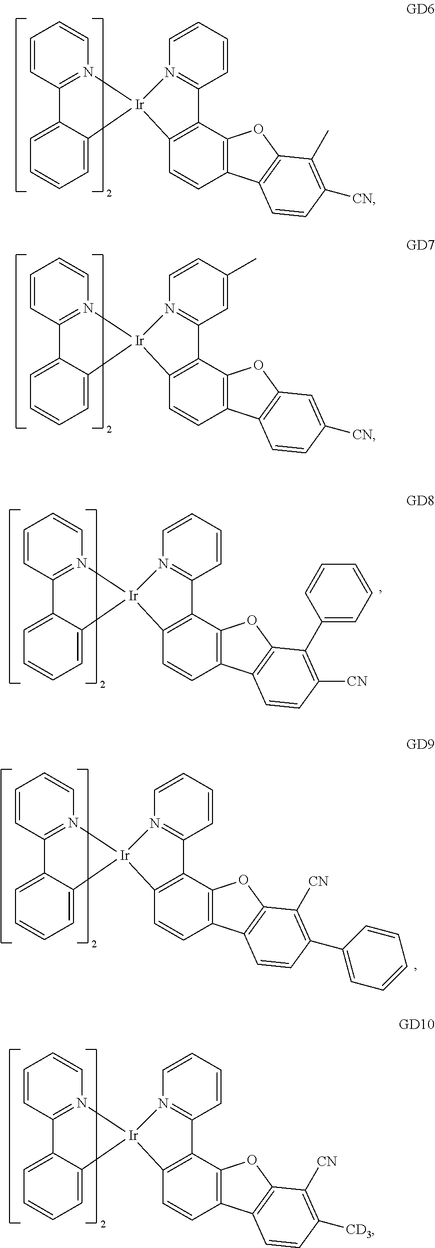

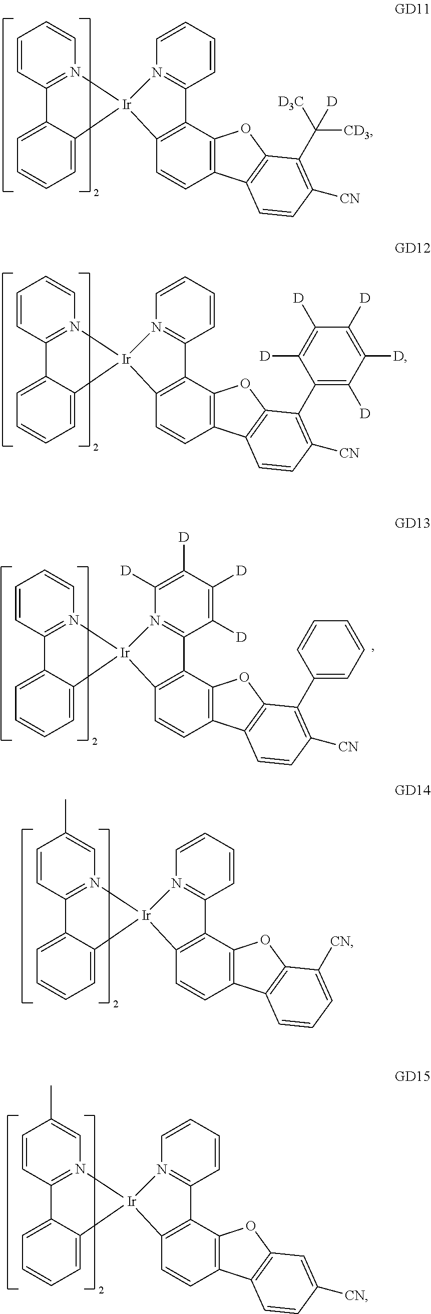

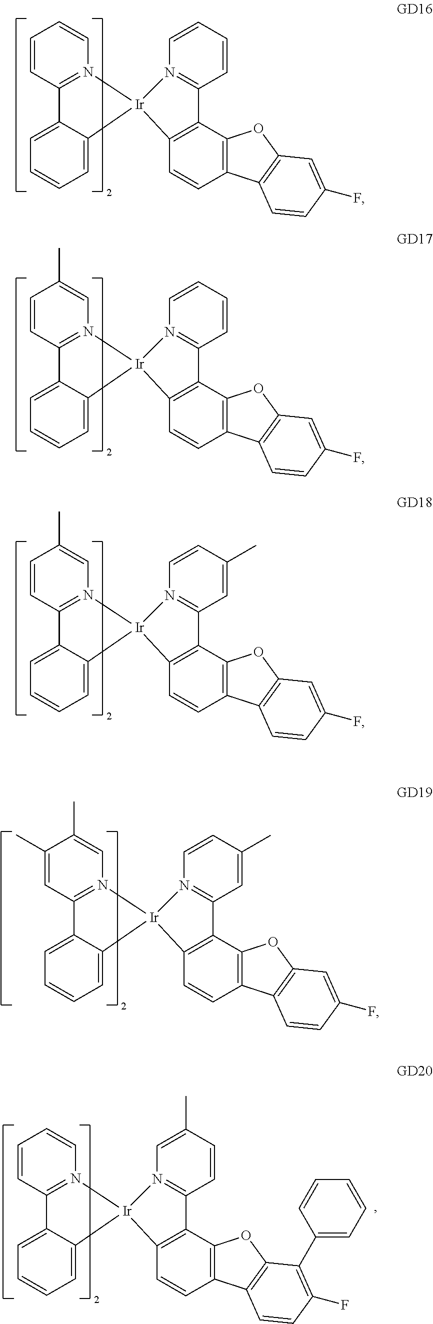

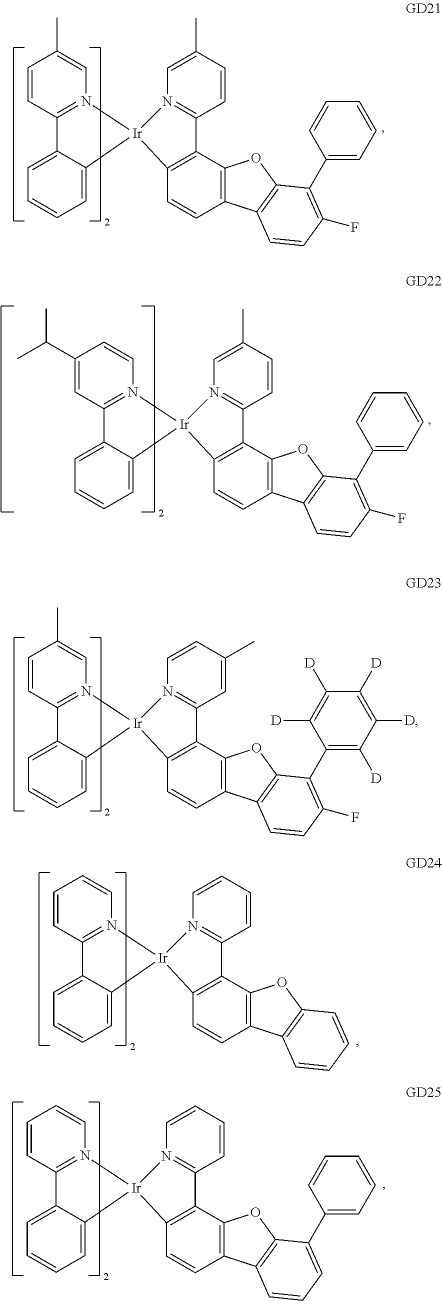

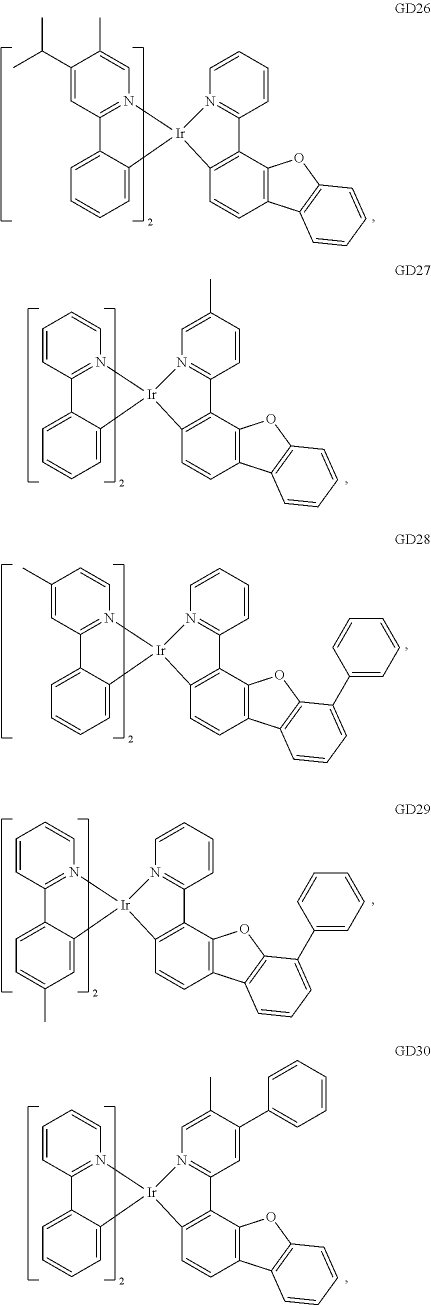

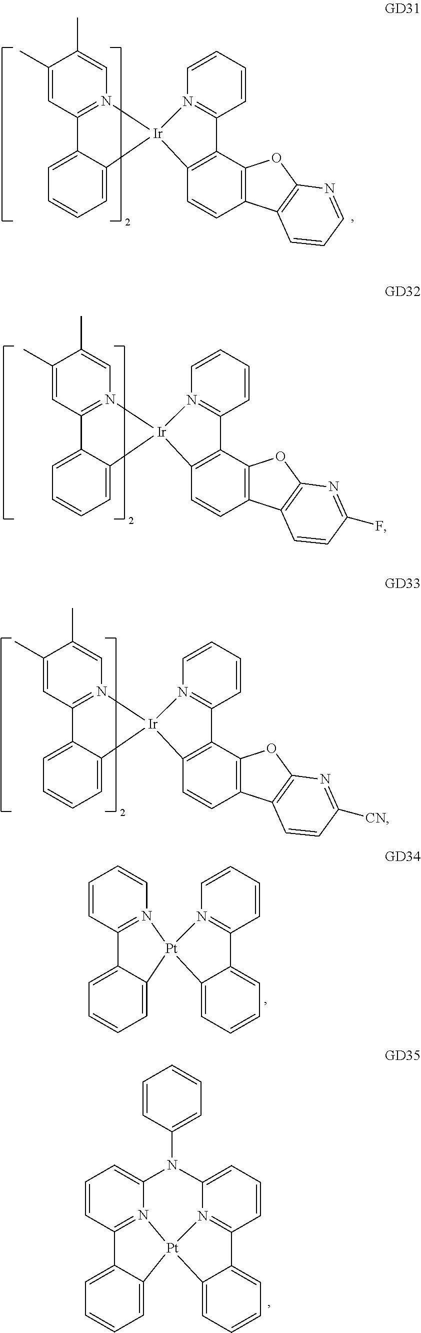

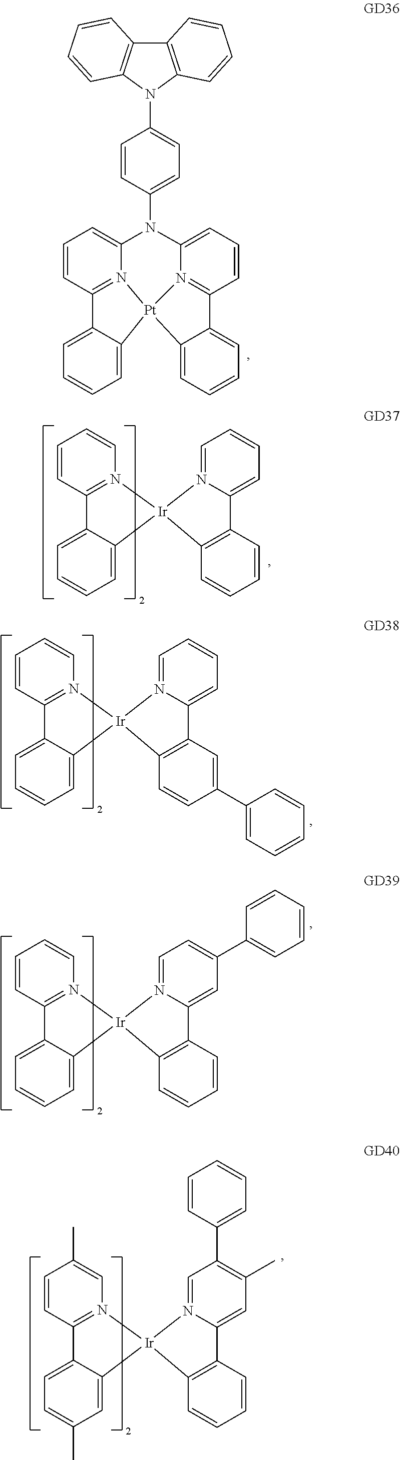

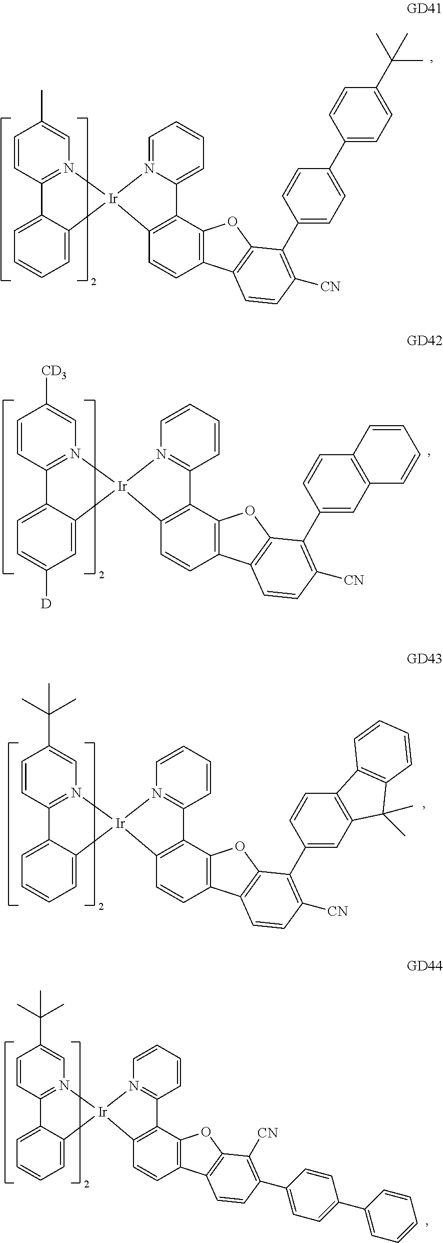

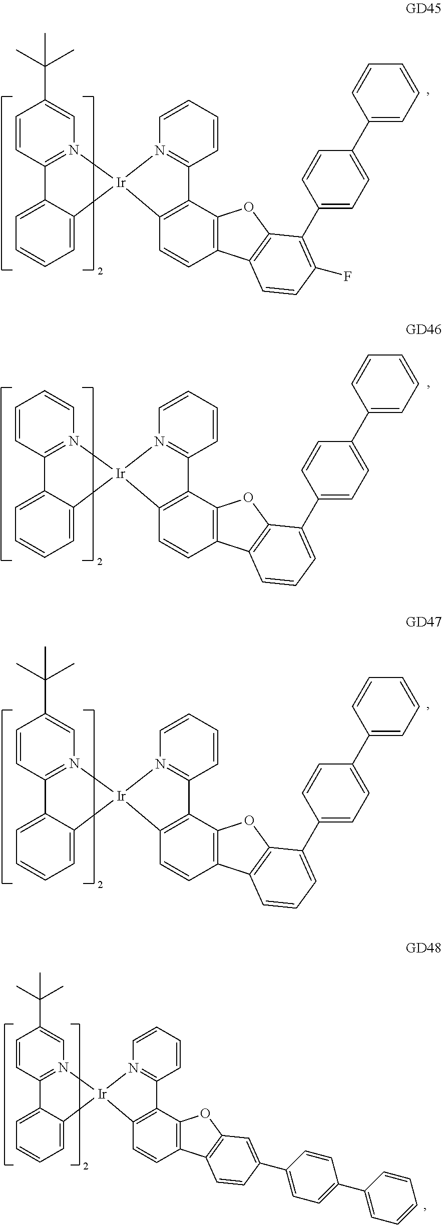

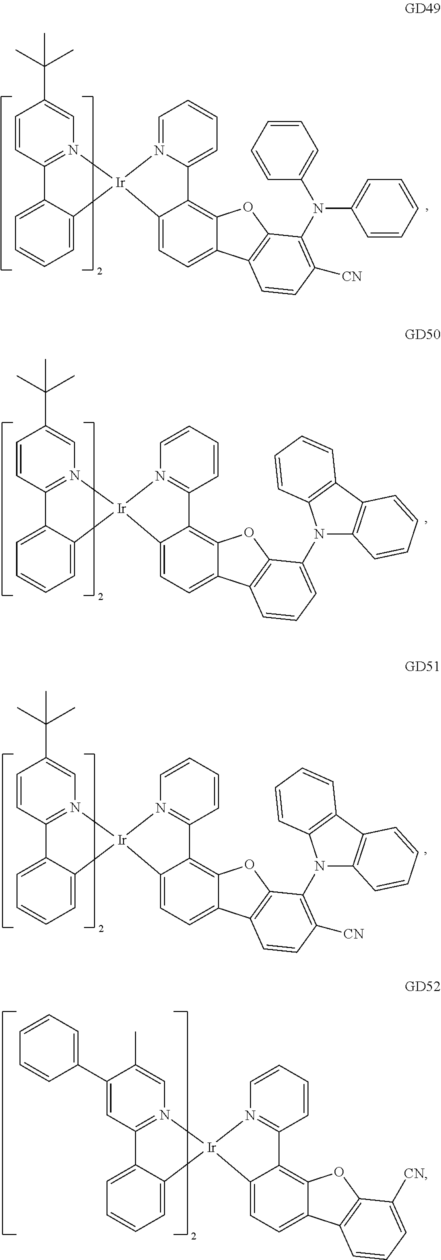

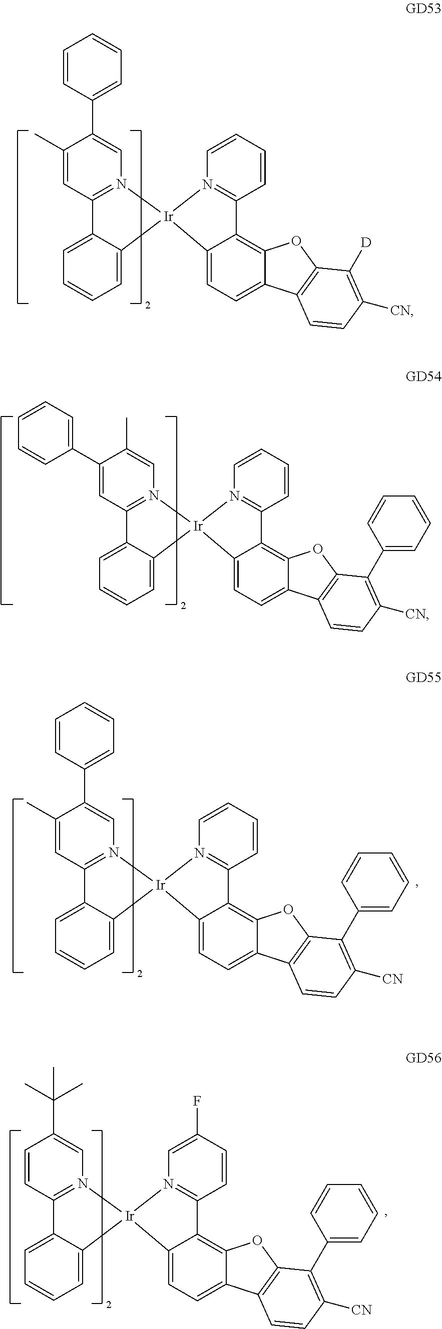

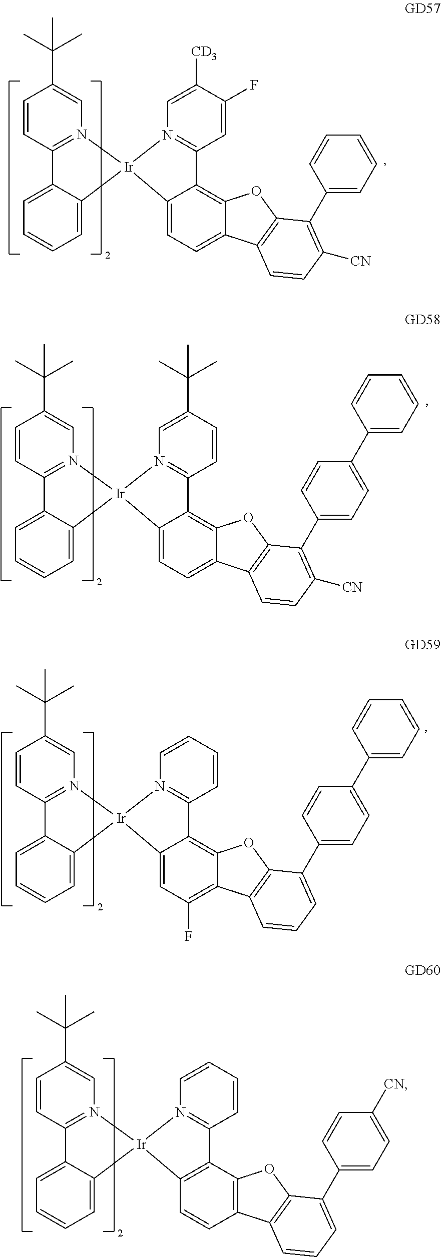

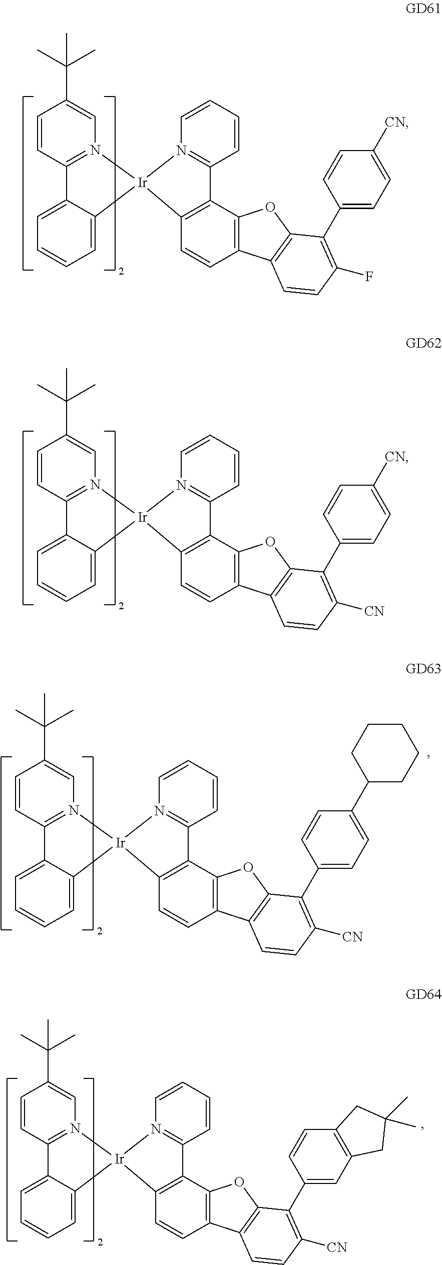

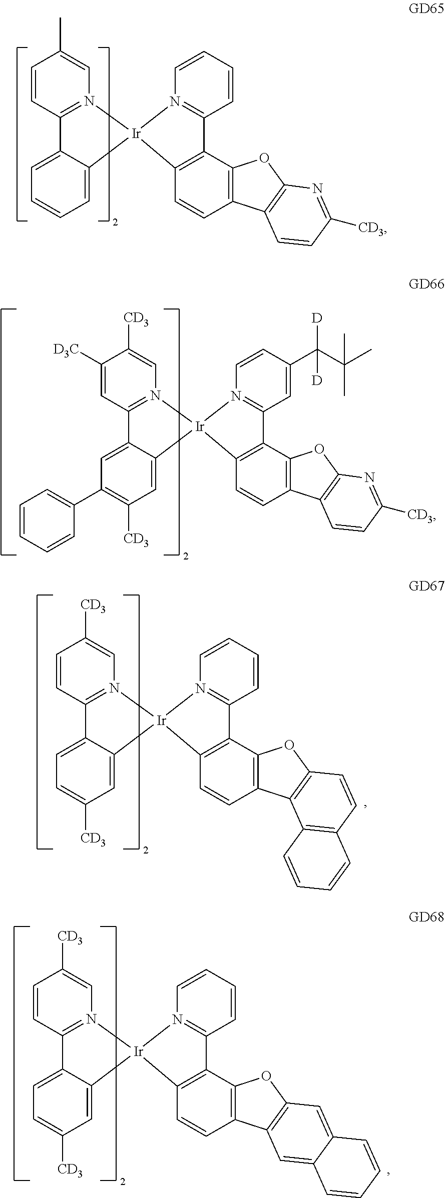

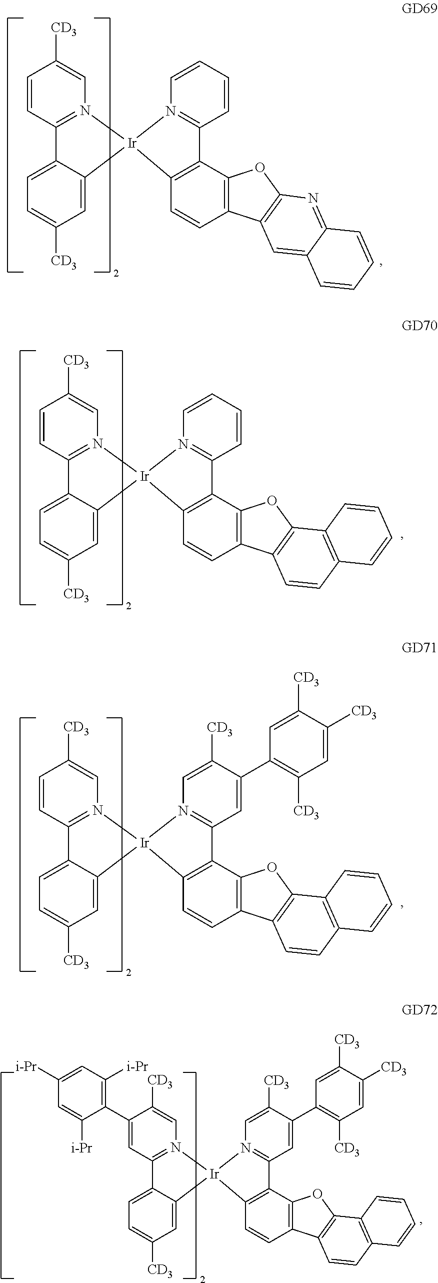

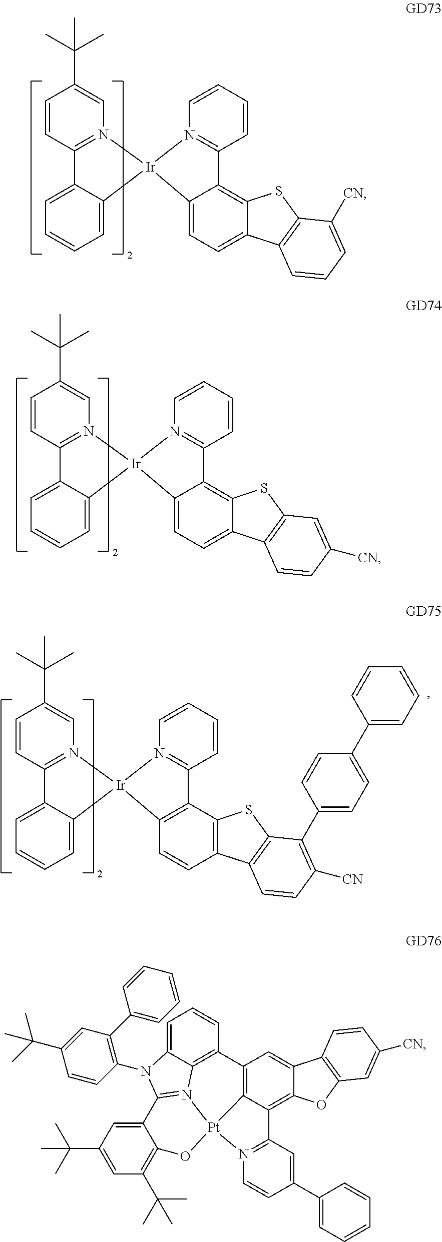

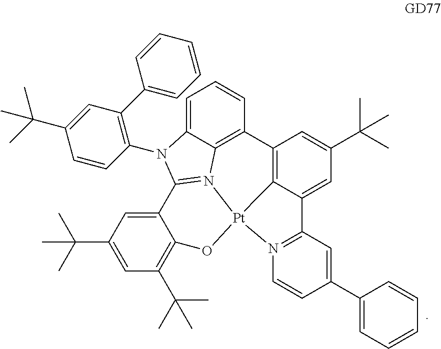

According to an embodiment of the present disclosure, hydrogens in Compound GD1 to Compound GD77 can be partially or fully substituted with deuterium.

According to an embodiment of the present disclosure, the organic electroluminescent device emits green light.

According to an embodiment of the present disclosure, the organic electroluminescent device emits white light.

According to an embodiment of the present disclosure, the metal complex is doped with the compound represented by Formula 1 and the second host compound, and the metal complex accounts for 1% to 30% of the total weight of the emissive layer.

According to an embodiment of the present disclosure, the metal complex is doped with the compound represented by Formula 1 and the second host compound, and the metal complex accounts for 3% to 13% of the total weight of the emissive layer.

According to an embodiment of the present disclosure, the organic layer and organic electroluminescent device comprising the compound having a structure of Formula 1 of the present disclosure are prepared by a vacuum evaporation method and/or a solution method.

According to an embodiment of the present disclosure, further disclosed is a compound composition comprising a compound represented by Formula 1, wherein the compound is as shown in any one of the preceding embodiments.

Combination with Other Materials

The materials described in the present disclosure for a particular layer in an organic light-emitting device can be used in combination with various other materials present in the device. The combinations of these materials are described in more detail in U.S. Pat. Pub. No. 20160359122 at paragraphs 0132-0161, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

The materials described herein as useful for a particular layer in an organic light-emitting device may be used in combination with a variety of other materials present in the device. For example, dopants disclosed herein may be used in combination with a wide variety of hosts, transport layers, blocking layers, injection layers, electrodes and other layers that may be present. The combination of these materials is described in detail in paragraphs 0080-0101 of U.S. Pat. Pub. No. 20150349273, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

In the embodiments of material synthesis, all reactions were performed under nitrogen protection unless otherwise stated. All reaction solvents were anhydrous and used as received from commercial sources. Synthetic products were structurally confirmed and tested for properties using one or more conventional equipment in the art (including, but not limited to, nuclear magnetic resonance instrument produced by BRUKER, liquid chromatograph produced by SHIMADZU, liquid chromatograph-mass spectrometry produced by SHIMADZU, gas chromatograph-mass spectrometry produced by SHIMADZU, differential Scanning calorimeters produced by SHIMADZU, fluorescence spectrophotometer produced by SHANGHAI LENGGUANG TECH., electrochemical workstation produced by WUHAN CORRTEST, and sublimation apparatus produced by ANHUI BEQ, etc.) by methods well known to the persons skilled in the art. In the embodiments of the device, the characteristics of the device were also tested using conventional equipment in the art (including, but not limited to, evaporator produced by ANGSTROM ENGINEERING, optical testing system produced by SUZHOU FSTAR, life testing system produced by SUZHOU FSTAR, and ellipsometer produced by BEIJING ELLITOP, etc.) by methods well known to the persons skilled in the art. As the persons skilled in the art are aware of the above-mentioned equipment use, test methods and other related contents, the inherent data of the sample can be obtained with certainty and without influence, so the above related contents are not further described in this patent.

### MATERIAL SYNTHESIS EXAMPLE

A method for preparing the compound of the present disclosure is not limited. Typically, the following compounds are used as examples without limitation, and the synthesis routes and preparation methods thereof are described below.

### Synthesis Example 1: Synthesis of Compound A-1

### Step 1: Synthesis of Intermediate C

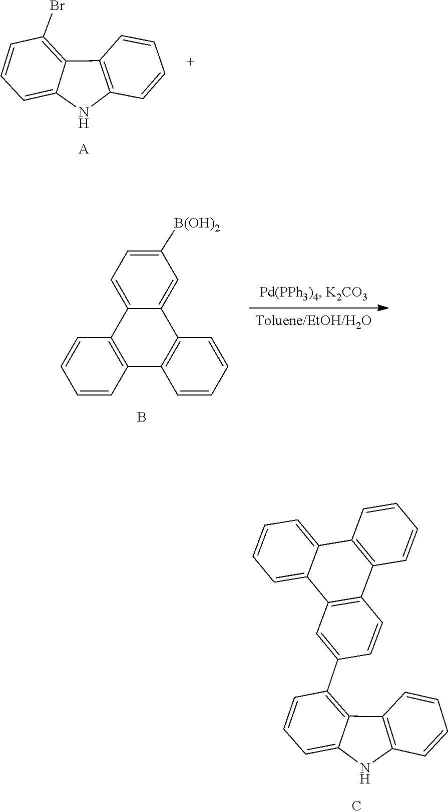

In a three-necked round-bottom flask, Intermediate A (7.4 g, 30.0 mmol), Intermediate B (8.6 g, 31.5 mmol), tetrakis(triphenylphosphine)palladium (Pd(PPh₃)₄) (1.04 g, 0.90 mmol), potassium carbonate (K₂CO₃) (8.29 g, 60.0 mmol), 120 mL of toluene, 30 mL of EtOH, and 30 mL of H₂O were added in sequence. Under N₂ protection, the system was heated and refluxed overnight. After the reaction was completed as confirmed by TLC, heating was stopped and the system was cooled to room temperature. The reaction solution was separated into layers, the aqueous phase was extracted with DCM, and the organic phases were combined, dried over anhydrous Na₂SO₄, filtered, and concentrated under reduced pressure to obtain a crude product. The crude product was purified through silica gel column chromatography (PE:DCM=10:1 to 3:1) to obtain Intermediate C (8.2 g, 20.8 mmol) as a white solid with a yield of 69.5%.

### Step 2: Synthesis of Intermediate F

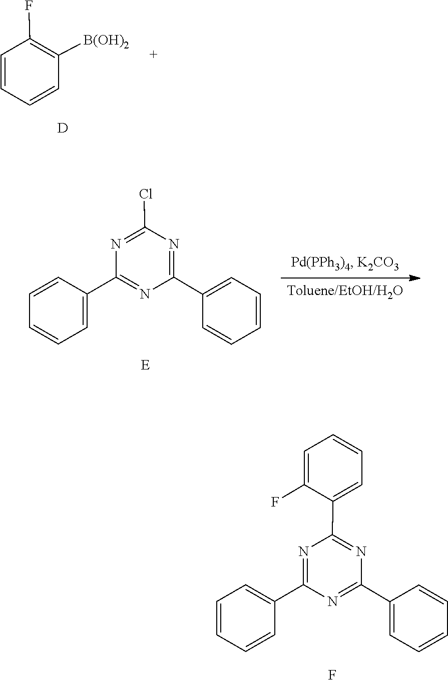

In a three-necked round-bottom flask, Intermediate D (4.2 g, 30.0 mmol), Intermediate E (8.0 g, 30.0 mmol), Pd(PPh₃)₄ (0.69 g, 0.60 mmol), K₂CO₃ (8.29 g, 60.0 mmol), 100 mL of toluene, 25 mL of EtOH, and 25 mL of H₂O were added in sequence. Under N₂ protection, the system was heated and refluxed overnight. After the reaction was completed as confirmed by TLC, heating was stopped and the system was cooled to room temperature. The reaction solution was separated into layers, the aqueous phase was extracted with DCM, and the organic phases were combined, dried over anhydrous Na₂SO₄, filtered, and concentrated under reduced pressure to obtain a crude product. The crude product was purified through silica gel column chromatography (PE:DCM=10:1 to 4:1) to obtain Intermediate F (9.0 g, 27.5 mmol) as a white solid with a yield of 91.6%.

### Step 3: Synthesis of Compound A-1

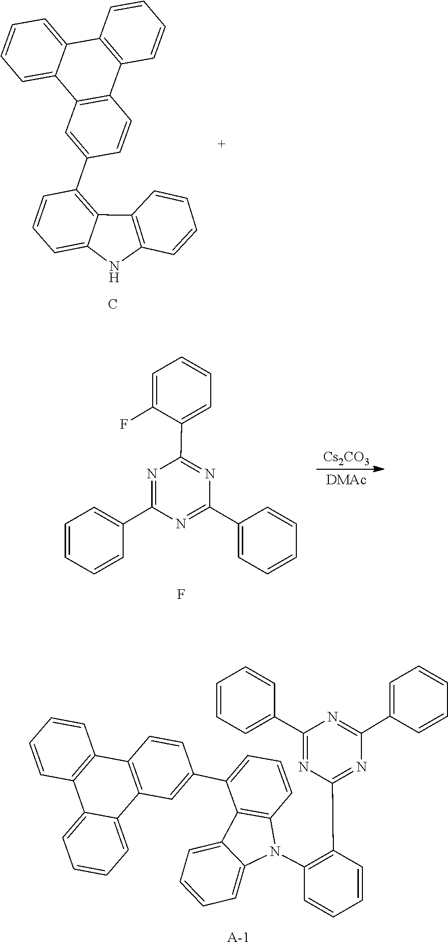

In a three-necked round-bottom flask, Intermediate C (3.25 g, 8.25 mmol), Intermediate F (2.45 g, 7.5 mmol), Cs₂CO₃ (4.9 g, 15.0 mmol), and 60 mL of N,N-dimethylacetamide (DMAc) were added in sequence. Under N₂ protection, the system was heated to reflux. After the reaction was completed as confirmed by TLC, heating was stopped and the system was cooled to room temperature. The reaction solution was poured into a large amount of water to precipitate a solid and suction-filtered under reduced pressure. The obtained solid was rinsed with water and methanol in sequence. The solid was purified through silica gel column chromatography (PE:DCM=10:1 to 3:1) to obtain a yellow solid (4.4 g, 6.28 mmol) with a yield of 83.7%. The product was confirmed as the target product A-1 with a molecular weight of 700.3.

### Synthesis Example 2: Synthesis of Compound A-201

### Step 1: Synthesis of Intermediate H

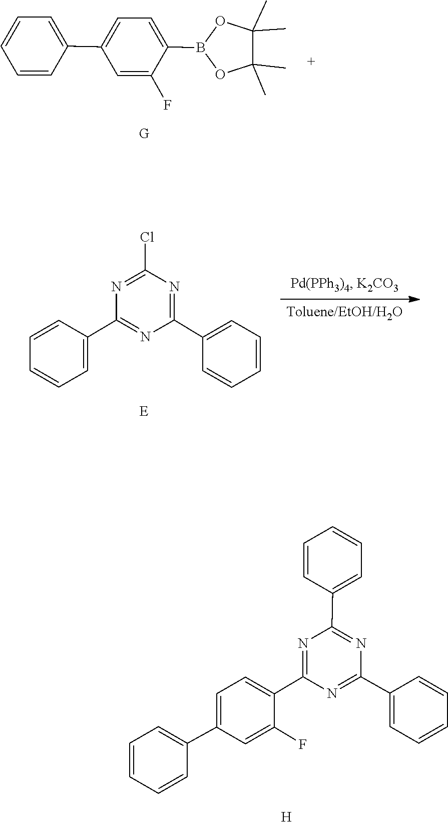

In a three-necked round-bottom flask, Intermediate G (13.4 g, 45.0 mmol), Intermediate E (12.0 g, 45.0 mmol), Pd(PPh₃)₄ (1.04 g, 0.60 mmol), K₂CO₃ (12.4 g, 90.0 mmol), 140 mL of toluene, 35 mL of EtOH, and 35 mL of H₂O were added in sequence. Under N₂ protection, the system was heated and refluxed overnight. After the reaction was completed as confirmed by TLC, heating was stopped and the system was cooled to room temperature and suction-filtered under reduced pressure. The obtained solid was rinsed with water and methanol in sequence. The solid was recrystallized from toluene to obtain Intermediate H (16.0 g, 39.7 mmol) as a white solid with a yield of 88.1%.

### Step 2: Synthesis of Compound A-201

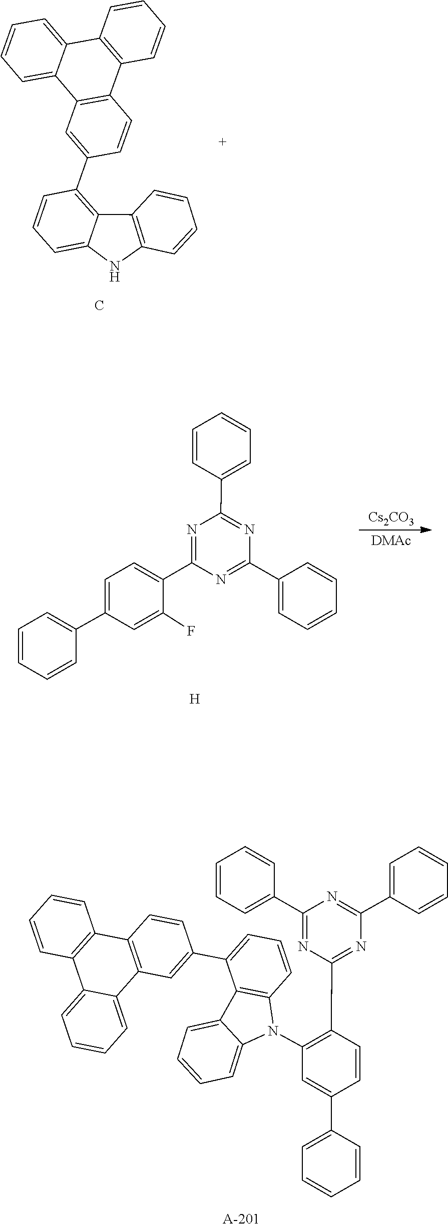

In a three-necked round-bottom flask, Intermediate C (2.92 g, 7.44 mmol), Intermediate H (2.5 g, 6.2 mmol), Cs₂CO₃ (4.04 g, 12.4 mmol), and 60 mL of N,N-dimethylacetamide (DMAc) were added in sequence. Under N₂ protection, the system was heated to reflux. After the reaction was completed as confirmed by TLC, heating was stopped and the system was cooled to room temperature. The reaction solution was poured into a large amount of water to precipitate a solid and suction-filtered under reduced pressure. The obtained solid was rinsed with water and methanol in sequence. The solid was purified through silica gel column chromatography (PE:DCM=10:1 to 3:1) to obtain a yellow solid (4.2 g, 5.4 mmol) with a yield of 87.2%. The product was confirmed as the target product A-201 with a molecular weight of 776.3.

### Synthesis Example 3: Synthesis of Compound A-205

### Step 1: Synthesis of Intermediate J

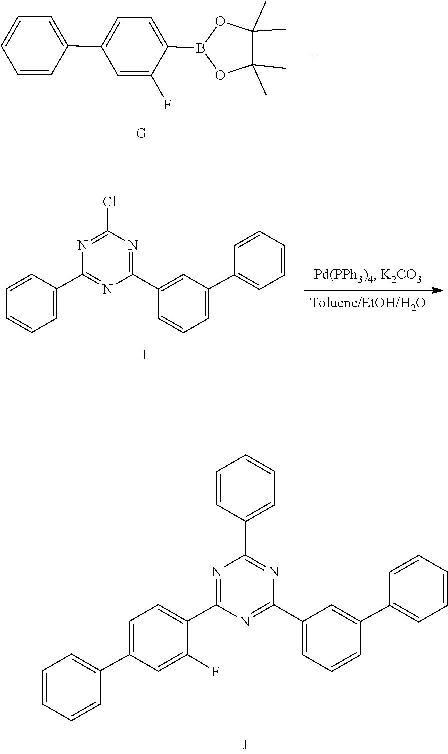

In a three-necked round-bottom flask, Intermediate G (3.64 g, 12.2 mmol), Intermediate I (3.5 g, 10.2 mmol), Pd(PPh₃)₄ (0.24 g, 0.21 mmol), K₂CO₃ (2.81 g, 20.4 mmol), 40 mL of toluene, 10 mL of EtOH, and 10 mL of H₂O were added in sequence. Under N₂ protection, the system was heated and refluxed overnight. After the reaction was completed as confirmed by TLC, heating was stopped and the system was cooled to room temperature. The reaction solution was separated into layers, the aqueous phase was extracted with DCM, and the organic phases were combined, dried over anhydrous Na₂SO₄, filtered, and concentrated under reduced pressure to obtain a crude product. The crude product was purified through silica gel column chromatography (PE:DCM=10:1 to 3:1) to obtain Intermediate J (4.3 g, 9.0 mmol) as a white solid with a yield of 88.1%.

### Step 2: Synthesis of Compound A-205

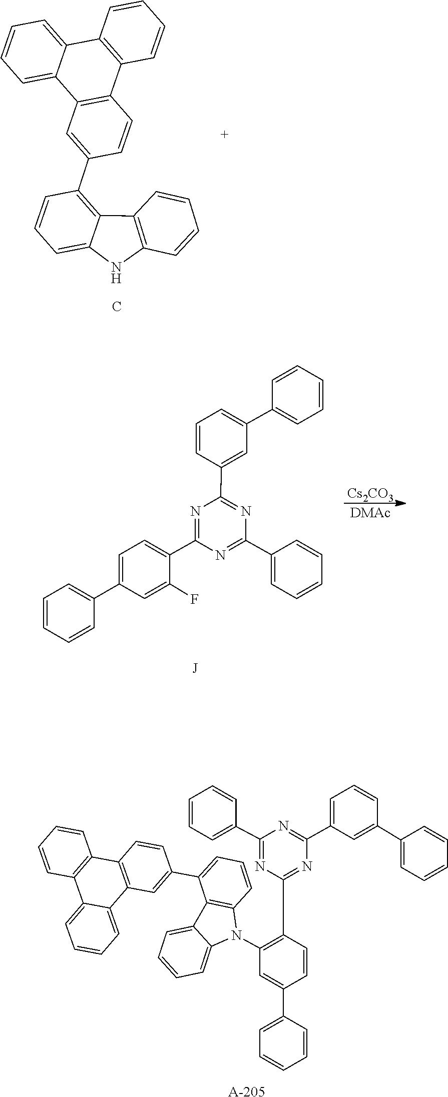

In a three-necked round-bottom flask, Intermediate C (2.96 g, 7.51 mmol), Intermediate J (3.0 g, 6.26 mmol), Cs₂CO₃ (4.08 g, 12.5 mmol), and 30 mL of N,N-dimethylacetamide (DMAc) were added in sequence. Under N₂ protection, the system was heated to reflux. After the reaction was completed as confirmed by TLC, heating was stopped and the system was cooled to room temperature. The reaction solution was poured into a large amount of water to precipitate a solid and suction-filtered under reduced pressure. The obtained solid was rinsed with water and methanol in sequence. The solid was purified through silica gel column chromatography (PE:DCM=10:1 to 3:1) to obtain a yellow solid (3.71 g, 4.35 mmol) with a yield of 69.5%. The product was confirmed as the target product A-205 with a molecular weight of 852.3.

### Synthesis Example 4: Synthesis of Compound A-411

### Step 1: Synthesis of Intermediate L

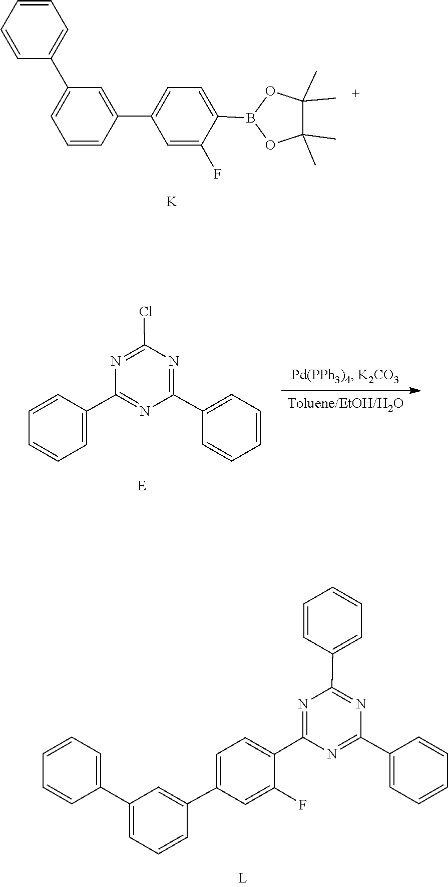

In a three-necked round-bottom flask, Intermediate K (9.36 g, 25.0 mmol), Intermediate E (6.69 g, 25.0 mmol), Pd(PPh₃)₄ (0.87 g, 0.75 mmol), K₂CO₃ (6.91 g, 50.0 mmol), 100 mL of toluene, 25 mL of EtOH, and 25 mL of H₂O were added in sequence. Under N₂ protection, the system was heated and refluxed overnight. After the reaction was completed as confirmed by TLC, heating was stopped and the system was cooled to room temperature. The reaction solution was separated into layers, the aqueous phase was extracted with DCM, and the organic phases were combined, dried over anhydrous Na₂SO₄, filtered, and concentrated under reduced pressure to obtain a crude product. The crude product was purified through silica gel column chromatography (PE:DCM=10:1 to 7:2) to obtain Intermediate L (10.7 g, 22.3 mmol) as a white solid with a yield of 89.2%.

### Step 2: Synthesis of Compound A-411

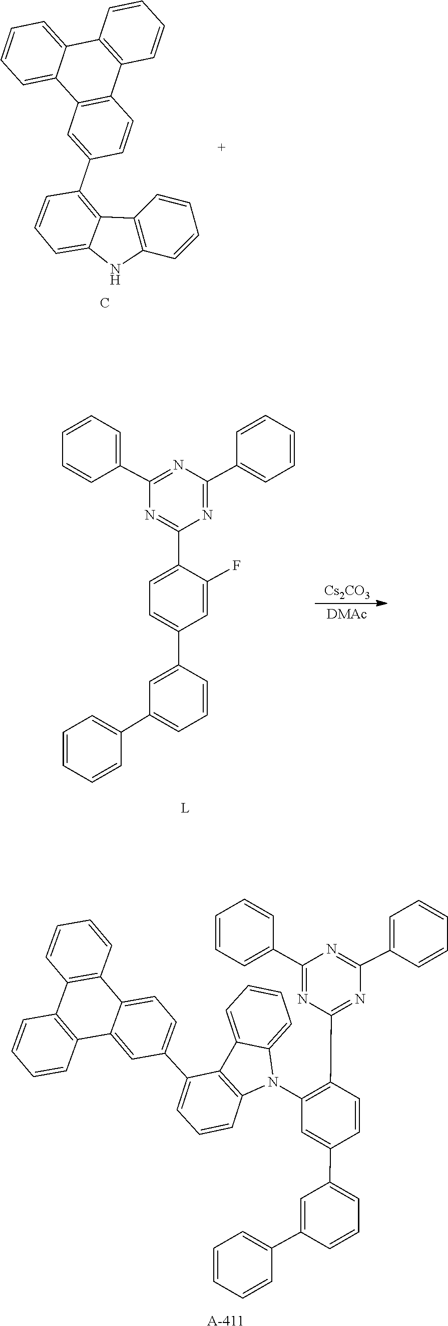

In a three-necked round-bottom flask, Intermediate C (2.60 g, 6.6 mmol), Intermediate L (2.88 g, 6.0 mmol), Cs₂CO₃ (3.91 g, 12.0 mmol), and 60 mL of N,N-dimethylacetamide (DMAc) were added in sequence. Under N₂ protection, the system was heated to reflux. After the reaction was completed as confirmed by TLC, heating was stopped and the system was cooled to room temperature. The reaction solution was poured into a large amount of water to precipitate a solid and suction-filtered under reduced pressure. The obtained solid was rinsed with water and methanol in sequence. The solid was purified through silica gel column chromatography (PE:DCM=10:1 to 3:1) to obtain a yellow solid (4.7 g, 5.5 mmol) with a yield of 91.8%. The product was confirmed as the target product A-411 with a molecular weight of 852.3.

### Synthesis Example 5: Synthesis of Compound A-481

### Step 1: Synthesis of Intermediate N

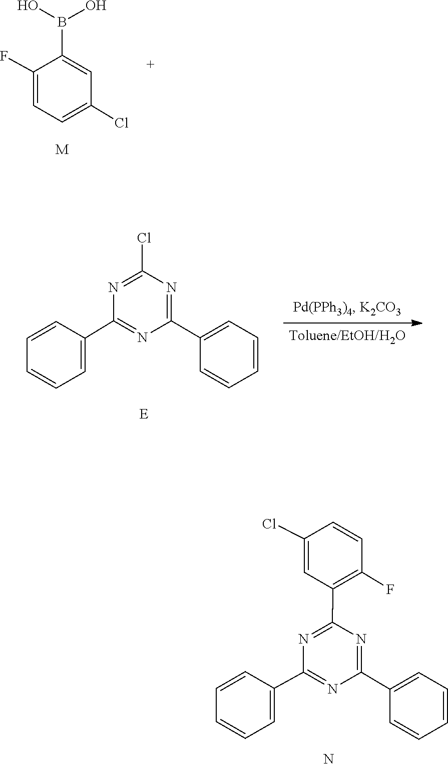

In a three-necked round-bottom flask, Intermediate M (4.36 g, 25.0 mmol), Intermediate E (7.03 g, 25.0 mmol), Pd(PPh₃)₄ (0.87 g, 0.75 mmol), K₂CO₃ (6.91 g, 50.0 mmol), 80 mL of toluene, 20 mL of EtOH, and 20 mL of H₂O were added in sequence. Under N₂ protection, the system was heated and refluxed overnight. After the reaction was completed as confirmed by TLC, heating was stopped and the system was cooled to room temperature. The reaction solution was separated into layers, the aqueous phase was extracted with DCM, and the organic phases were combined, dried over anhydrous Na₂SO₄, filtered, and concentrated under reduced pressure to obtain a crude product. The crude product was purified through silica gel column chromatography (PE:DCM=10:1 to 5:1) to obtain Intermediate N (8.4 g, 23.2 mmol) as a white solid with a yield of 92.9%.

### Step 2: Synthesis of Intermediate O

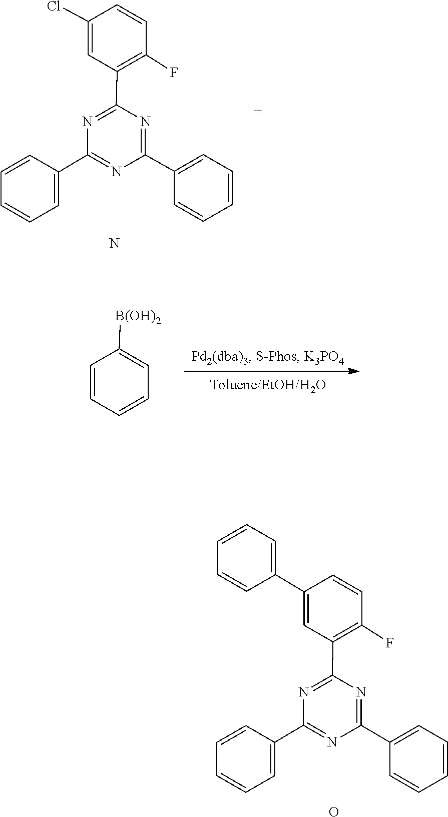

In a three-necked round-bottom flask, Intermediate N (8.7 g, 23.2 mmol), phenylboronic acid (3.11 g, 25.5 mmol), tris(dibenzylideneacetone)dipalladium (Pd₂(dba)₃) (0.42 g, 0.46 mmol), 2-dicyclohexylphosphino-2′,6′-dimethoxybiphenyl (S-Phos) (0.76 g, 1.86 mmol), K₃PO₄ (9.85 g, 46.4 mmol), 80 mL of toluene, 20 mL of EtOH, and 20 mL of H₂O were added in sequence. Under N₂ protection, the system was heated and refluxed overnight. After the reaction was completed as confirmed by TLC, heating was stopped and the system was cooled to room temperature. The reaction solution was separated into layers, the aqueous phase was extracted with DCM, and the organic phases were combined, dried over anhydrous Na₂SO₄, filtered, and concentrated under reduced pressure to obtain a crude product. The crude product was purified through silica gel column chromatography (PE:DCM=10:1 to 5:1) to obtain Intermediate O (5.1 g, 12.6 mmol) as a white solid with a yield of 54.5%.

### Step 3: Synthesis of Compound A-481

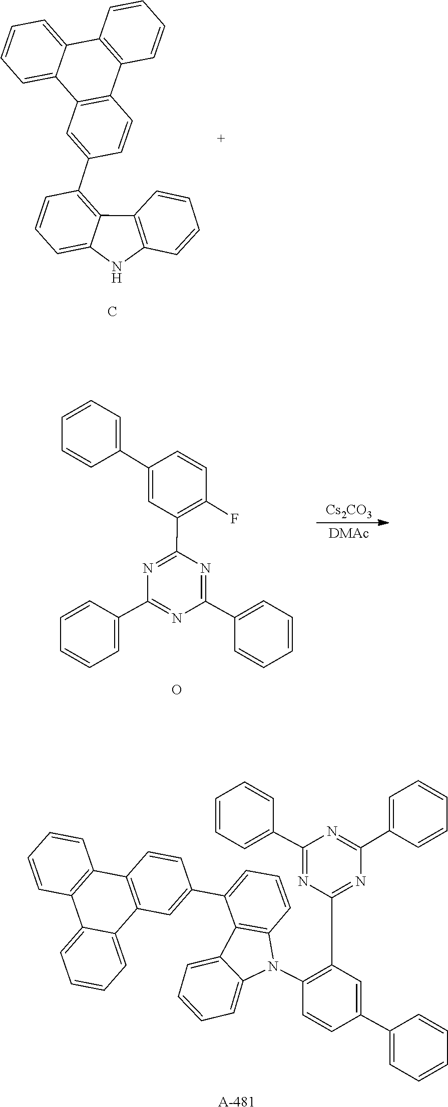

In a three-necked round-bottom flask, Intermediate C (3.30 g, 8.4 mmol), Intermediate O (2.82 g, 7.0 mmol), Cs₂CO₃ (4.56 g, 14.0 mmol), and 70 mL of N,N-dimethylacetamide (DMAc) were added in sequence. Under N₂ protection, the system was heated to reflux. After the reaction was completed as confirmed by TLC, heating was stopped and the system was cooled to room temperature. The reaction solution was poured into a large amount of water to precipitate a solid and suction-filtered under reduced pressure. The obtained solid was rinsed with water and methanol in sequence. The solid was purified through silica gel column chromatography (PE:DCM=10:1 to 3:1) to obtain a yellow solid (4.6 g, 5.9 mmol) with a yield of 84.6%. The product was confirmed as the target product A-481 with a molecular weight of 776.3.

Those skilled in the art will appreciate that the above preparation methods are merely illustrative. Those skilled in the art can obtain other compound structures of the present disclosure through modifications of the preparation methods.

In the present disclosure, the glass transition temperatures (Tg) of the compounds of the present disclosure and comparative compounds were tested through differential scanning calorimetry (DSC). The specific method was as follows: a sample (approximately 3 mg) was weighed and tested using an instrument, Shimadzu DSC-60A plus, under the conditions of a carrier gas (N₂) with a flowrate of 50 mL/min, a temperature raising rate of 10° C./min, and a temperature range of 20-500° C. Tg of the compounds of the present disclosure and the comparative compounds were measured by the above method, and the results are shown in Table 1 below.

TABLE 1
Tg of the compounds of the present disclosure
and the comparative compounds

| Compound No. | Tg (° C.) |
|---|---|
| A-1 | 145.94 |
| A-201 | 162.74 |
| A-205 | 160.04 |
| A-411 | 157.11 |
| A-481 | 160.14 |
| C-1 | 94.38 |
| C-2 | 115.71 |

As can be seen from the data in Table 1, compared with Comparative Compounds C-1 and C-2, the compounds of the present disclosure having a particular structure represented by Formula 1 have significantly higher Tg and exhibit superior thermal stability. During preparation of OLEDs, the compounds have advantages in preparing OLED devices by a solution method in addition to a vacuum thermal evaporation method (vacuum evaporation method).

A method for preparing an electroluminescent device is not limited. The preparation methods of the following examples are merely illustrative and are not to be construed as limiting. Based on the related art, those skilled in the art can make reasonable improvements on the preparation methods of the following examples. For example, the proportions of various materials in an emissive layer are not particularly limited. Those skilled in the art can reasonably select the proportions within a certain range based on the related art. For example, taking the total weight of the materials in the emissive layer for reference, a host material may account for 80% to 99% and a light-emitting material may account for 1% to 20%; or the host material may account for 90% to 99% and the light-emitting material may account for 1% to 10%; or the host material may account for 94% to 99% and the light-emitting material may account for 1% to 6%. Further, the host material may include one material or two materials, where the ratio of two host materials may be 100:0 to 1:99, may be 80:20 to 20:80, or may be 60:40 to 40:60.

### DEVICE EXAMPLE

### Device Example 1

Firstly, a glass substrate having an indium tin oxide (ITO) anode with a thickness of 80 nm was cleaned and then treated with oxygen plasma and UV ozone. After the treatment, the substrate was dried in a glovebox to remove moisture. Then, the substrate was mounted on a substrate holder and placed in a vacuum chamber. The organic layers specified below were sequentially deposited through vacuum thermal evaporation on the ITO anode at a rate of 0.1-2 Angstroms per second and a vacuum degree of about 10⁻⁸ Torr. Compound HT and Compound HT1 were co-deposited (at a weight ratio of 97:3) for use as a hole injection layer (HIL) with a thickness of 100 Å. Compound HT was used as a hole transporting layer (HTL) with a thickness of 350 Å. Compound PH-1 was used as an electron blocking layer (EBL) with a thickness of 50 Å. Compound GD75 was doped in Compound PH-24 and Compound A-1 of the present disclosure and co-deposited (at a weight ratio of 6:52:42) for use as an emissive layer (EML) with a thickness of 400 Å. Compound HB was used as a hole blocking layer (HBL) with a thickness of 50 Å. On the HBL, Compound ET and 8-hydroxyquinolinolato-lithium (Liq) were co-deposited (at a weight ratio of 40:60) for use as an electron transporting layer (ETL) with a thickness of 350 Å. Finally, Liq was deposited for use as an electron injection layer with a thickness of 1 nm and Al was deposited for use as a cathode with a thickness of 120 nm. The device was transferred back to the glovebox and encapsulated with a glass lid to complete the device.

### Device Example 2

Device Example 2 was prepared by the same method as Device Example 1 except that in the EML, Compound A-1 was replaced with Compound A-201.

### Device Example 3

Device Example 3 was prepared by the same method as Device Example 1 except that in the EML, Compound A-1 was replaced with Compound A-205 and PH-24:A-205:GD75=56:38:6.

### Device Example 4

Device Example 4 was prepared by the same method as Device Example 3 except that in the EML, Compound A-205 was replaced with Compound A-411.

### Device Example 5

Device Example 5 was prepared by the same method as Device Example 3 except that in the EML, Compound A-205 was replaced with Compound A-481.

### Device Comparative Example 1

Device Comparative Example 1 was prepared by the same method as Device Example 1 except that in the EML, Compound A-1 was replaced with Compound C-1.

### Device Comparative Example 2

Device Comparative Example 2 was prepared by the same method as Device Example 1 except that in the EML, Compound A-1 was replaced with Compound C-2.

Detailed structures and thicknesses of layers of the devices are shown in Table 2. A layer using more than one material is obtained by doping different compounds at their weight ratio as recorded.

TABLE 2
Part of device structures of Device Examples 1 to 5 and Comparative Examples 1 and 2

| Device ID | HIL | HTL | EBL | EML | HBL | ETL |
|---|---|---|---|---|---|---|
| Example 1 | Compound | Compound | Compound | Compound PH- | Compound | Compound |
| | HT:Compound | HT (350 | PH-1 (50 | 24:Compound | HB (50 Å) | ET:Liq |
| | HT1 (97:3) | Å) | Å) | A-1:Compound | | (40:60) |
| | (100 Å) | | | GD75 (52:42:6) | | (350 Å) |
| | | | | (400 Å) | | |
| Example 2 | Compound | Compound | Compound | Compound PH- | Compound | Compound |
| | HT:Compound | HT (350 | PH-1 (50 | 24:Compound | HB (50 Å) | ET:Liq |
| | HT1 (97:3) | Å) | Å) | A- | | (40:60) |
| | (100 Å) | | | 201:Compound | | (350 Å) |
| | | | | GD75 (52:42:6) | | |
| | | | | (400 Å) | | |
| Example 3 | Compound | Compound | Compound | Compound PH- | Compound | Compound |
| | HT:Compound | HT (350 | PH-1 (50 | 24:Compound | HB (50 Å) | ET:Liq |
| | HT1 (97:3) | Å) | Å) | A- | | (40:60) |
| | (100 Å) | | | 205:Compound | | (350 Å) |
| | | | | GD75 (56:38:6) | | |
| | | | | (400 Å) | | |
| Example 4 | Compound | Compound | Compound | Compound PH- | Compound | Compound |
| | HT:Compound | HT (350 | PH-1 (50 | 24:Compound | HB (50 Å) | ET:Liq |
| | HT1 (97:3) | Å) | Å) | A- | | (40:60) |
| | (100 Å) | | | 411:Compound | | (350 Å) |
| | | | | GD75 (56:38:6) | | |
| | | | | (400 Å) | | |
| Example 5 | Compound | Compound | Compound | Compound PH- | Compound | Compound |
| | HT:Compound | HT (350 | PH-1 (50 | 24:Compound | HB (50 Å) | ET:Liq |
| | HT1 (97:3) | Å) | Å) | A- | | (40:60) |
| | (100 Å) | | | 481:Compound | | (350 Å) |
| | | | | GD75 (56:38:6) | | |
| | | | | (400 Å) | | |
| Comparative | Compound | Compound | Compound | Compound PH- | Compound | Compound |
| Example 1 | HT:Compound | HT (350 | PH-1 (50 | 24:Compound | HB (50 Å) | ET:Liq |
| | HT1 (97:3) | Å) | Å) | C-1:Compound | | (40:60) |
| | (100 Å) | | | GD75 (52:42:6) | | (350 Å) |
| | | | | (400 Å) | | |
| Comparative | Compound | Compound | Compound | Compound PH- | Compound | Compound |
| Example 2 | HT:Compound | HT (350 | PH-1 (50 | 24:Compound | HB (50 Å) | ET:Liq |
| | HT1 (97:3) | Å) | Å) | C-2:Compound | | (40:60) |
| | (100 Å) | | | GD75 (52:42:6) | | (350 Å) |
| | | | | (400 Å) | | |

The materials used in the devices have the following structures:

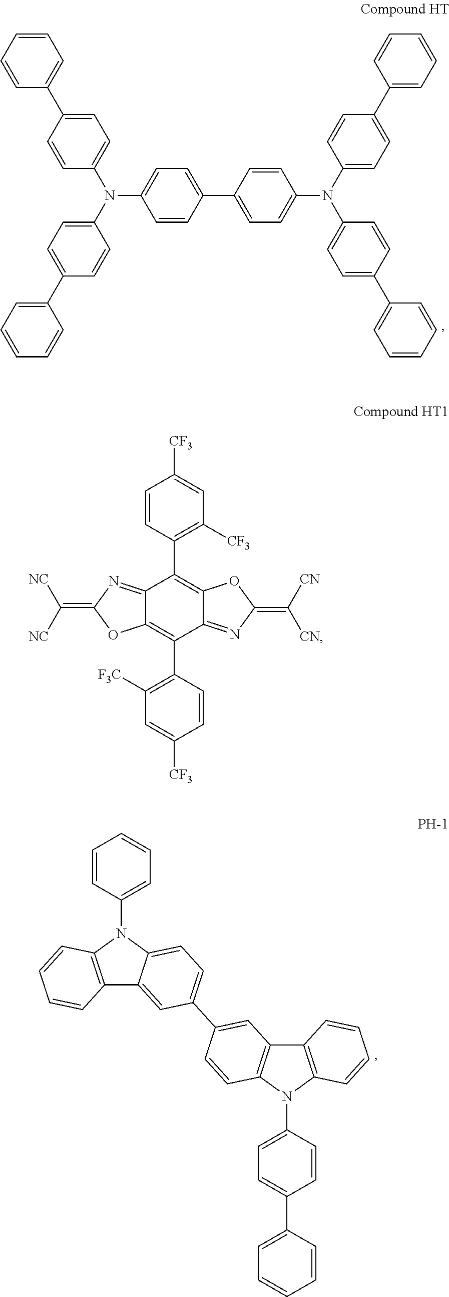 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; N#CC(C#N)=c1nc2c(-c3ccc(C(F)(F)F)cc3C(F)(F)F)c3oc(=C(C#N)C#N)nc3c(-c3ccc(C(F)(F)F)cc3C(F)(F)F)c2o1 ; c1ccc(-c2ccc(-n3c4ccccc4c4cc(-c5ccc6c(c5)c5ccccc5n6-c5ccccc5)ccc43)cc2)cc1]

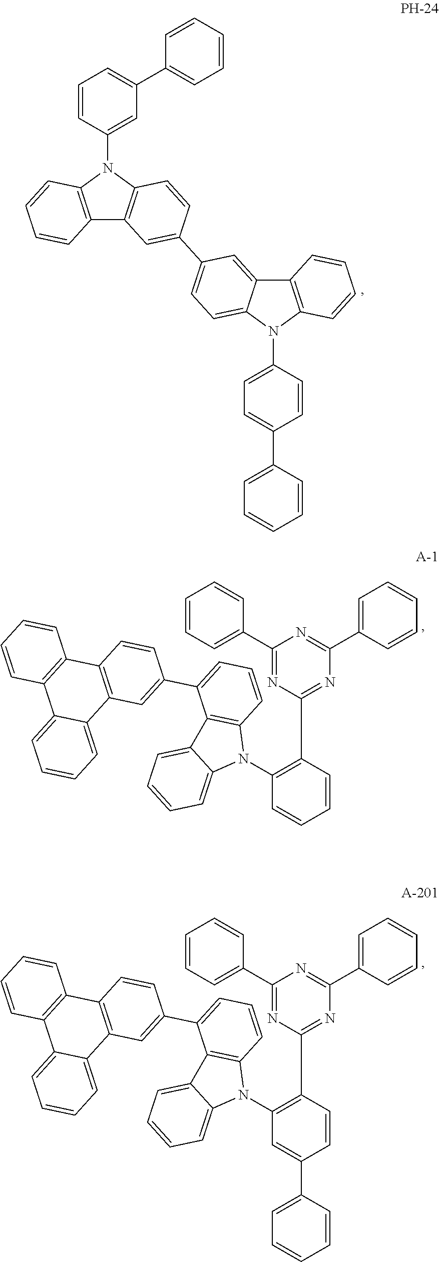 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2nc(-c3ccccc3)nc(-c3ccccc3-n3c4ccccc4c4c(-c5ccc6c7ccccc7c7ccccc7c6c5)cccc43)n2)cc1 ; c1ccc(-c2ccc(-c3nc(-c4ccccc4)nc(-c4ccccc4)n3)c(-n3c4ccccc4c4c(-c5ccc6c7ccccc7c7ccccc7c6c5)cccc43)c2)cc1]

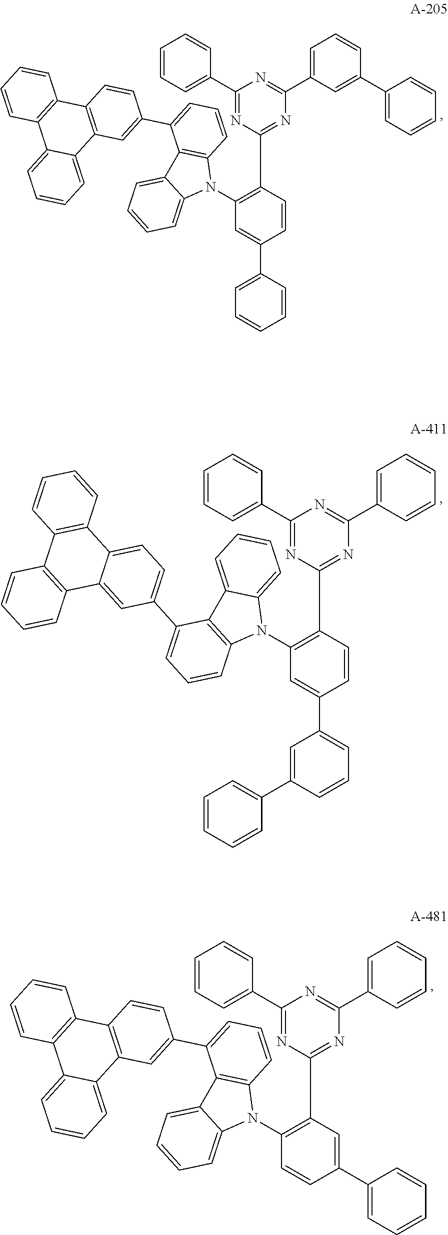 [SMILES: c1ccc(-c2cccc(-c3nc(-c4ccccc4)nc(-c4ccc(-c5ccccc5)cc4-n4c5ccccc5c5c(-c6ccc7c8ccccc8c8ccccc8c7c6)cccc54)n3)c2)cc1 ; c1ccc(-c2cccc(-c3ccc(-c4nc(-c5ccccc5)nc(-c5ccccc5)n4)c(-n4c5ccccc5c5c(-c6ccc7c8ccccc8c8ccccc8c7c6)cccc54)c3)c2)cc1 ; c1ccc(-c2ccc(-n3c4ccccc4c4c(-c5ccc6c7ccccc7c7ccccc7c6c5)cccc43)c(-c3nc(-c4ccccc4)nc(-c4ccccc4)n3)c2)cc1]

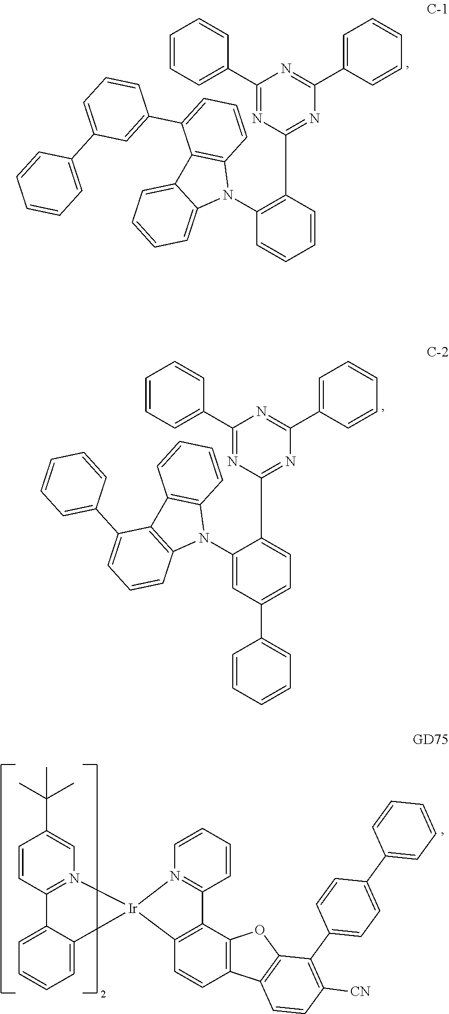

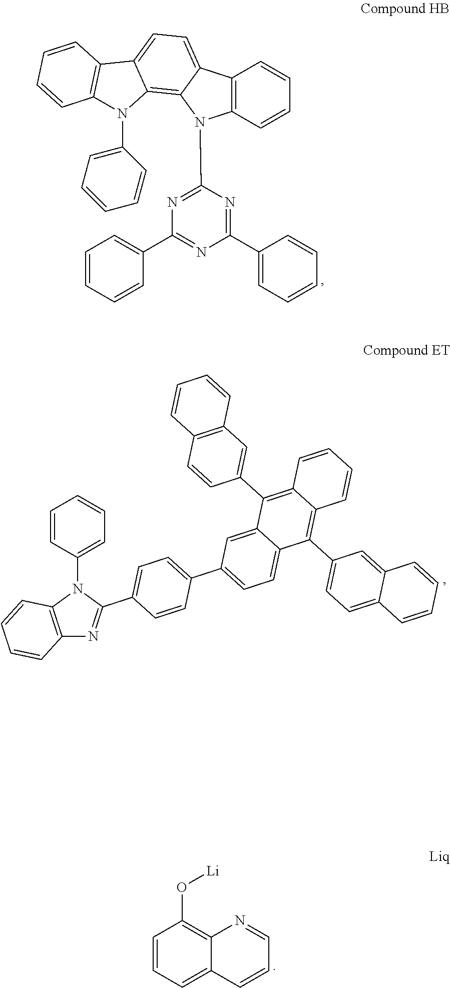

Table 3 shows the CIE data, external quantum efficiency (EQE), and current efficiency (CE) measured at a constant current density of 15 mA/cm² and the device lifetimes (LT97) measured at a constant current density of 80 mA/cm². The device lifetimes (LT97) were normalized based on the data of Comparative Example 1.

TABLE 3
Data of Device Examples 1 to 5 and
Comparative Examples 1 and 2

| | | | EQE | CE | |
|---|---|---|---|---|---|
| Device ID | EML | CIE (x, y) | (%) | (cd/A) | LT97 |
| Example 1 | PH-24:A-1:GD75 | (0.343, 0.634) | 24.5 | 96.3 | 3.42 |
| | (52:42:6) | | | | |
| Example 2 | PH-24:A- | (0.343, 0.634) | 24.8 | 97.7 | 5.51 |
| | 201:GD75 | | | | |
| | (52:42:6) | | | | |
| Example 3 | PH-24:A- | (0.343, 0.633) | 25.2 | 99.1 | 3.85 |
| | 205:GD75 | | | | |
| | (56:38:6) | | | | |
| Example 4 | PH-24:A- | (0.341, 0.635) | 25.1 | 98.9 | 3.93 |
| | 411:GD75 | | | | |
| | (56:38:6) | | | | |
| Example 5 | PH-24:A- | (0.344, 0.633) | 24.6 | 96.6 | 3.98 |
| | 481:GD75 | | | | |
| | (56:38:6) | | | | |
| Comparative | PH-24:C-1:GD75 | (0.343, 0.634) | 24.0 | 94.6 | 1.0 |
| Example 1 | (52:42:6) | | | | |
| Comparative | PH-24:C-2:GD75 | (0.344, 0.633) | 24.3 | 96.0 | 3.13 |
| Example 2 | (52:42:6) | | | | |

As can be seen from Table 2 and Table 3, Compound A-1 of the present disclosure and Comparative Compound C-1 respectively used in Example 1 and Comparative Example 1 differ only in different substituents on carbazole: Compound A-1 of the present disclosure has triphenylenyl as a substituent on carbazole, while Comparative Compound C-1 has biphenyl as a substituent on carbazole. Comparative Example 1 has already been at very high levels of device efficiency (EQE and CE). Compared with Comparative Example 1, Example 1 has further improved device efficiency, and more importantly, Example 1 has a device lifetime increased by 2.42 times. This indicates that, compared with compounds with other substituents on carbazole in the related art, the compound of Formula 1 of the present disclosure having triphenylenyl (and similar structures) as the substituent on carbazole, when applied to an electroluminescent device, can improve the overall performance of the device and, in particular, can significantly increase the device lifetime, where such a significant improvement in device lifetime is unexpected.

Similarly, Compound A-201 of the present disclosure and Comparative Compound C-2 respectively used in Example 2 and Comparative Example 2 differ only in different substituents on carbazole: Compound A-201 of the present disclosure further introduces a triphenylene substituent into carbazole, while Comparative Compound C-2 has a phenyl substituent on carbazole. Comparative Example 2 has already been at very high levels of device efficiency (EQE and CE). Compared with Comparative Example 2, Example 2 has further improved device efficiency, and more importantly, Example 2 has a device lifetime greatly increased by 76.0%. This indicates that, compared with the compounds with other substituents on carbazole in the related art, the compound of Formula 1 of the present disclosure having triphenylenyl (and similar structures) as the substituent on carbazole, when applied to the electroluminescent device, can improve the overall performance of the device and, in particular, can significantly increase the device lifetime, where such a significant improvement in device lifetime is unexpected.

Further, the Applicant has synthesized and implemented more compounds of the present disclosure having a structure of Formula 1, such as Compounds A-205, A-411, and A-481 of the present disclosure having the structure of Formula 1, which are used in Examples 3 to 5, respectively. The data in Table 3 indicate that, similar to Examples 1 and 2, Examples 3 to 5 also exhibit very high device efficiency (EQE and CE) and very long device lifetimes which are significantly increased compared with those of Comparative Examples 1 and 2. This further proves the excellent performance of the compound of the present disclosure having a particular structure represented by Formula 1.

To conclude, the compound of the present disclosure having a structure of Formula 1, when applied to an organic electroluminescent device, improves an electron and hole transport balance capability and can significantly improve the overall performance of the device: very high device efficiency (EQE and CE) and an unexpectedly significant improvement in device lifetime, demonstrating wide application values.

It should be understood that various embodiments described herein are merely examples and not intended to limit the scope of the present disclosure. Therefore, it is apparent to the persons skilled in the art that the present disclosure as claimed may include variations from specific embodiments and preferred embodiments described herein. Many of materials and structures described herein may be substituted with other materials and structures without departing from the spirit of the present disclosure. It should be understood that various theories as to why the present disclosure works are not intended to be limitative.

What is claimed is:

1. A compound having a structure of Formula 1: 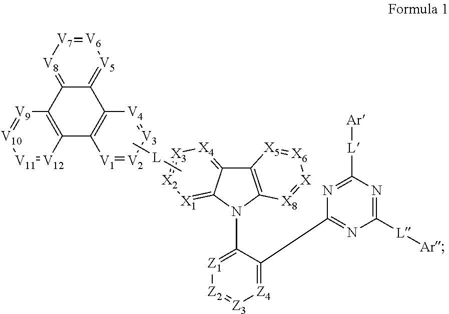
wherein
V₁ to V₄ are, at each occurrence identically or differently, selected from C, CRᵥ, or N, and one of V₁ to V₄ is selected from C and joined to L;
V₅ to V₁₂ are, at each occurrence identically or differently, selected from CRᵥ or N;
X₁ to X₄ are, at each occurrence identically or differently, selected from C, CRₓ, or N, and one of X₁ to X₄ is selected from C and joined to L;
X₅ to X₈ are, at each occurrence identically or differently, selected from CRₓ or N;
Z₁ to Z₄ are, at each occurrence identically or differently, selected from CR_{z} or N;
L, L′, and L″ are, at each occurrence identically or differently, selected from a single bond, substituted or unsubstituted alkylene having 1 to 30 carbon atoms, substituted or unsubstituted cycloalkylene having 3 to 30 carbon atoms, substituted or unsubstituted arylene having 6 to 30 carbon atoms, substituted or unsubstituted heteroarylene having 3 to 30 carbon atoms, or a combination thereof;
Ar′ and Ar″ are, at each occurrence identically or differently, selected from substituted or unsubstituted aryl having 6 to 30 carbon atoms, substituted or unsubstituted heteroaryl having 3 to 30 carbon atoms, or a combination thereof;
Rᵥ and R_{z} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
Rₓ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, an isocyano group, a hydroxyl group, a sulfanyl group, a sulfinyl group, a sulfonyl group, a phosphino group, and combinations thereof;
adjacent substituents Rᵥ can be optionally joined to form a ring;
adjacent substituents Rₓ can be optionally joined to form a ring; and
adjacent substituents R_{z} can be optionally joined to form a ring.

2. The compound of claim 1, wherein V₂ is selected from C and joined to L in Formula 1;
preferably, V₂ is selected from C and joined to L in Formula 1, and X₃ or X₄ is selected from C and joined to L in Formula 1.

3. The compound of claim 1, wherein at least one of Z₁ to Z₄ is selected from CR_{z}, and the R_{z} is, at each occurrence identically or differently, selected from substituted or unsubstituted aryl having 6 to 30 carbon atoms, substituted or unsubstituted heteroaryl having 3 to 30 carbon atoms, or a combination thereof;
preferably, Z₂ or Z₃ is selected from CR_{z}, and the R_{z} is, at each occurrence identically or differently, selected from substituted or unsubstituted aryl having 6 to 20 carbon atoms, substituted or unsubstituted heteroaryl having 3 to 20 carbon atoms, or a combination thereof;
more preferably, the R_{z} is, at each occurrence identically or differently, selected from the group consisting of: substituted or unsubstituted phenyl, substituted or unsubstituted naphthyl, substituted or unsubstituted phenanthryl, substituted or unsubstituted biphenyl, substituted or unsubstituted terphenyl, substituted or unsubstituted triphenylenyl, substituted or unsubstituted fluorenyl, substituted or unsubstituted pyridyl, substituted or unsubstituted carbazolyl, substituted or unsubstituted dibenzofuryl, substituted or unsubstituted dibenzothienyl, and combinations thereof.

4. The compound of claim 1, wherein V₁ to V₄ are, at each occurrence identically or differently, selected from C or CRᵥ; and/or V₅ to V₁₂ are, at each occurrence identically or differently, selected from CRᵥ; and/or X₁ to X₄ are, at each occurrence identically or differently, selected from C or CRₓ; and/or X₅ to X₈ are, at each occurrence identically or differently, selected from CRₓ; and/or Z₁ to Z₄ are, at each occurrence identically or differently, selected from CR_{z}.

5. The compound of claim 1, wherein Ar′ and Ar″ are, at each occurrence identically or differently, selected from substituted or unsubstituted aryl having 6 to 20 carbon atoms, substituted or unsubstituted heteroaryl having 3 to 20 carbon atoms, or a combination thereof;
preferably, Ar′ and Ar″ are, at each occurrence identically or differently, selected from the group consisting of: substituted or unsubstituted phenyl, substituted or unsubstituted naphthyl, substituted or unsubstituted phenanthryl, substituted or unsubstituted biphenyl, substituted or unsubstituted terphenyl, substituted or unsubstituted triphenylenyl, substituted or unsubstituted fluorenyl, substituted or unsubstituted pyridyl, substituted or unsubstituted pyrimidinyl, substituted or unsubstituted carbazolyl, substituted or unsubstituted dibenzofuryl, substituted or unsubstituted dibenzothienyl, and combinations thereof.

6. The compound of claim 1, wherein L, L′, and L″ are, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms, or a combination thereof;
preferably, L, L′, and L″ are, at each occurrence identically or differently, selected from a single bond, substituted or unsubstituted phenylene, substituted or unsubstituted biphenylylene, substituted or unsubstituted naphthylene, substituted or unsubstituted pyridylene, or a combination thereof;
more preferably, L, L′, and L″ are, at each occurrence identically or differently, selected from a single bond or phenylene.

7. The compound of claim 1, wherein Rᵥ and R_{z} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 20 carbon atoms, substituted or unsubstituted heteroaryl having 3 to 20 carbon atoms, cyano, and combinations thereof;
preferably, Rᵥ and R_{z} are, at each occurrence identically or differently, selected from the group consisting of: hydrogen, deuterium, fluorine, substituted or unsubstituted phenyl, substituted or unsubstituted naphthyl, substituted or unsubstituted biphenyl, substituted or unsubstituted terphenyl, substituted or unsubstituted triphenylenyl, substituted or unsubstituted fluorenyl, substituted or unsubstituted pyridyl, substituted or unsubstituted carbazolyl, substituted or unsubstituted dibenzofuryl, substituted or unsubstituted dibenzothienyl, cyano, and combinations thereof.

8. The compound of claim 1, wherein Rₓ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 20 carbon atoms, substituted or unsubstituted heteroaryl having 3 to 20 carbon atoms, and combinations thereof;
preferably, Rₓ is, at each occurrence identically or differently, selected from the group consisting of: hydrogen, deuterium, fluorine, substituted or unsubstituted phenyl, substituted or unsubstituted naphthyl, substituted or unsubstituted biphenyl, substituted or unsubstituted terphenyl, substituted or unsubstituted triphenylenyl, substituted or unsubstituted fluorenyl, substituted or unsubstituted pyridyl, substituted or unsubstituted carbazolyl, substituted or unsubstituted dibenzofuryl, substituted or unsubstituted dibenzothienyl, and combinations thereof.

9. The compound of claim 1, wherein the compound is selected from the group consisting of Compound A-1 to Compound A-882 having the following structures: 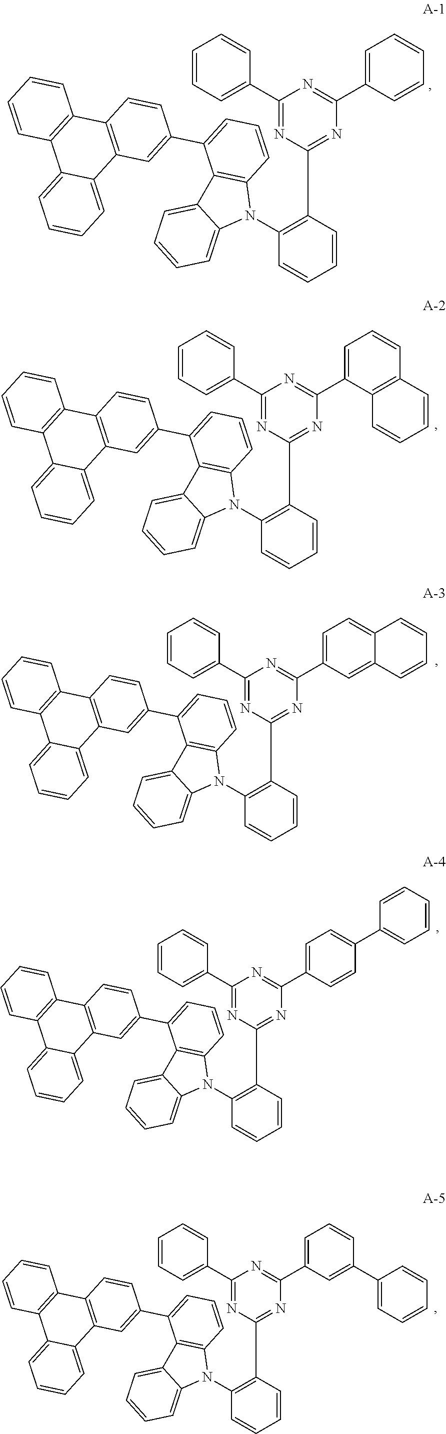 [SMILES: c1ccc(-c2nc(-c3ccccc3)nc(-c3ccccc3-n3c4ccccc4c4c(-c5ccc6c7ccccc7c7ccccc7c6c5)cccc43)n2)cc1 ; c1ccc(-c2nc(-c3ccccc3-n3c4ccccc4c4c(-c5ccc6c7ccccc7c7ccccc7c6c5)cccc43)nc(-c3cccc4ccccc34)n2)cc1 ; c1ccc(-c2nc(-c3ccc4ccccc4c3)nc(-c3ccccc3-n3c4ccccc4c4c(-c5ccc6c7ccccc7c7ccccc7c6c5)cccc43)n2)cc1 ; c1ccc(-c2ccc(-c3nc(-c4ccccc4)nc(-c4ccccc4-n4c5ccccc5c5c(-c6ccc7c8ccccc8c8ccccc8c7c6)cccc54)n3)cc2)cc1 ; c1ccc(-c2cccc(-c3nc(-c4ccccc4)nc(-c4ccccc4-n4c5ccccc5c5c(-c6ccc7c8ccccc8c8ccccc8c7c6)cccc54)n3)c2)cc1] 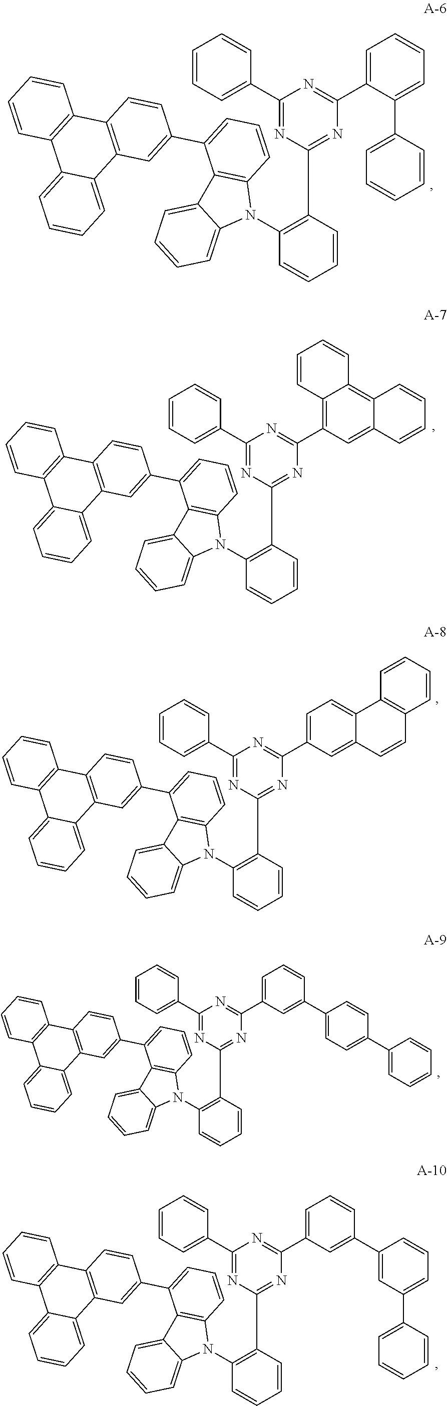 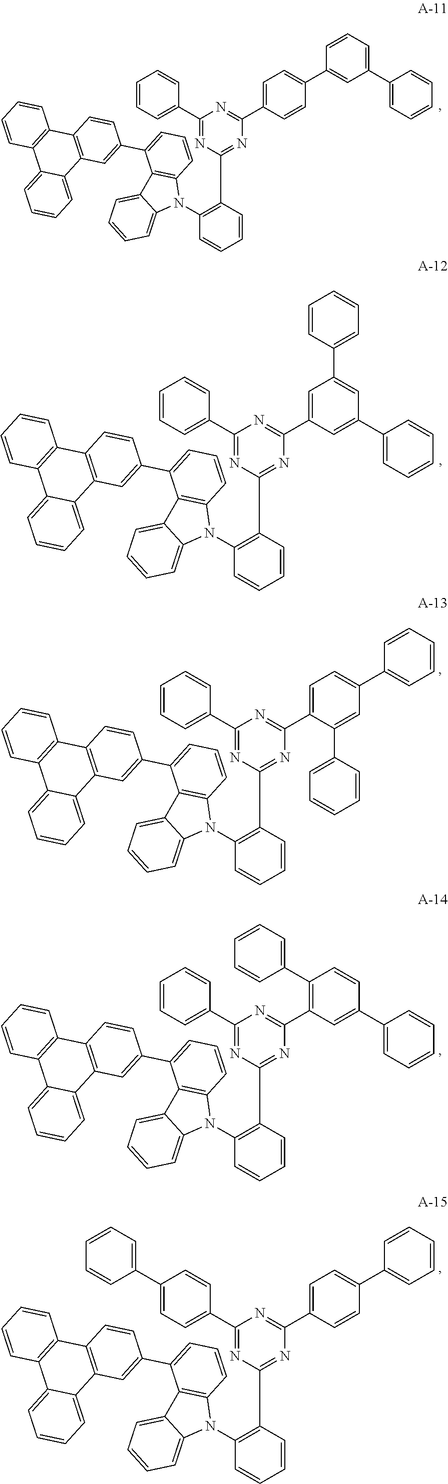 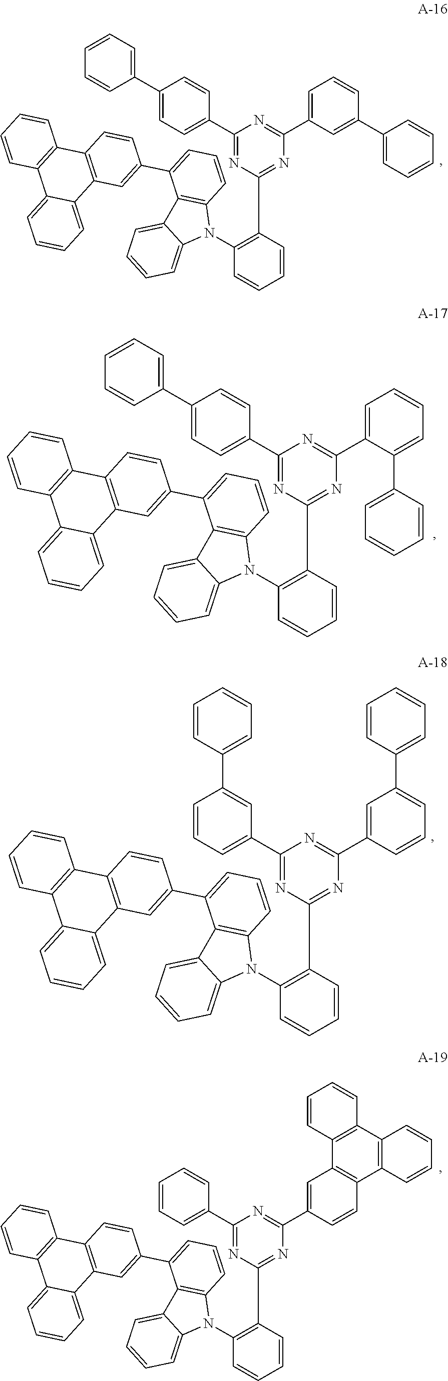 [SMILES: c1ccc(-c2ccc(-c3nc(-c4cccc(-c5ccccc5)c4)nc(-c4ccccc4-n4c5ccccc5c5c(-c6ccc7c8ccccc8c8ccccc8c7c6)cccc54)n3)cc2)cc1 ; c1ccc(-c2ccc(-c3nc(-c4ccccc4-c4ccccc4)nc(-c4ccccc4-n4c5ccccc5c5c(-c6ccc7c8ccccc8c8ccccc8c7c6)cccc54)n3)cc2)cc1 ; c1ccc(-c2cccc(-c3nc(-c4cccc(-c5ccccc5)c4)nc(-c4ccccc4-n4c5ccccc5c5c(-c6ccc7c8ccccc8c8ccccc8c7c6)cccc54)n3)c2)cc1 ; c1ccc(-c2nc(-c3ccc4c5ccccc5c5ccccc5c4c3)nc(-c3ccccc3-n3c4ccccc4c4c(-c5ccc6c7ccccc7c7ccccc7c6c5)cccc43)n2)cc1] 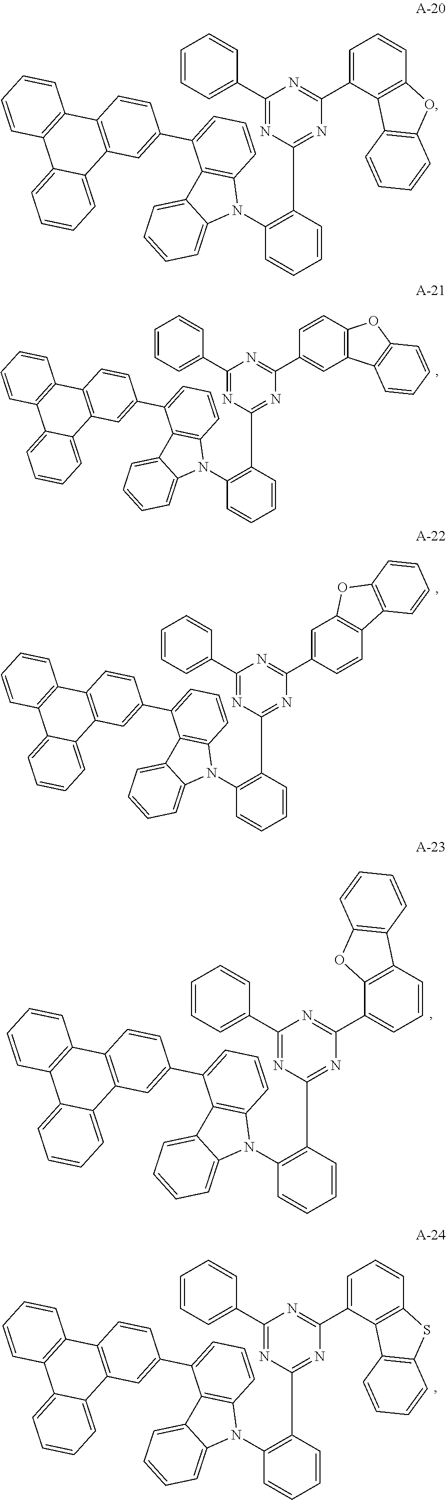 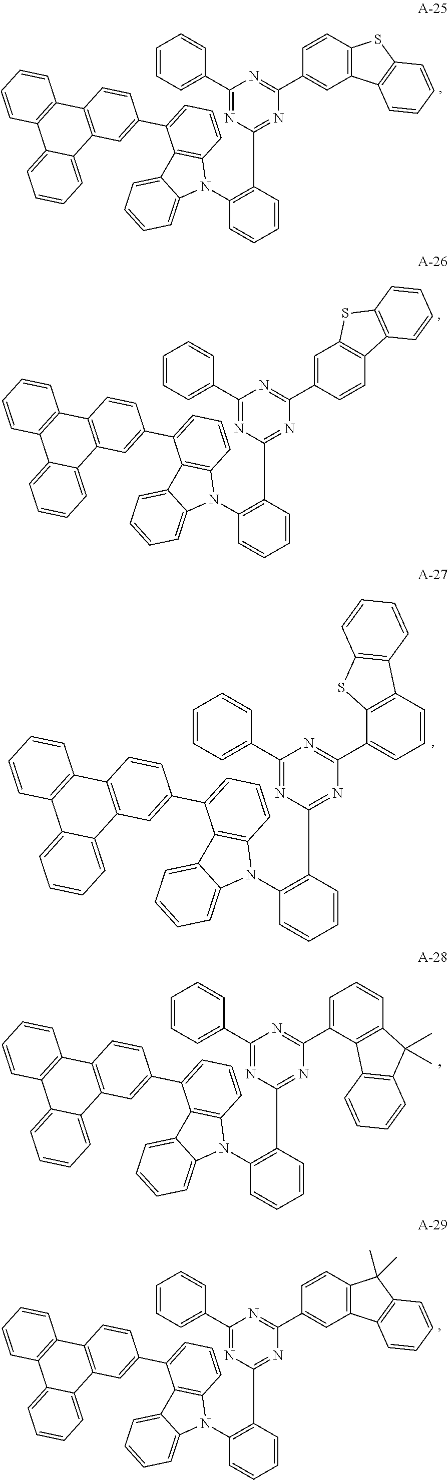 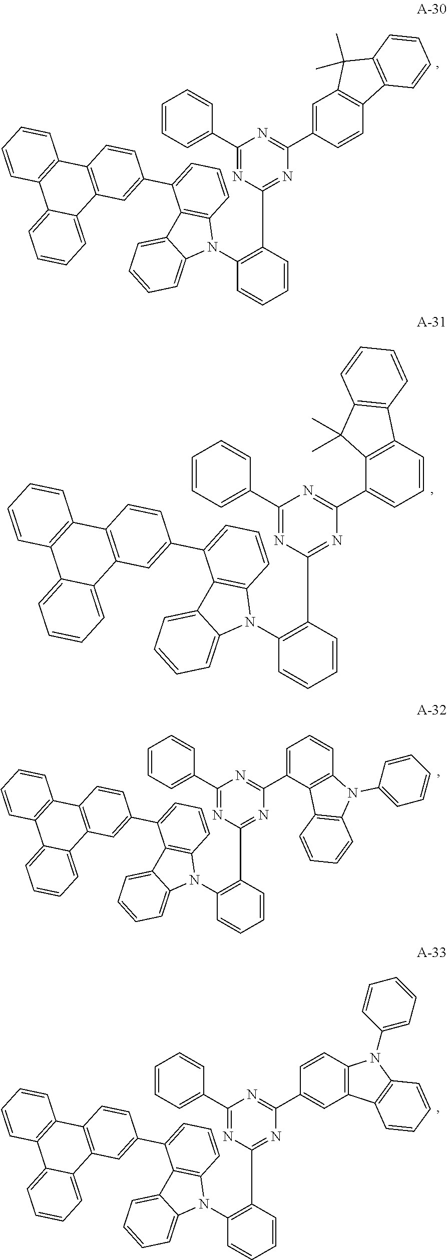 [SMILES: CC1(C)c2ccccc2-c2ccc(-c3nc(-c4ccccc4)nc(-c4ccccc4-n4c5ccccc5c5c(-c6ccc7c8ccccc8c8ccccc8c7c6)cccc54)n3)cc21 ; CC1(C)c2ccccc2-c2cccc(-c3nc(-c4ccccc4)nc(-c4ccccc4-n4c5ccccc5c5c(-c6ccc7c8ccccc8c8ccccc8c7c6)cccc54)n3)c21 ; c1ccc(-c2nc(-c3ccccc3-n3c4ccccc4c4c(-c5ccc6c7ccccc7c7ccccc7c6c5)cccc43)nc(-c3cccc4c3c3ccccc3n4-c3ccccc3)n2)cc1 ; c1ccc(-c2nc(-c3ccc4c(c3)c3ccccc3n4-c3ccccc3)nc(-c3ccccc3-n3c4ccccc4c4c(-c5ccc6c7ccccc7c7ccccc7c6c5)cccc43)n2)cc1] 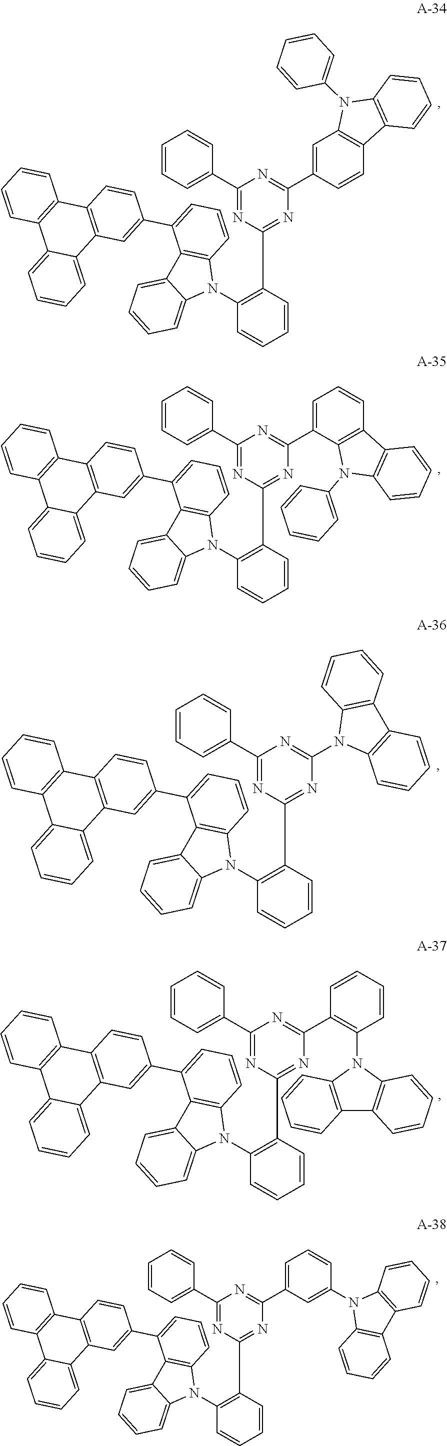 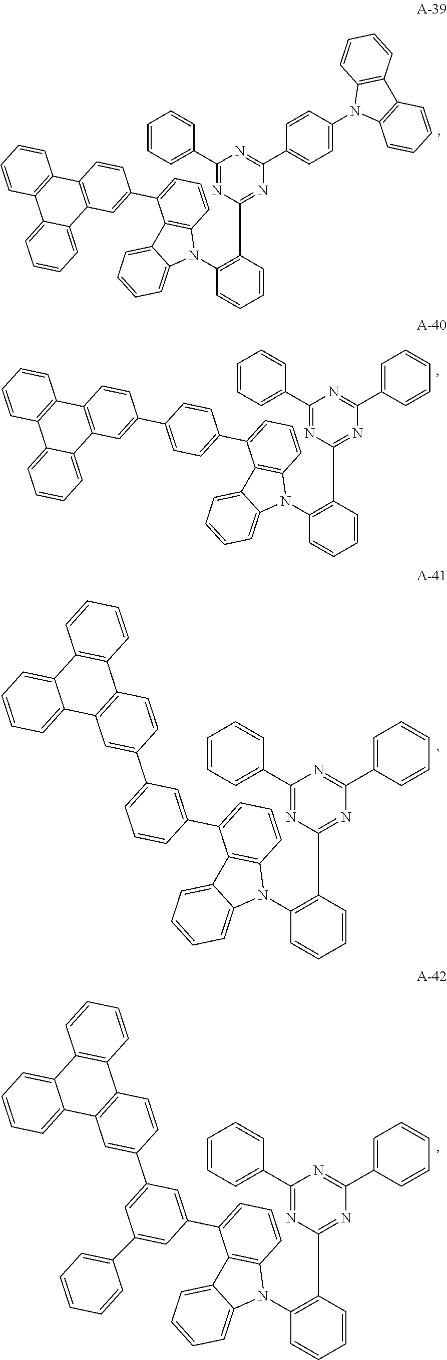 [SMILES: c1ccc(-c2nc(-c3ccc(-n4c5ccccc5c5ccccc54)cc3)nc(-c3ccccc3-n3c4ccccc4c4c(-c5ccc6c7ccccc7c7ccccc7c6c5)cccc43)n2)cc1 ; c1ccc(-c2nc(-c3ccccc3)nc(-c3ccccc3-n3c4ccccc4c4c(-c5ccc(-c6ccc7c8ccccc8c8ccccc8c7c6)cc5)cccc43)n2)cc1 ; c1ccc(-c2nc(-c3ccccc3)nc(-c3ccccc3-n3c4ccccc4c4c(-c5cccc(-c6ccc7c8ccccc8c8ccccc8c7c6)c5)cccc43)n2)cc1 ; c1ccc(-c2cc(-c3ccc4c5ccccc5c5ccccc5c4c3)cc(-c3cccc4c3c3ccccc3n4-c3ccccc3-c3nc(-c4ccccc4)nc(-c4ccccc4)n3)c2)cc1] 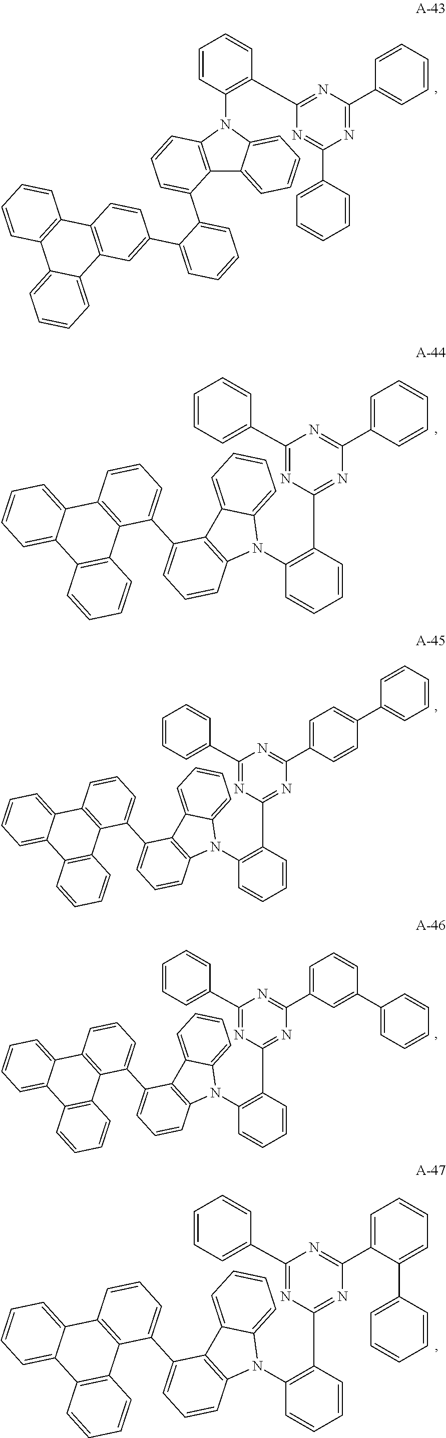 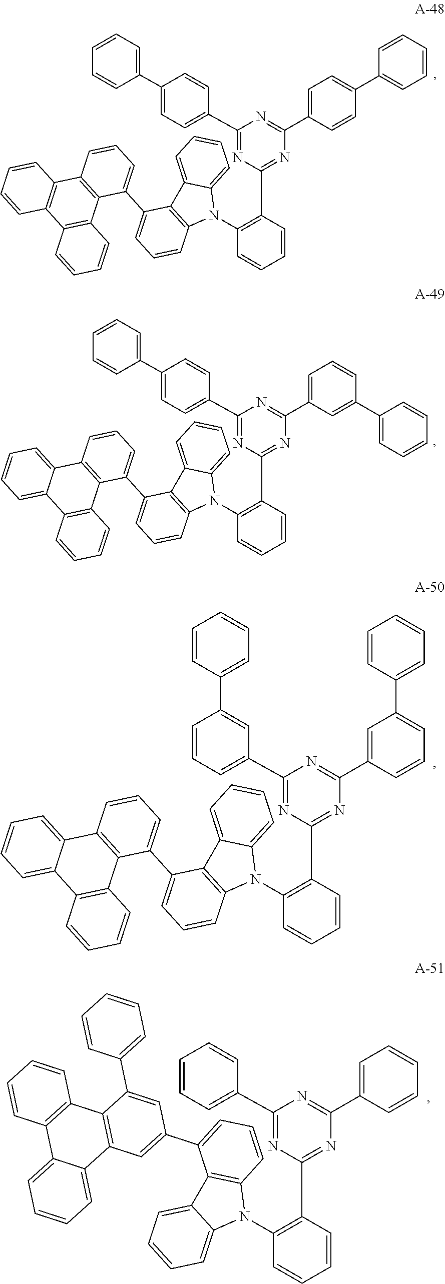 [SMILES: c1ccc(-c2ccc(-c3nc(-c4ccc(-c5ccccc5)cc4)nc(-c4ccccc4-n4c5ccccc5c5c(-c6cccc7c8ccccc8c8ccccc8c67)cccc54)n3)cc2)cc1 ; c1ccc(-c2ccc(-c3nc(-c4cccc(-c5ccccc5)c4)nc(-c4ccccc4-n4c5ccccc5c5c(-c6cccc7c8ccccc8c8ccccc8c67)cccc54)n3)cc2)cc1 ; c1ccc(-c2cccc(-c3nc(-c4cccc(-c5ccccc5)c4)nc(-c4ccccc4-n4c5ccccc5c5c(-c6cccc7c8ccccc8c8ccccc8c67)cccc54)n3)c2)cc1 ; c1ccc(-c2nc(-c3ccccc3)nc(-c3ccccc3-n3c4ccccc4c4c(-c5cc(-c6ccccc6)c6c7ccccc7c7ccccc7c6c5)cccc43)n2)cc1] 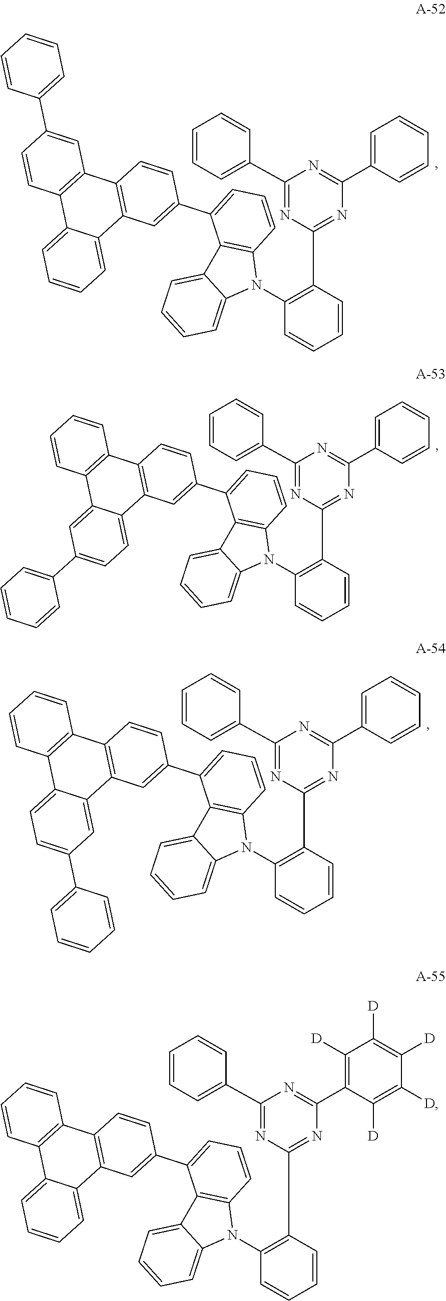 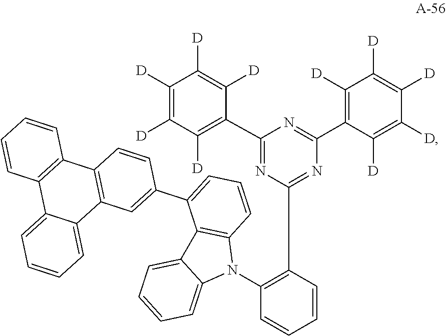 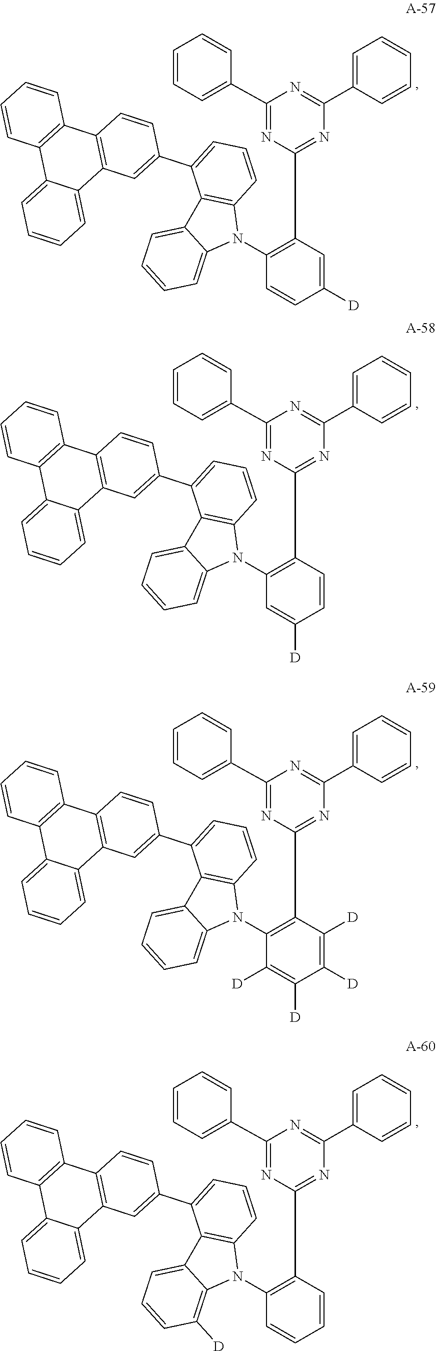 [SMILES: [2H]c1ccc(-n2c3ccccc3c3c(-c4ccc5c6ccccc6c6ccccc6c5c4)cccc32)c(-c2nc(-c3ccccc3)nc(-c3ccccc3)n2)c1 ; [2H]c1ccc(-c2nc(-c3ccccc3)nc(-c3ccccc3)n2)c(-n2c3ccccc3c3c(-c4ccc5c6ccccc6c6ccccc6c5c4)cccc32)c1 ; [2H]c1c([2H])c([2H])c(-n2c3ccccc3c3c(-c4ccc5c6ccccc6c6ccccc6c5c4)cccc32)c(-c2nc(-c3ccccc3)nc(-c3ccccc3)n2)c1[2H] ; [2H]c1cccc2c3c(-c4ccc5c6ccccc6c6ccccc6c5c4)cccc3n(-c3ccccc3-c3nc(-c4ccccc4)nc(-c4ccccc4)n3)c12] 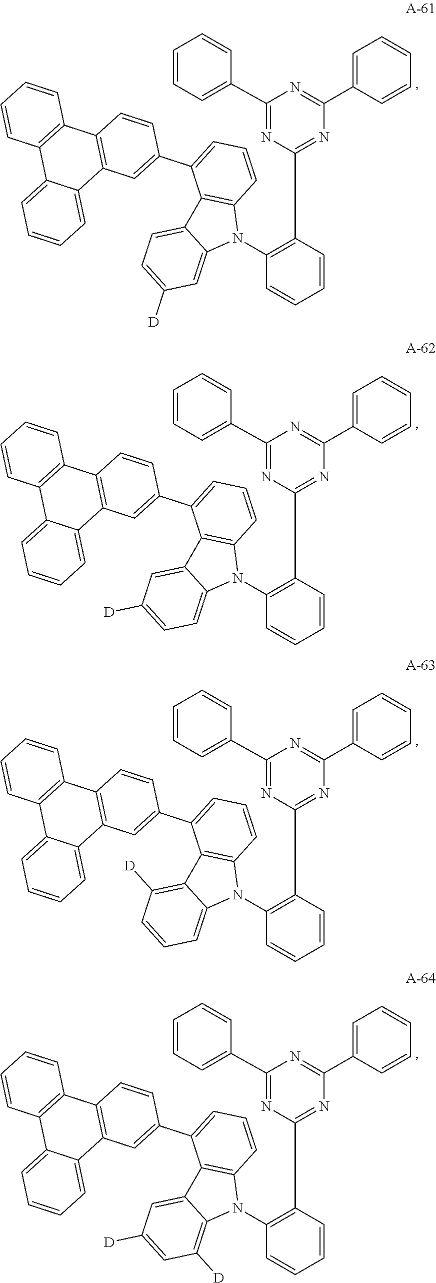 [SMILES: [2H]c1ccc2c3c(-c4ccc5c6ccccc6c6ccccc6c5c4)cccc3n(-c3ccccc3-c3nc(-c4ccccc4)nc(-c4ccccc4)n3)c2c1 ; [2H]c1ccc2c(c1)c1c(-c3ccc4c5ccccc5c5ccccc5c4c3)cccc1n2-c1ccccc1-c1nc(-c2ccccc2)nc(-c2ccccc2)n1 ; [2H]c1cccc2c1c1c(-c3ccc4c5ccccc5c5ccccc5c4c3)cccc1n2-c1ccccc1-c1nc(-c2ccccc2)nc(-c2ccccc2)n1 ; [2H]c1cc([2H])c2c(c1)c1c(-c3ccc4c5ccccc5c5ccccc5c4c3)cccc1n2-c1ccccc1-c1nc(-c2ccccc2)nc(-c2ccccc2)n1] 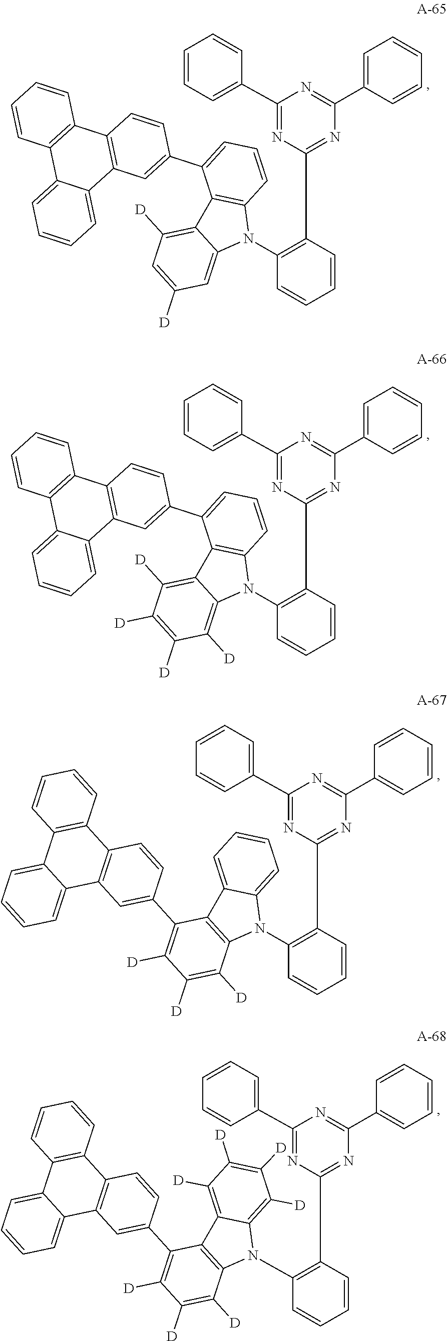 [SMILES: [2H]c1cc([2H])c2c3c(-c4ccc5c6ccccc6c6ccccc6c5c4)cccc3n(-c3ccccc3-c3nc(-c4ccccc4)nc(-c4ccccc4)n3)c2c1 ; [2H]c1c([2H])c([2H])c2c(c1[2H])c1c(-c3ccc4c5ccccc5c5ccccc5c4c3)cccc1n2-c1ccccc1-c1nc(-c2ccccc2)nc(-c2ccccc2)n1 ; [2H]c1c([2H])c([2H])c2c(c1-c1ccc3c4ccccc4c4ccccc4c3c1)c1ccccc1n2-c1ccccc1-c1nc(-c2ccccc2)nc(-c2ccccc2)n1 ; [2H]c1c([2H])c([2H])c2c(c1[2H])c1c(-c3ccc4c5ccccc5c5ccccc5c4c3)c([2H])c([2H])c([2H])c1n2-c1ccccc1-c1nc(-c2ccccc2)nc(-c2ccccc2)n1] 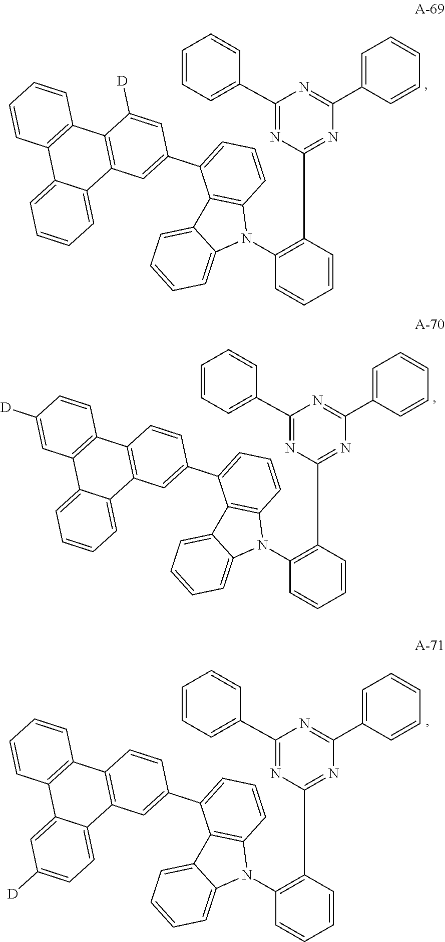 [SMILES: [2H]c1cc(-c2cccc3c2c2ccccc2n3-c2ccccc2-c2nc(-c3ccccc3)nc(-c3ccccc3)n2)cc2c3ccccc3c3ccccc3c12 ; [2H]c1ccc2c3ccc(-c4cccc5c4c4ccccc4n5-c4ccccc4-c4nc(-c5ccccc5)nc(-c5ccccc5)n4)cc3c3ccccc3c2c1 ; [2H]c1ccc2c(c1)c1ccccc1c1ccc(-c3cccc4c3c3ccccc3n4-c3ccccc3-c3nc(-c4ccccc4)nc(-c4ccccc4)n3)cc12] 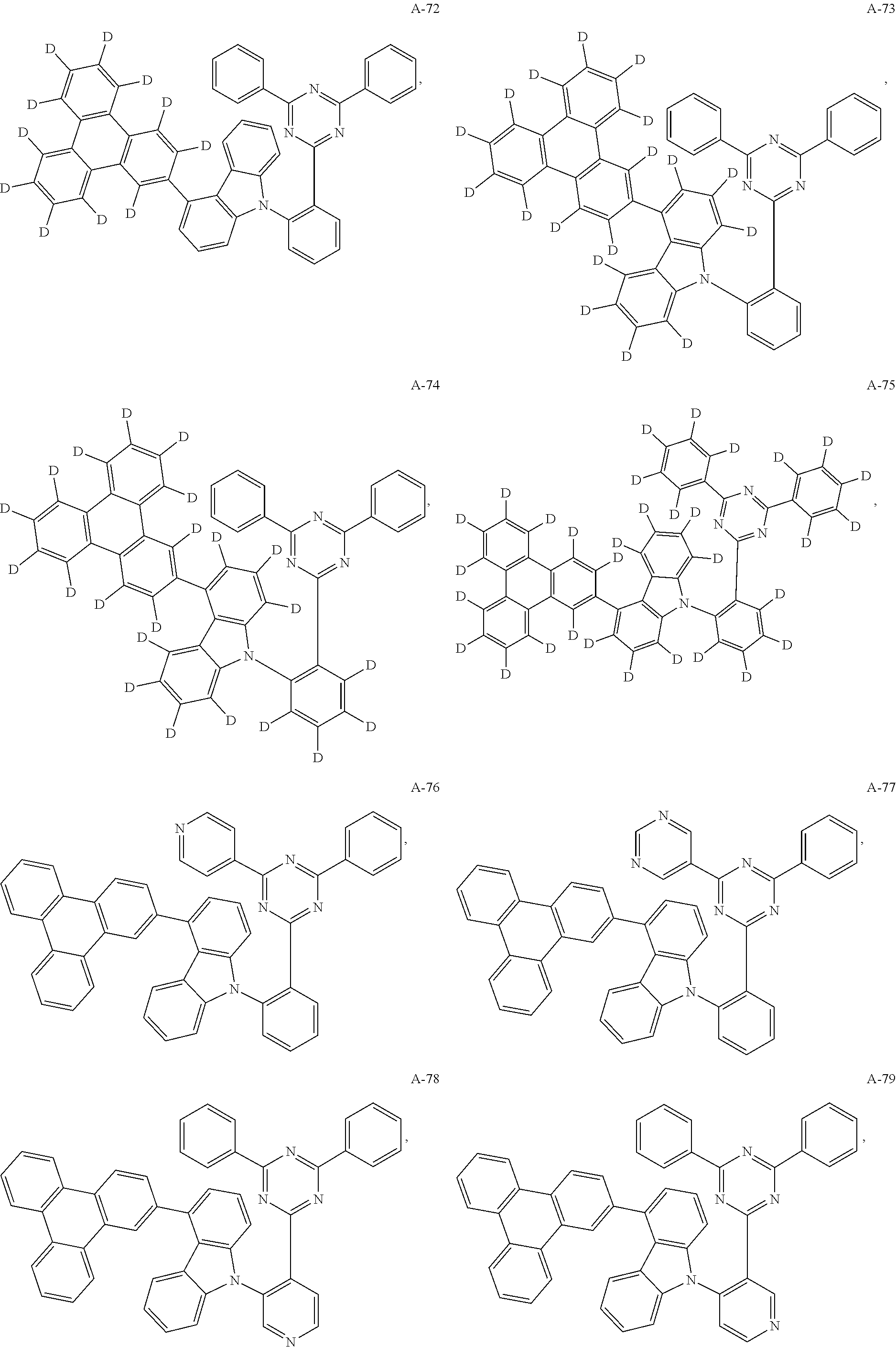 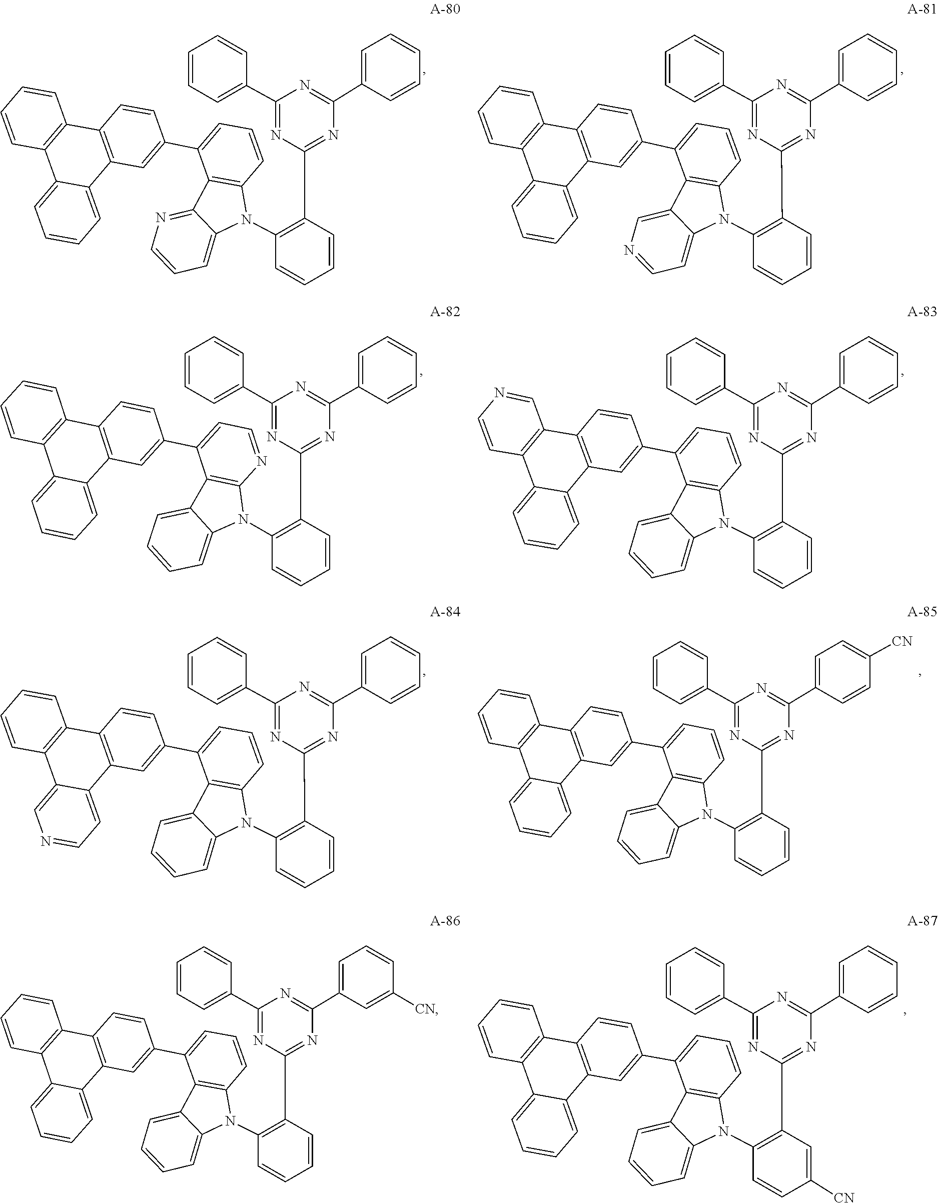 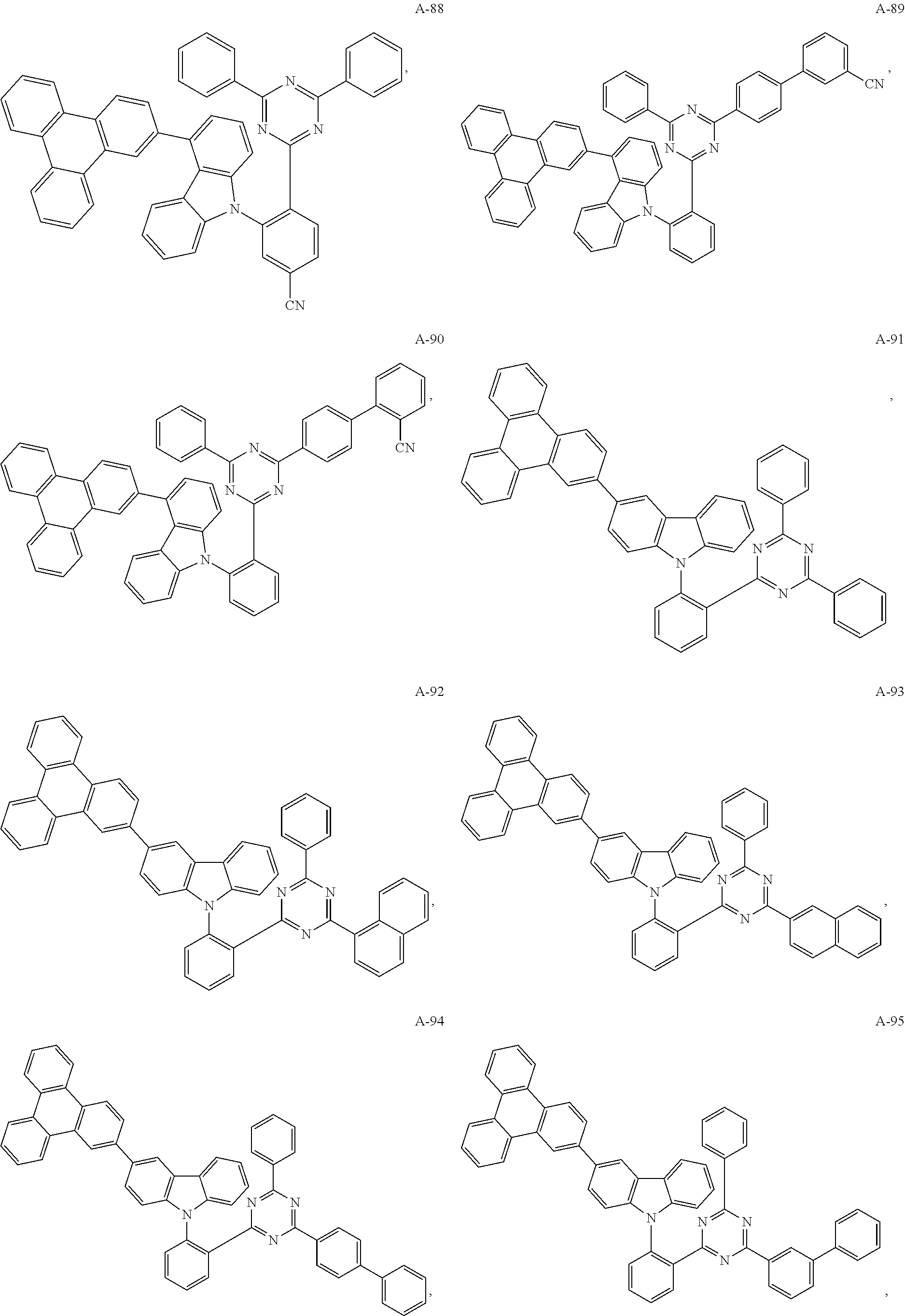 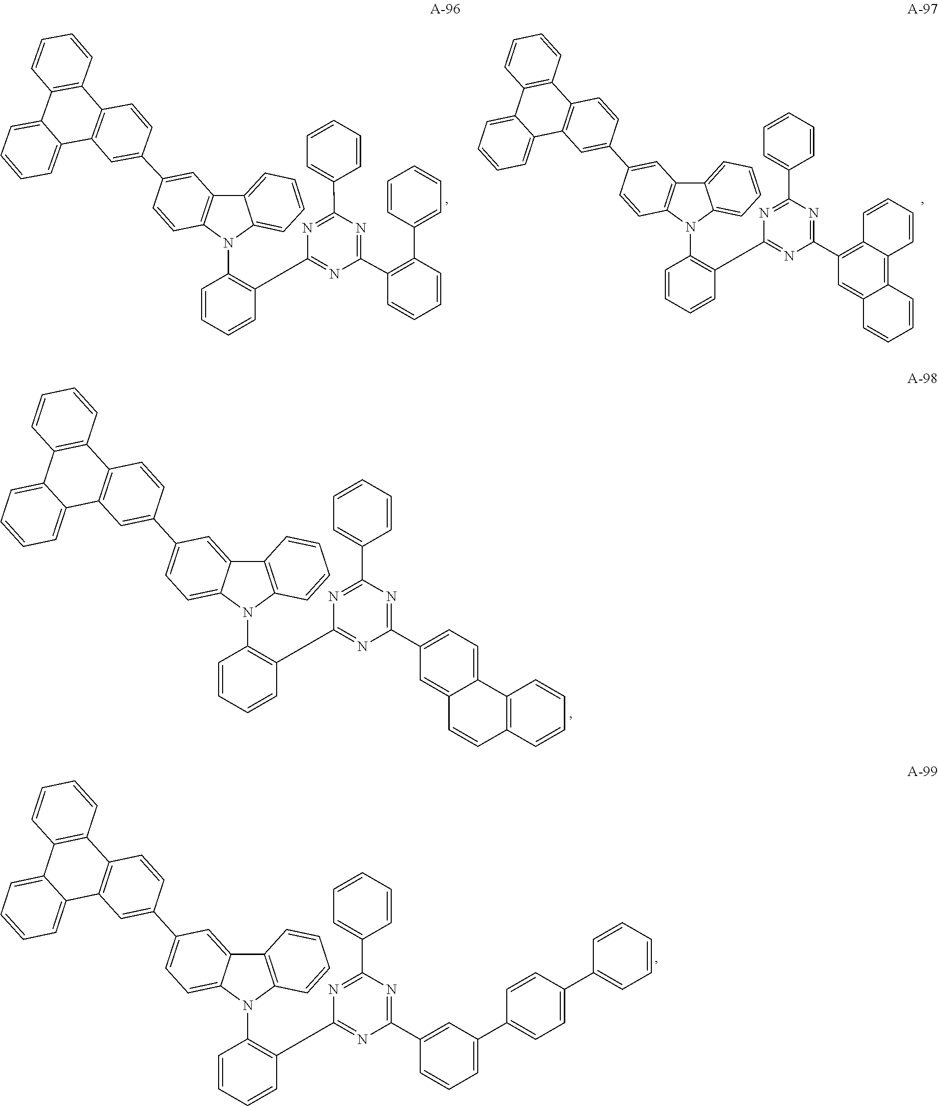 [SMILES: c1ccc(-c2nc(-c3ccccc3-c3ccccc3)nc(-c3ccccc3-n3c4ccccc4c4cc(-c5ccc6c7ccccc7c7ccccc7c6c5)ccc43)n2)cc1 ; c1ccc(-c2nc(-c3ccccc3-n3c4ccccc4c4cc(-c5ccc6c7ccccc7c7ccccc7c6c5)ccc43)nc(-c3cc4ccccc4c4ccccc34)n2)cc1 ; c1ccc(-c2nc(-c3ccc4c(ccc5ccccc54)c3)nc(-c3ccccc3-n3c4ccccc4c4cc(-c5ccc6c7ccccc7c7ccccc7c6c5)ccc43)n2)cc1 ; c1ccc(-c2ccc(-c3cccc(-c4nc(-c5ccccc5)nc(-c5ccccc5-n5c6ccccc6c6cc(-c7ccc8c9ccccc9c9ccccc9c8c7)ccc65)n4)c3)cc2)cc1] 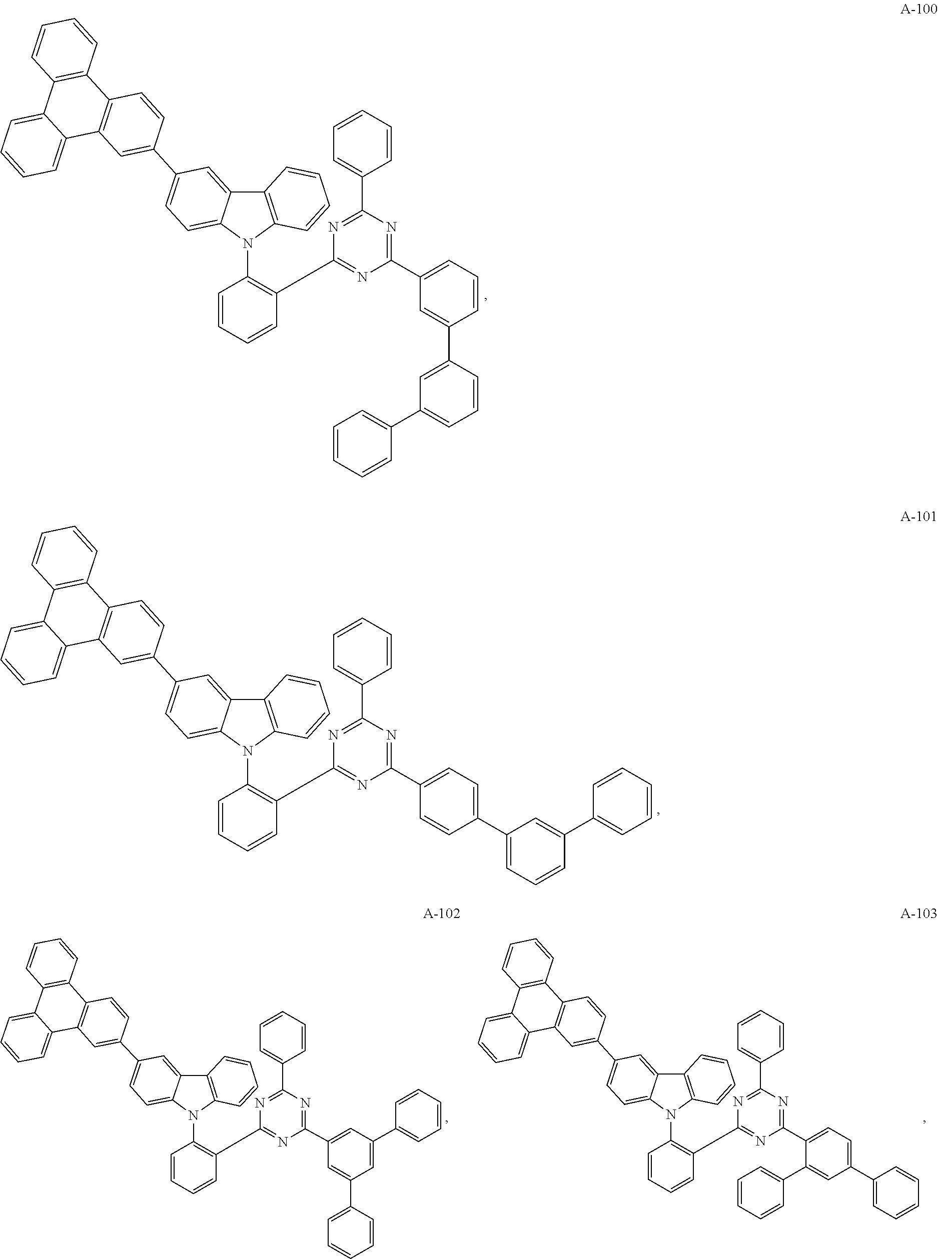 [SMILES: c1ccc(-c2cccc(-c3cccc(-c4nc(-c5ccccc5)nc(-c5ccccc5-n5c6ccccc6c6cc(-c7ccc8c9ccccc9c9ccccc9c8c7)ccc65)n4)c3)c2)cc1 ; c1ccc(-c2cccc(-c3ccc(-c4nc(-c5ccccc5)nc(-c5ccccc5-n5c6ccccc6c6cc(-c7ccc8c9ccccc9c9ccccc9c8c7)ccc65)n4)cc3)c2)cc1 ; c1ccc(-c2cc(-c3ccccc3)cc(-c3nc(-c4ccccc4)nc(-c4ccccc4-n4c5ccccc5c5cc(-c6ccc7c8ccccc8c8ccccc8c7c6)ccc54)n3)c2)cc1 ; c1ccc(-c2ccc(-c3nc(-c4ccccc4)nc(-c4ccccc4-n4c5ccccc5c5cc(-c6ccc7c8ccccc8c8ccccc8c7c6)ccc54)n3)c(-c3ccccc3)c2)cc1] 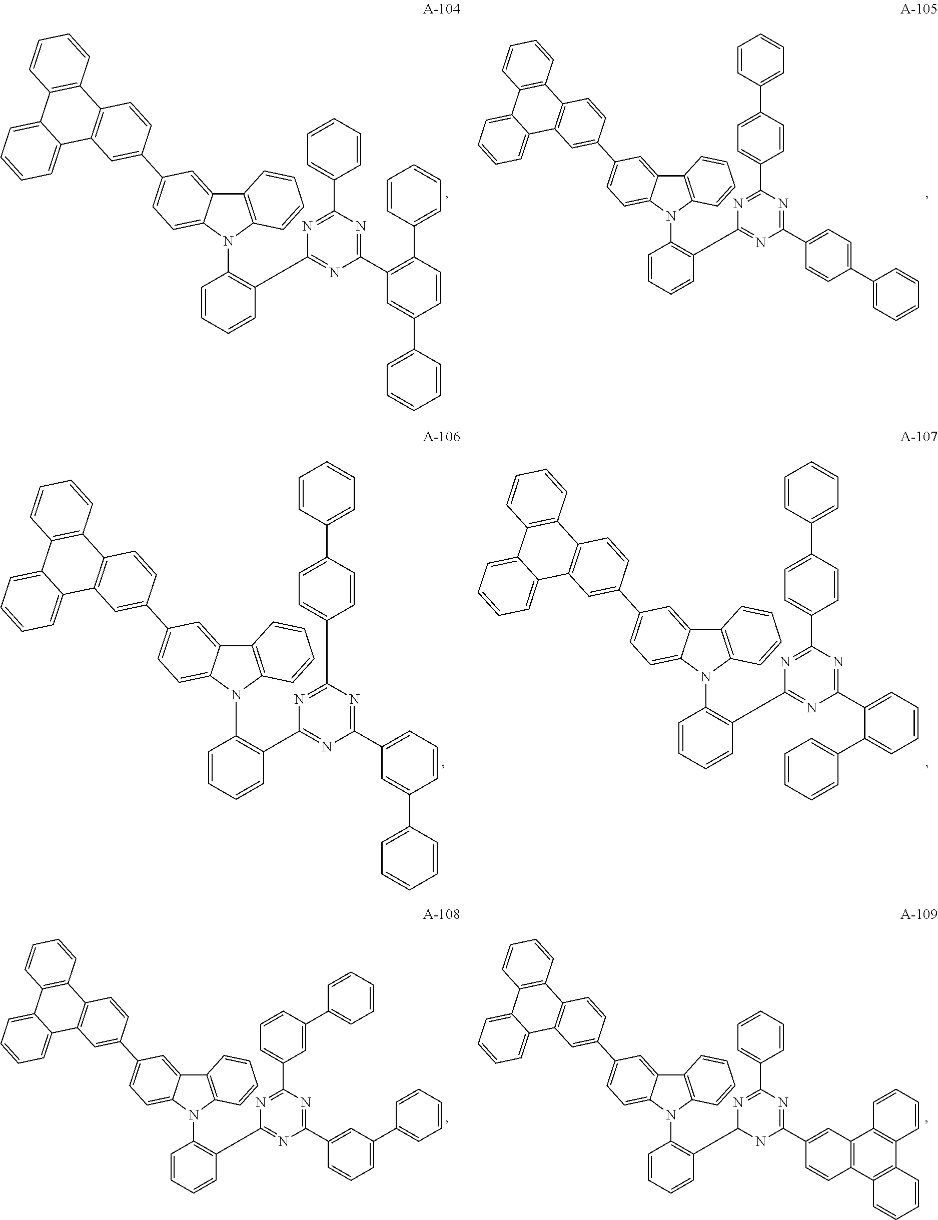 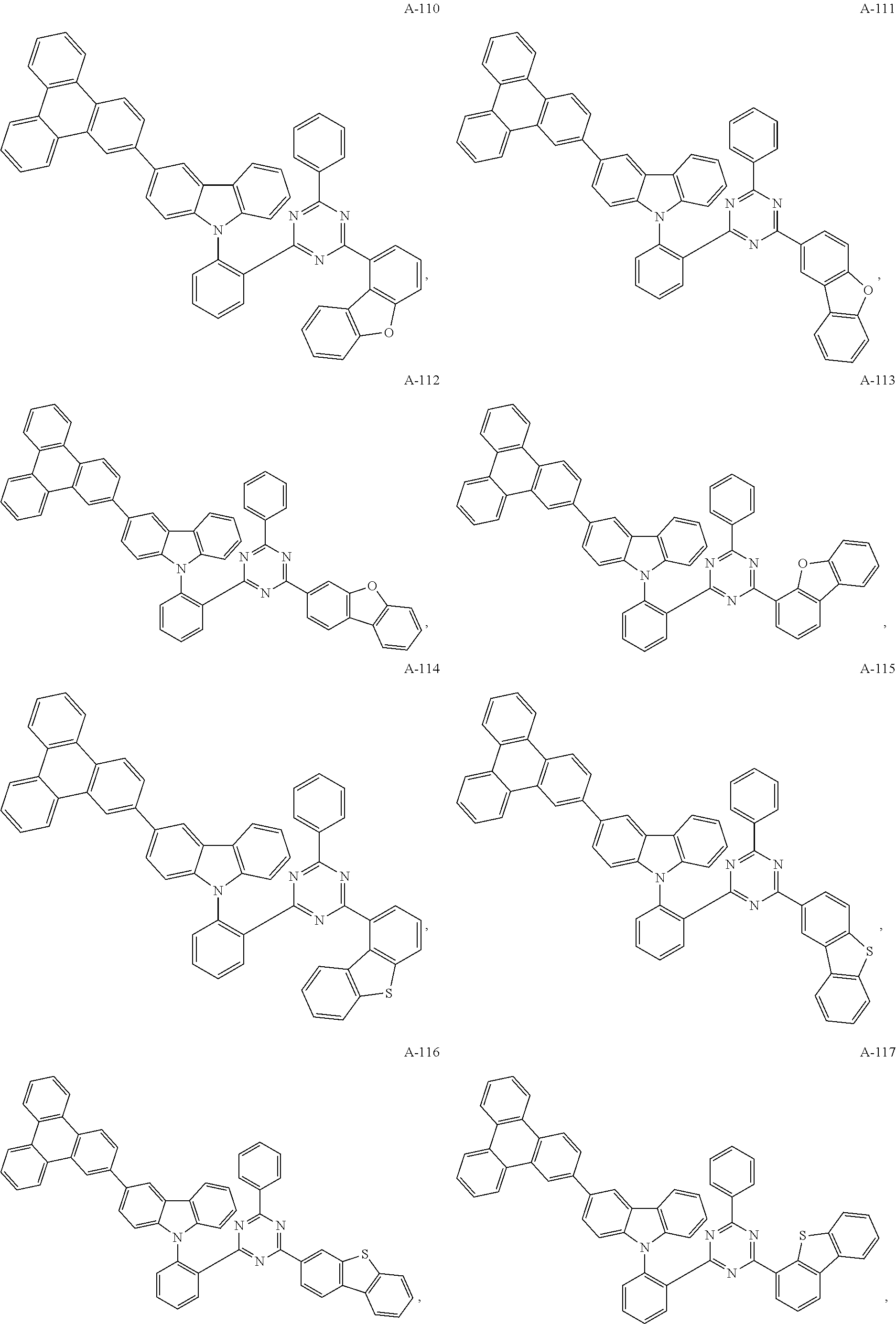 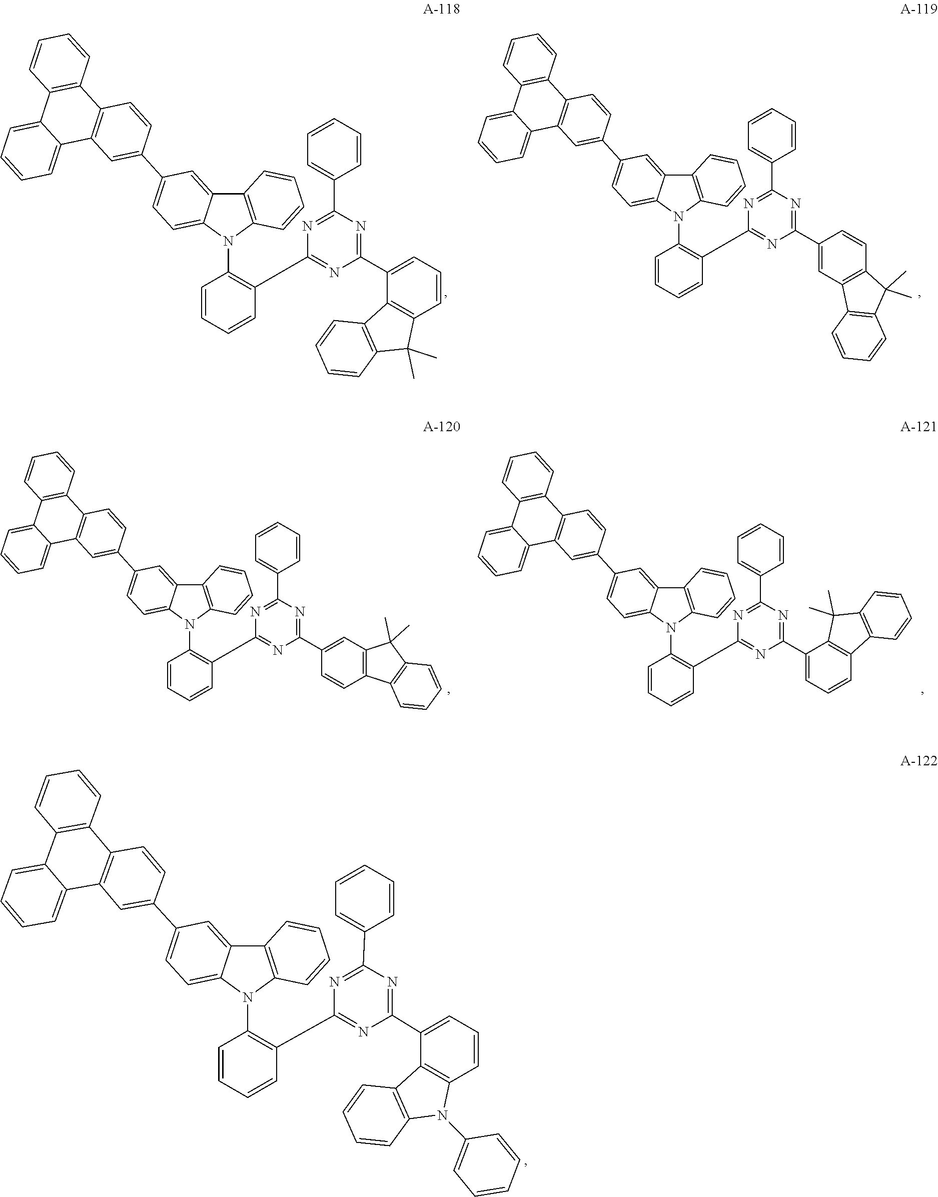 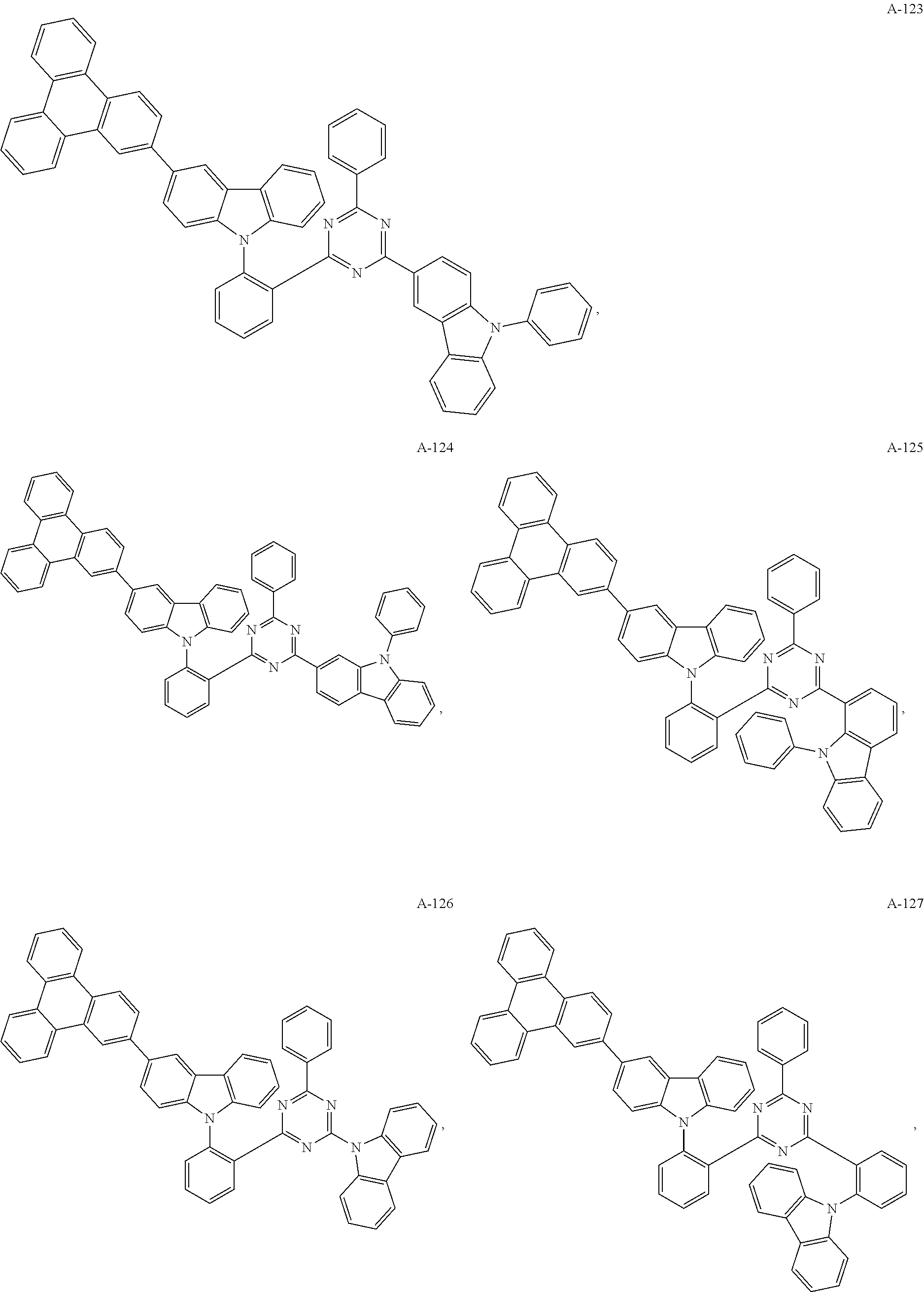 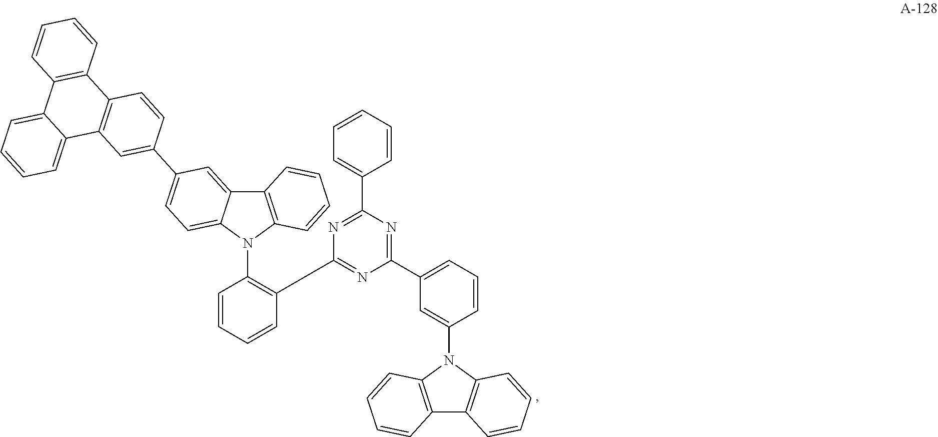 [SMILES: c1ccc(-c2nc(-c3cccc(-n4c5ccccc5c5ccccc54)c3)nc(-c3ccccc3-n3c4ccccc4c4cc(-c5ccc6c7ccccc7c7ccccc7c6c5)ccc43)n2)cc1] 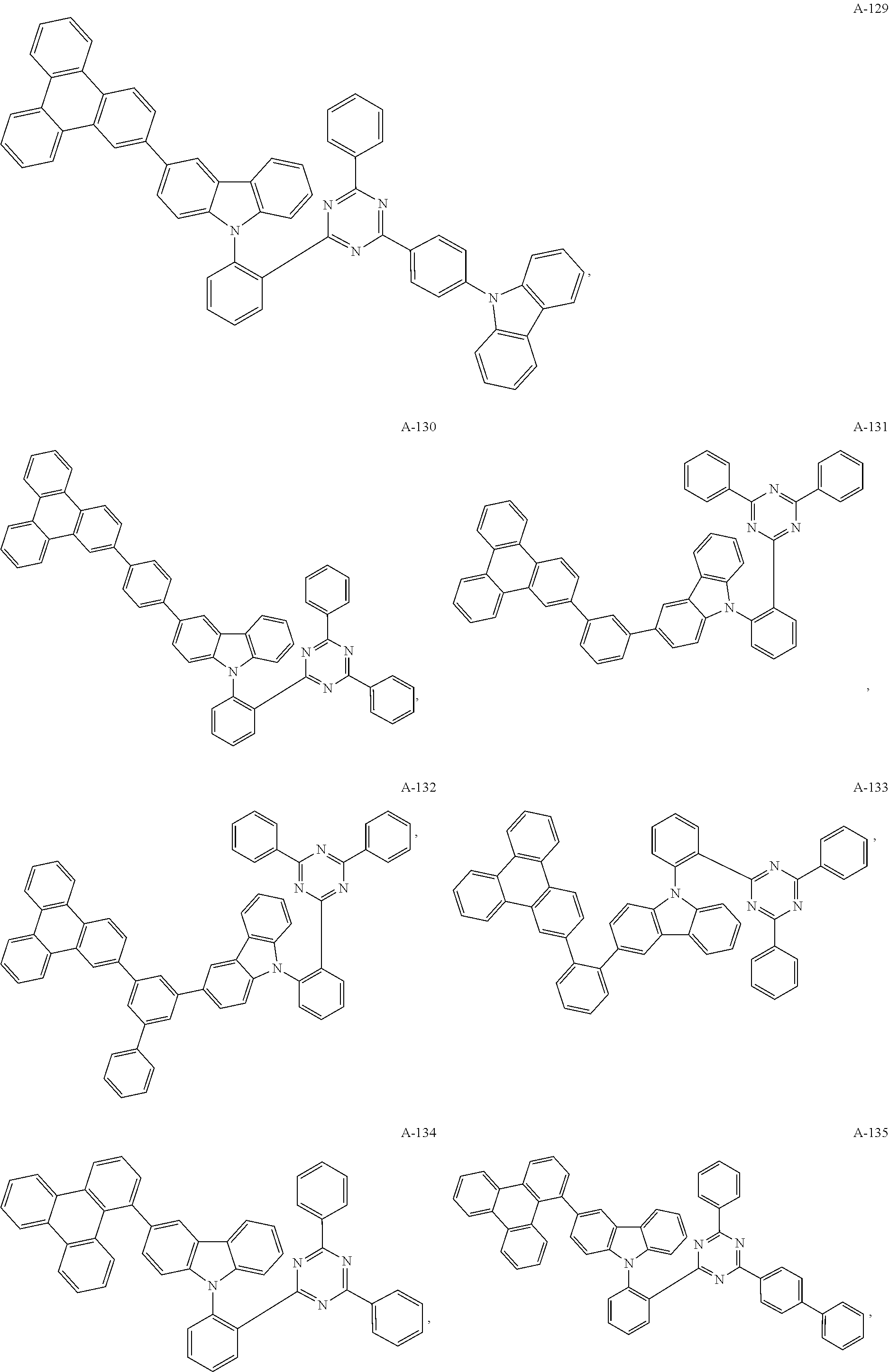 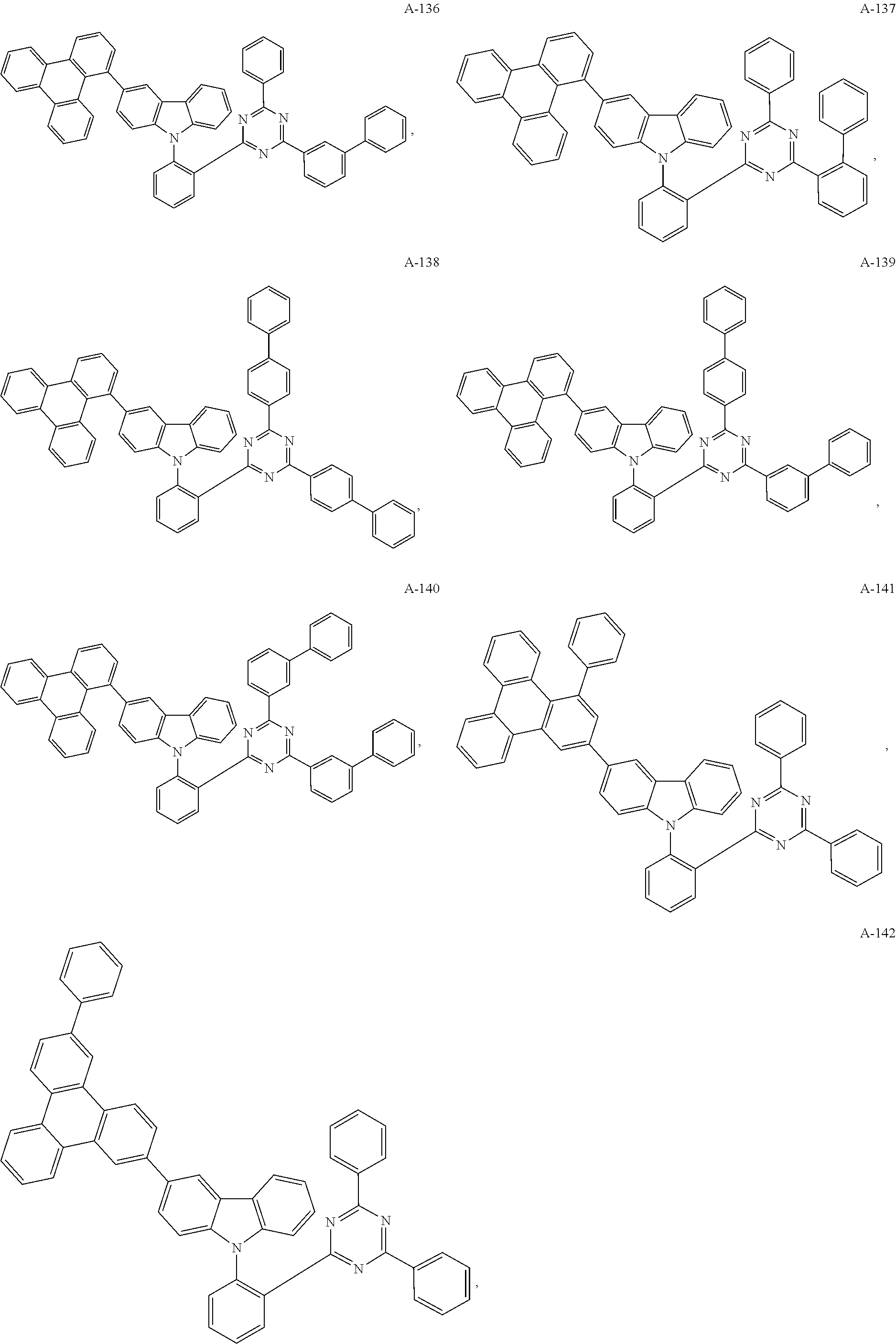 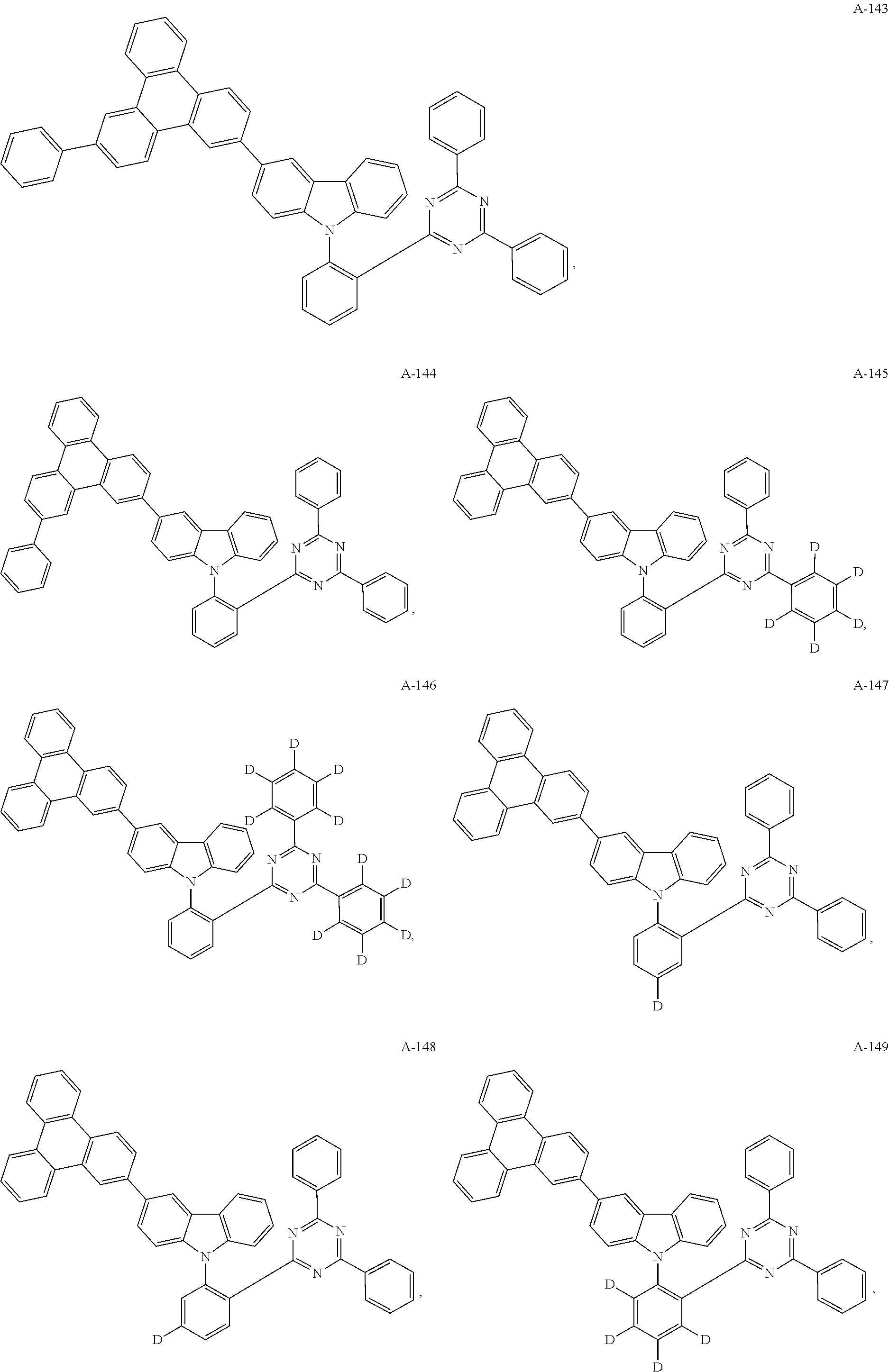 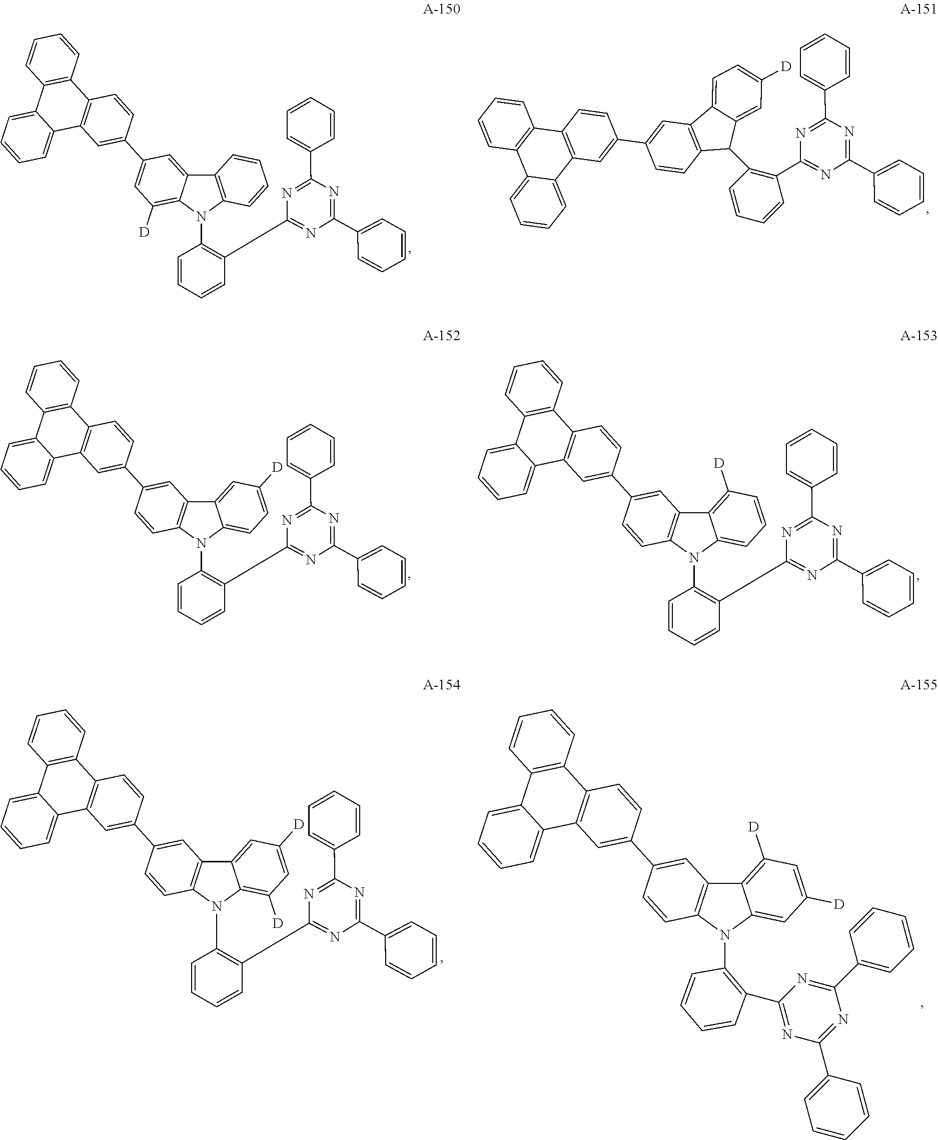 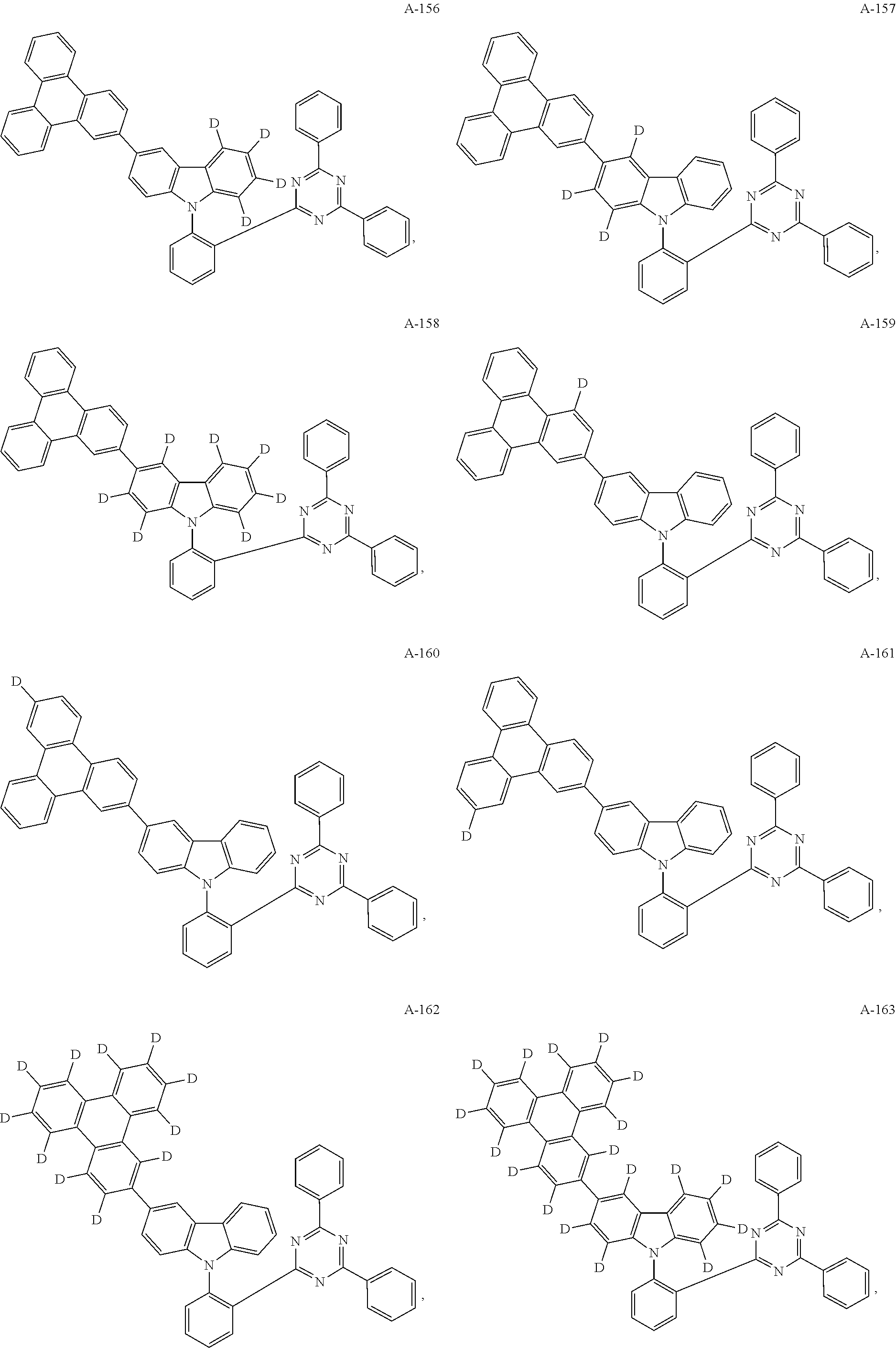 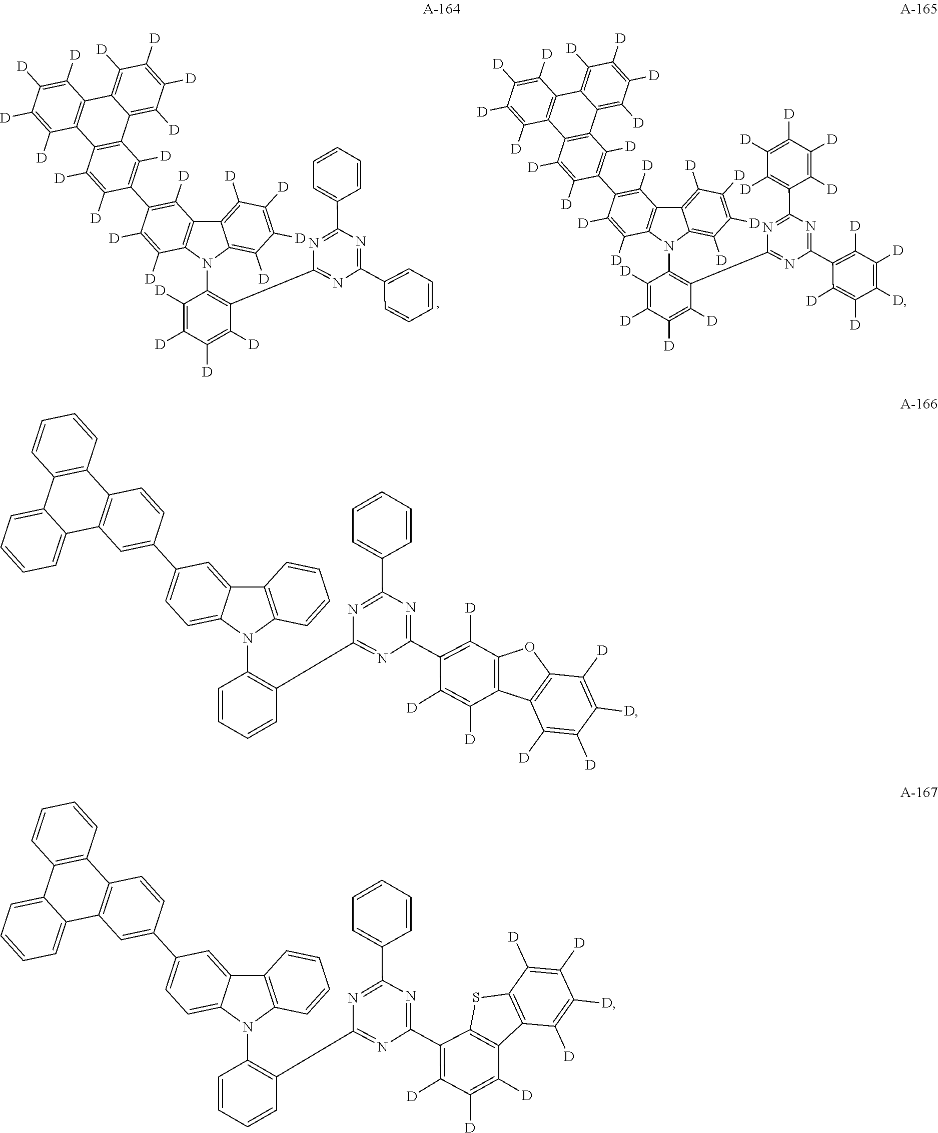 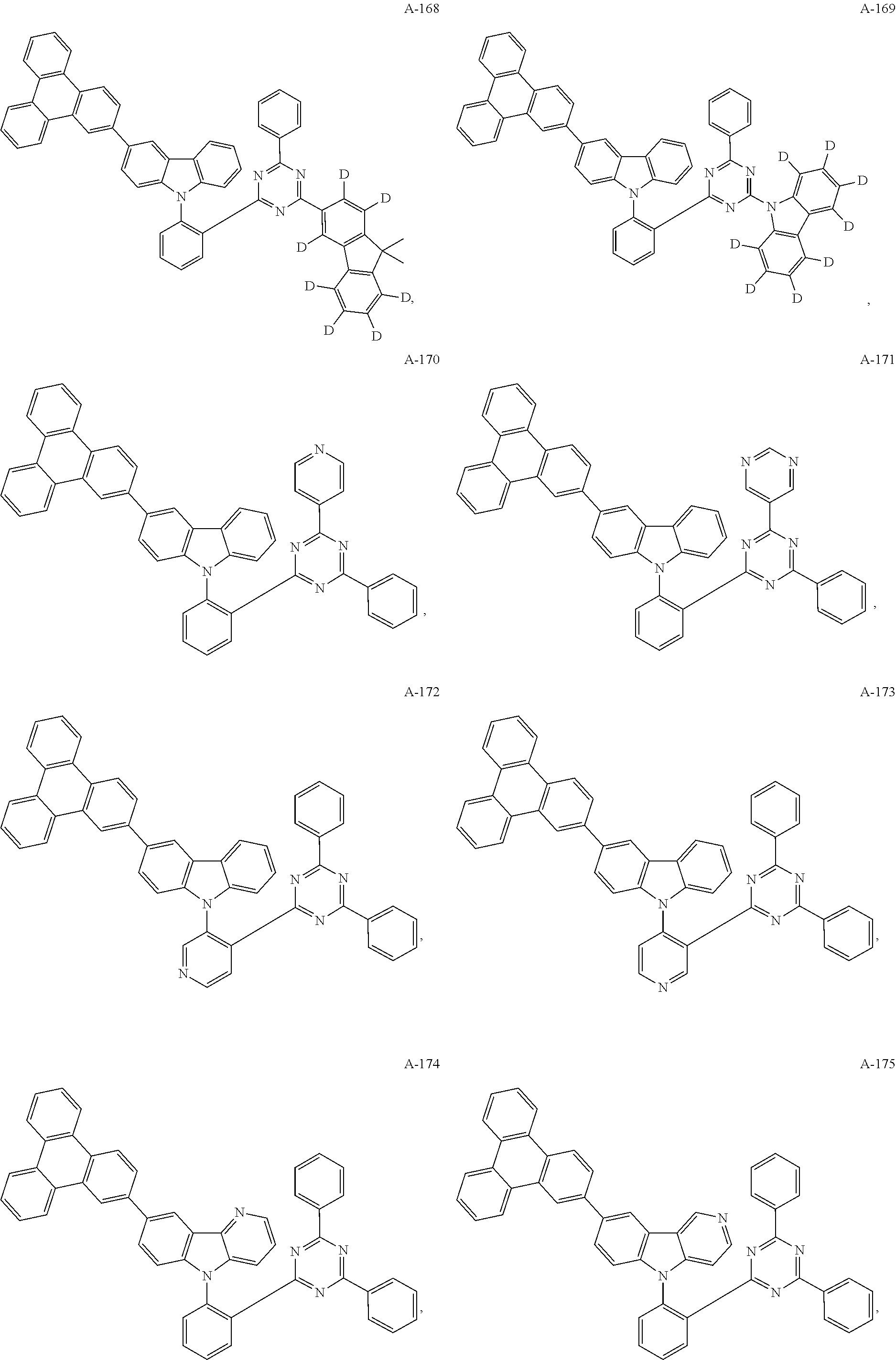 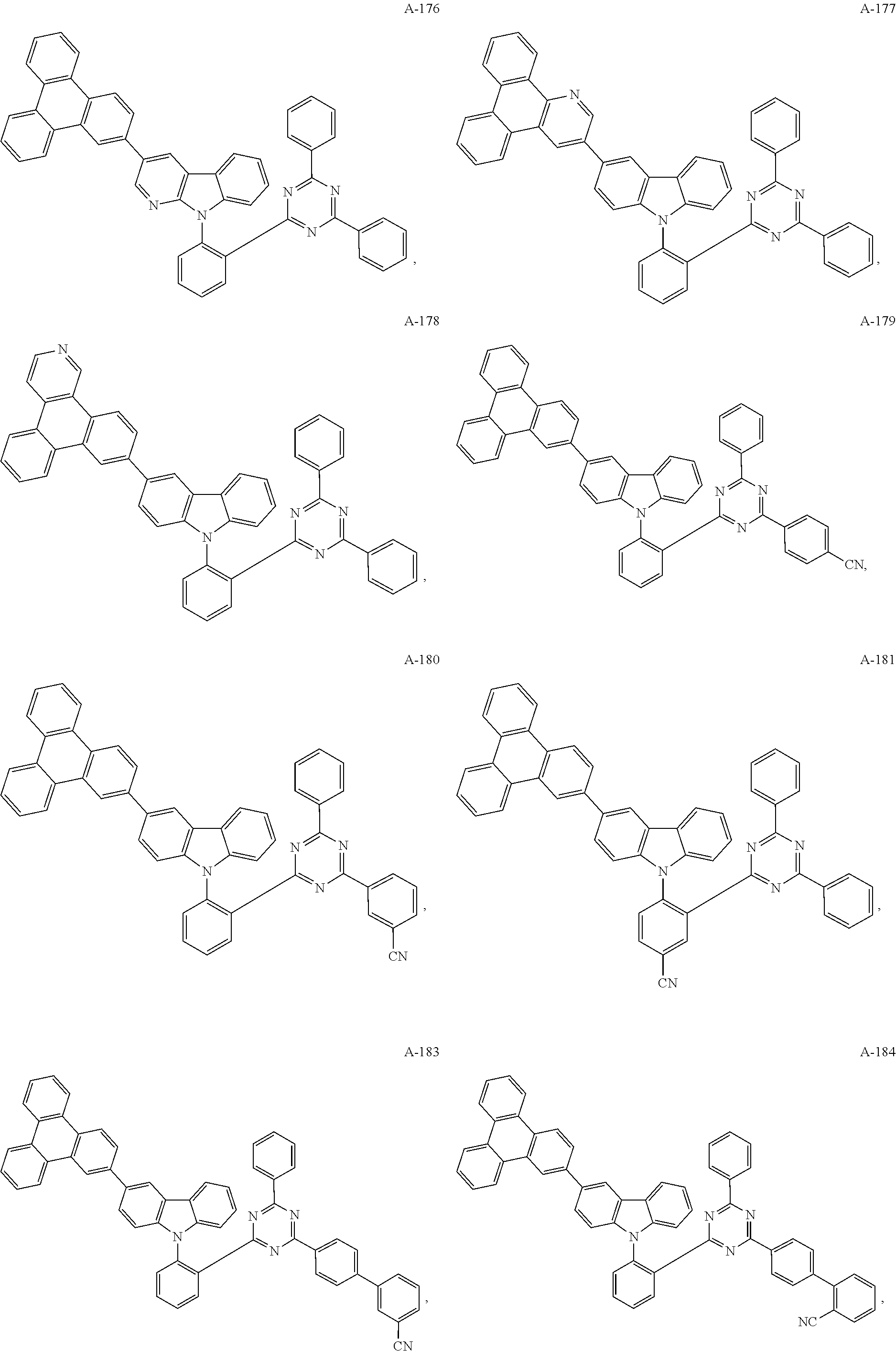 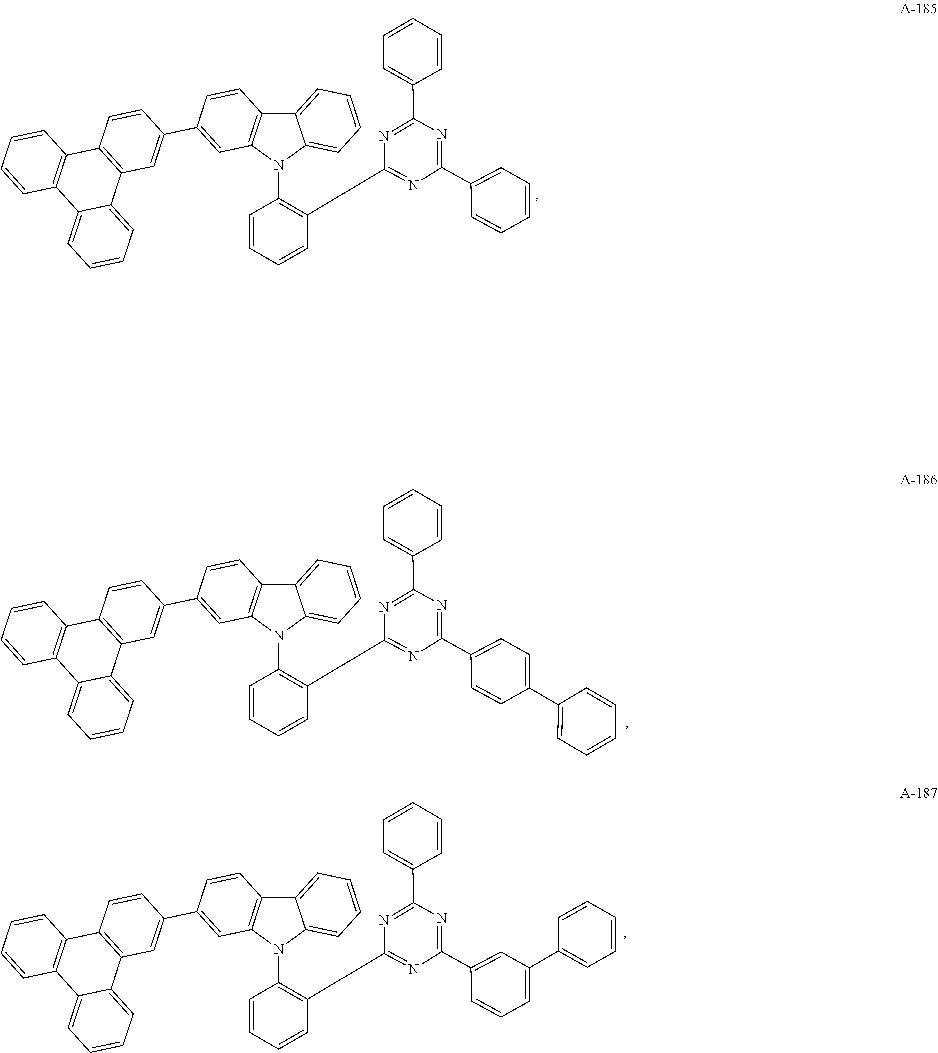 [SMILES: c1ccc(-c2nc(-c3ccccc3)nc(-c3ccccc3-n3c4ccccc4c4ccc(-c5ccc6c7ccccc7c7ccccc7c6c5)cc43)n2)cc1 ; c1ccc(-c2ccc(-c3nc(-c4ccccc4)nc(-c4ccccc4-n4c5ccccc5c5ccc(-c6ccc7c8ccccc8c8ccccc8c7c6)cc54)n3)cc2)cc1 ; c1ccc(-c2cccc(-c3nc(-c4ccccc4)nc(-c4ccccc4-n4c5ccccc5c5ccc(-c6ccc7c8ccccc8c8ccccc8c7c6)cc54)n3)c2)cc1] 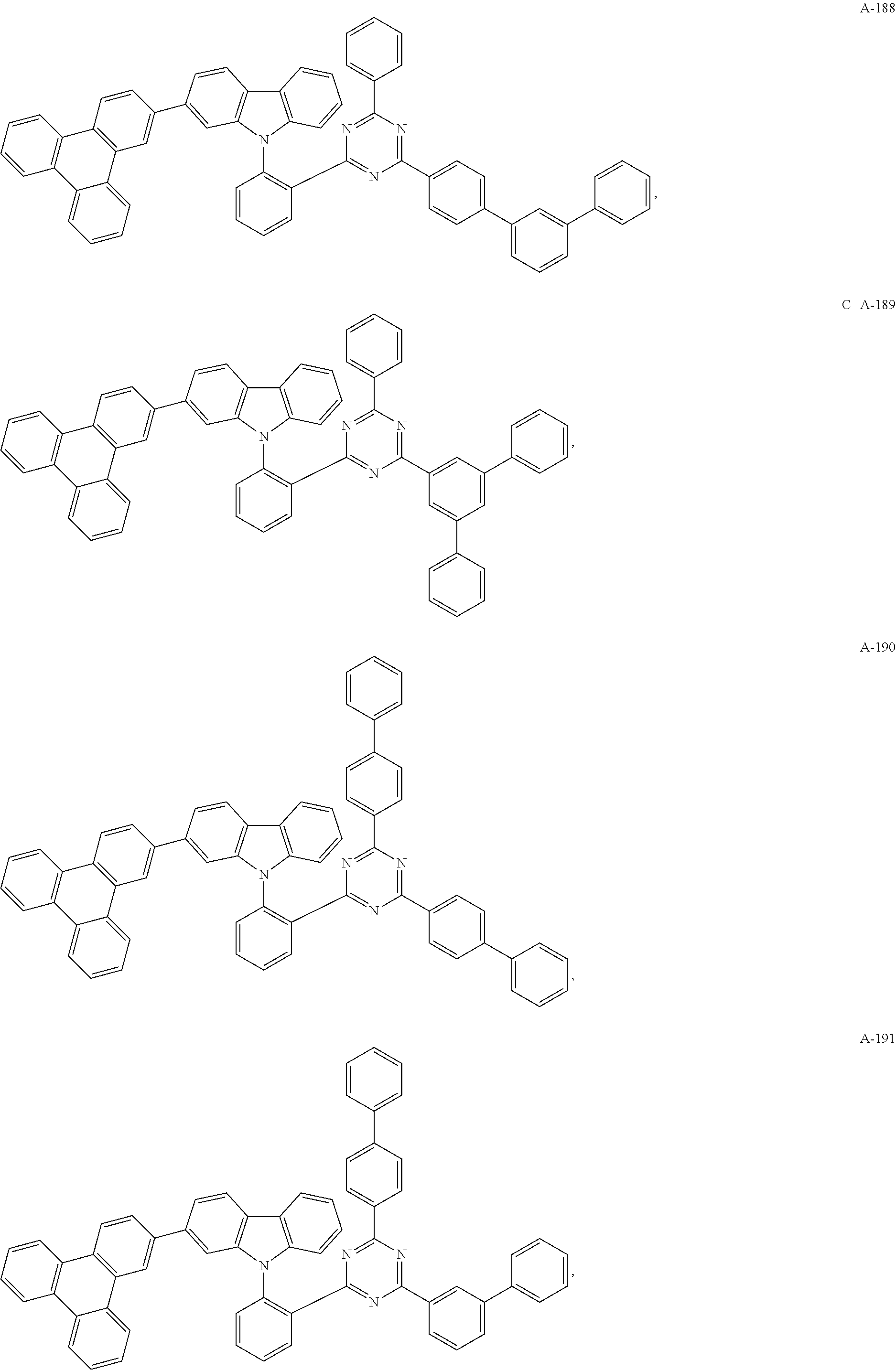 [SMILES: c1ccc(-c2cccc(-c3ccc(-c4nc(-c5ccccc5)nc(-c5ccccc5-n5c6ccccc6c6ccc(-c7ccc8c9ccccc9c9ccccc9c8c7)cc65)n4)cc3)c2)cc1 ; c1ccc(-c2cc(-c3ccccc3)cc(-c3nc(-c4ccccc4)nc(-c4ccccc4-n4c5ccccc5c5ccc(-c6ccc7c8ccccc8c8ccccc8c7c6)cc54)n3)c2)cc1 ; c1ccc(-c2ccc(-c3nc(-c4ccc(-c5ccccc5)cc4)nc(-c4ccccc4-n4c5ccccc5c5ccc(-c6ccc7c8ccccc8c8ccccc8c7c6)cc54)n3)cc2)cc1 ; c1ccc(-c2ccc(-c3nc(-c4cccc(-c5ccccc5)c4)nc(-c4ccccc4-n4c5ccccc5c5ccc(-c6ccc7c8ccccc8c8ccccc8c7c6)cc54)n3)cc2)cc1] 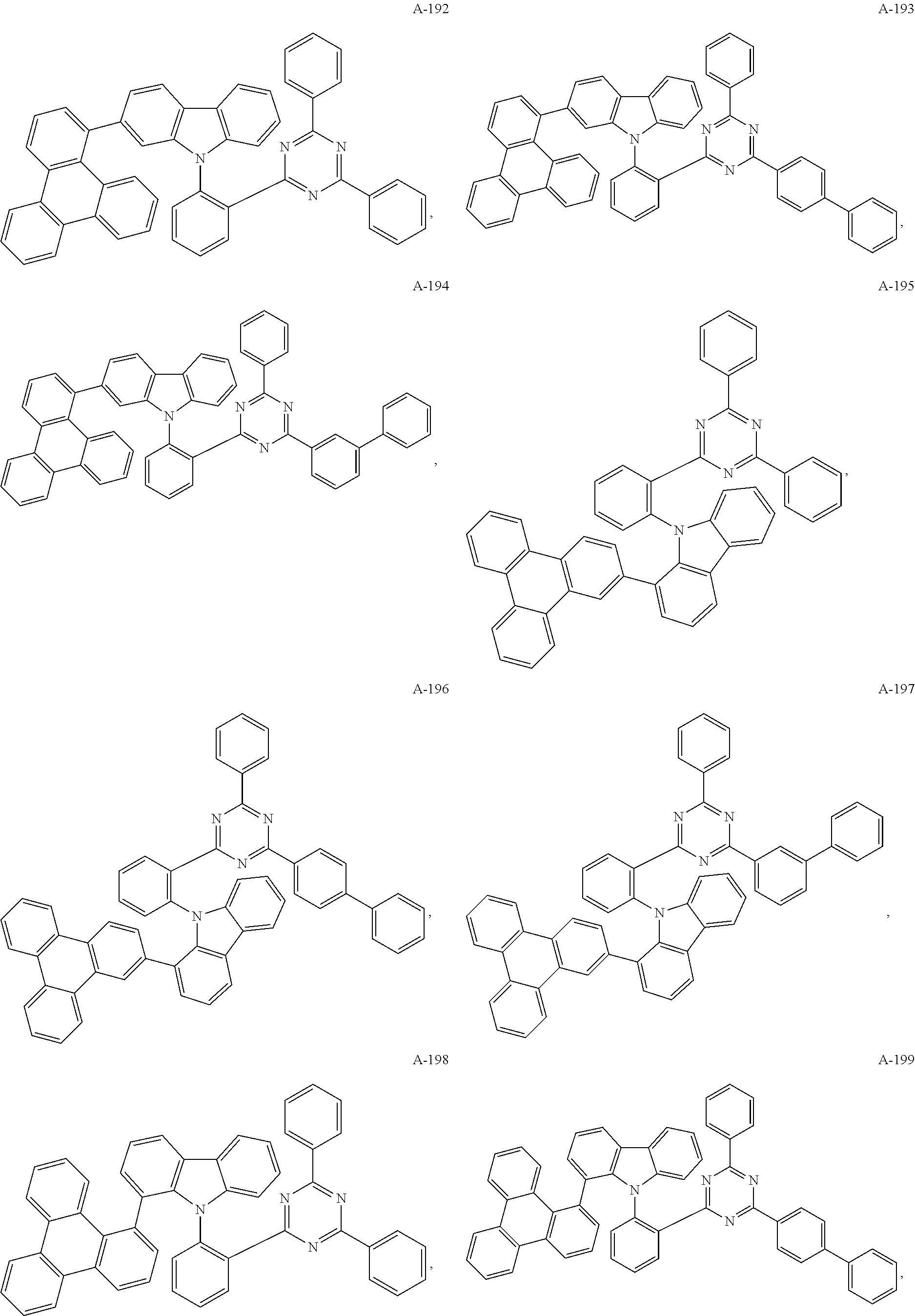 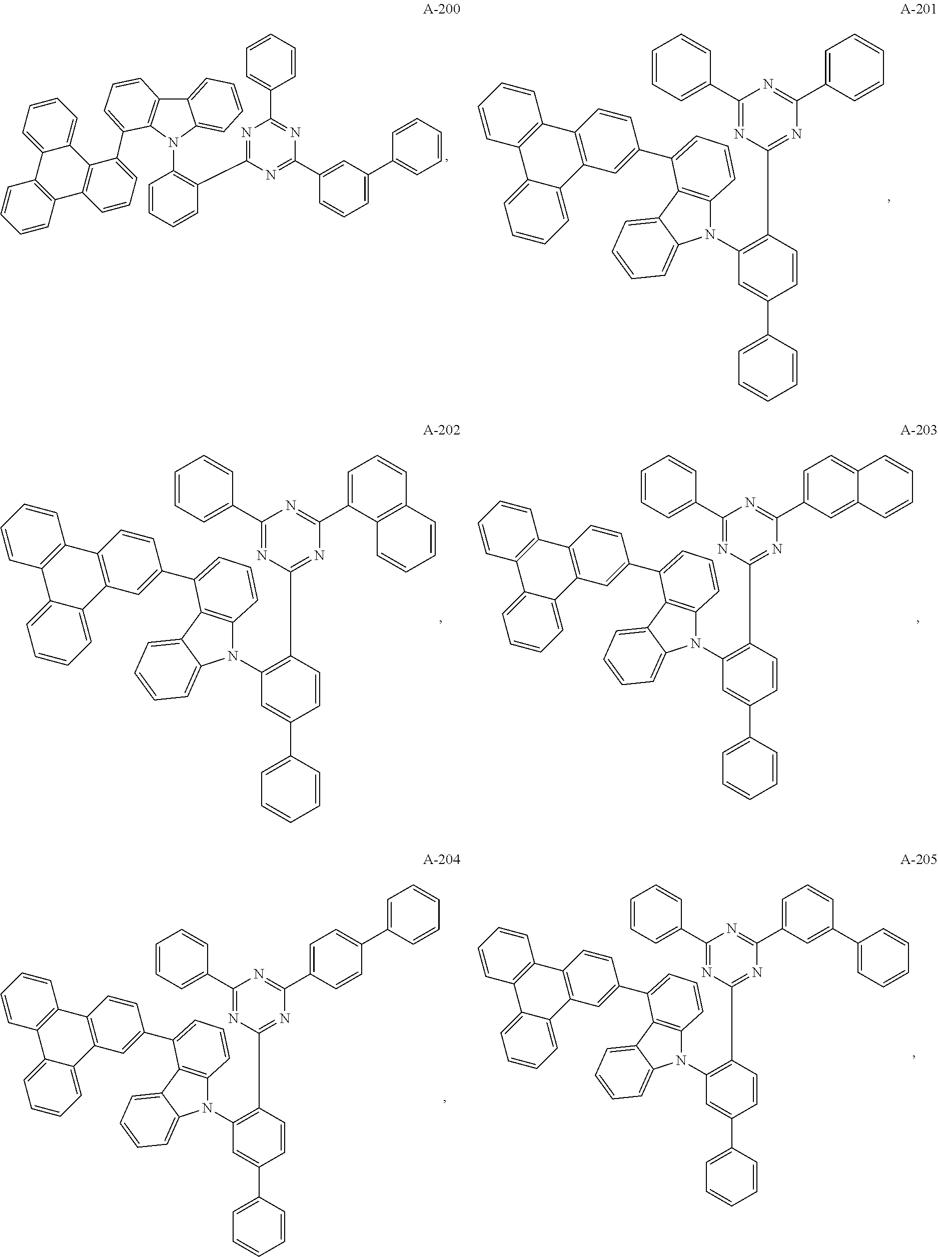 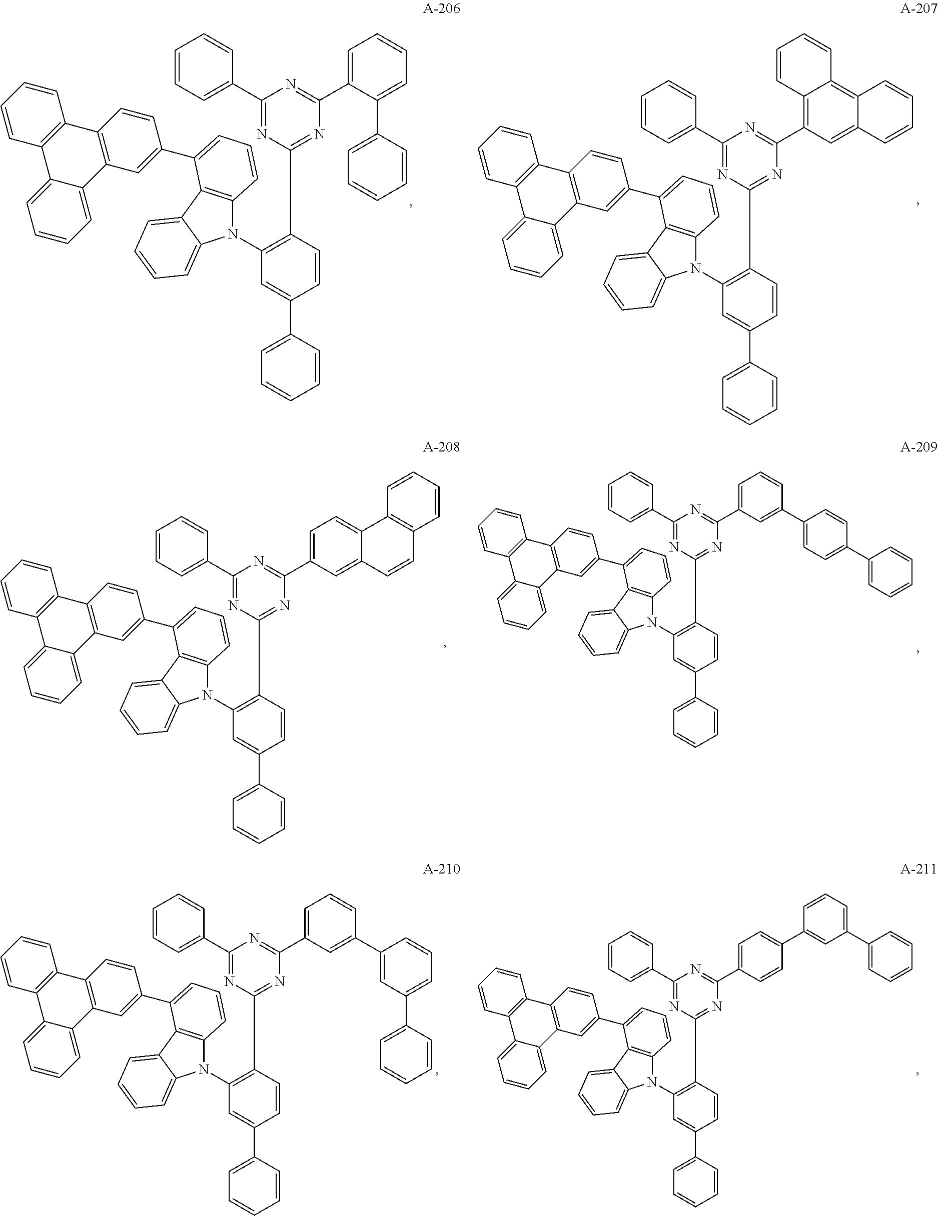 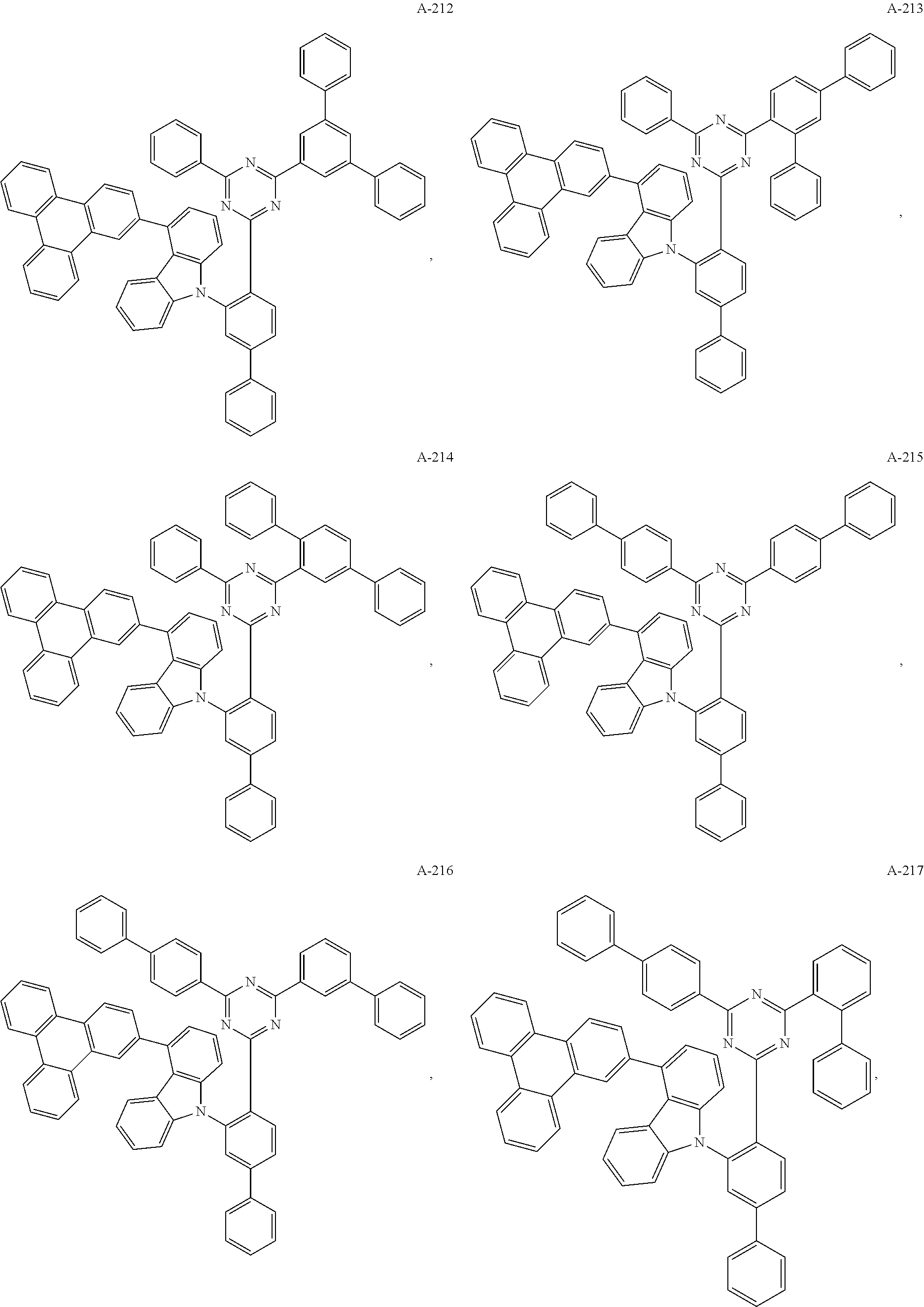 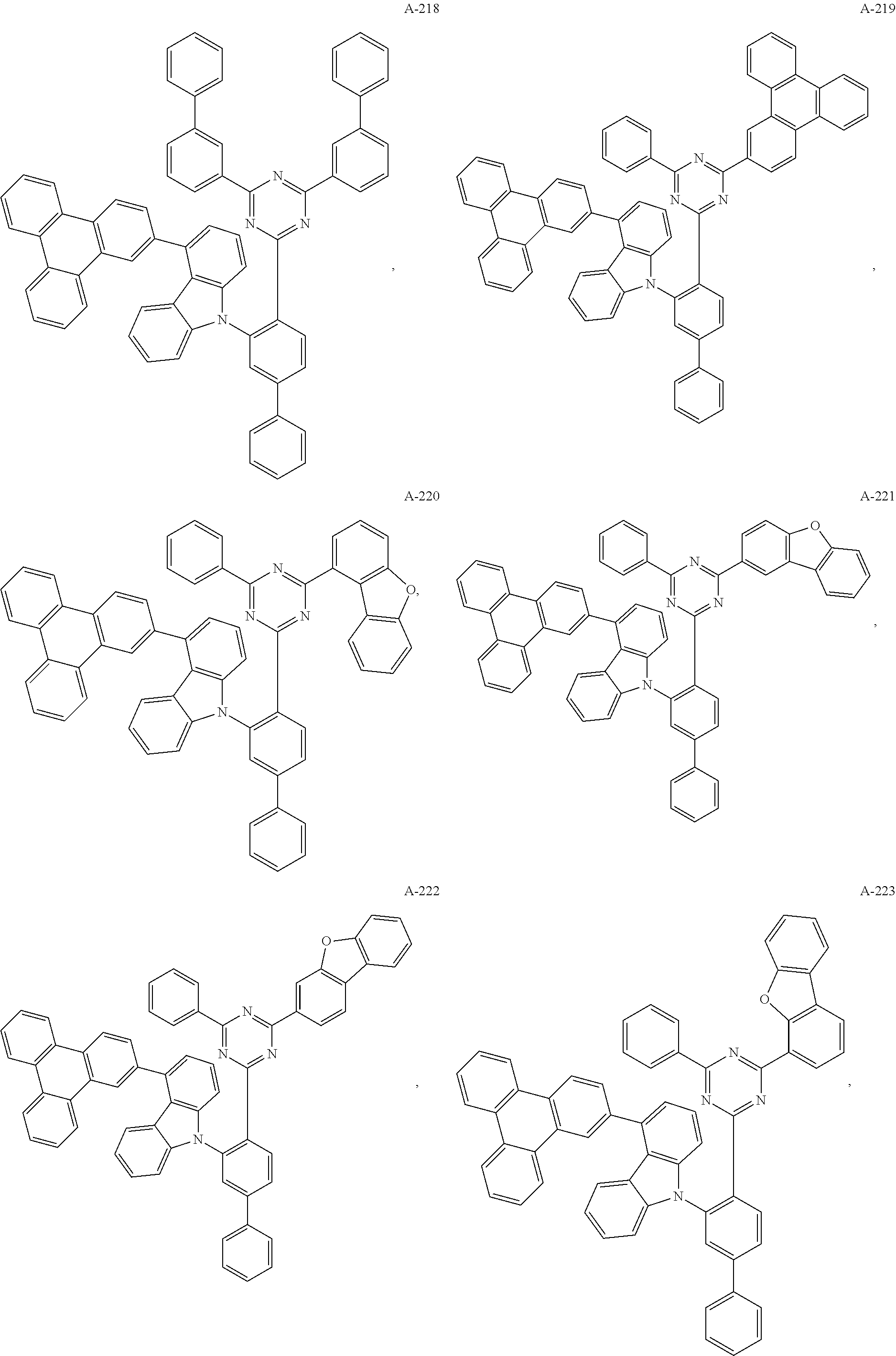 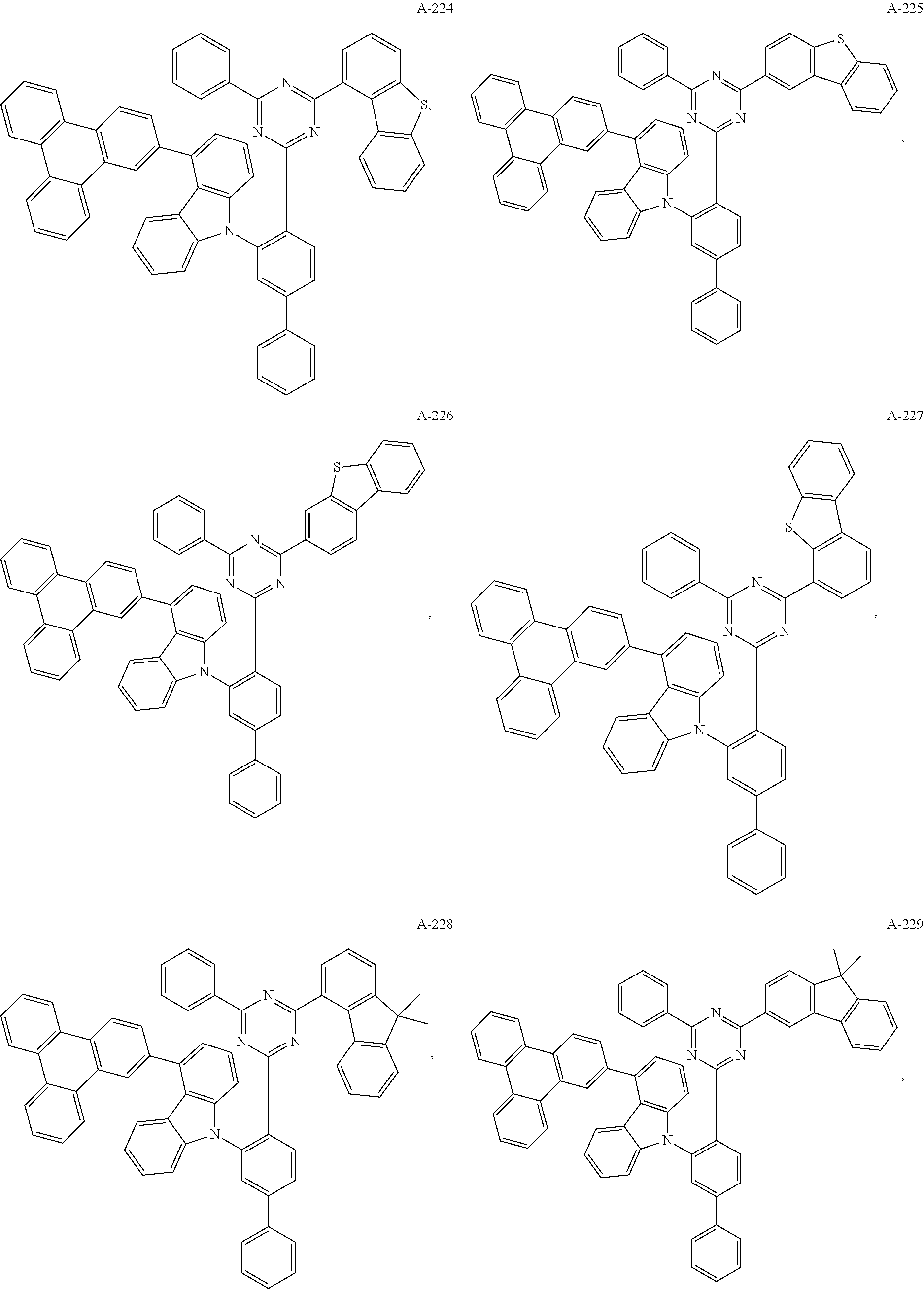 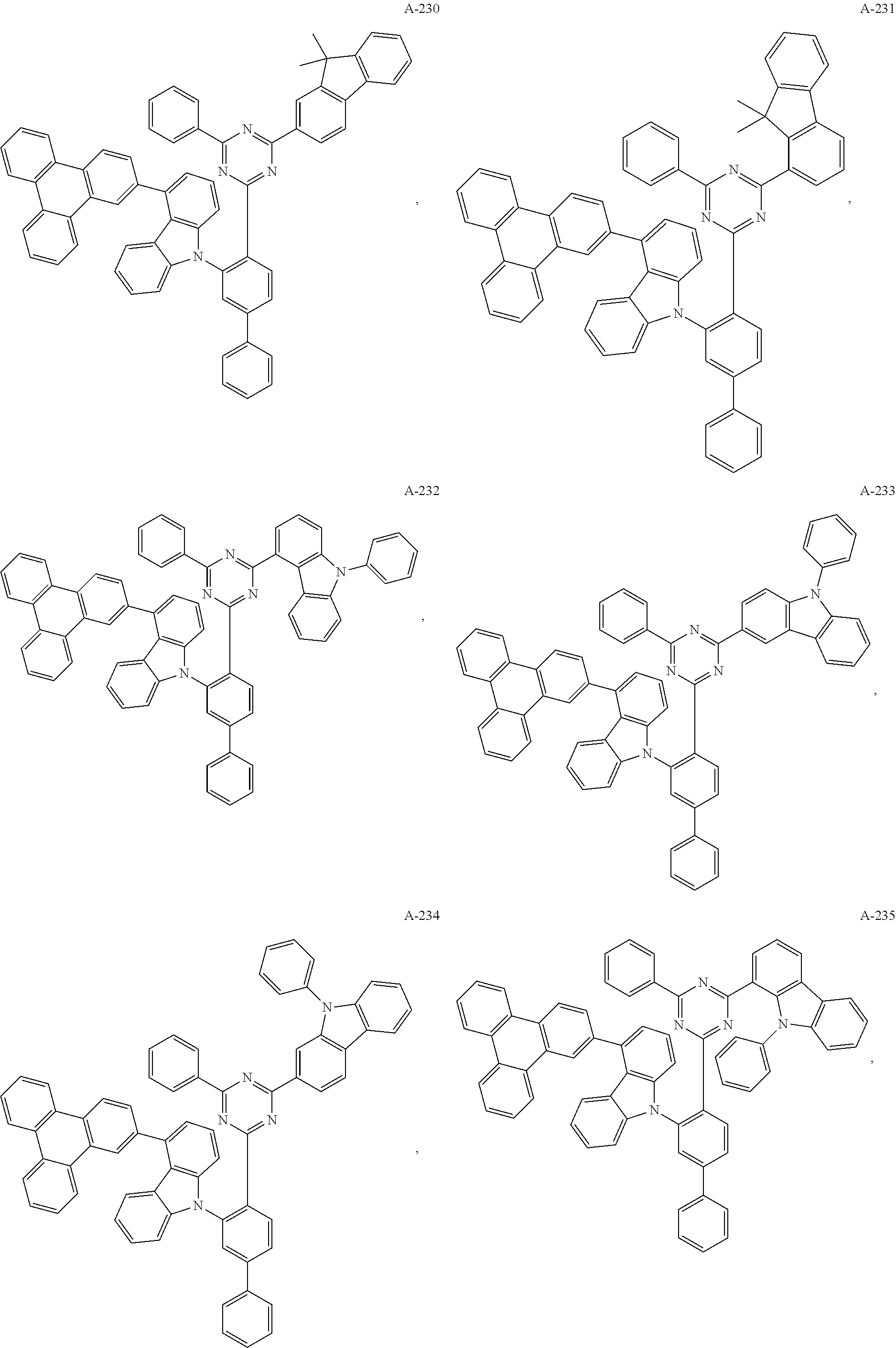 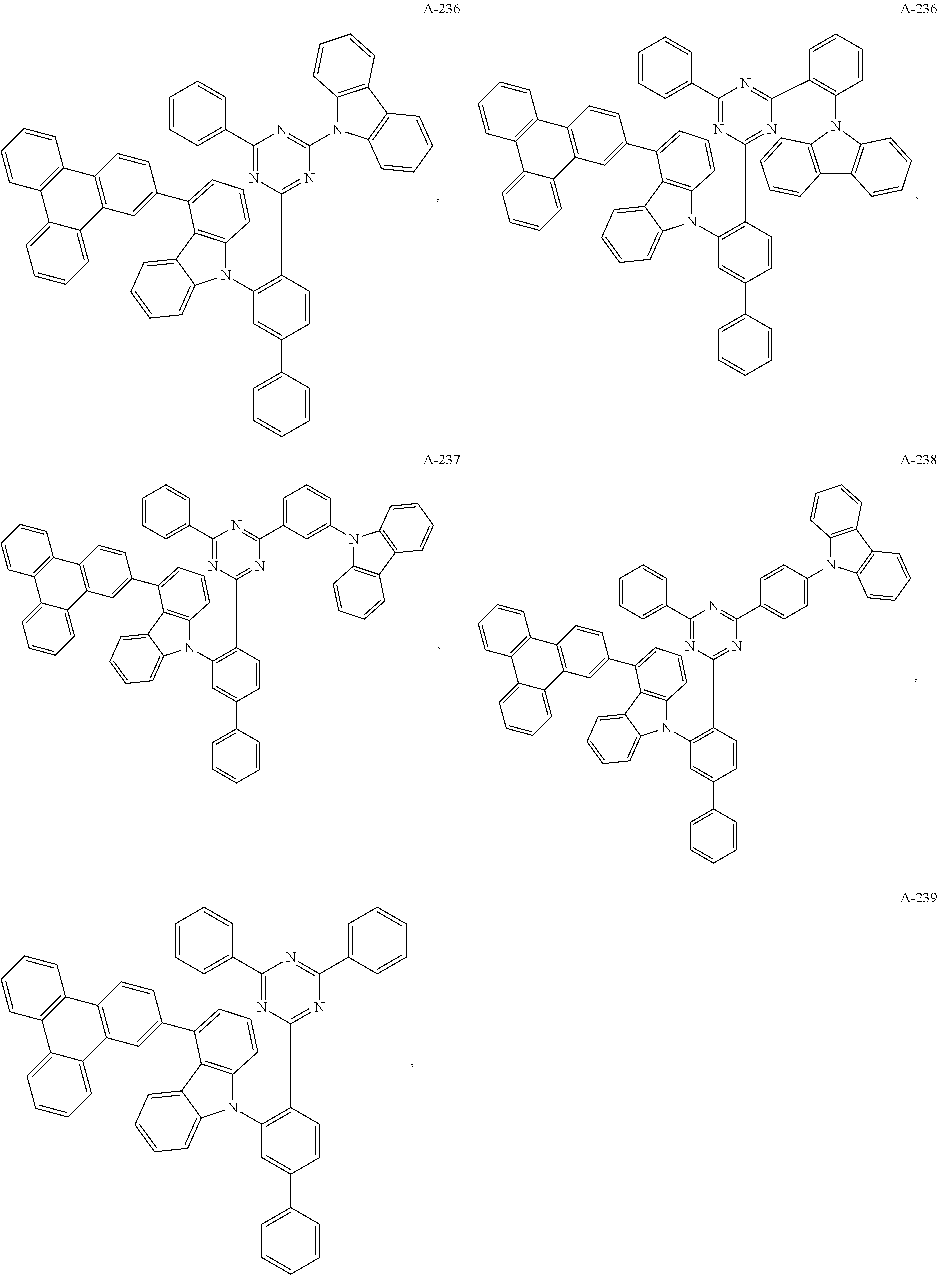 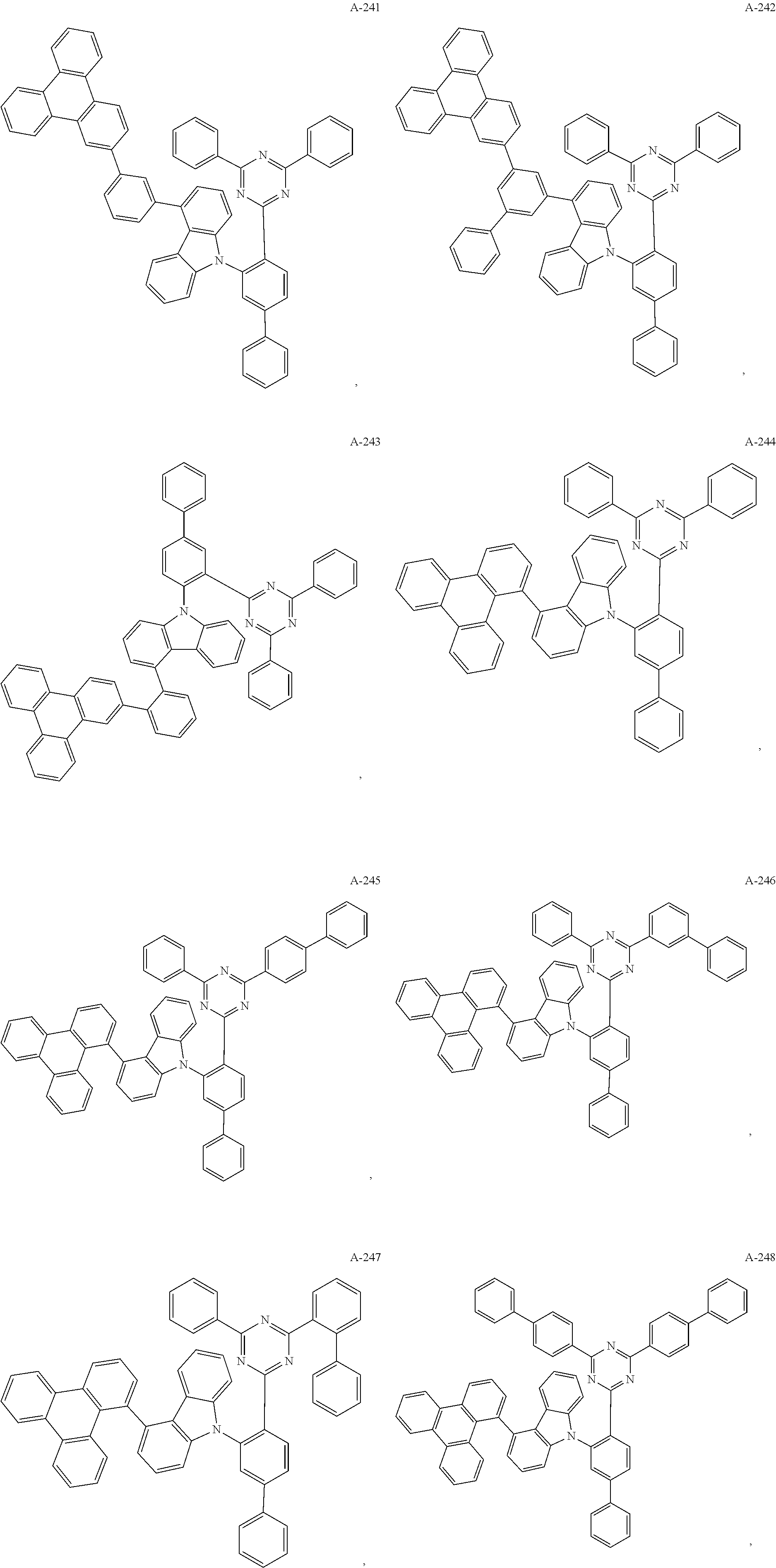 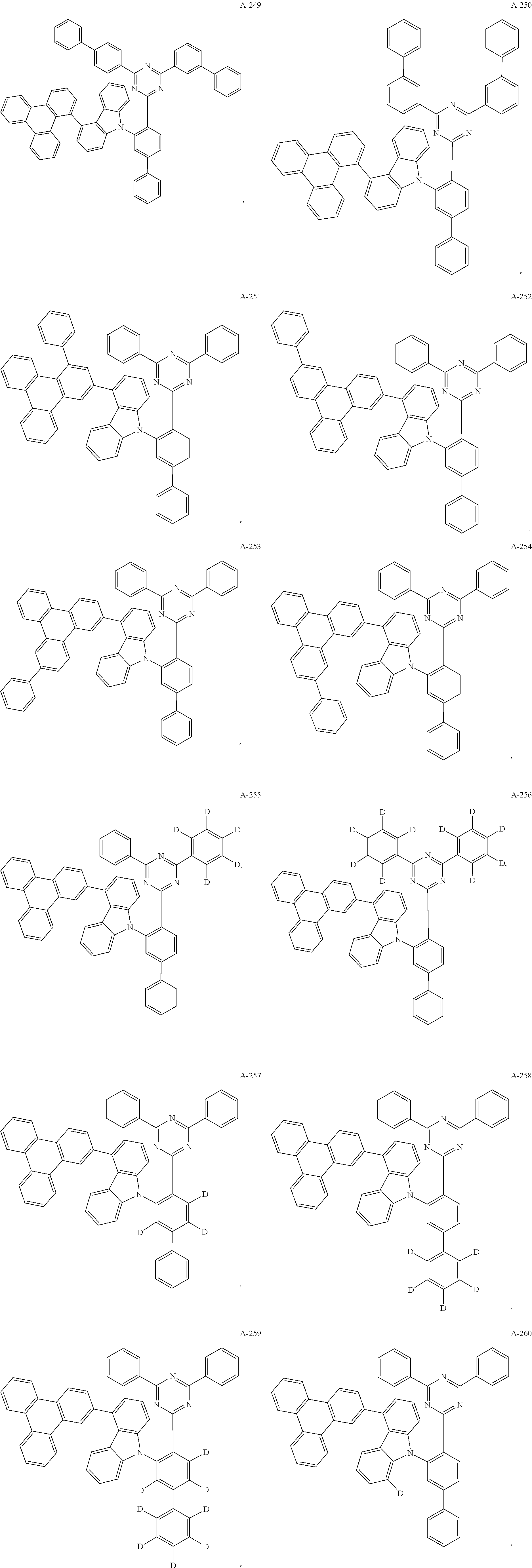 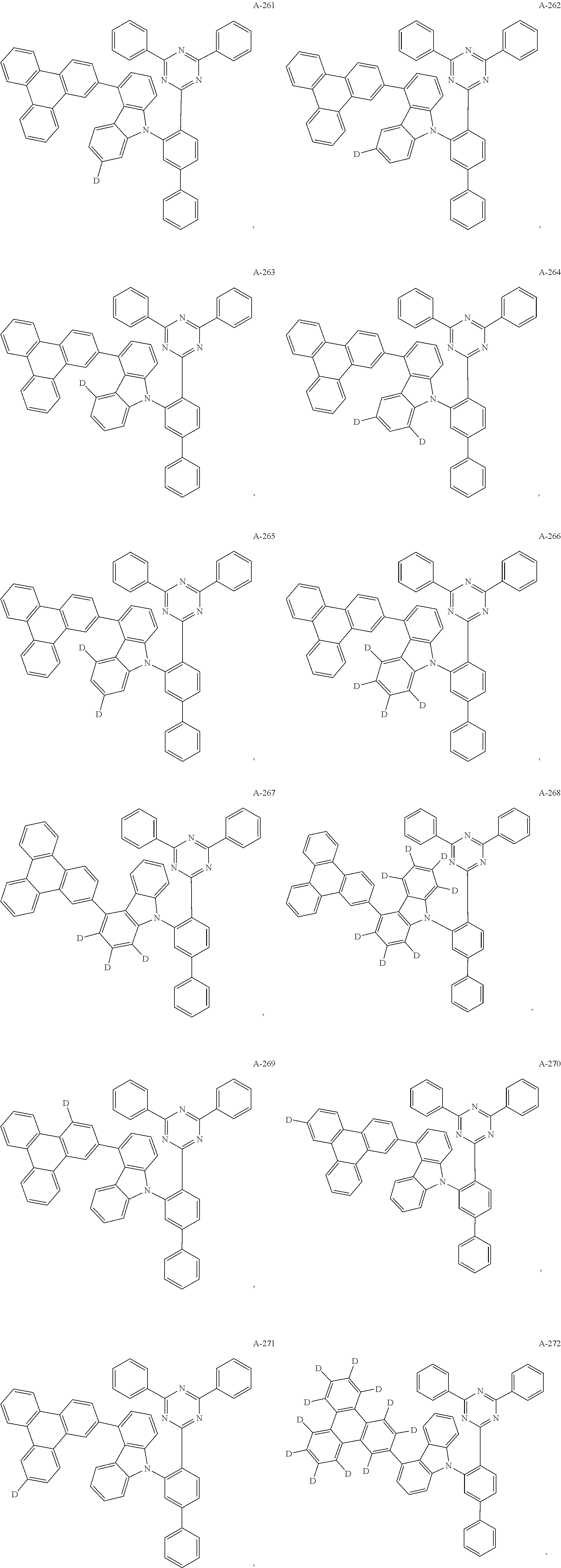 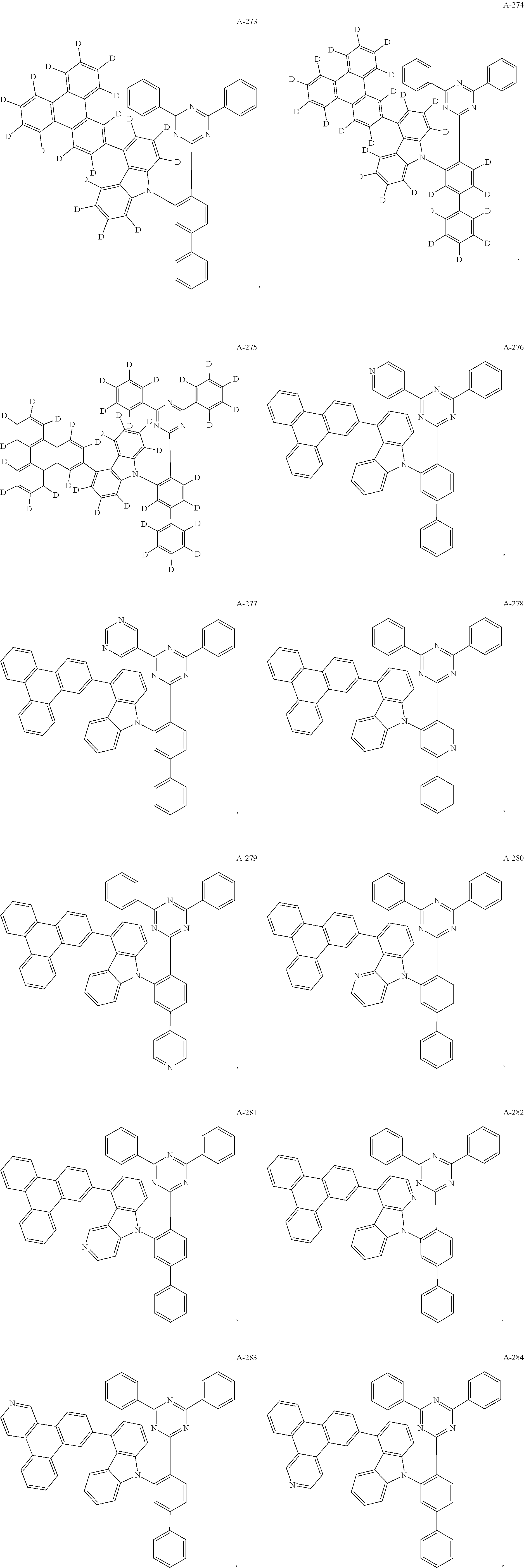 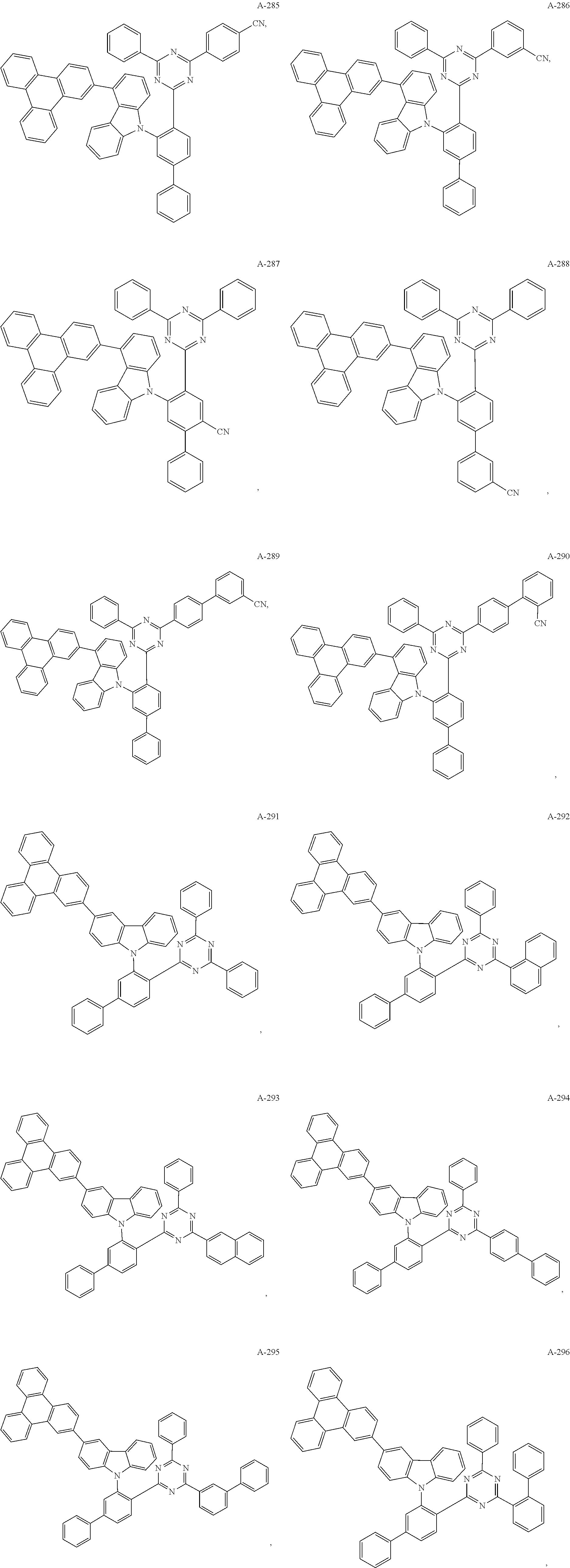 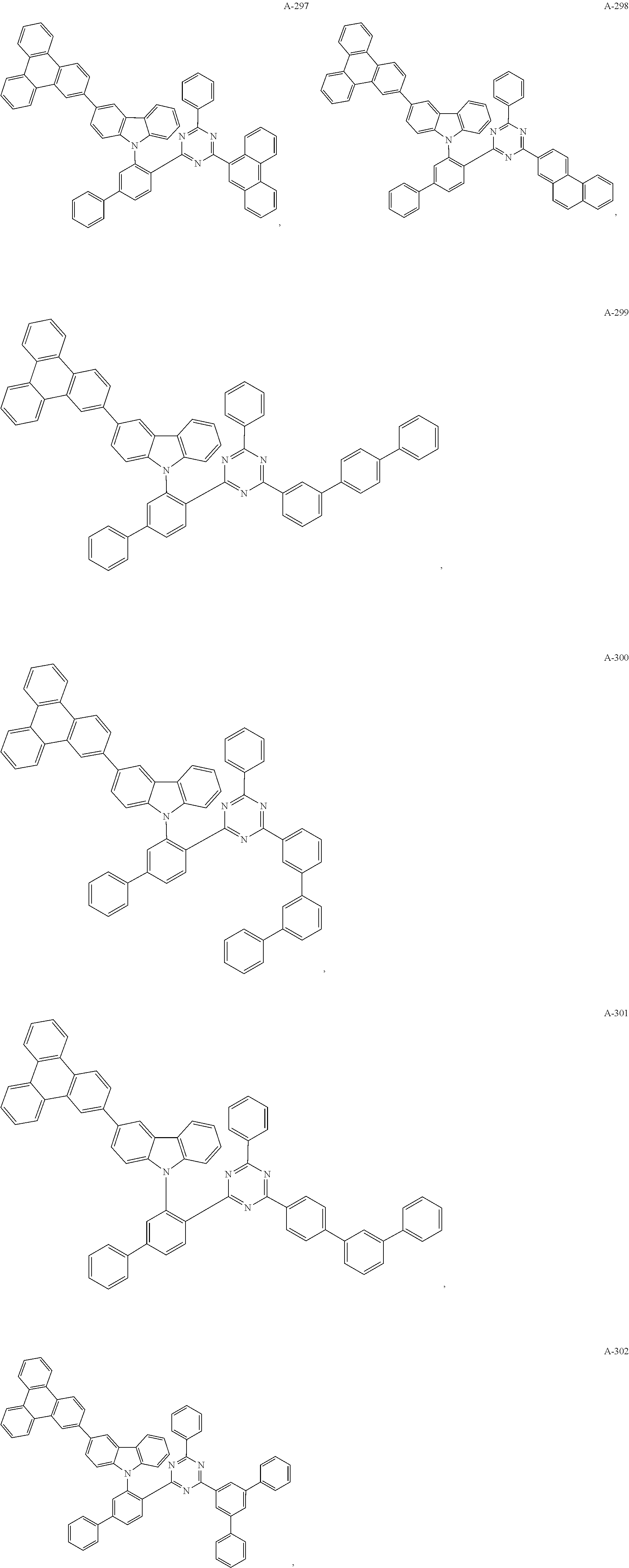 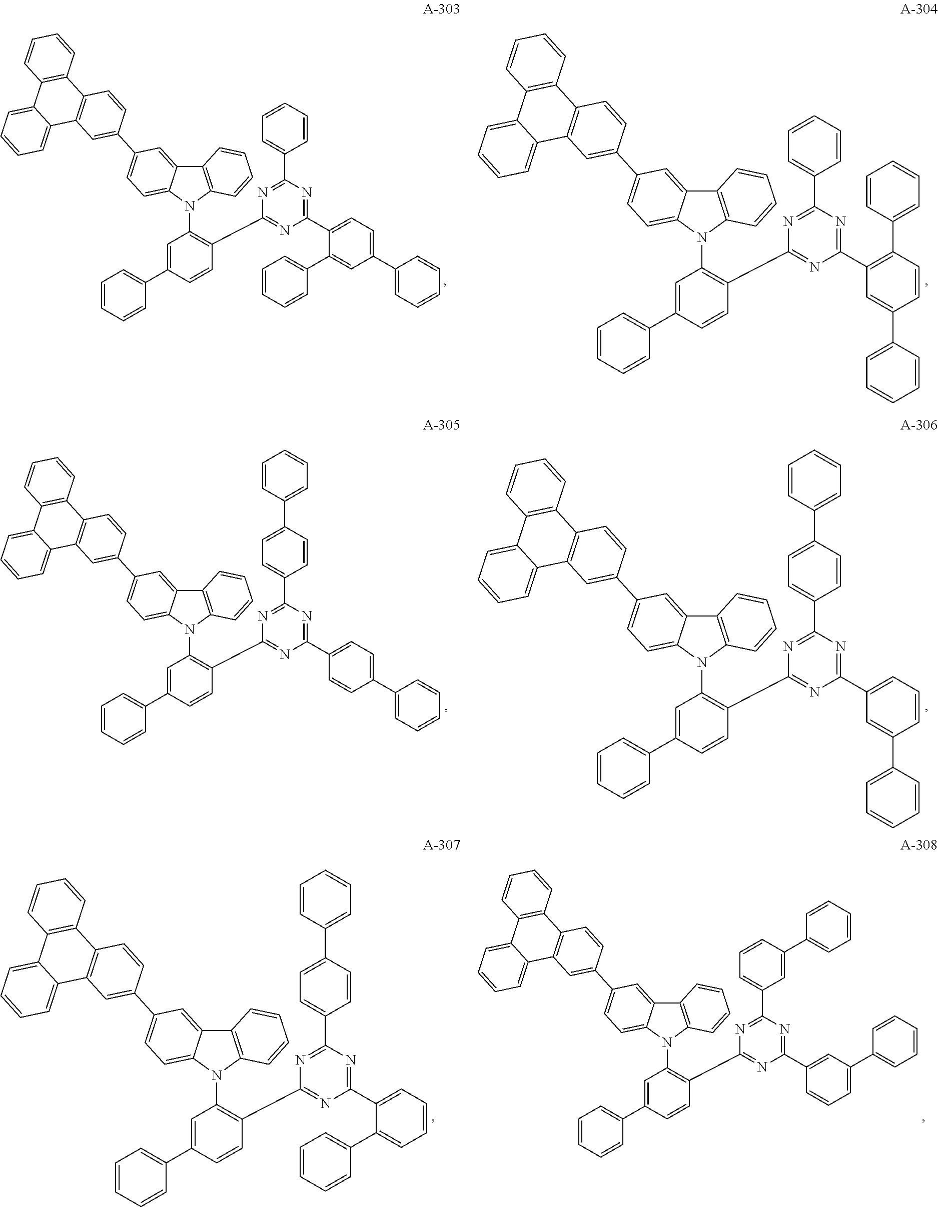 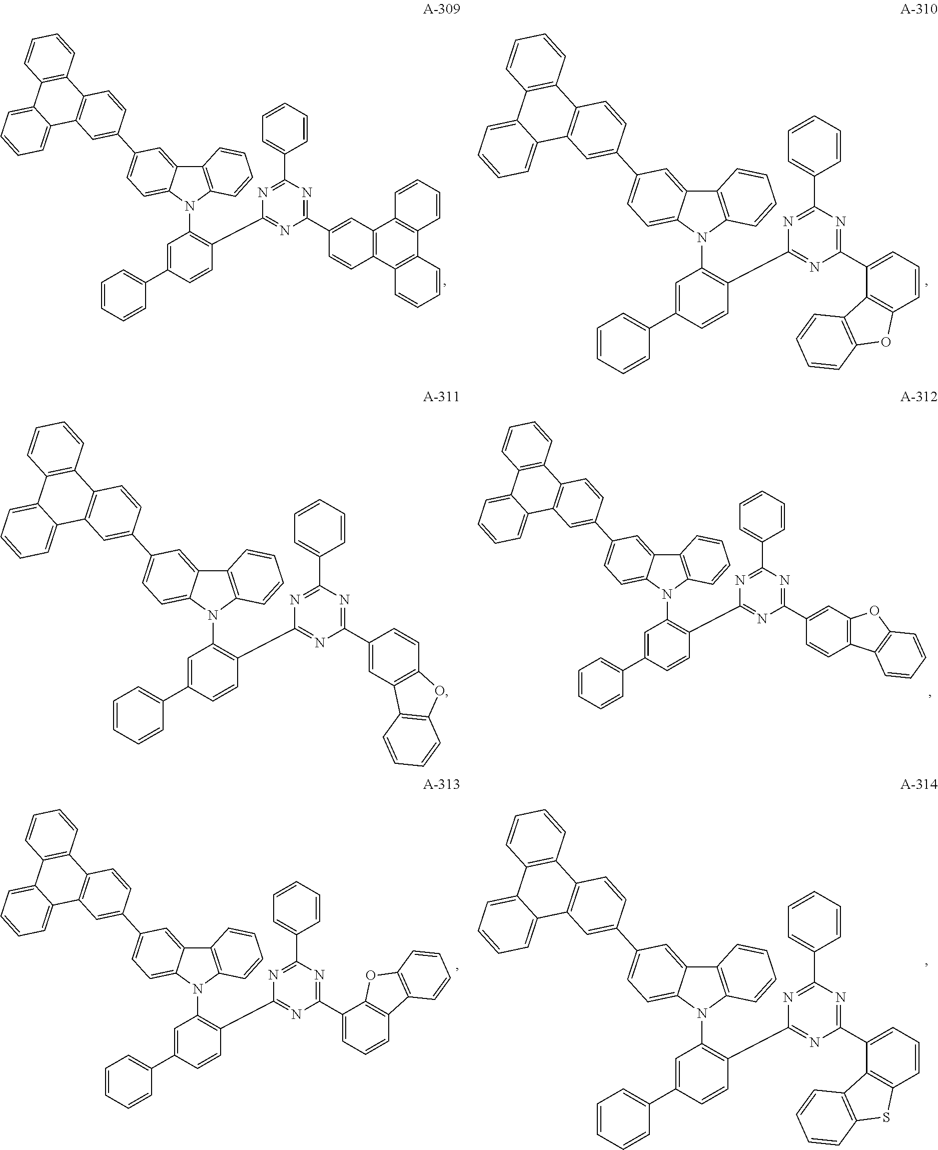 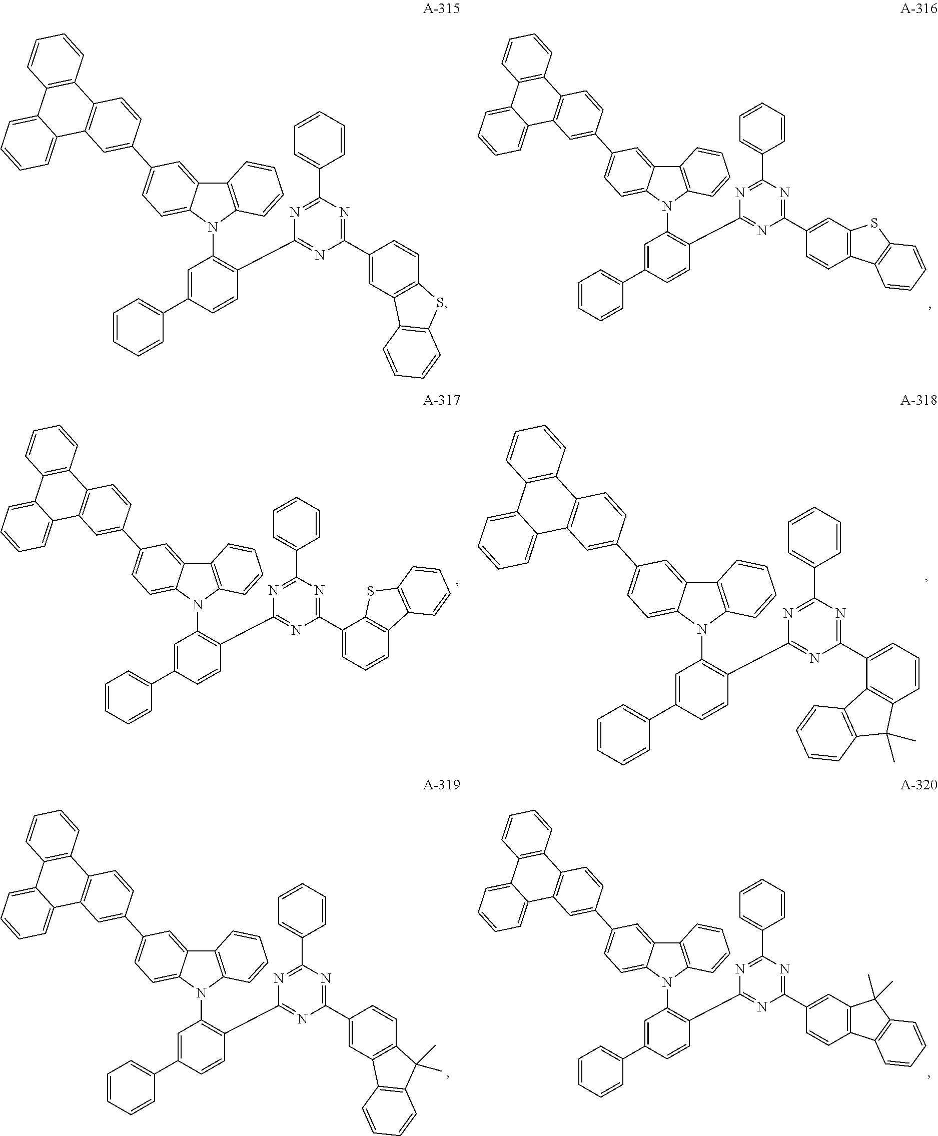 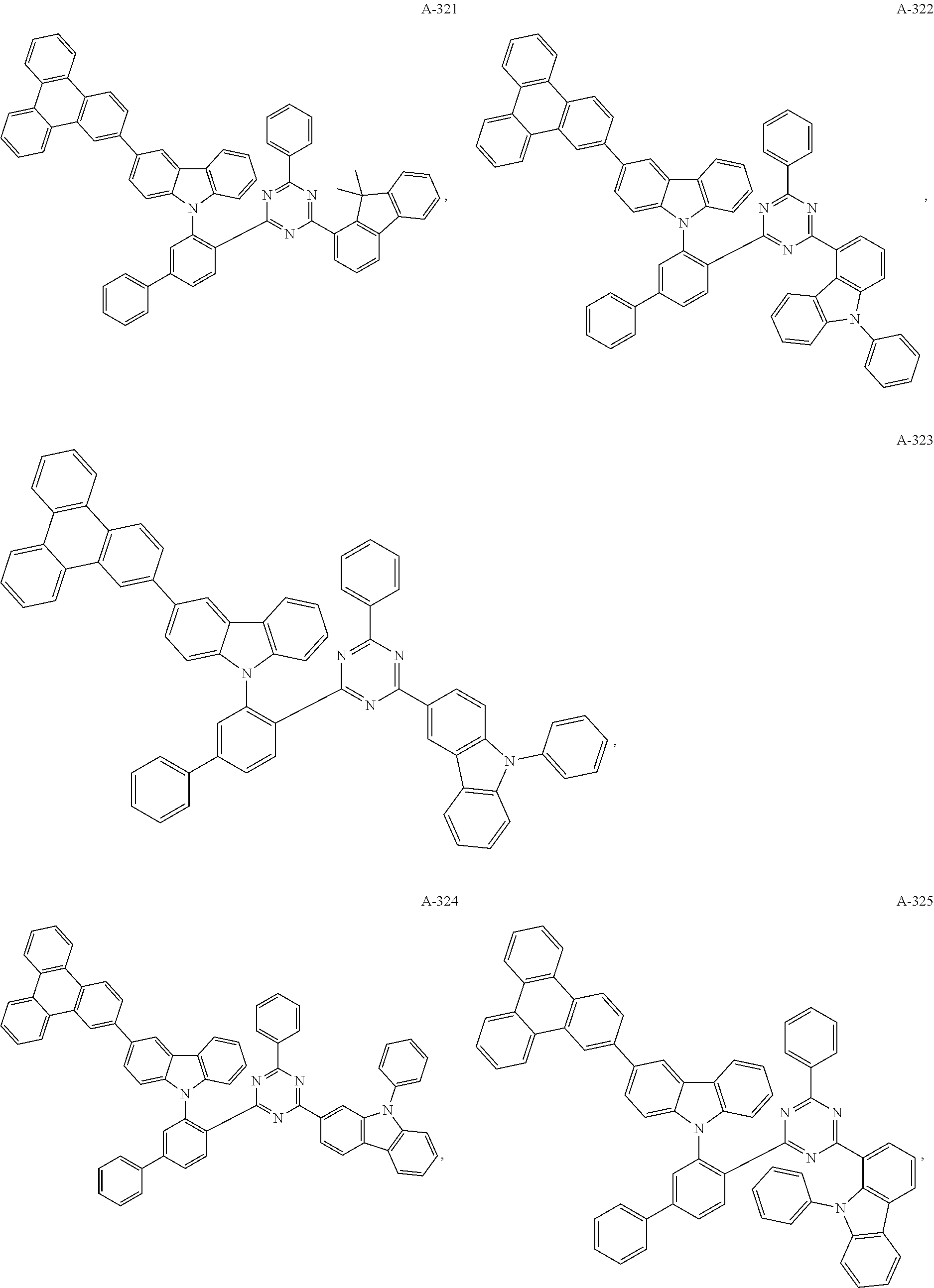 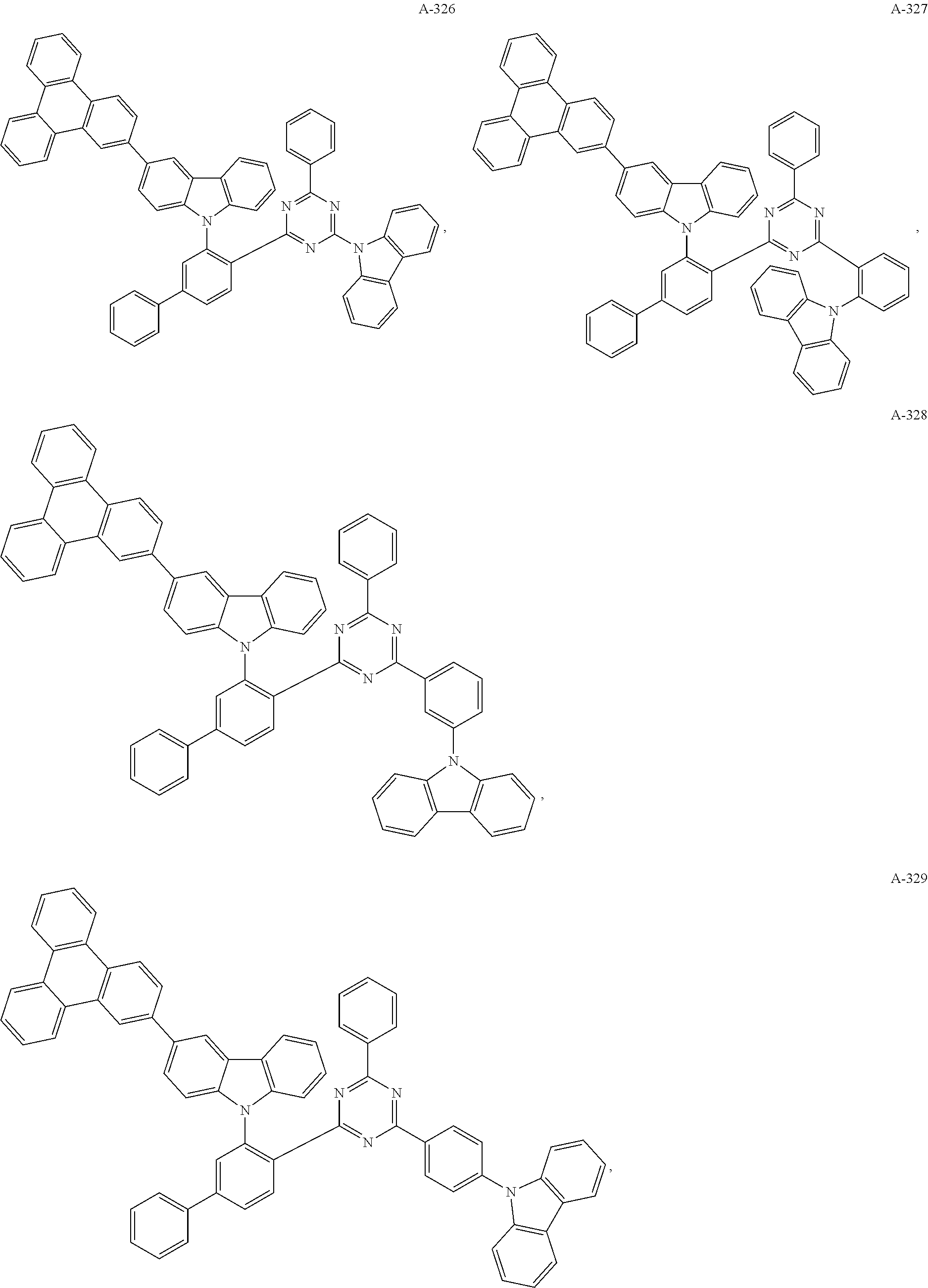 [SMILES: c1ccc(-c2ccc(-c3nc(-c4ccccc4)nc(-n4c5ccccc5c5ccccc54)n3)c(-n3c4ccccc4c4cc(-c5ccc6c7ccccc7c7ccccc7c6c5)ccc43)c2)cc1 ; c1ccc(-c2ccc(-c3nc(-c4ccccc4)nc(-c4ccccc4-n4c5ccccc5c5ccccc54)n3)c(-n3c4ccccc4c4cc(-c5ccc6c7ccccc7c7ccccc7c6c5)ccc43)c2)cc1 ; c1ccc(-c2ccc(-c3nc(-c4ccccc4)nc(-c4cccc(-n5c6ccccc6c6ccccc65)c4)n3)c(-n3c4ccccc4c4cc(-c5ccc6c7ccccc7c7ccccc7c6c5)ccc43)c2)cc1 ; c1ccc(-c2ccc(-c3nc(-c4ccccc4)nc(-c4ccc(-n5c6ccccc6c6ccccc65)cc4)n3)c(-n3c4ccccc4c4cc(-c5ccc6c7ccccc7c7ccccc7c6c5)ccc43)c2)cc1] 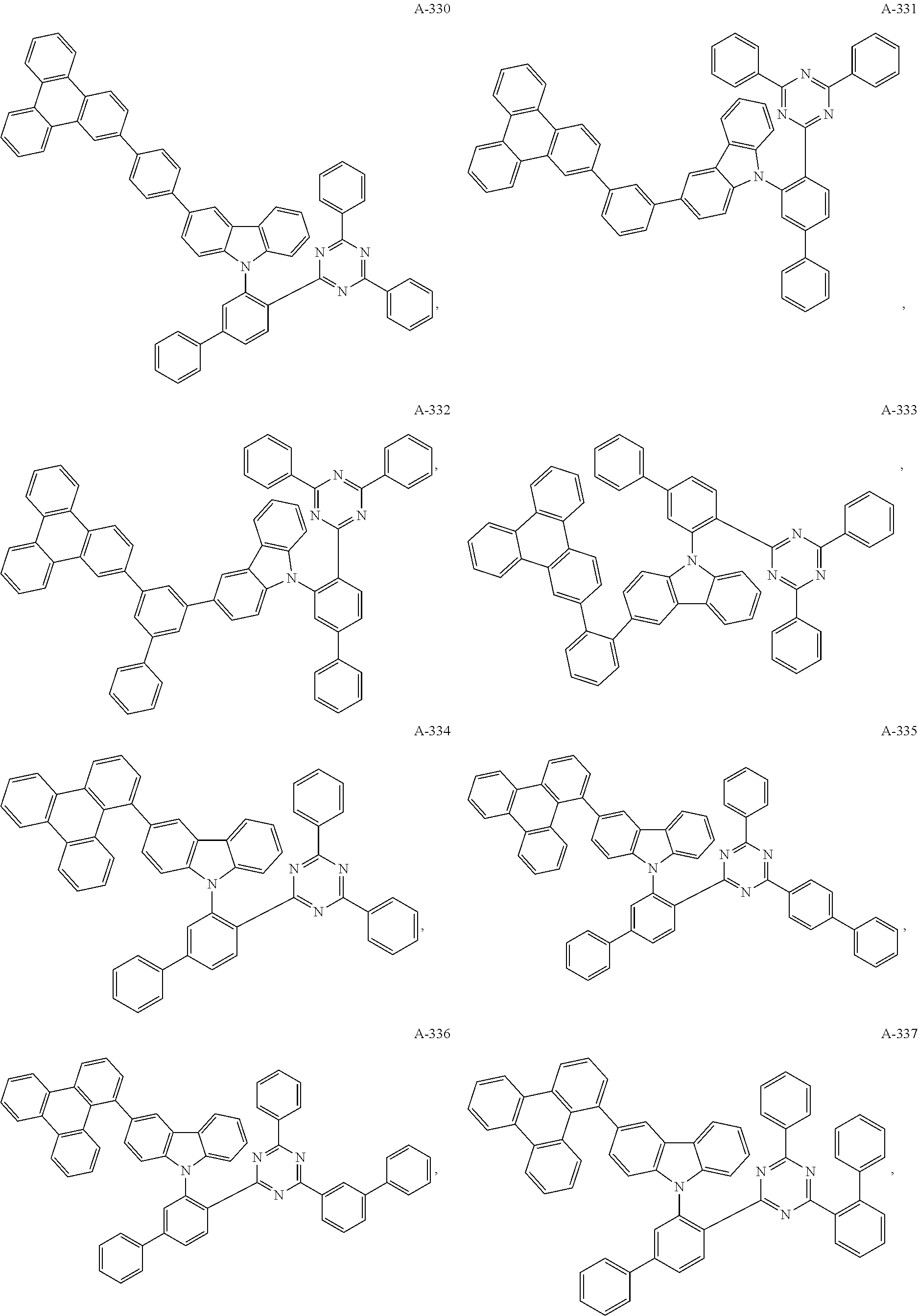 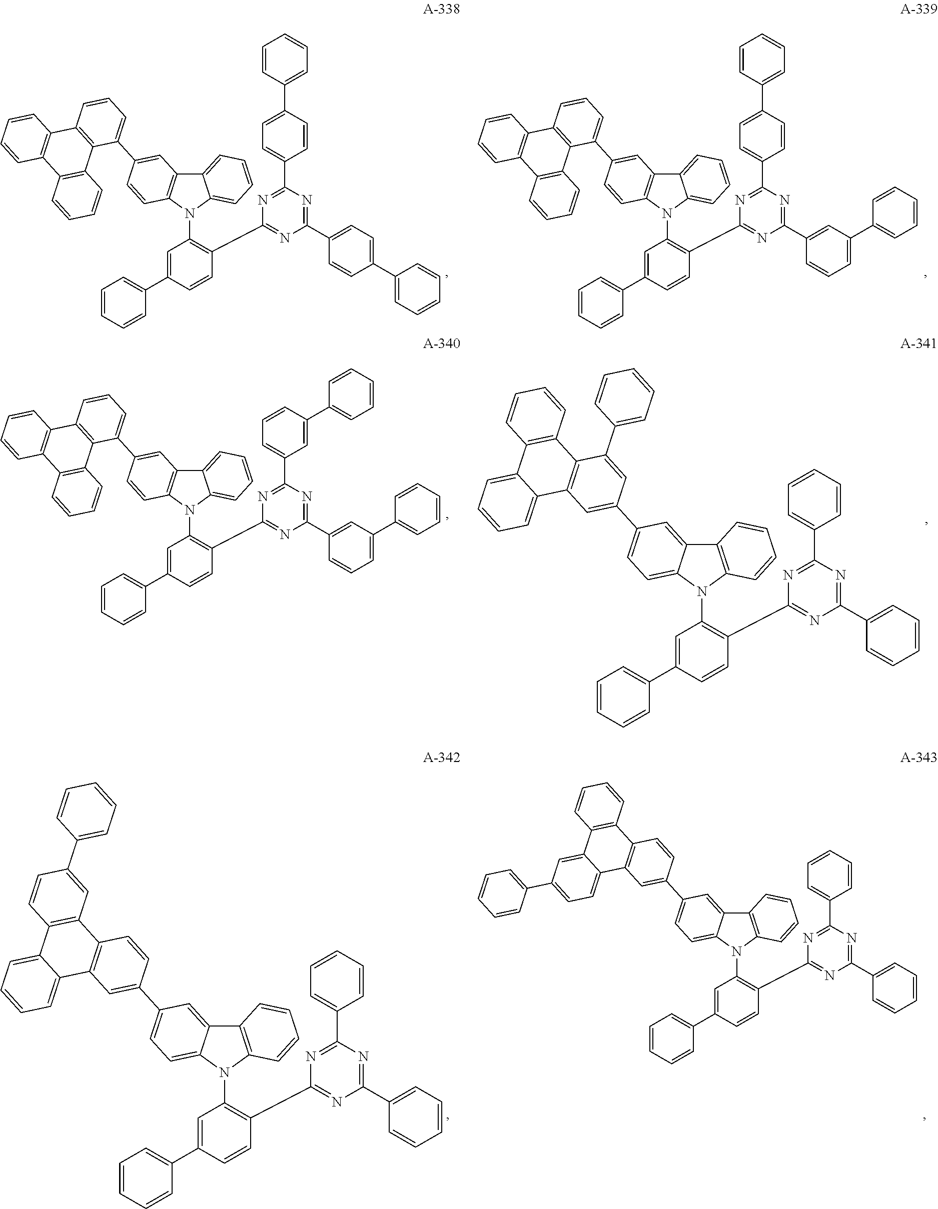 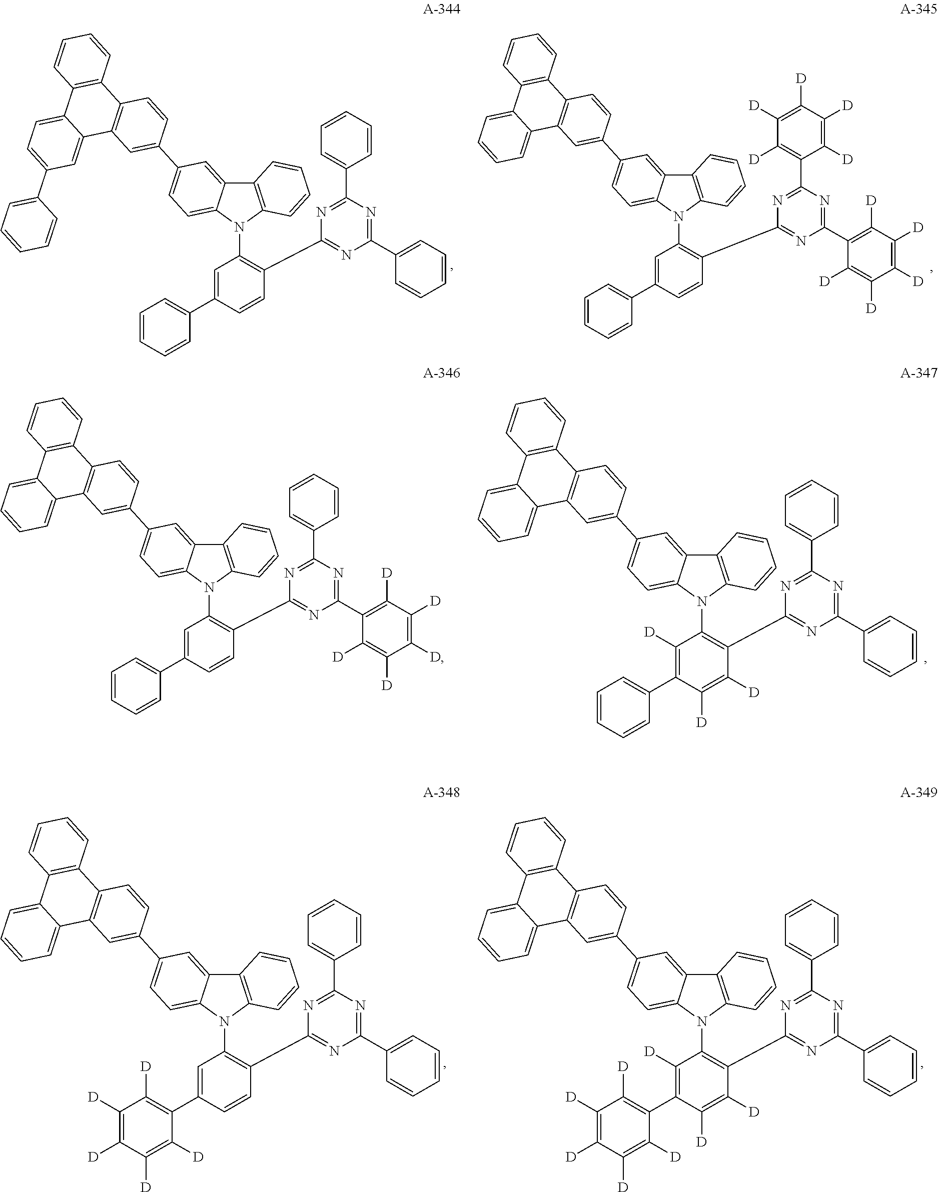 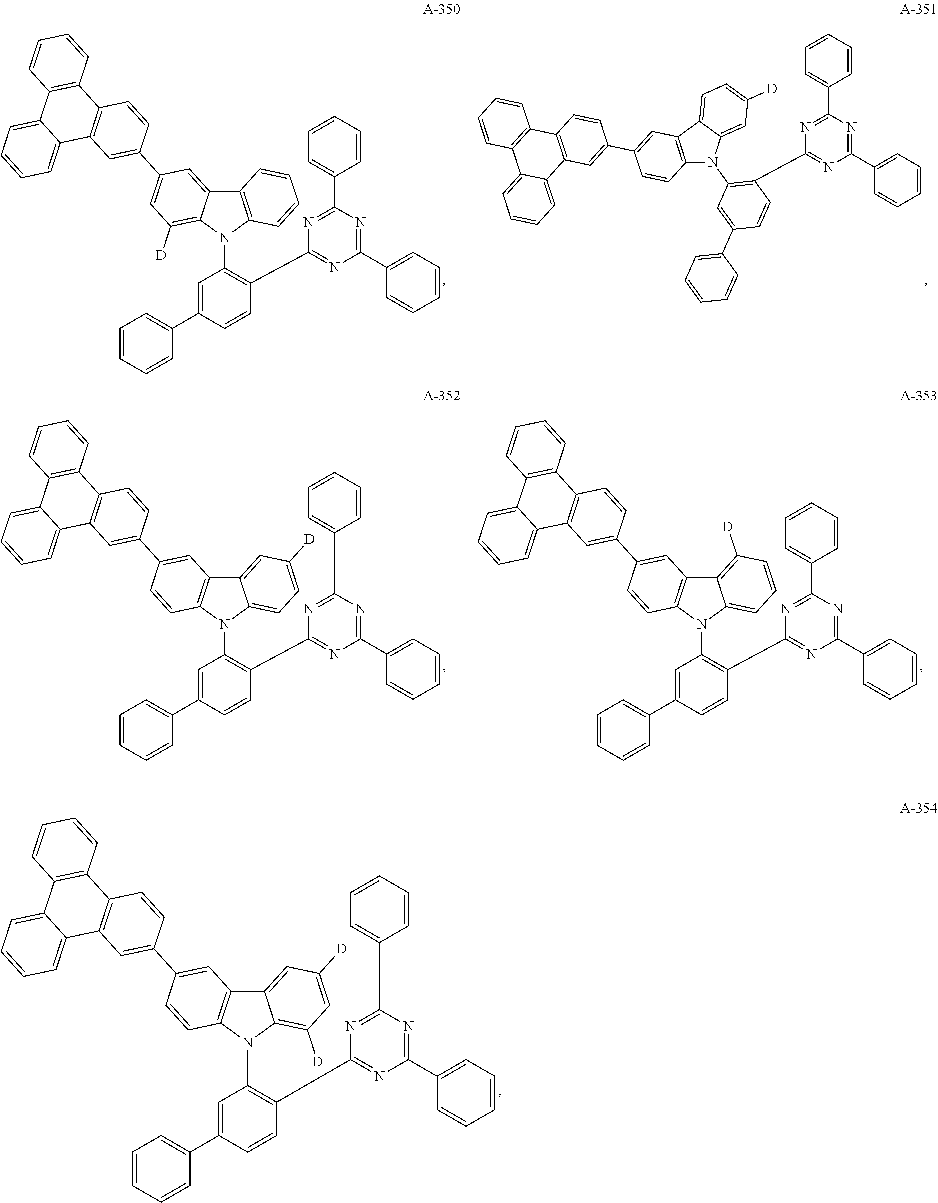 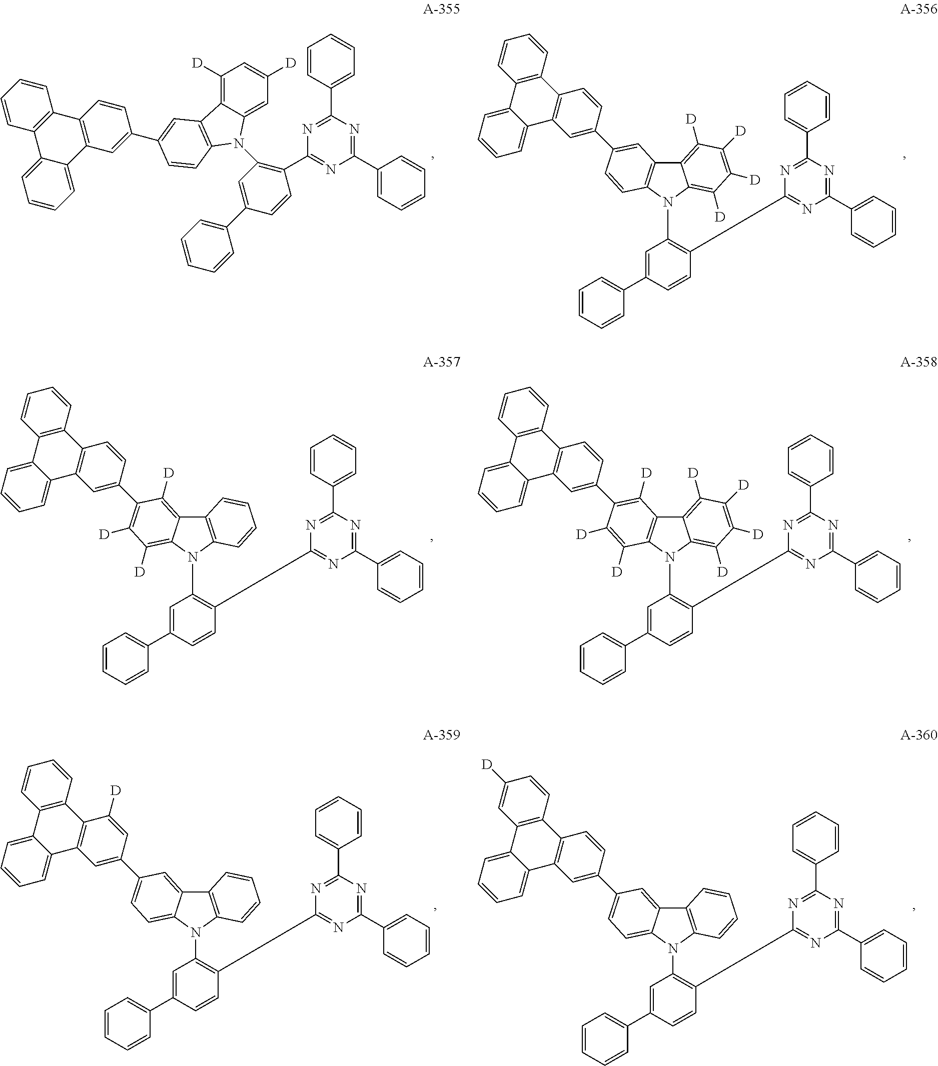 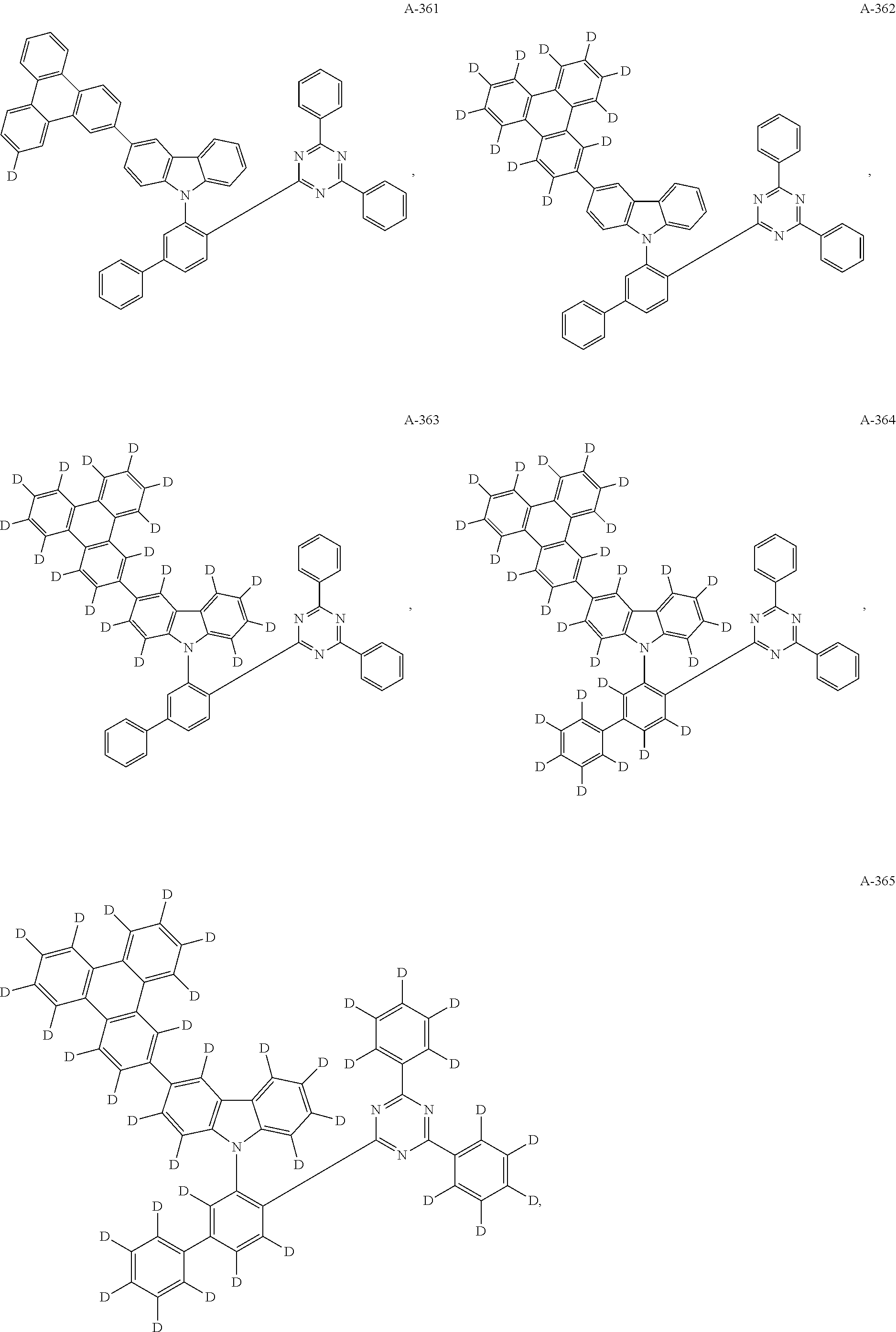 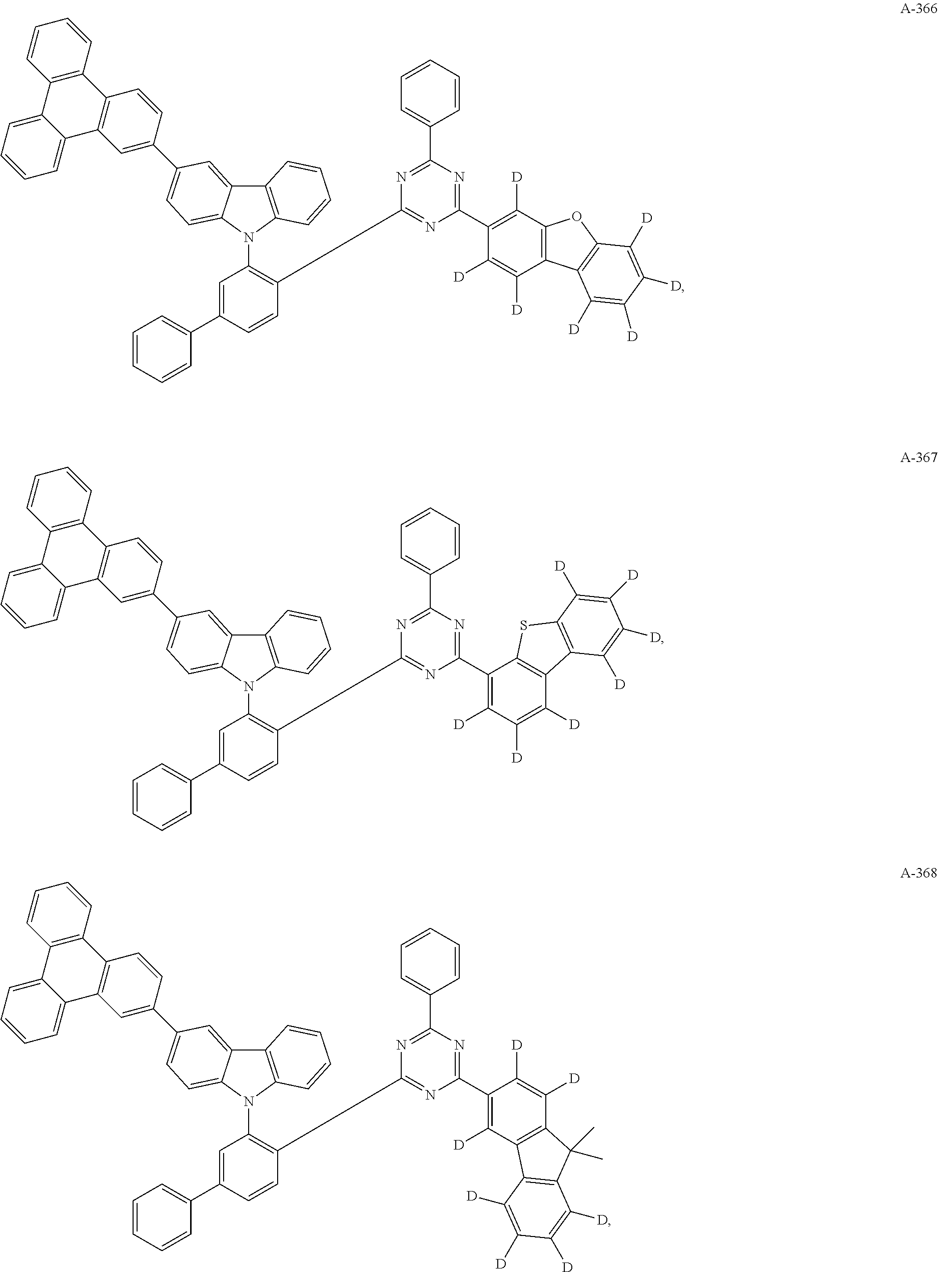 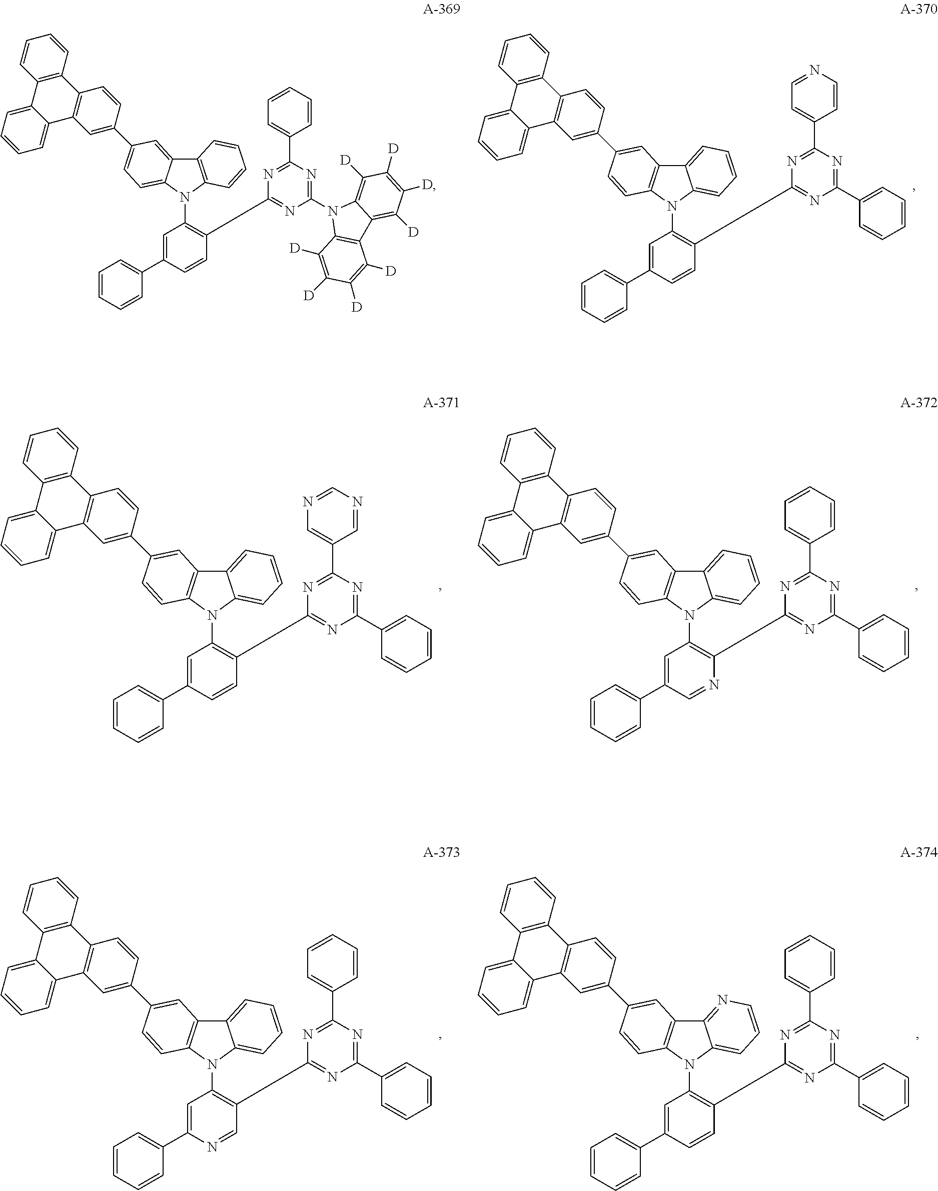 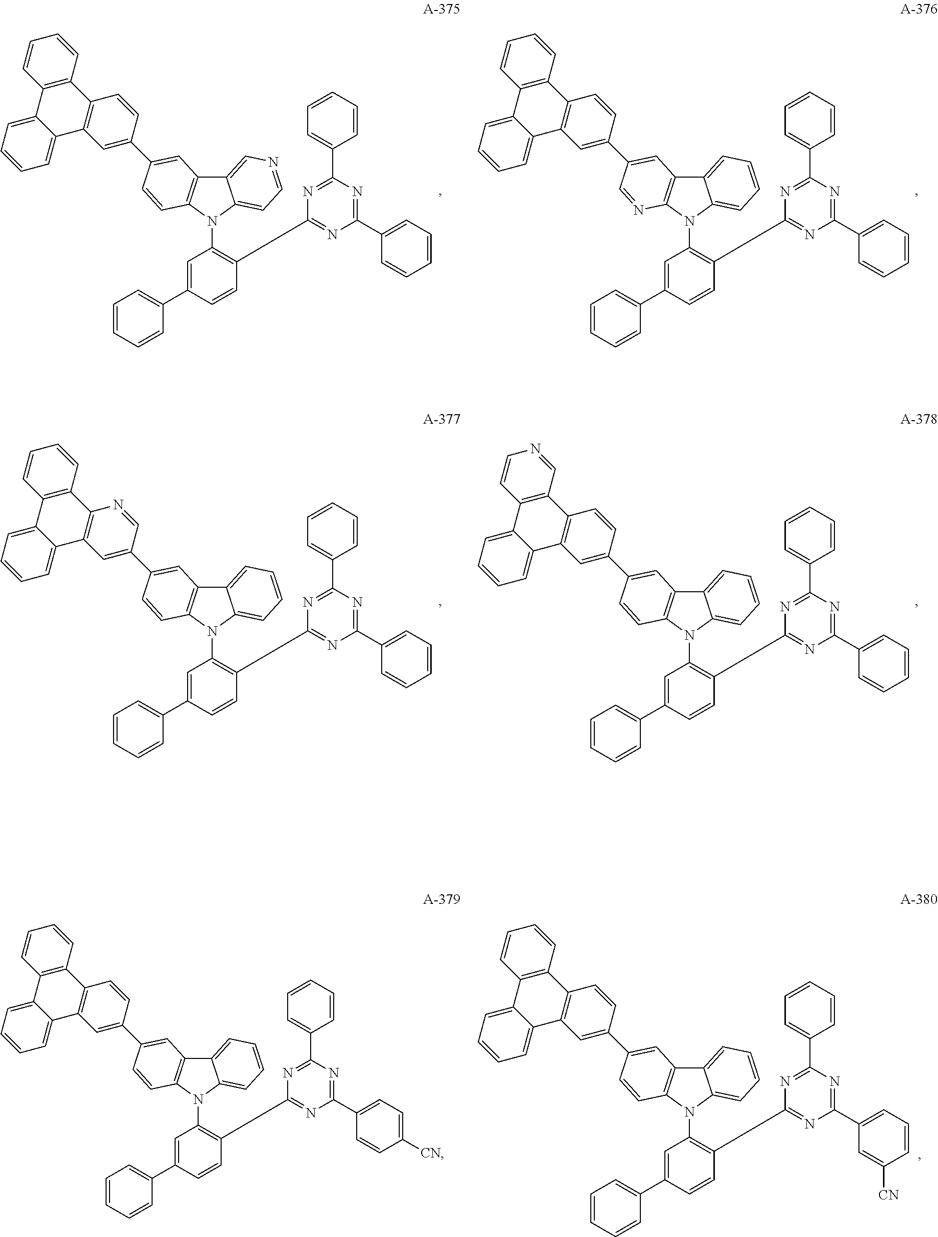 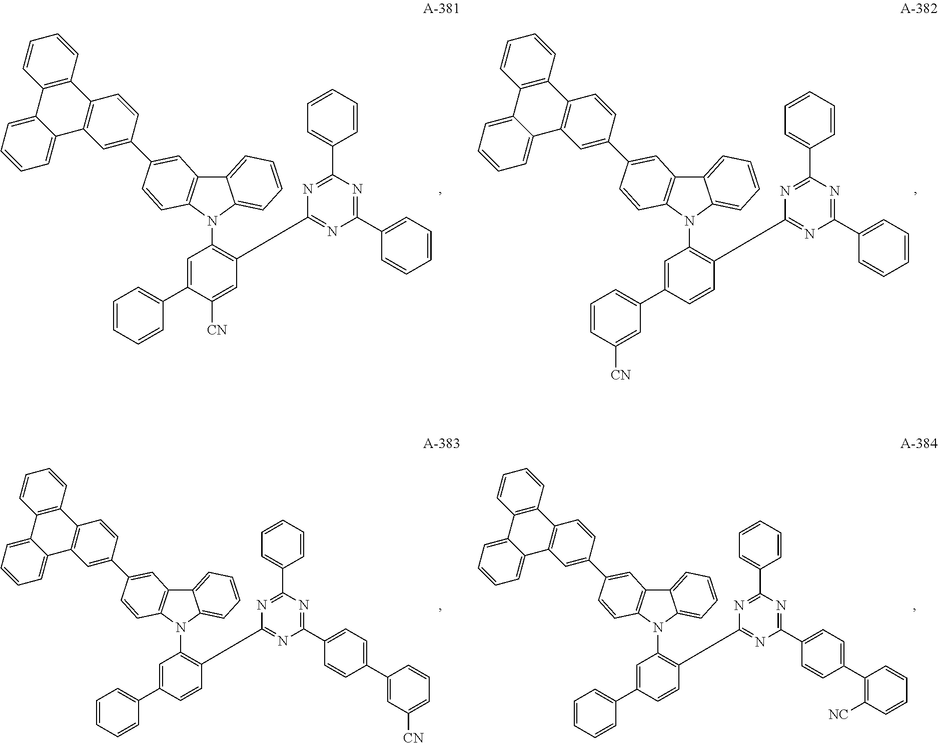 [SMILES: N#Cc1cc(-c2nc(-c3ccccc3)nc(-c3ccccc3)n2)c(-n2c3ccccc3c3cc(-c4ccc5c6ccccc6c6ccccc6c5c4)ccc32)cc1-c1ccccc1 ; N#Cc1cccc(-c2ccc(-c3nc(-c4ccccc4)nc(-c4ccccc4)n3)c(-n3c4ccccc4c4cc(-c5ccc6c7ccccc7c7ccccc7c6c5)ccc43)c2)c1 ; N#Cc1cccc(-c2ccc(-c3nc(-c4ccccc4)nc(-c4ccc(-c5ccccc5)cc4-n4c5ccccc5c5cc(-c6ccc7c8ccccc8c8ccccc8c7c6)ccc54)n3)cc2)c1 ; N#Cc1ccccc1-c1ccc(-c2nc(-c3ccccc3)nc(-c3ccc(-c4ccccc4)cc3-n3c4ccccc4c4cc(-c5ccc6c7ccccc7c7ccccc7c6c5)ccc43)n2)cc1] 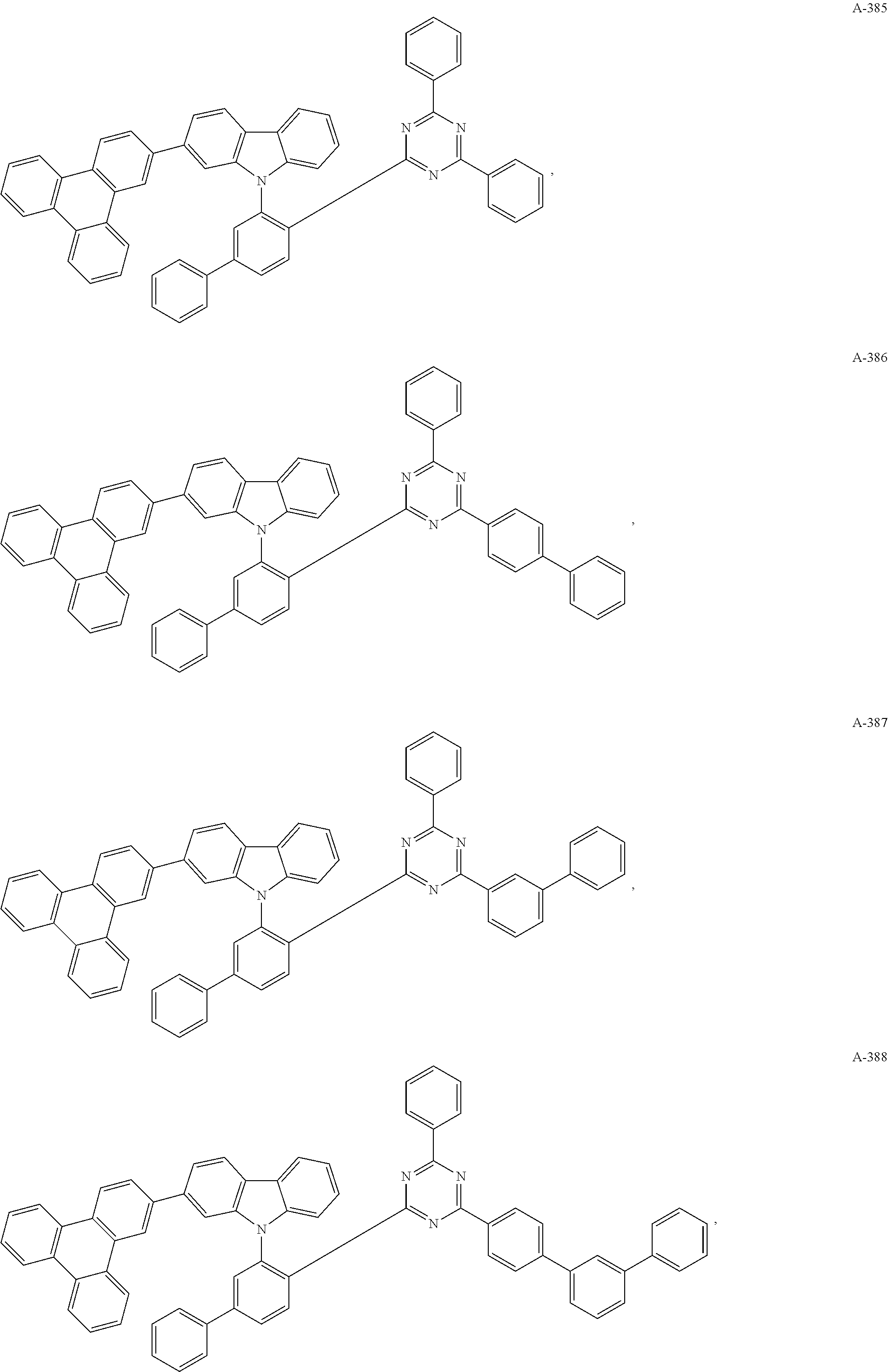 [SMILES: c1ccc(-c2ccc(-c3nc(-c4ccccc4)nc(-c4ccccc4)n3)c(-n3c4ccccc4c4ccc(-c5ccc6c7ccccc7c7ccccc7c6c5)cc43)c2)cc1 ; c1ccc(-c2ccc(-c3nc(-c4ccccc4)nc(-c4ccc(-c5ccccc5)cc4-n4c5ccccc5c5ccc(-c6ccc7c8ccccc8c8ccccc8c7c6)cc54)n3)cc2)cc1 ; c1ccc(-c2cccc(-c3nc(-c4ccccc4)nc(-c4ccc(-c5ccccc5)cc4-n4c5ccccc5c5ccc(-c6ccc7c8ccccc8c8ccccc8c7c6)cc54)n3)c2)cc1 ; c1ccc(-c2cccc(-c3ccc(-c4nc(-c5ccccc5)nc(-c5ccc(-c6ccccc6)cc5-n5c6ccccc6c6ccc(-c7ccc8c9ccccc9c9ccccc9c8c7)cc65)n4)cc3)c2)cc1] 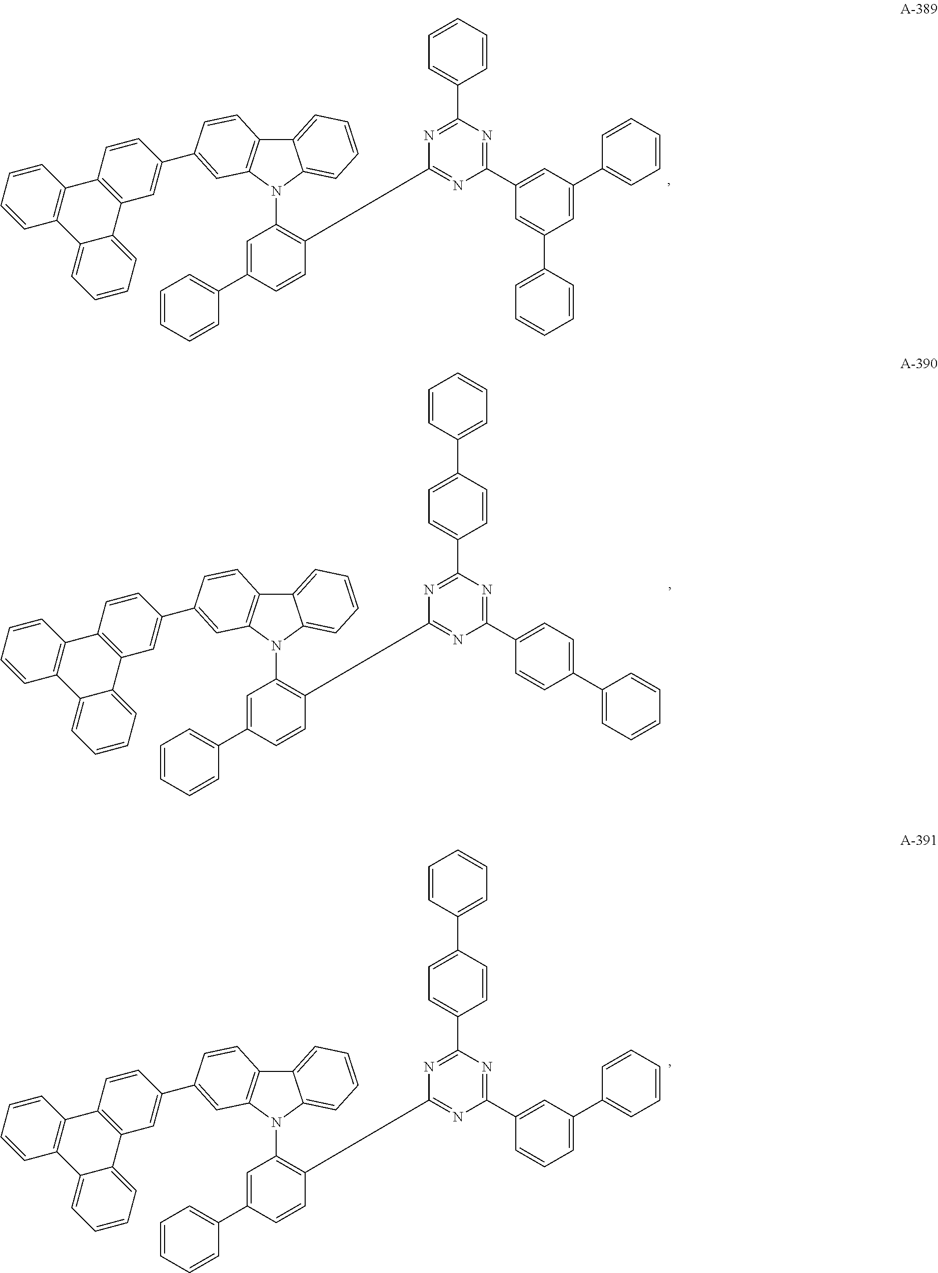 [SMILES: c1ccc(-c2cc(-c3ccccc3)cc(-c3nc(-c4ccccc4)nc(-c4ccc(-c5ccccc5)cc4-n4c5ccccc5c5ccc(-c6ccc7c8ccccc8c8ccccc8c7c6)cc54)n3)c2)cc1 ; c1ccc(-c2ccc(-c3nc(-c4ccc(-c5ccccc5)cc4)nc(-c4ccc(-c5ccccc5)cc4-n4c5ccccc5c5ccc(-c6ccc7c8ccccc8c8ccccc8c7c6)cc54)n3)cc2)cc1 ; c1ccc(-c2ccc(-c3nc(-c4cccc(-c5ccccc5)c4)nc(-c4ccc(-c5ccccc5)cc4-n4c5ccccc5c5ccc(-c6ccc7c8ccccc8c8ccccc8c7c6)cc54)n3)cc2)cc1] 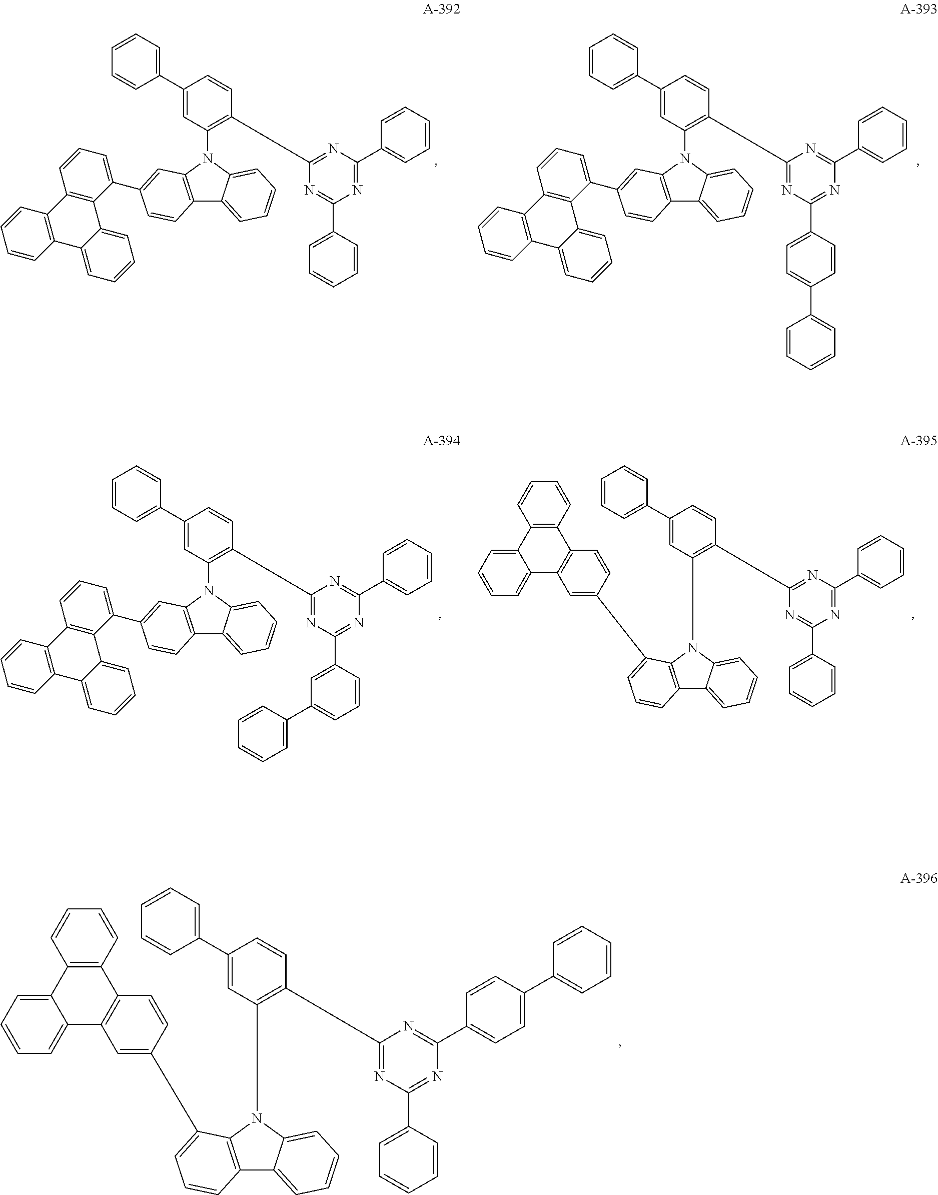 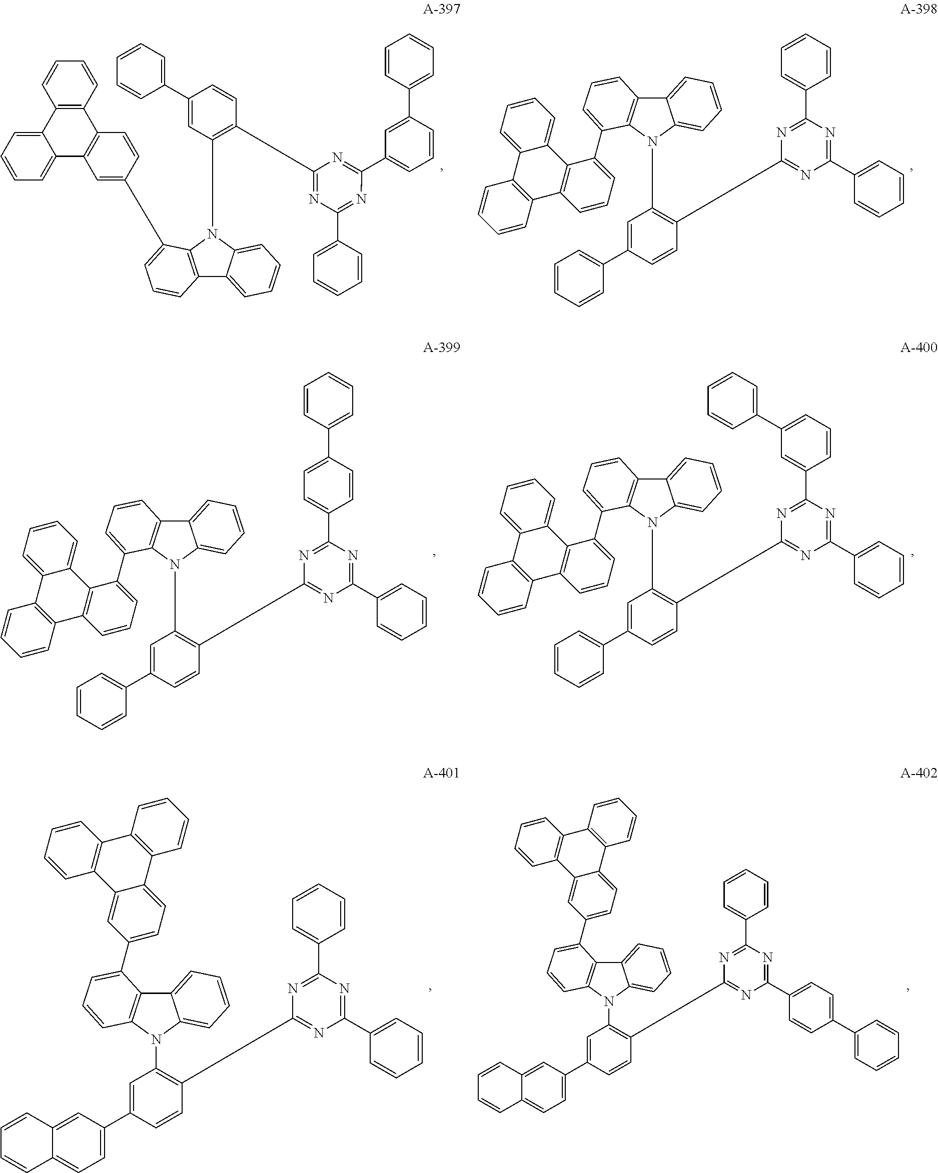 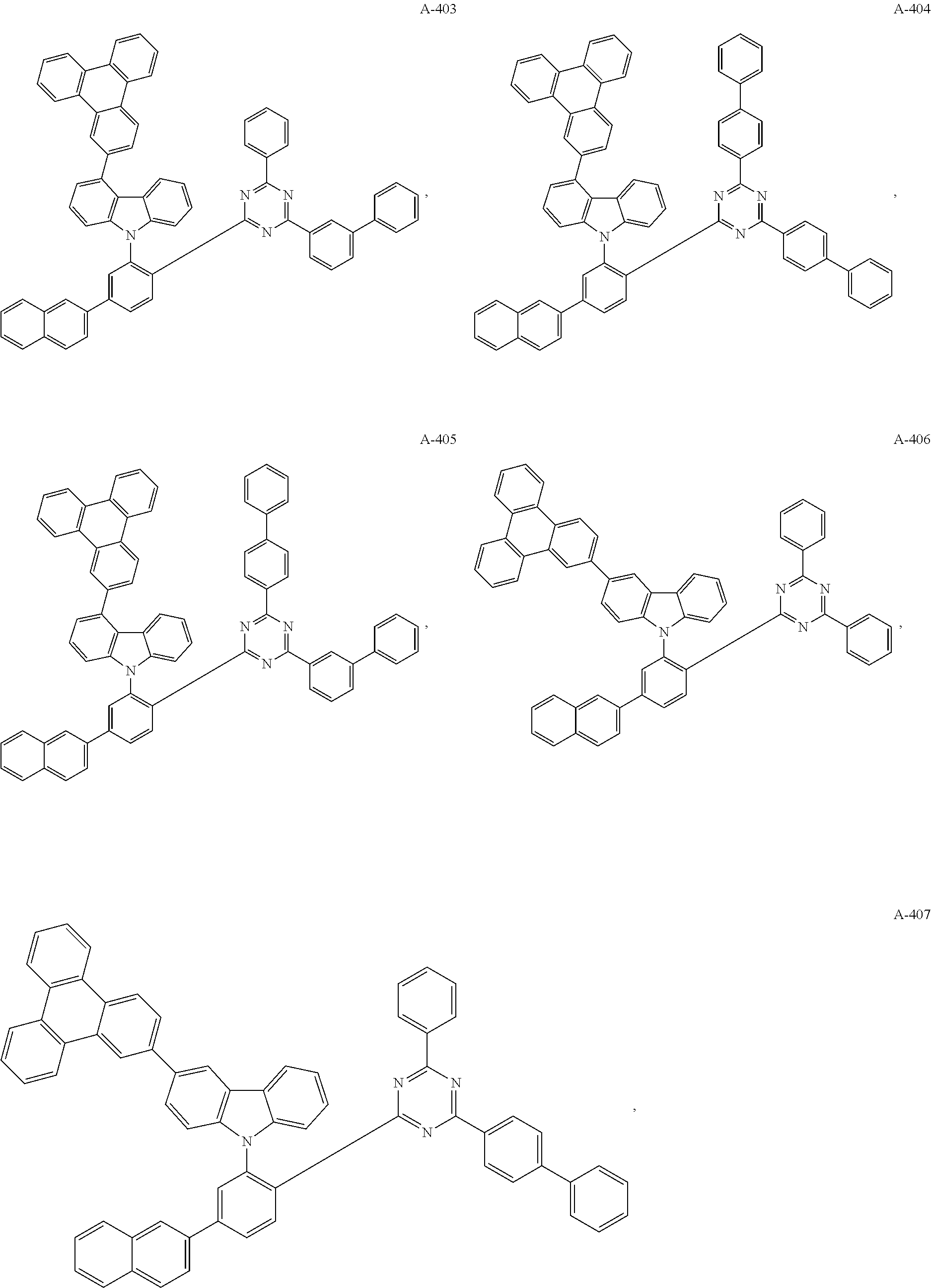 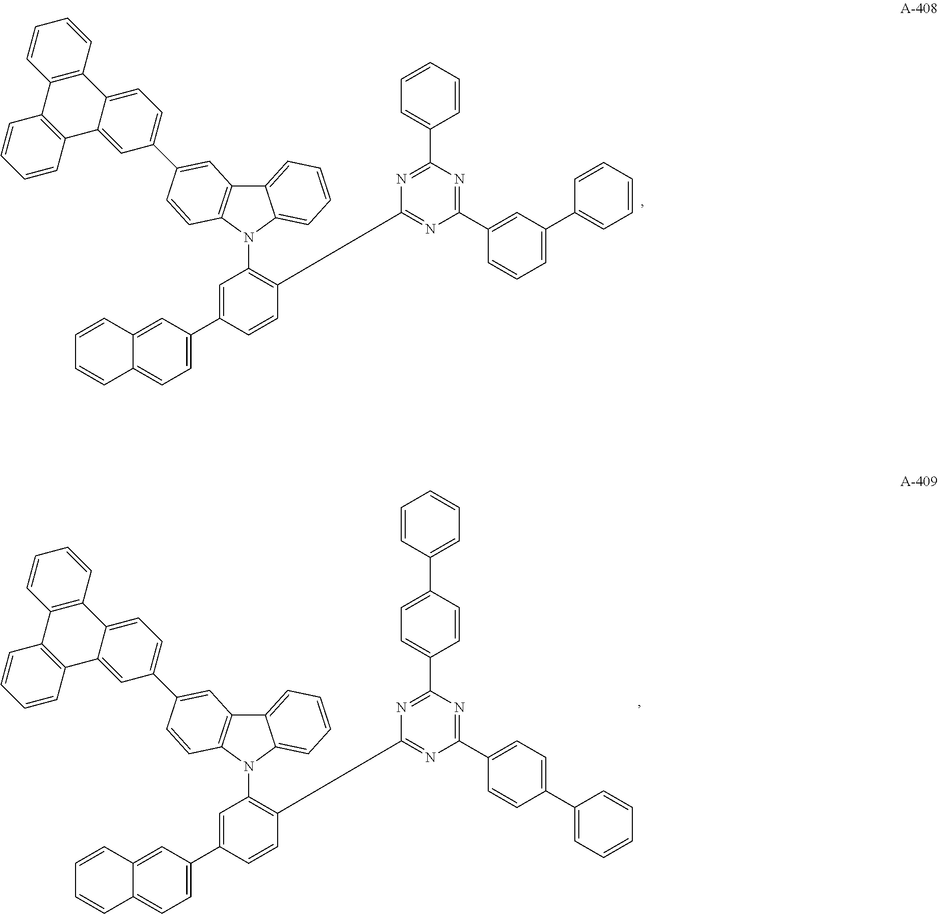 [SMILES: c1ccc(-c2cccc(-c3nc(-c4ccccc4)nc(-c4ccc(-c5ccc6ccccc6c5)cc4-n4c5ccccc5c5cc(-c6ccc7c8ccccc8c8ccccc8c7c6)ccc54)n3)c2)cc1 ; c1ccc(-c2ccc(-c3nc(-c4ccc(-c5ccccc5)cc4)nc(-c4ccc(-c5ccc6ccccc6c5)cc4-n4c5ccccc5c5cc(-c6ccc7c8ccccc8c8ccccc8c7c6)ccc54)n3)cc2)cc1] 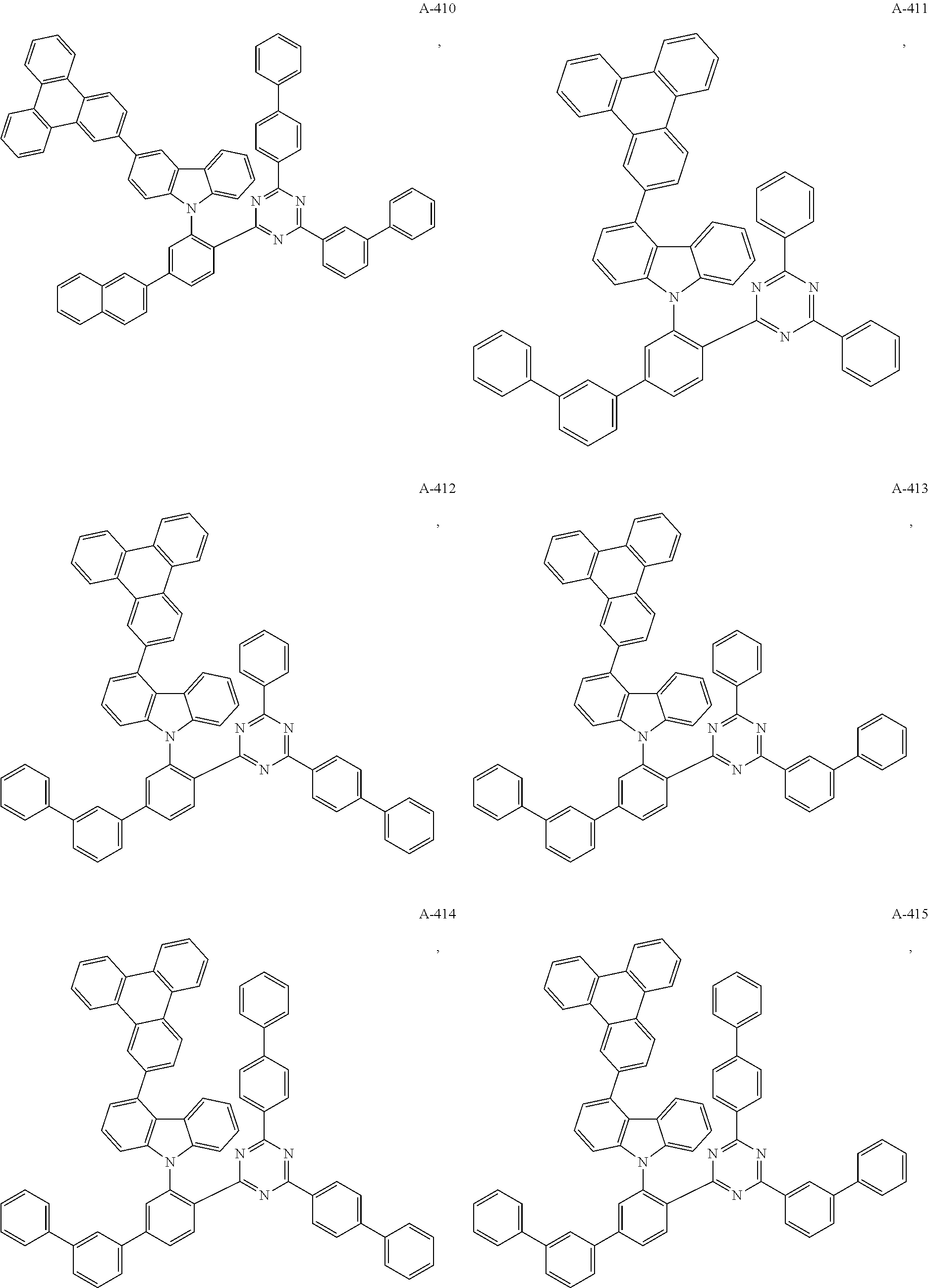 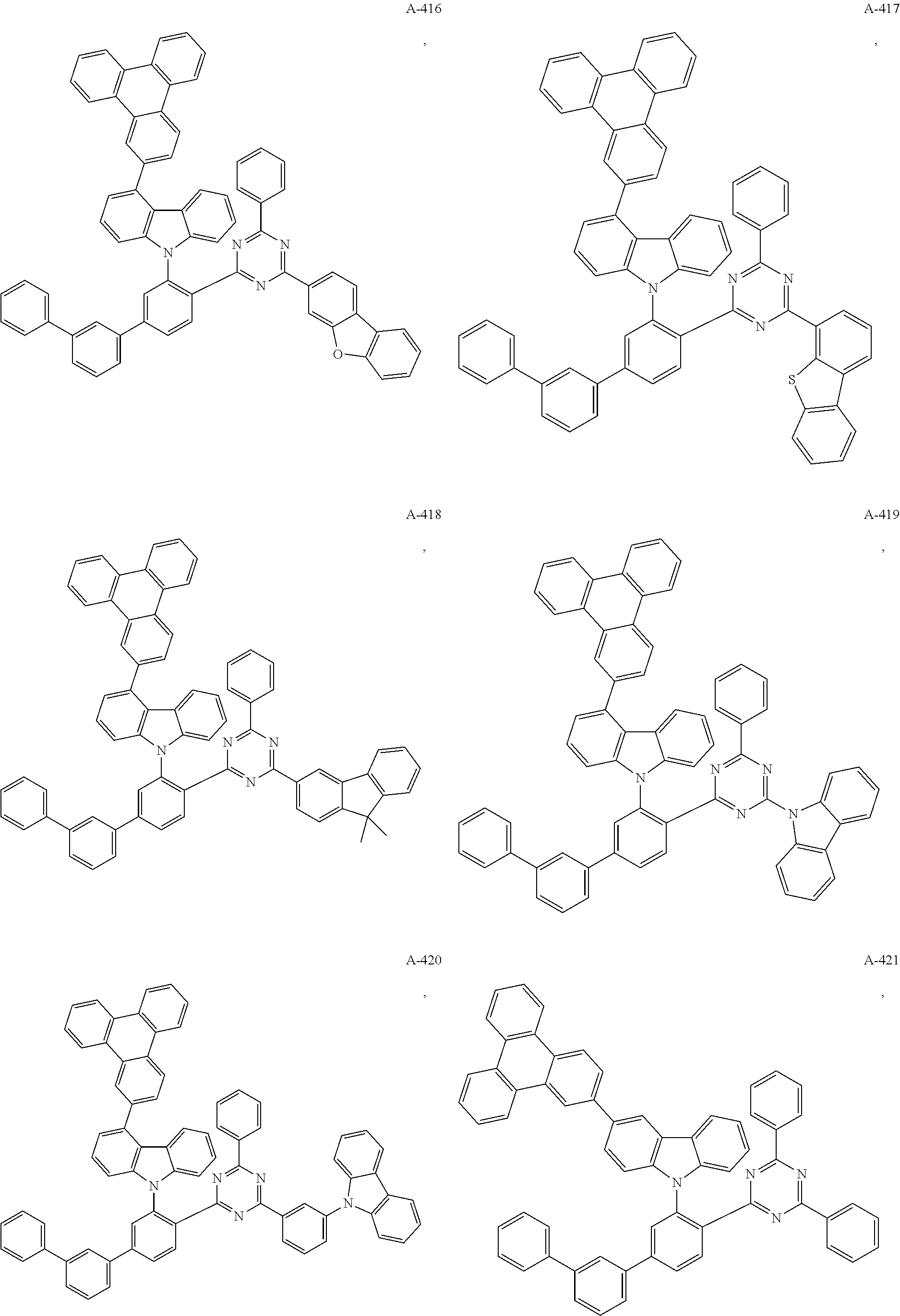 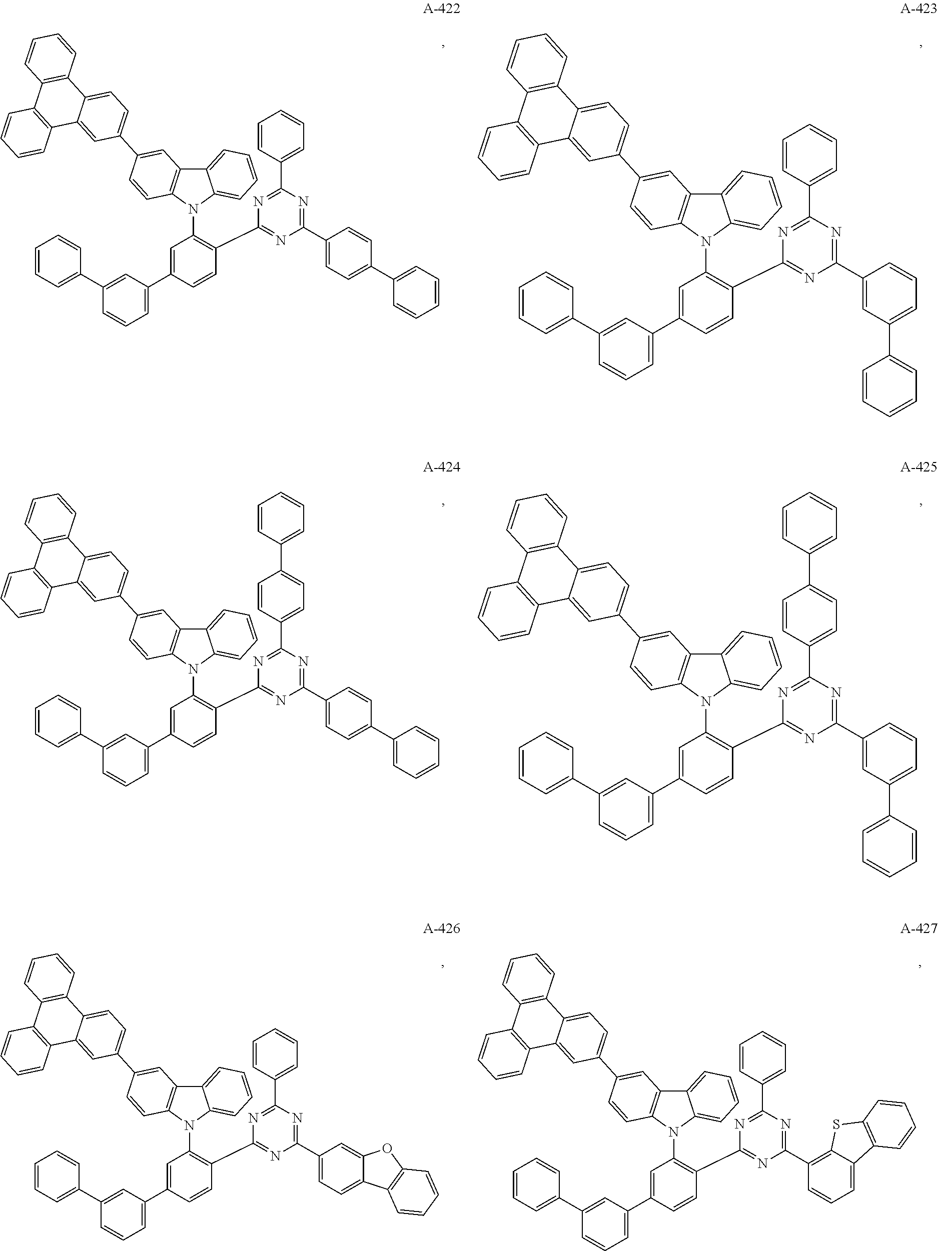 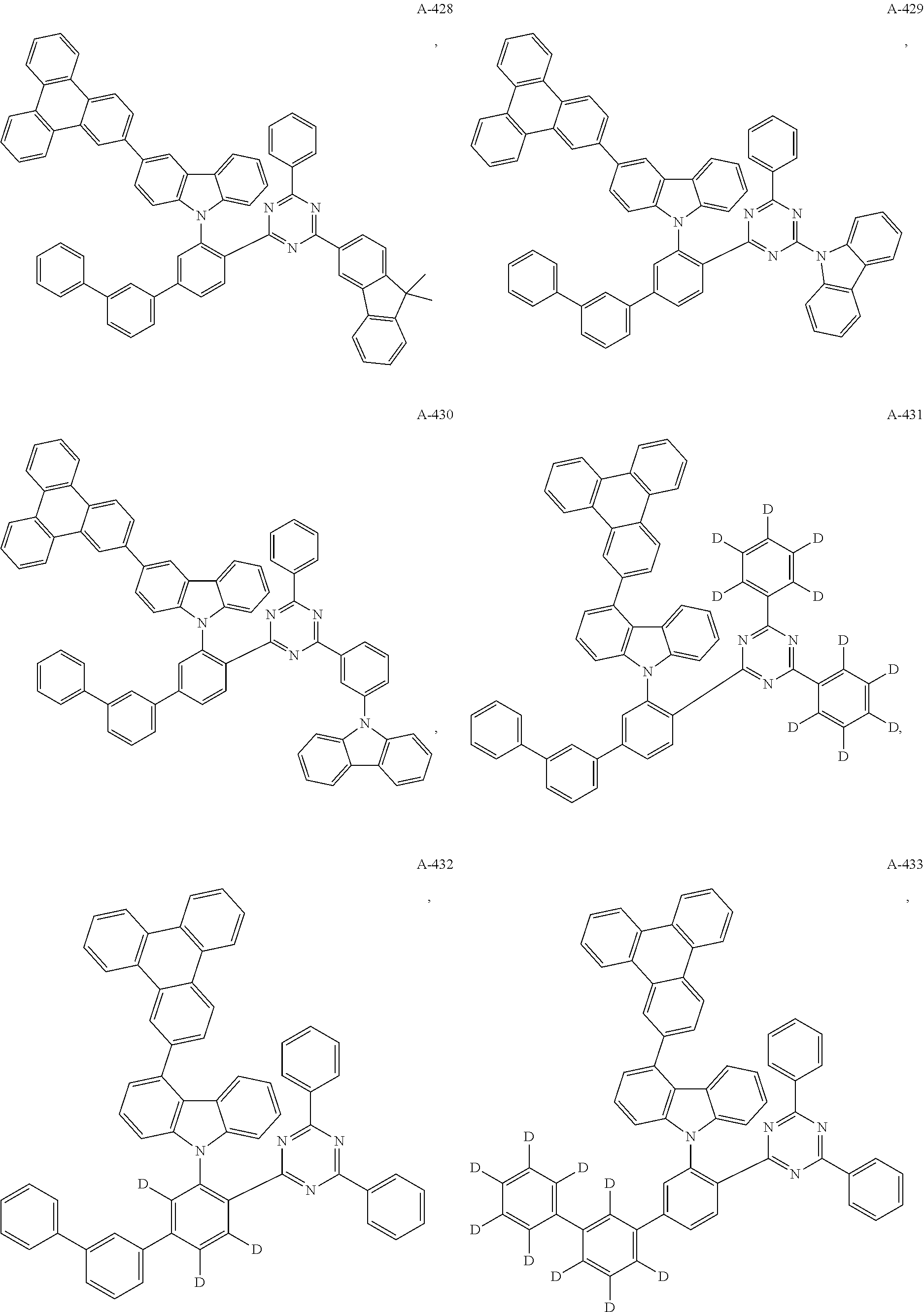 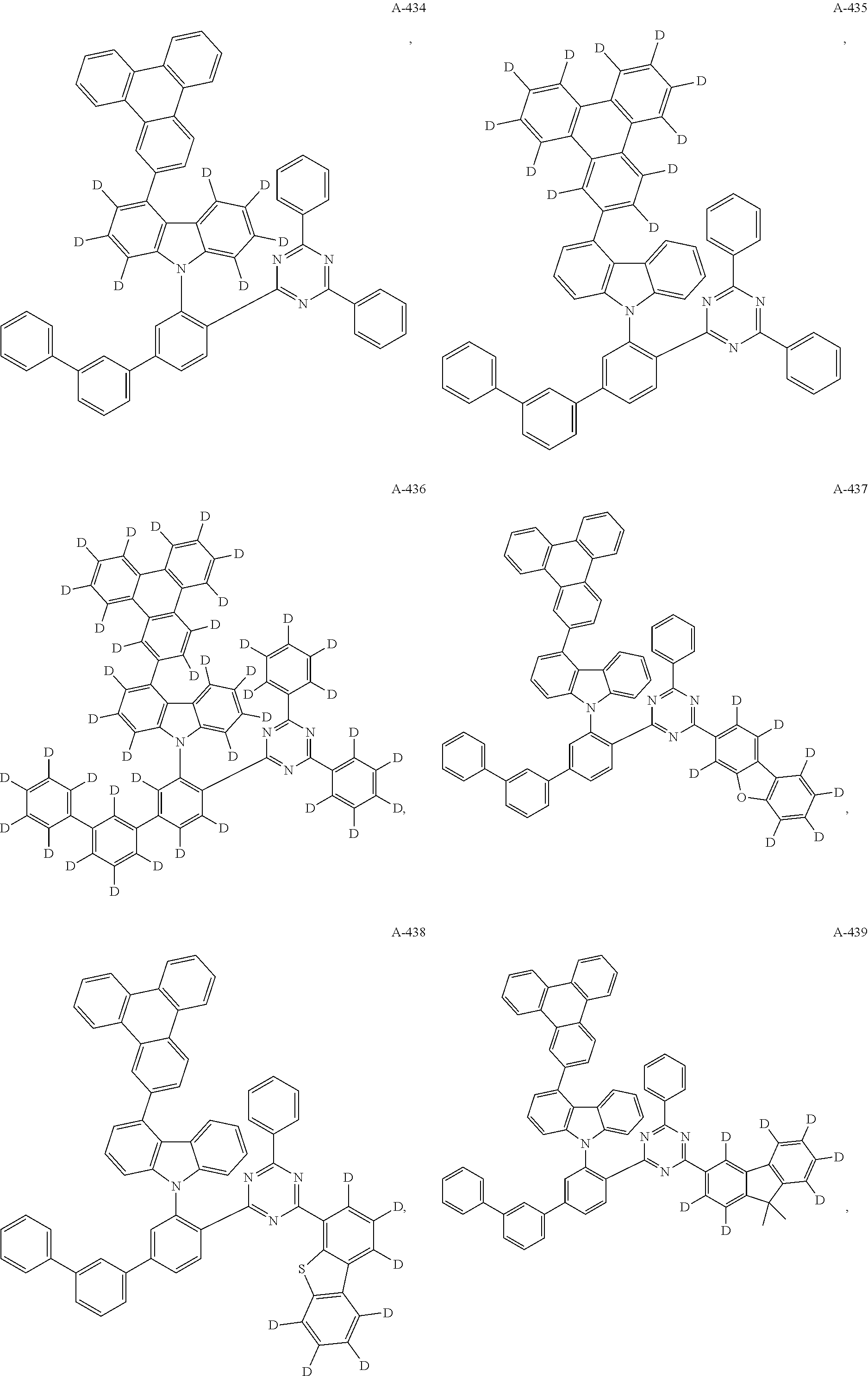 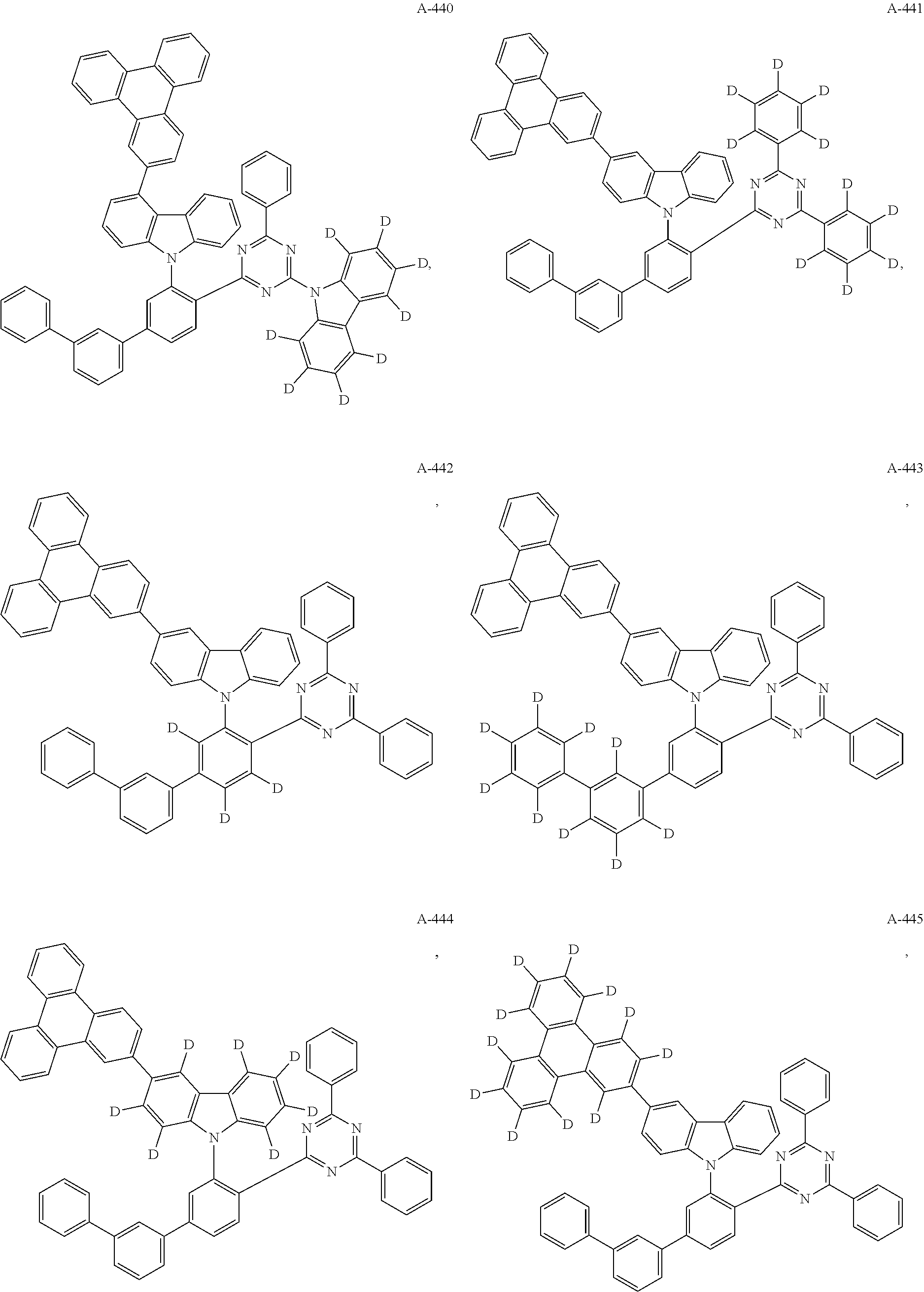 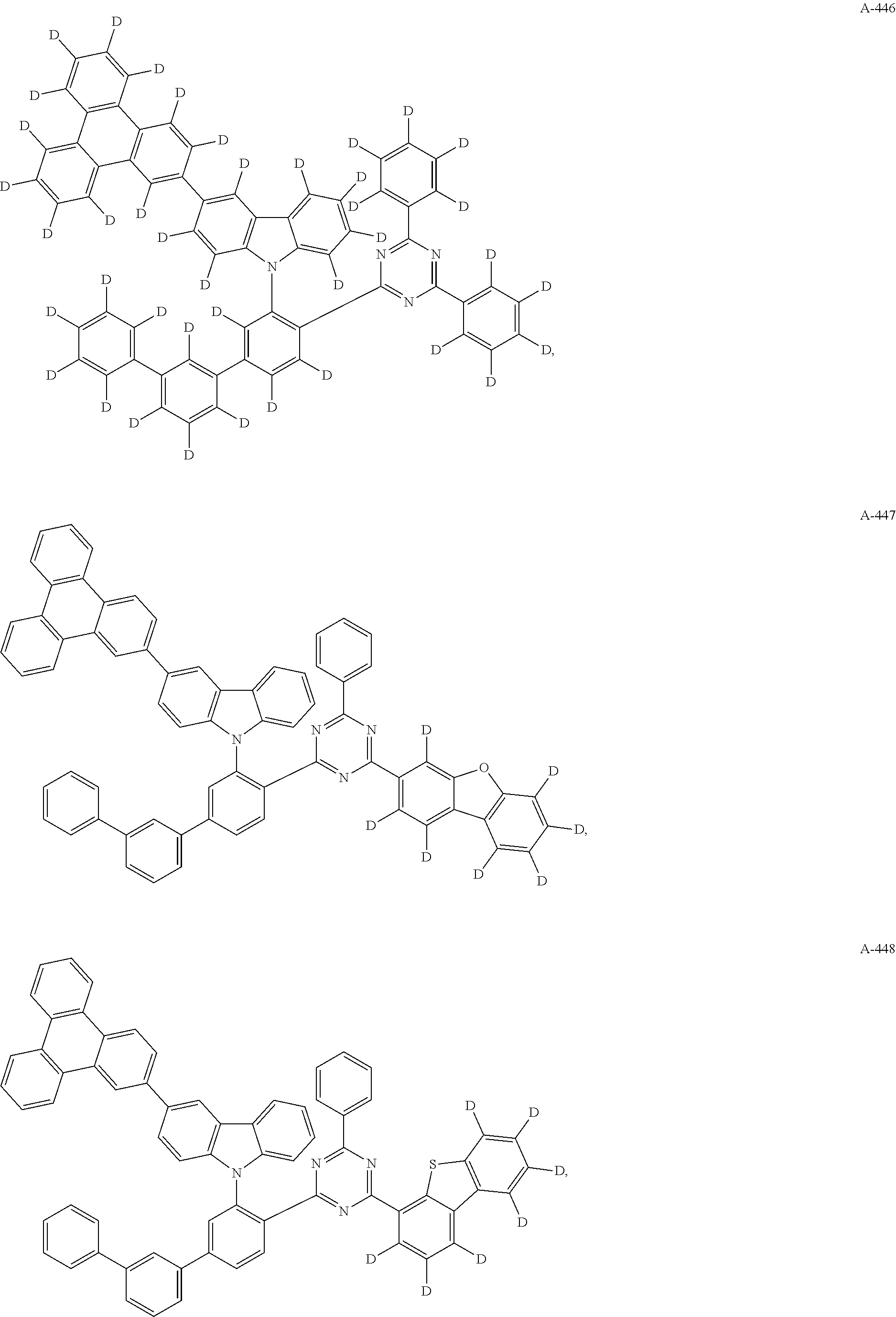 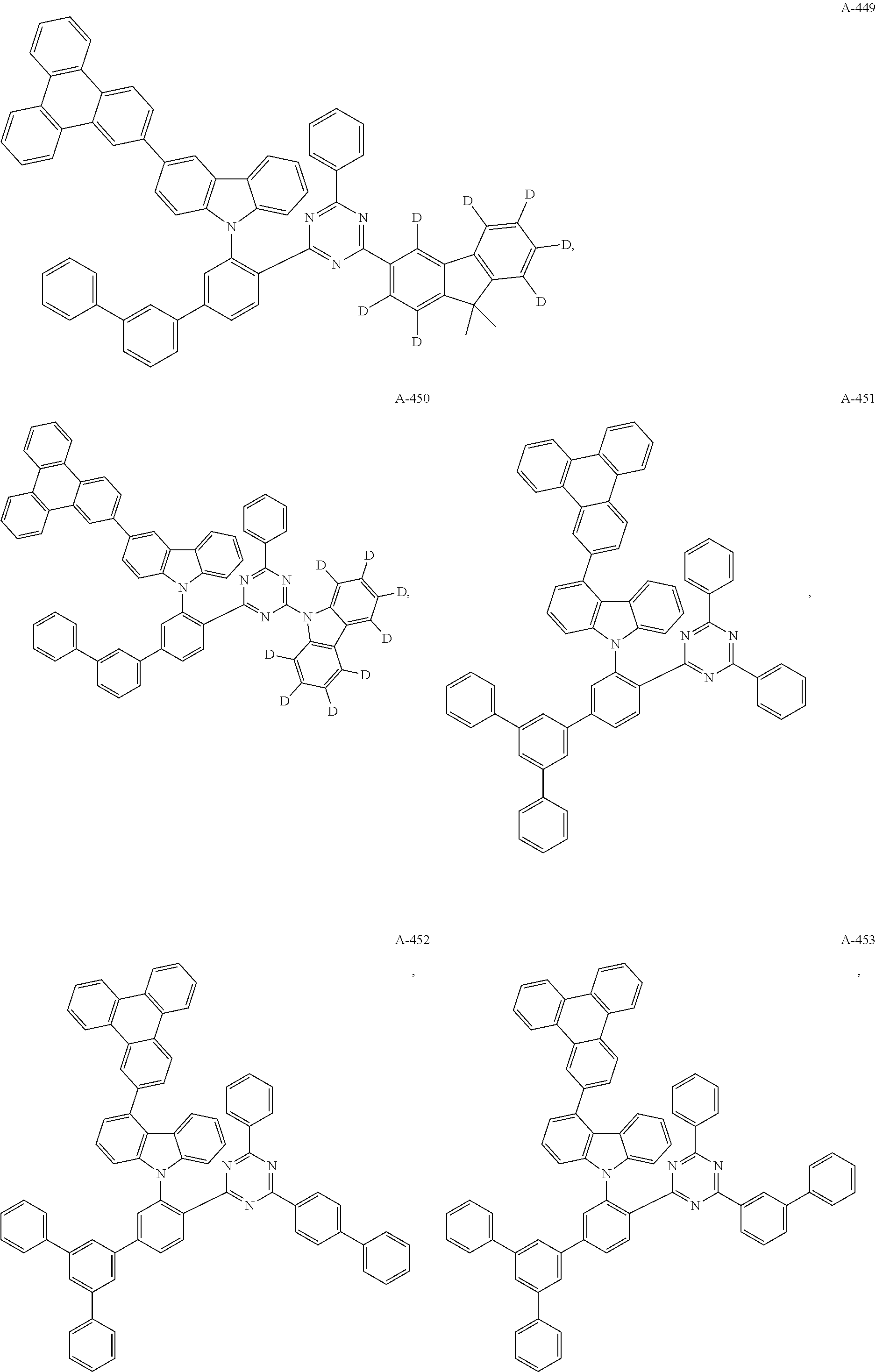 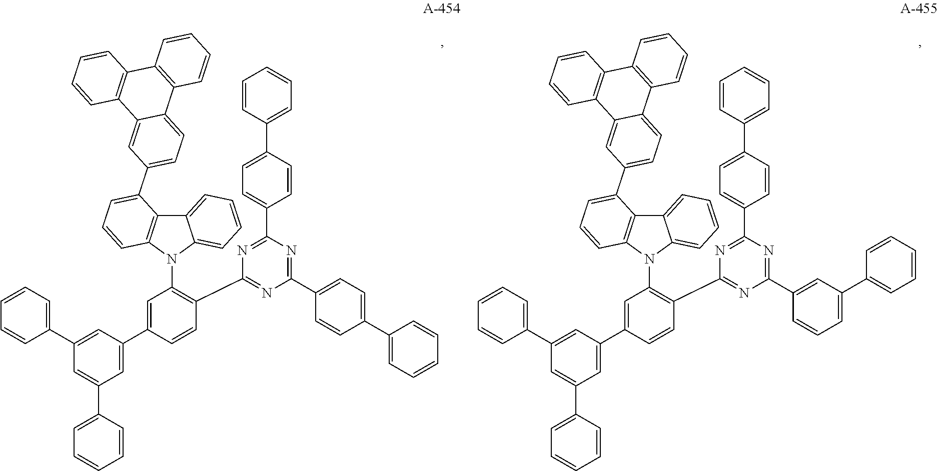 [SMILES: c1ccc(-c2ccc(-c3nc(-c4ccc(-c5ccccc5)cc4)nc(-c4ccc(-c5cc(-c6ccccc6)cc(-c6ccccc6)c5)cc4-n4c5ccccc5c5c(-c6ccc7c8ccccc8c8ccccc8c7c6)cccc54)n3)cc2)cc1 ; c1ccc(-c2ccc(-c3nc(-c4cccc(-c5ccccc5)c4)nc(-c4ccc(-c5cc(-c6ccccc6)cc(-c6ccccc6)c5)cc4-n4c5ccccc5c5c(-c6ccc7c8ccccc8c8ccccc8c7c6)cccc54)n3)cc2)cc1] 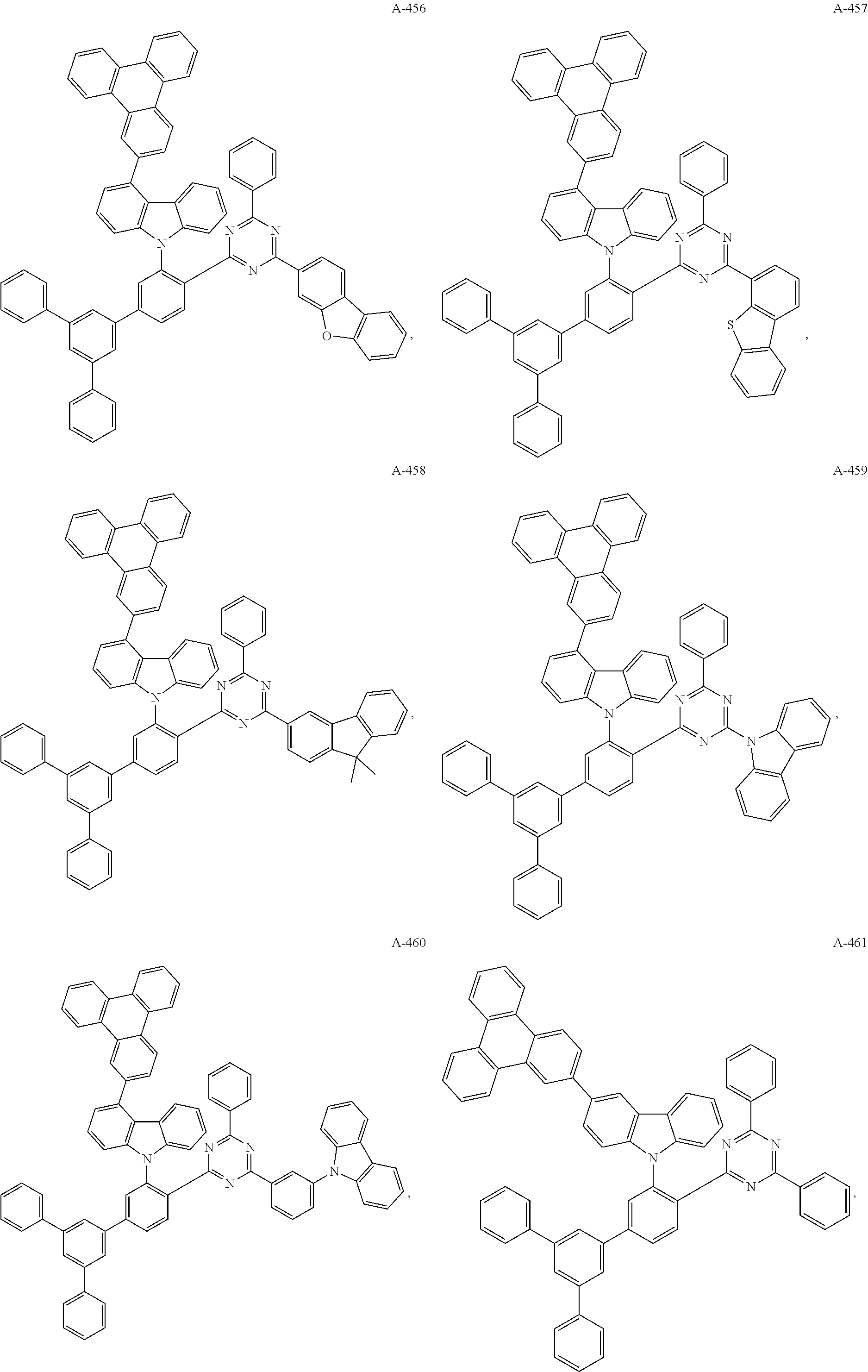 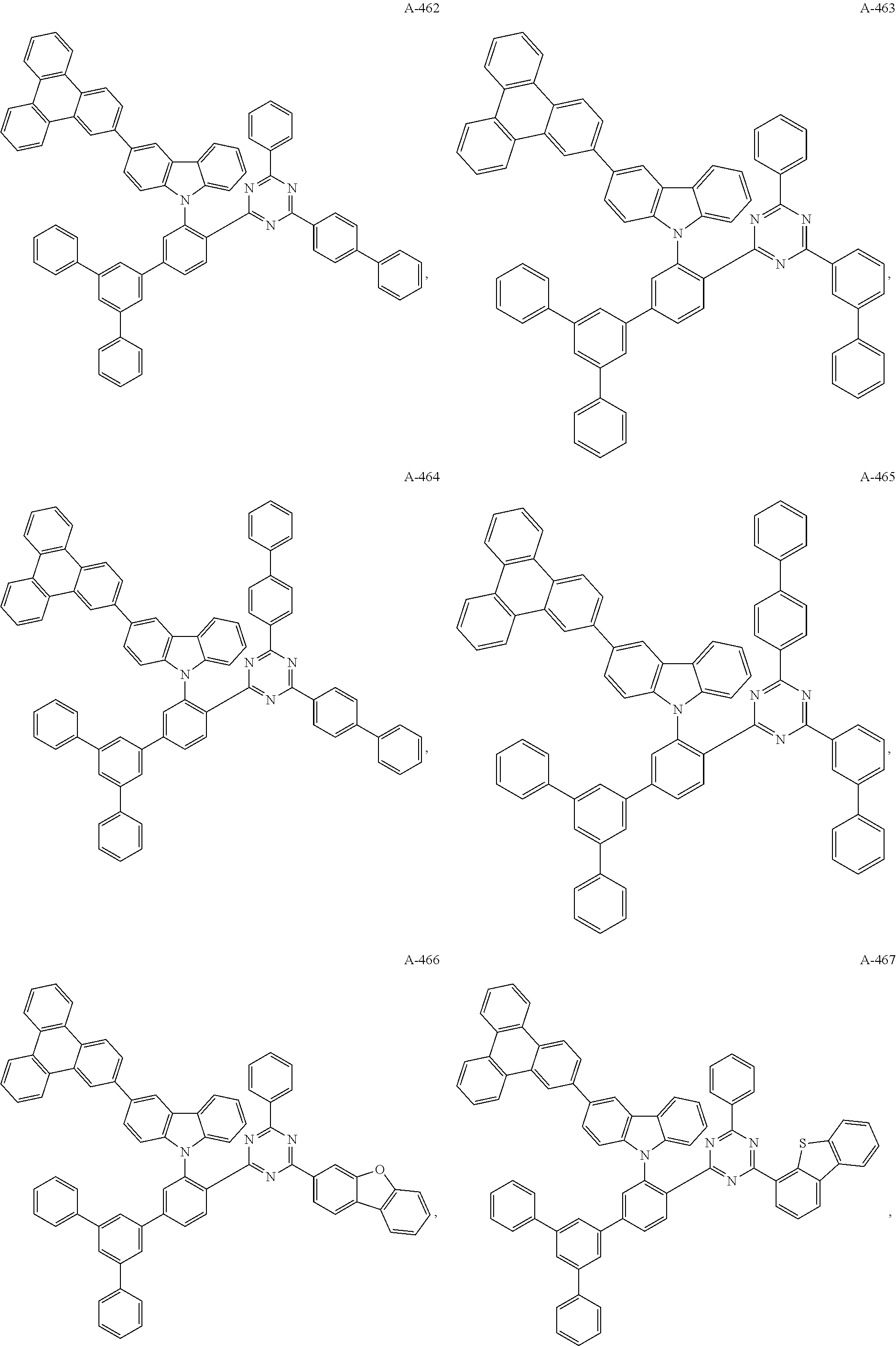 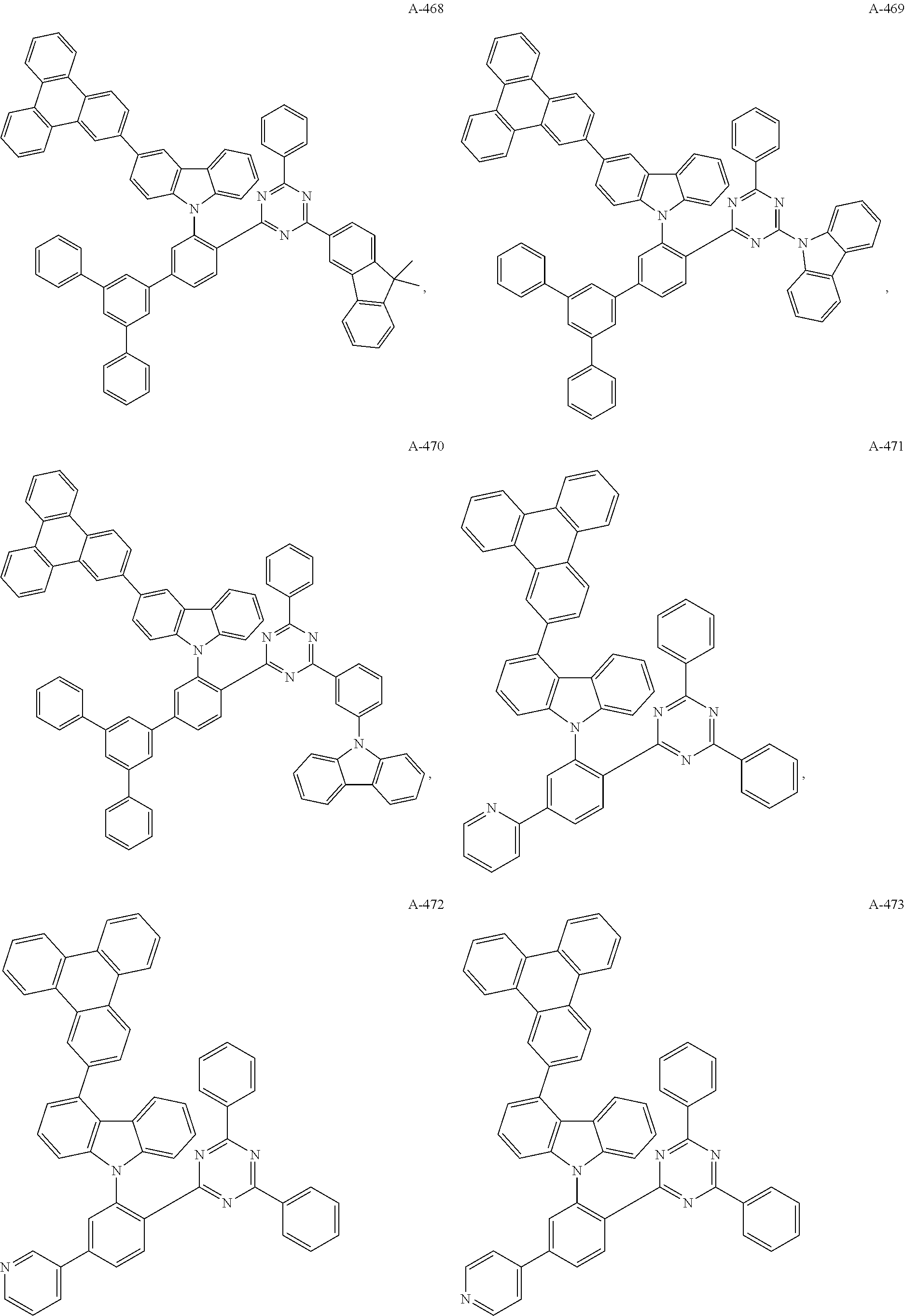 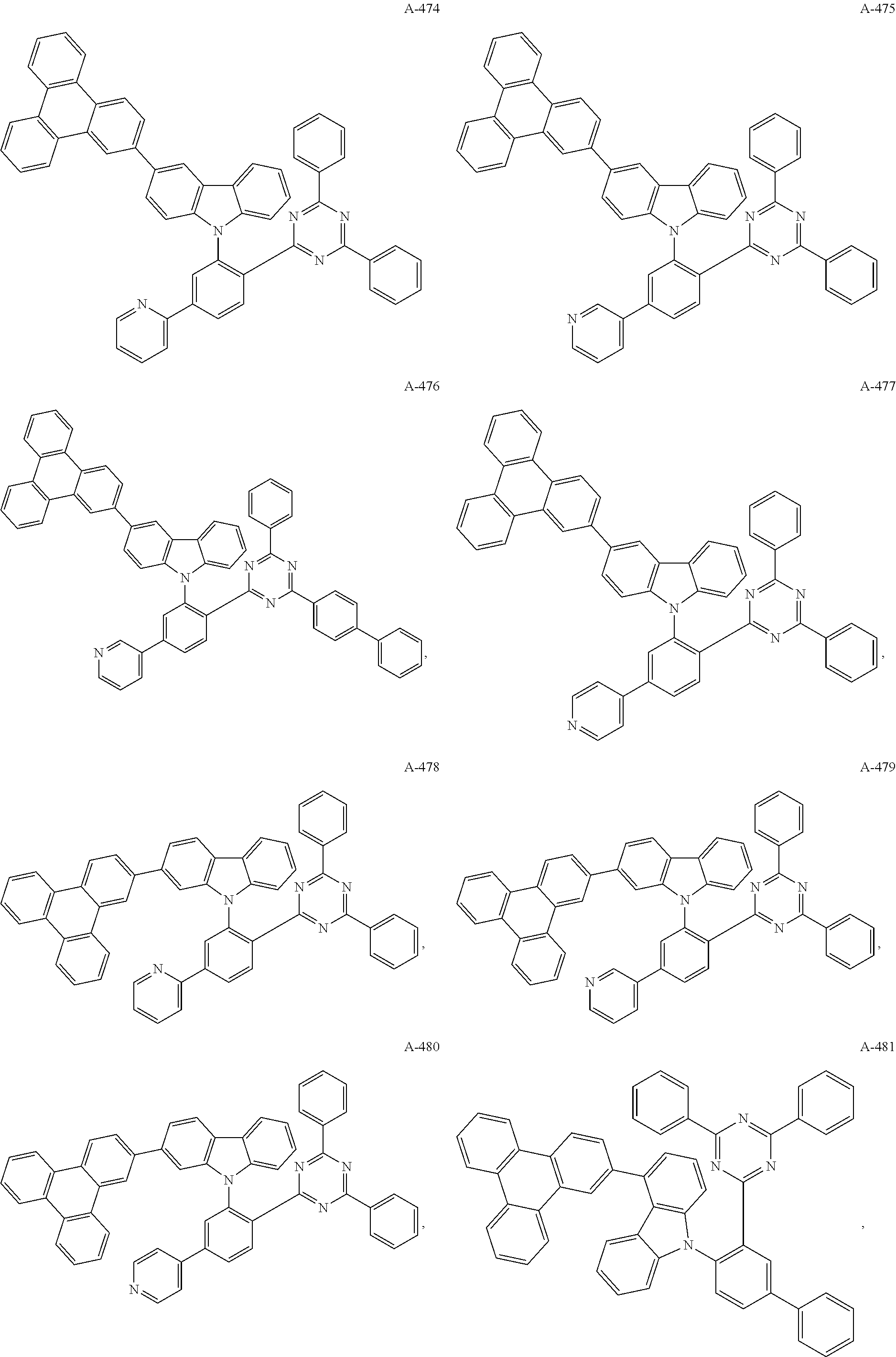 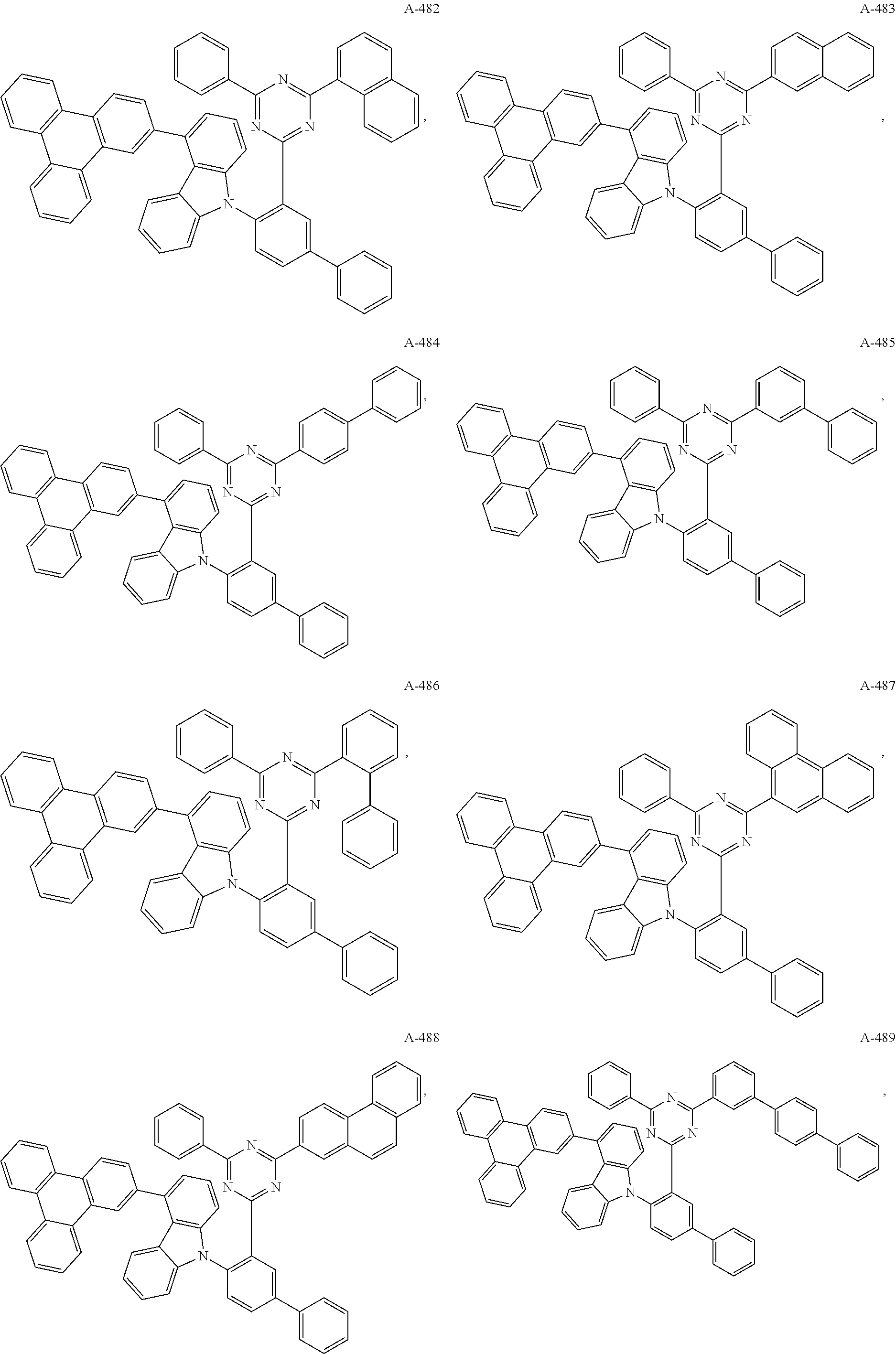 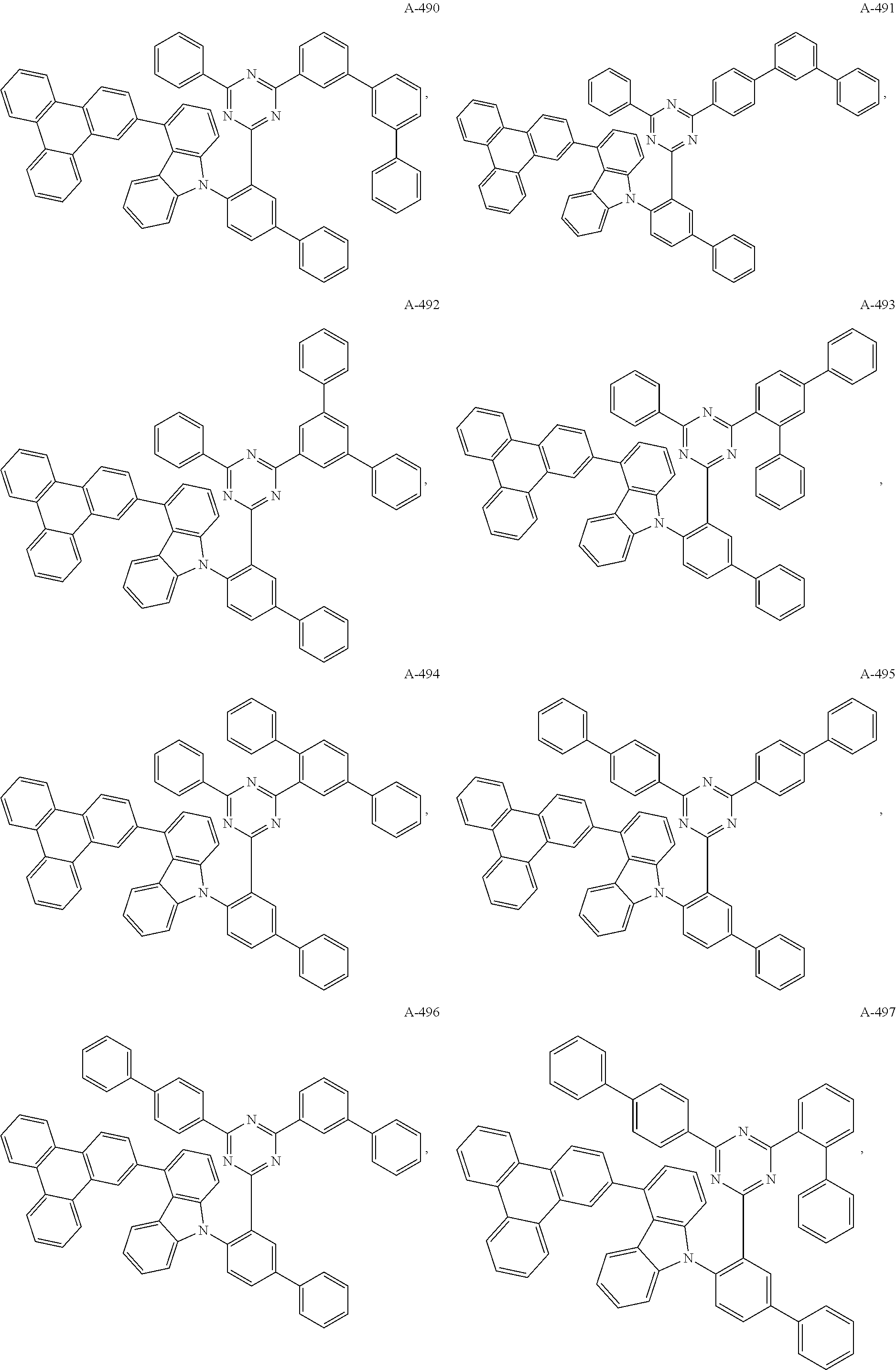 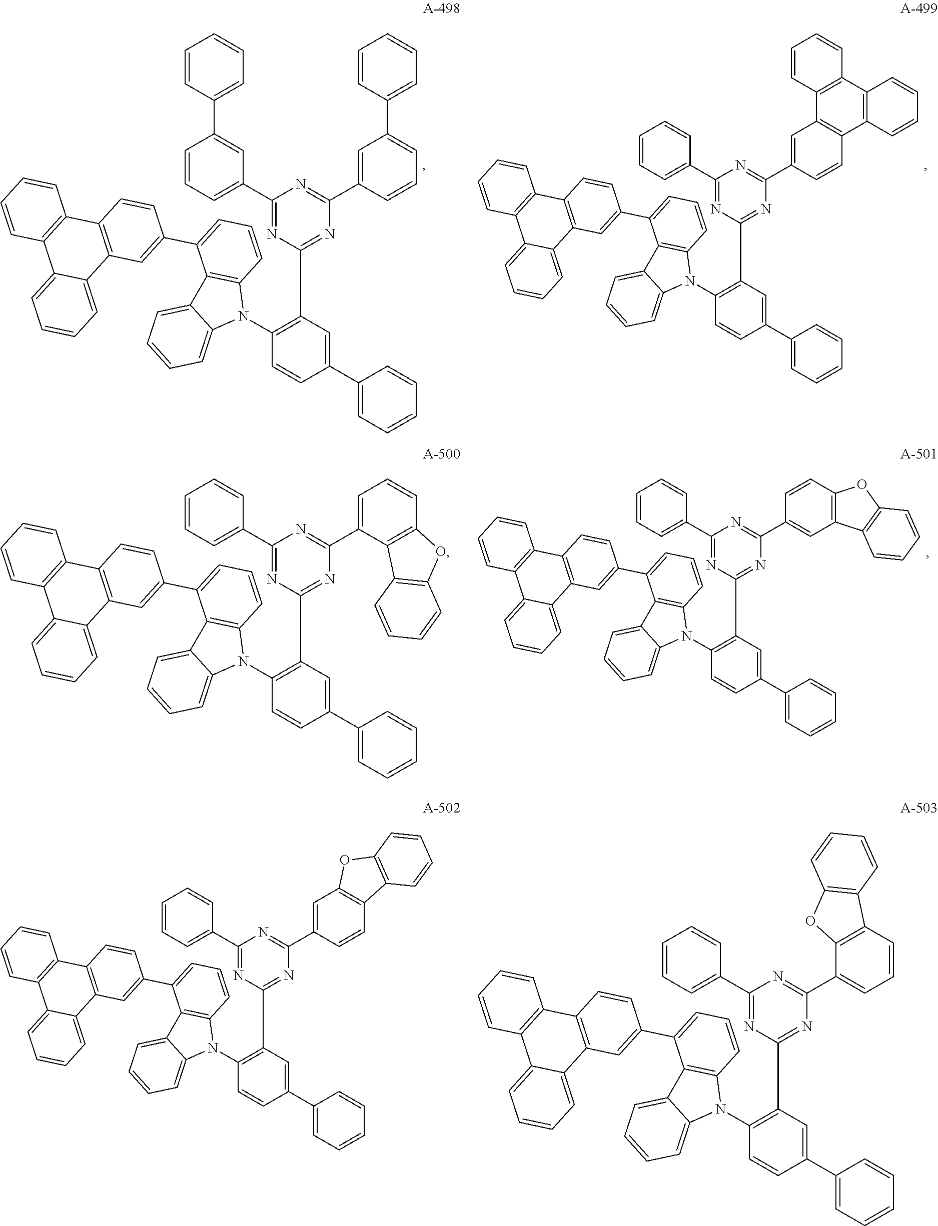 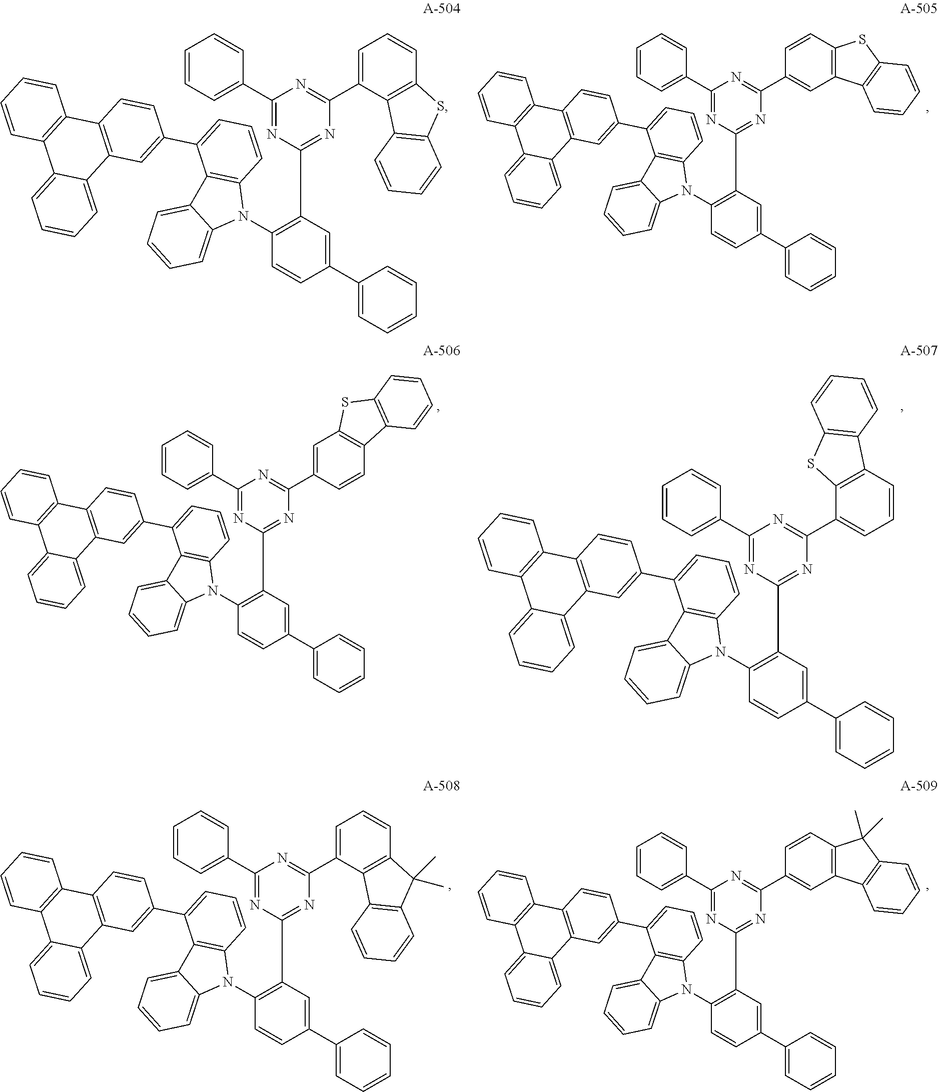 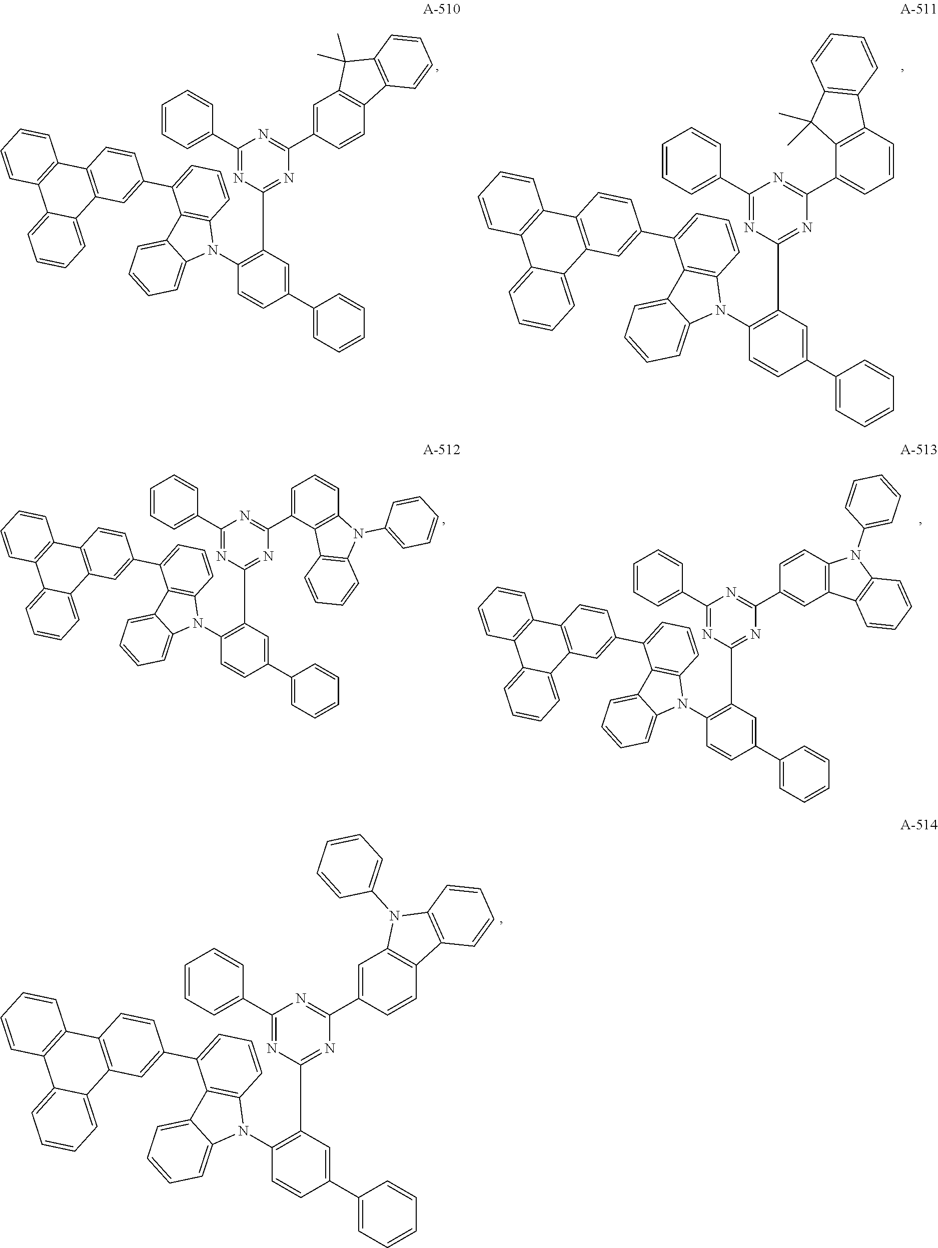 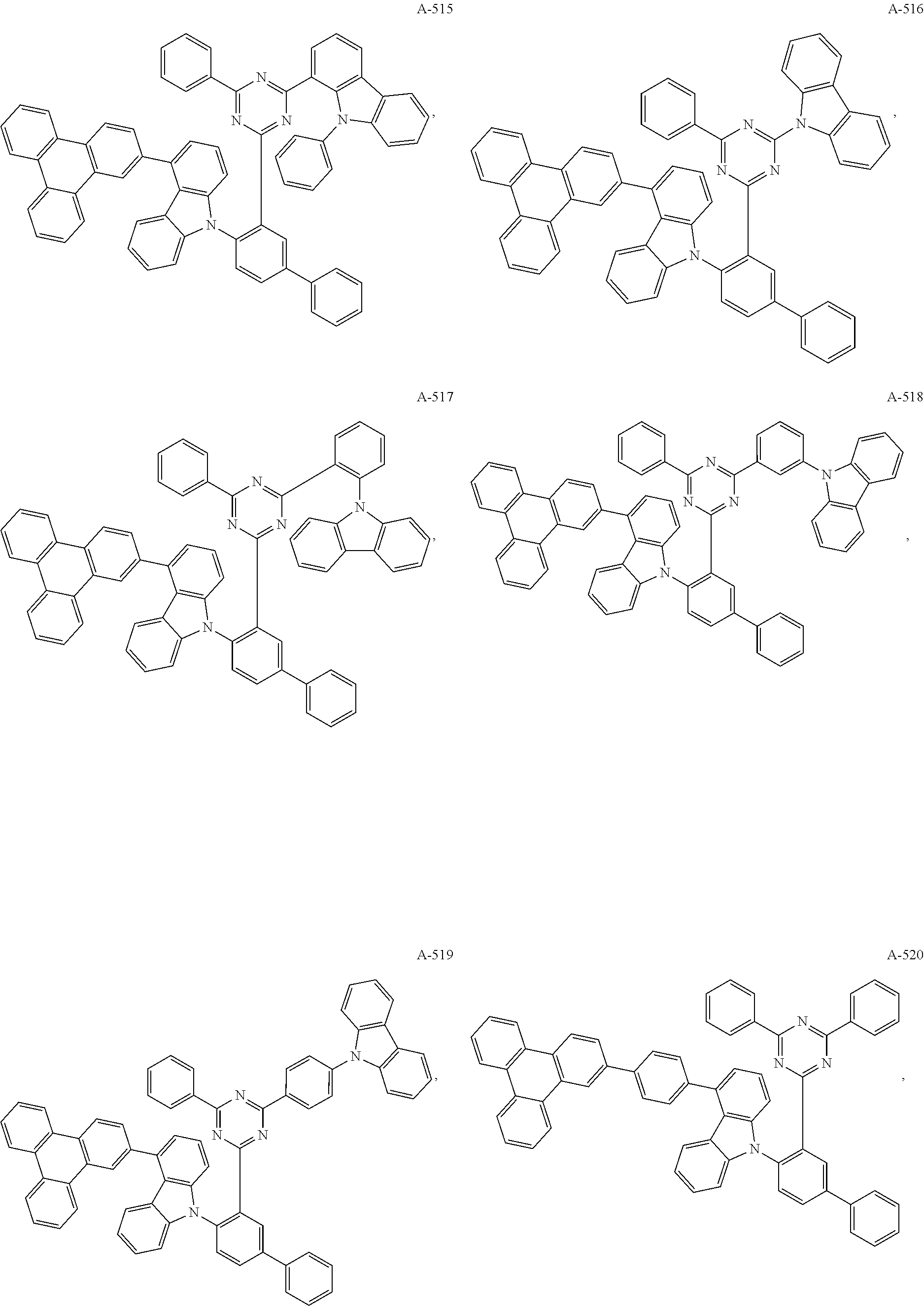 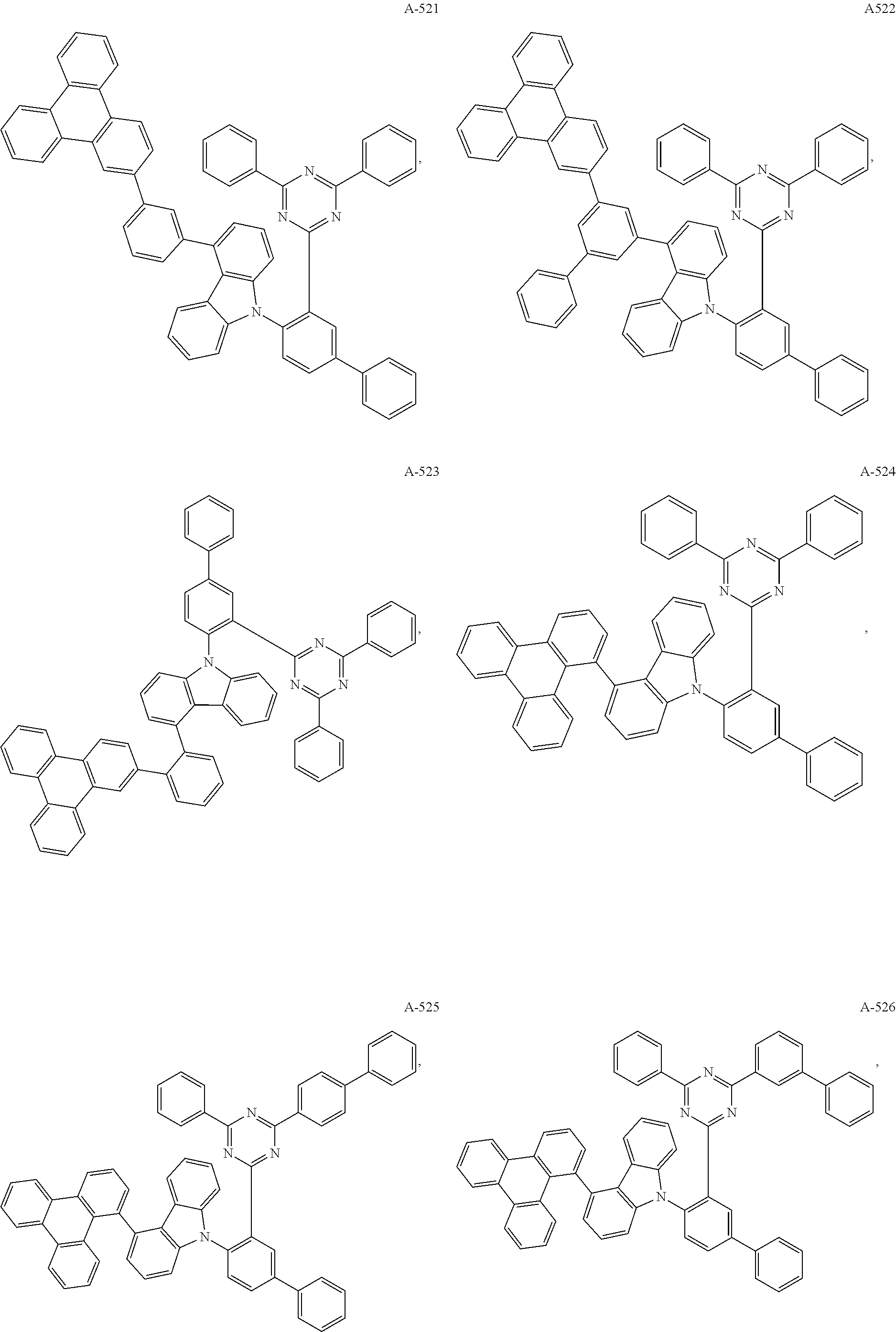 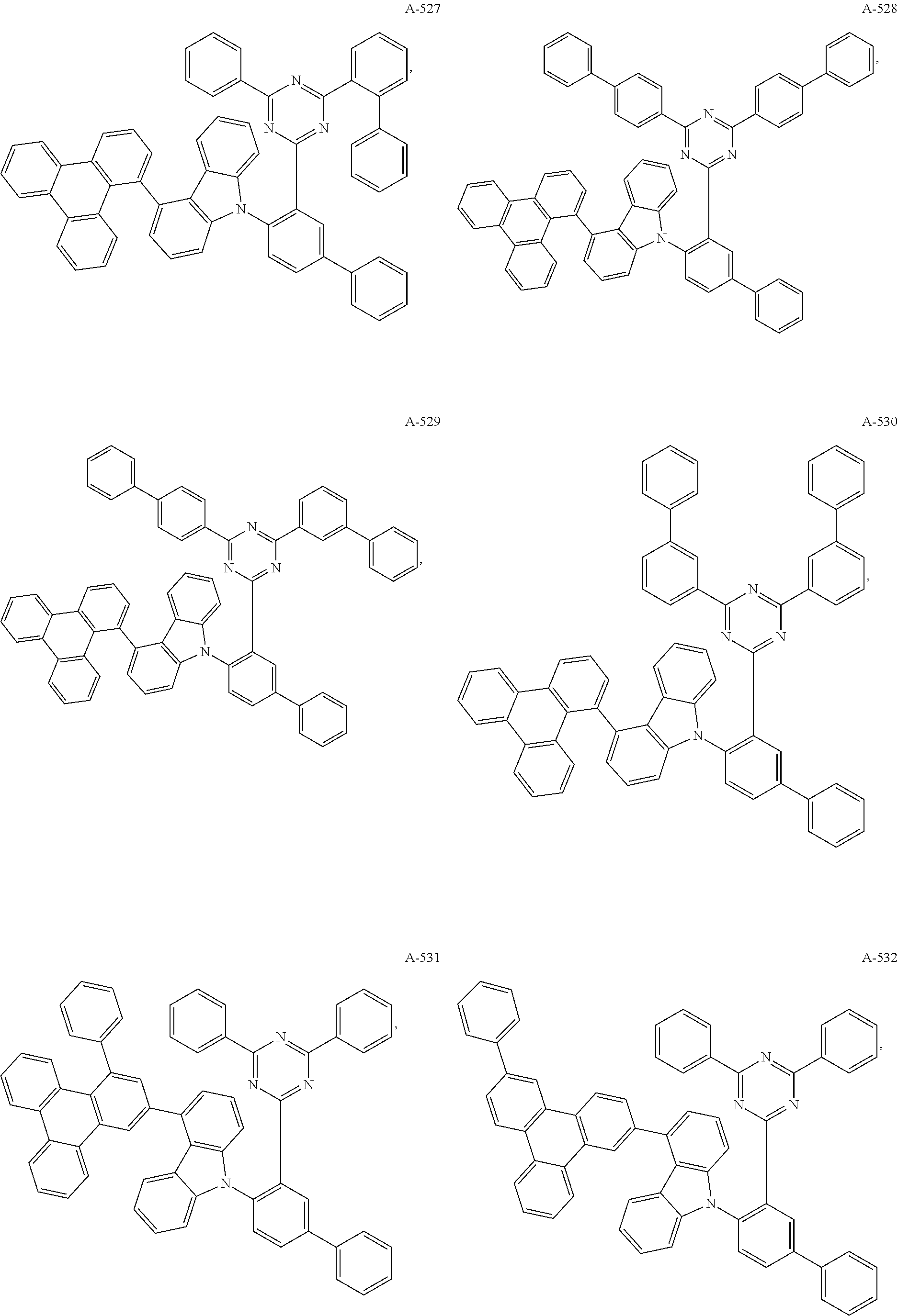 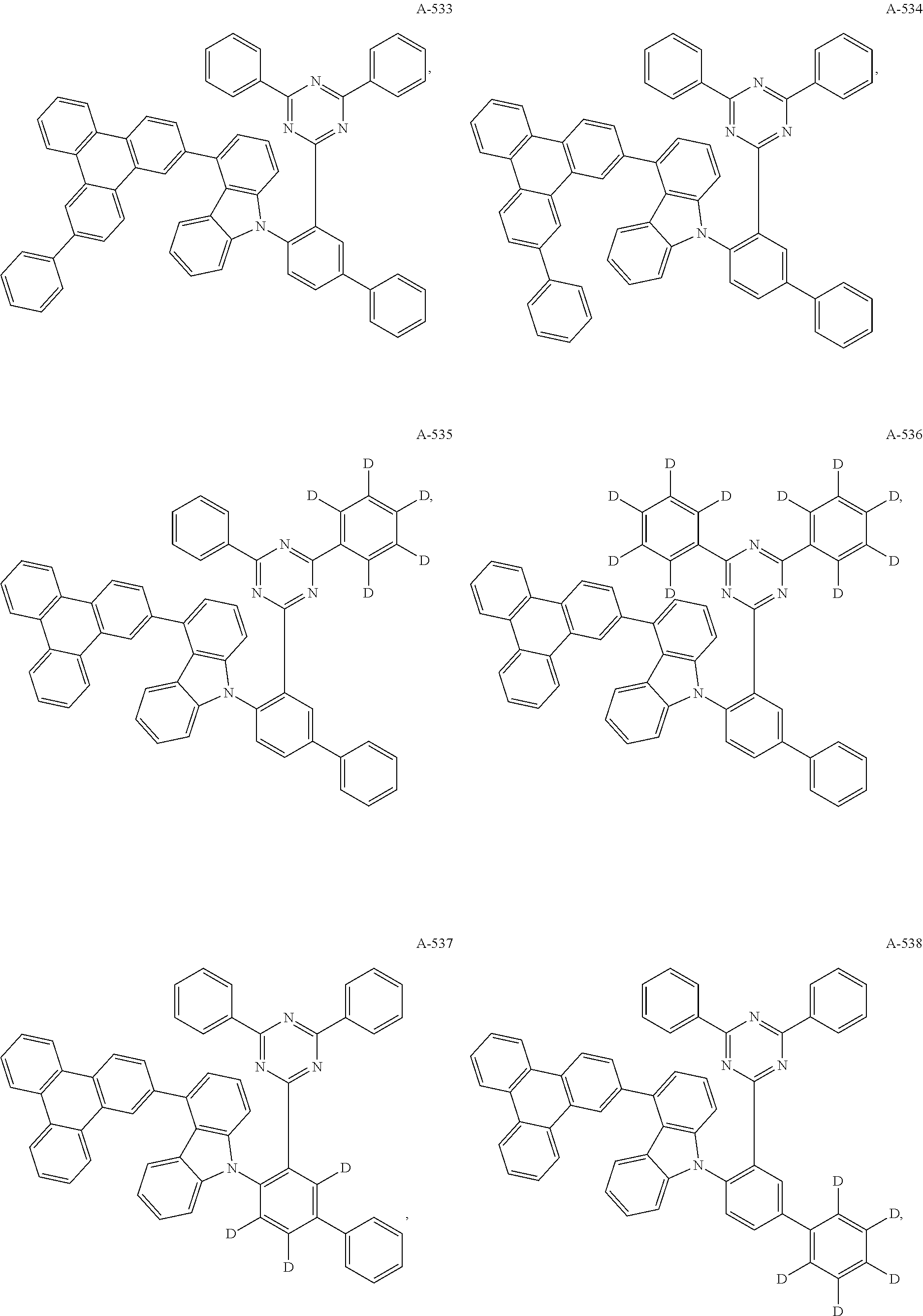 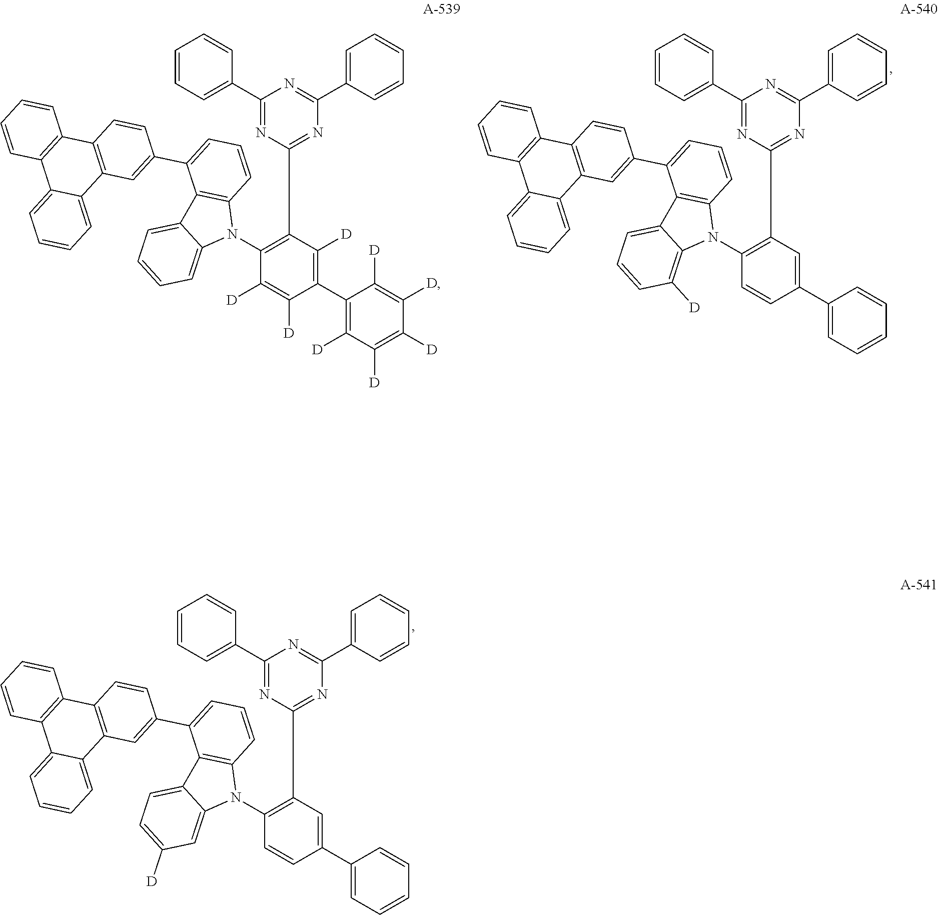 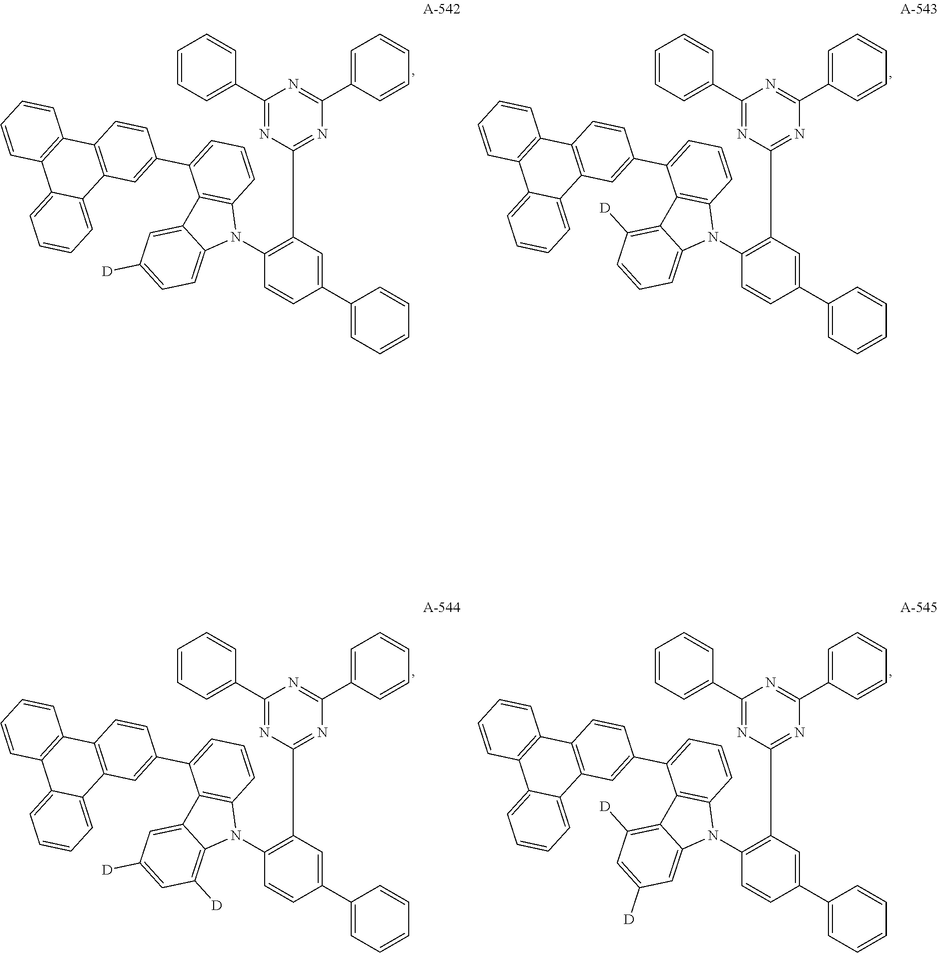 [SMILES: [2H]c1ccc2c(c1)c1c(-c3ccc4c5ccccc5c5ccccc5c4c3)cccc1n2-c1ccc(-c2ccccc2)cc1-c1nc(-c2ccccc2)nc(-c2ccccc2)n1 ; [2H]c1cccc2c1c1c(-c3ccc4c5ccccc5c5ccccc5c4c3)cccc1n2-c1ccc(-c2ccccc2)cc1-c1nc(-c2ccccc2)nc(-c2ccccc2)n1 ; [2H]c1cc([2H])c2c(c1)c1c(-c3ccc4c5ccccc5c5ccccc5c4c3)cccc1n2-c1ccc(-c2ccccc2)cc1-c1nc(-c2ccccc2)nc(-c2ccccc2)n1 ; [2H]c1cc([2H])c2c3c(-c4ccc5c6ccccc6c6ccccc6c5c4)cccc3n(-c3ccc(-c4ccccc4)cc3-c3nc(-c4ccccc4)nc(-c4ccccc4)n3)c2c1] 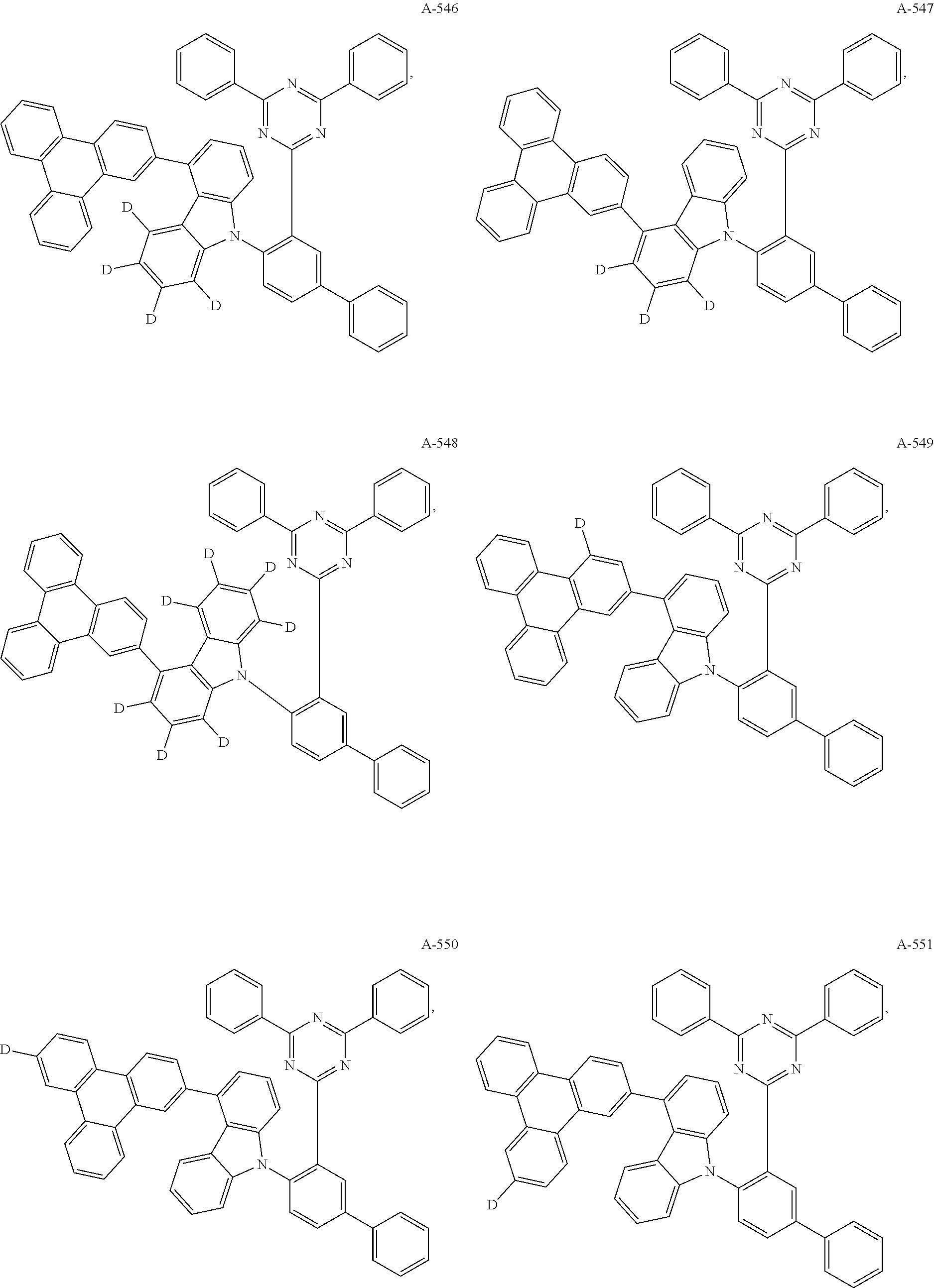 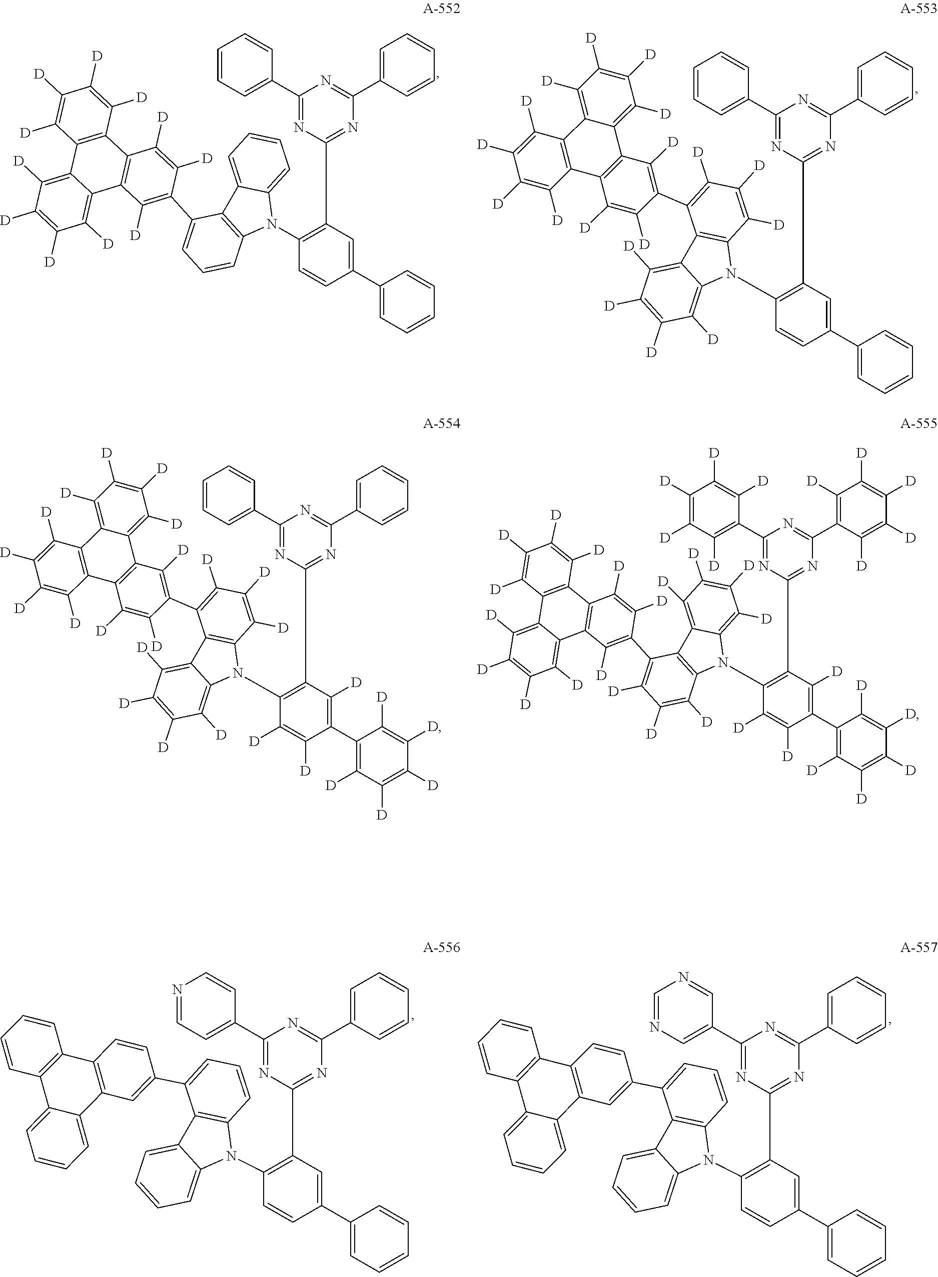 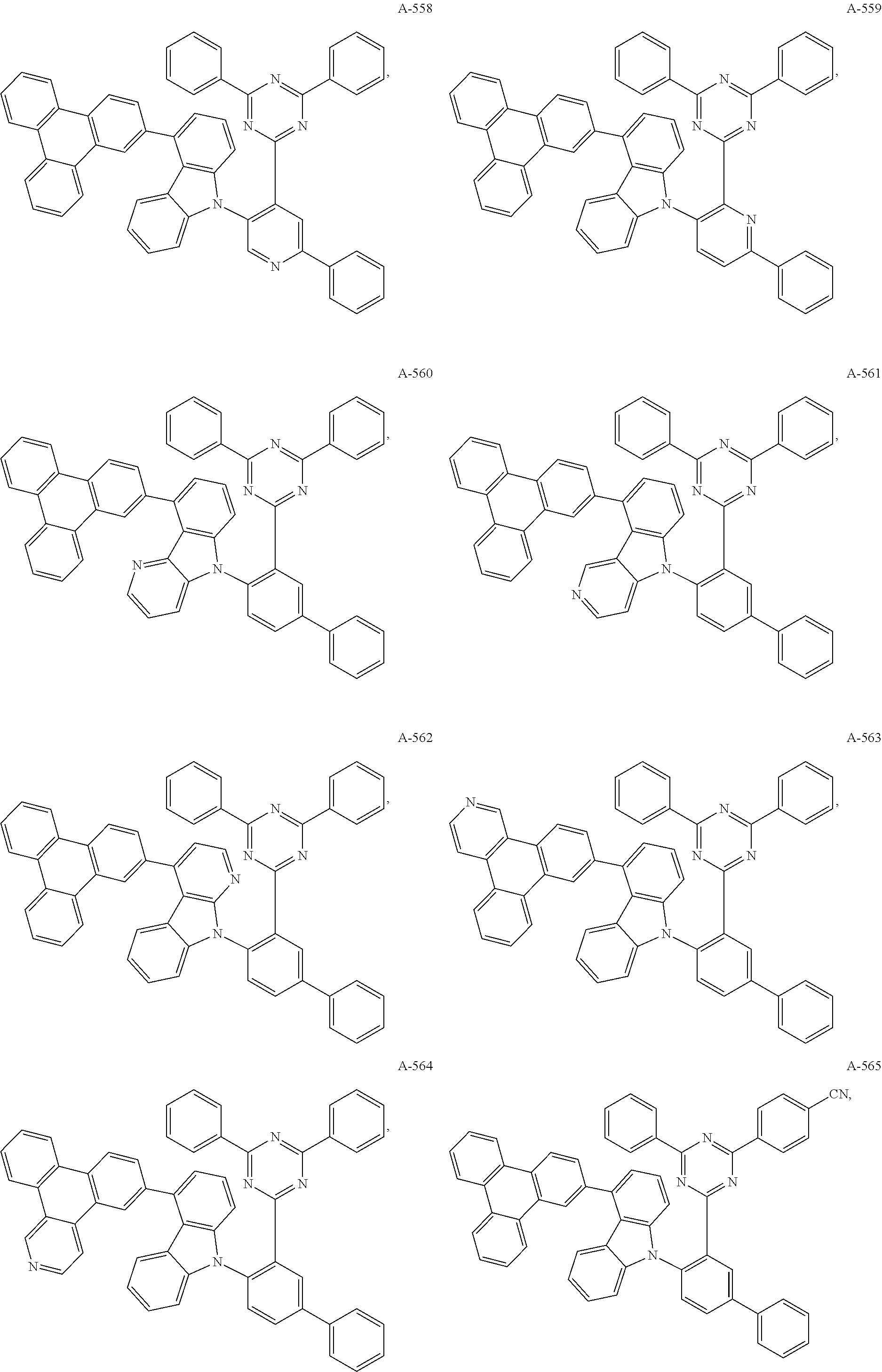 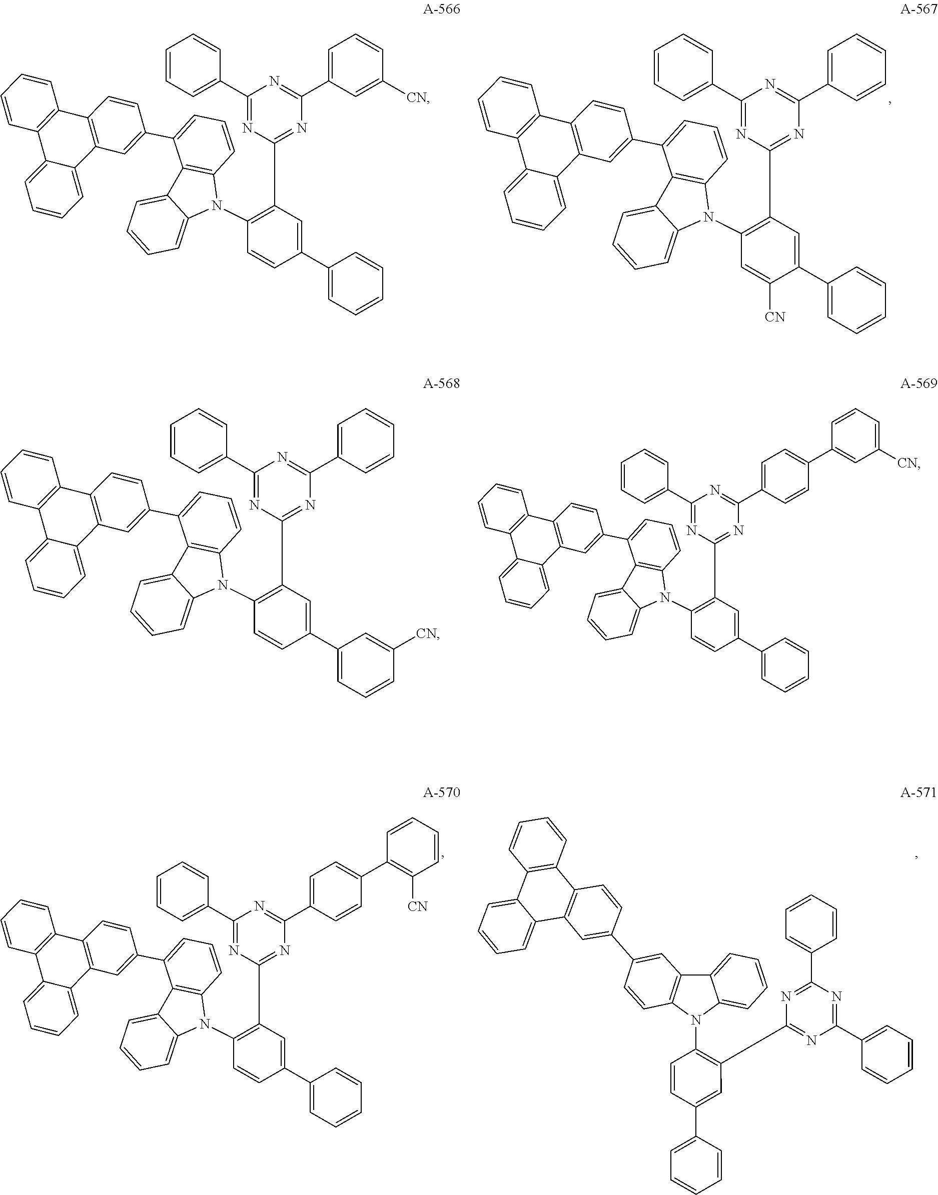 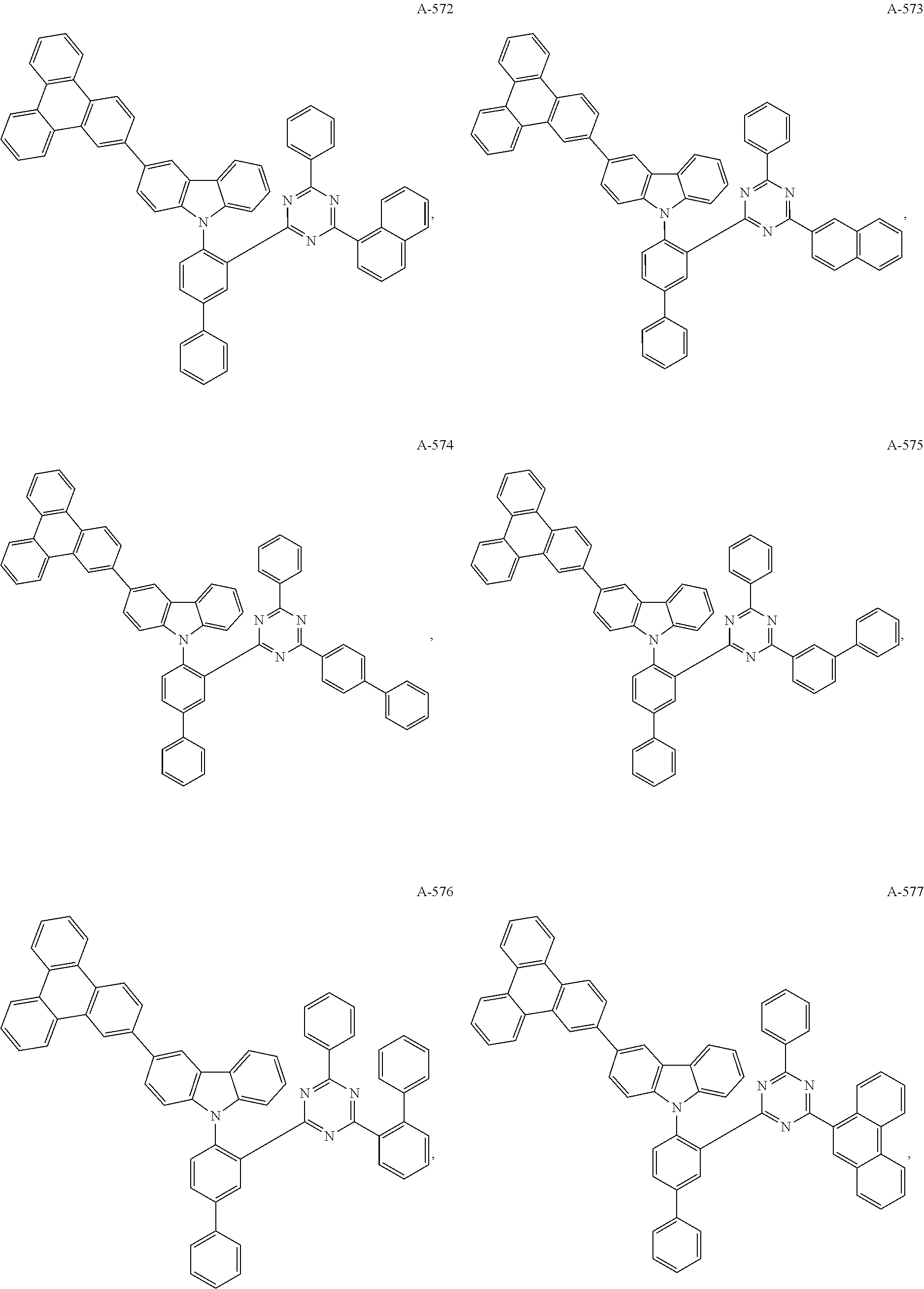 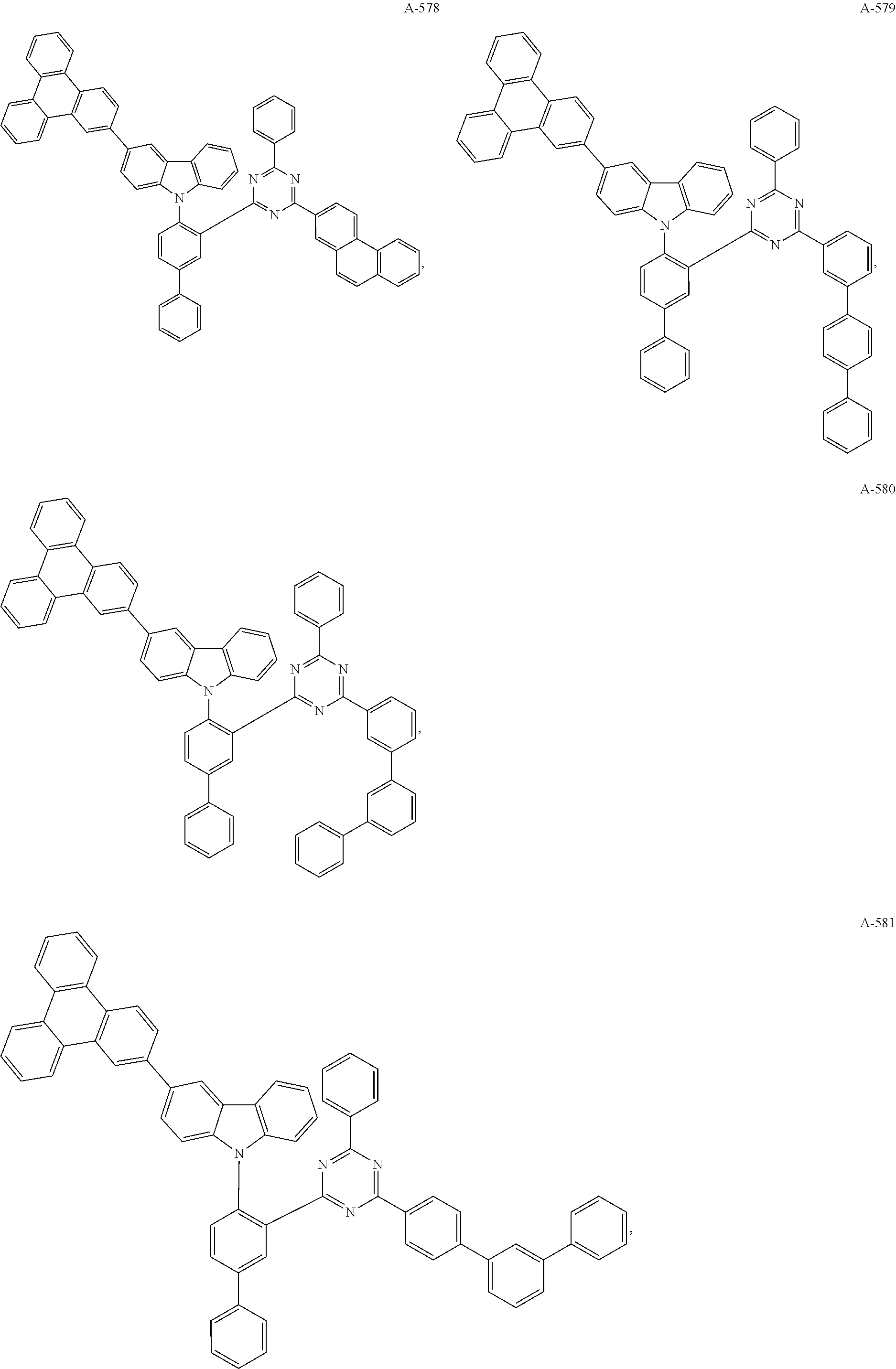 [SMILES: c1ccc(-c2ccc(-n3c4ccccc4c4cc(-c5ccc6c7ccccc7c7ccccc7c6c5)ccc43)c(-c3nc(-c4ccccc4)nc(-c4ccc5c(ccc6ccccc65)c4)n3)c2)cc1 ; c1ccc(-c2ccc(-c3cccc(-c4nc(-c5ccccc5)nc(-c5cc(-c6ccccc6)ccc5-n5c6ccccc6c6cc(-c7ccc8c9ccccc9c9ccccc9c8c7)ccc65)n4)c3)cc2)cc1 ; c1ccc(-c2cccc(-c3cccc(-c4nc(-c5ccccc5)nc(-c5cc(-c6ccccc6)ccc5-n5c6ccccc6c6cc(-c7ccc8c9ccccc9c9ccccc9c8c7)ccc65)n4)c3)c2)cc1 ; c1ccc(-c2cccc(-c3ccc(-c4nc(-c5ccccc5)nc(-c5cc(-c6ccccc6)ccc5-n5c6ccccc6c6cc(-c7ccc8c9ccccc9c9ccccc9c8c7)ccc65)n4)cc3)c2)cc1] 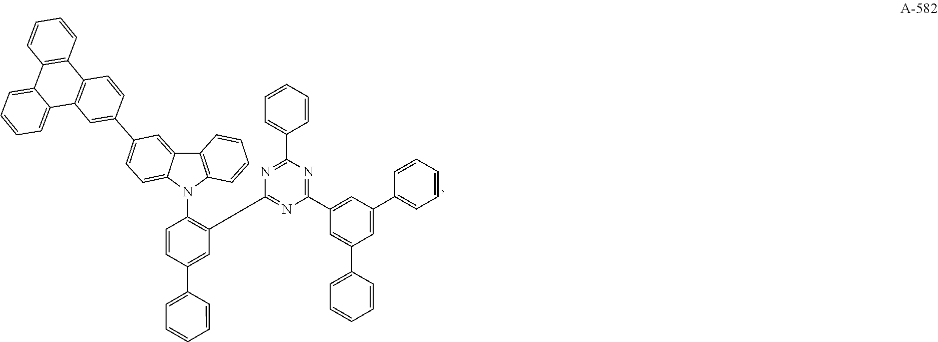 [SMILES: c1ccc(-c2cc(-c3ccccc3)cc(-c3nc(-c4ccccc4)nc(-c4cc(-c5ccccc5)ccc4-n4c5ccccc5c5cc(-c6ccc7c8ccccc8c8ccccc8c7c6)ccc54)n3)c2)cc1] 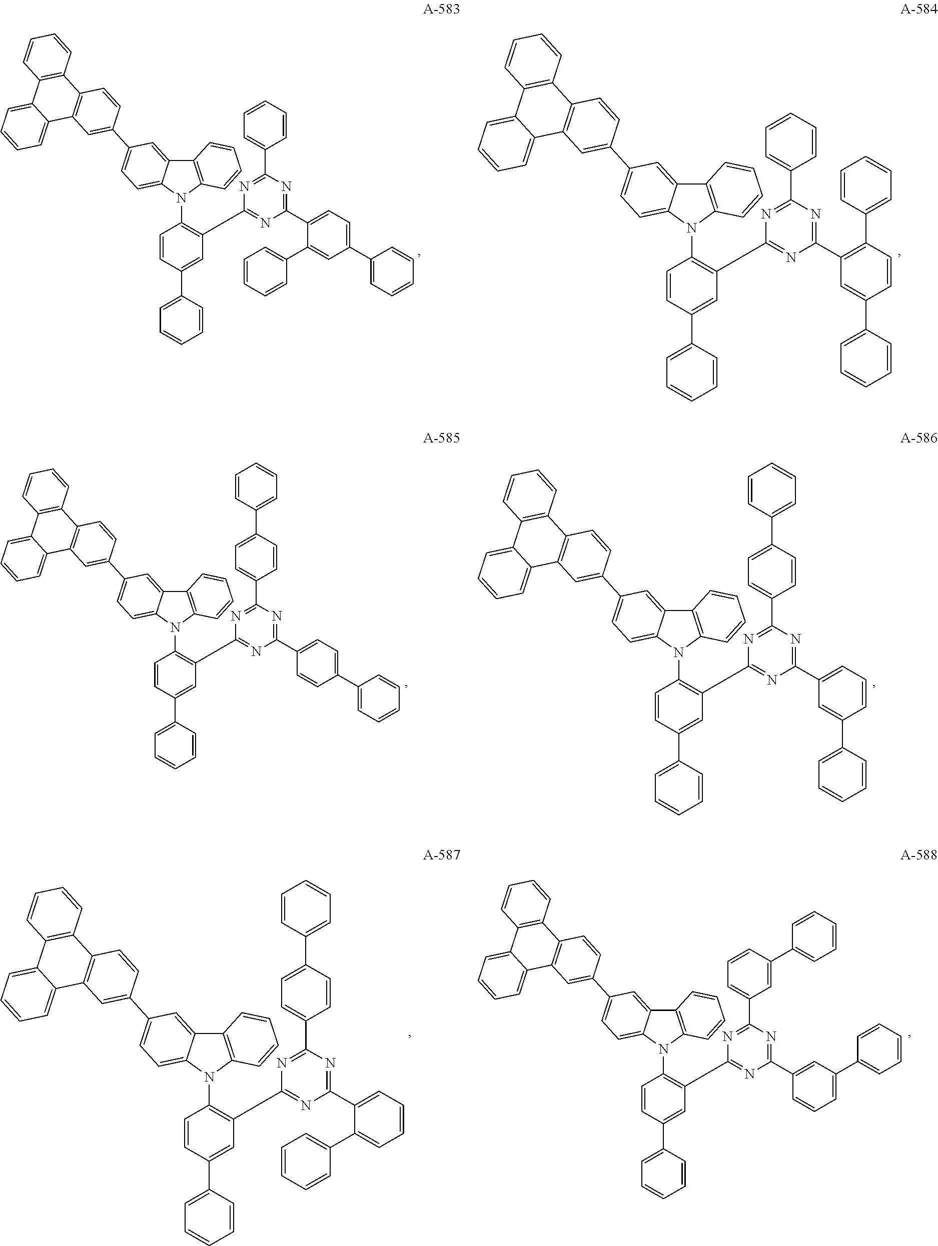 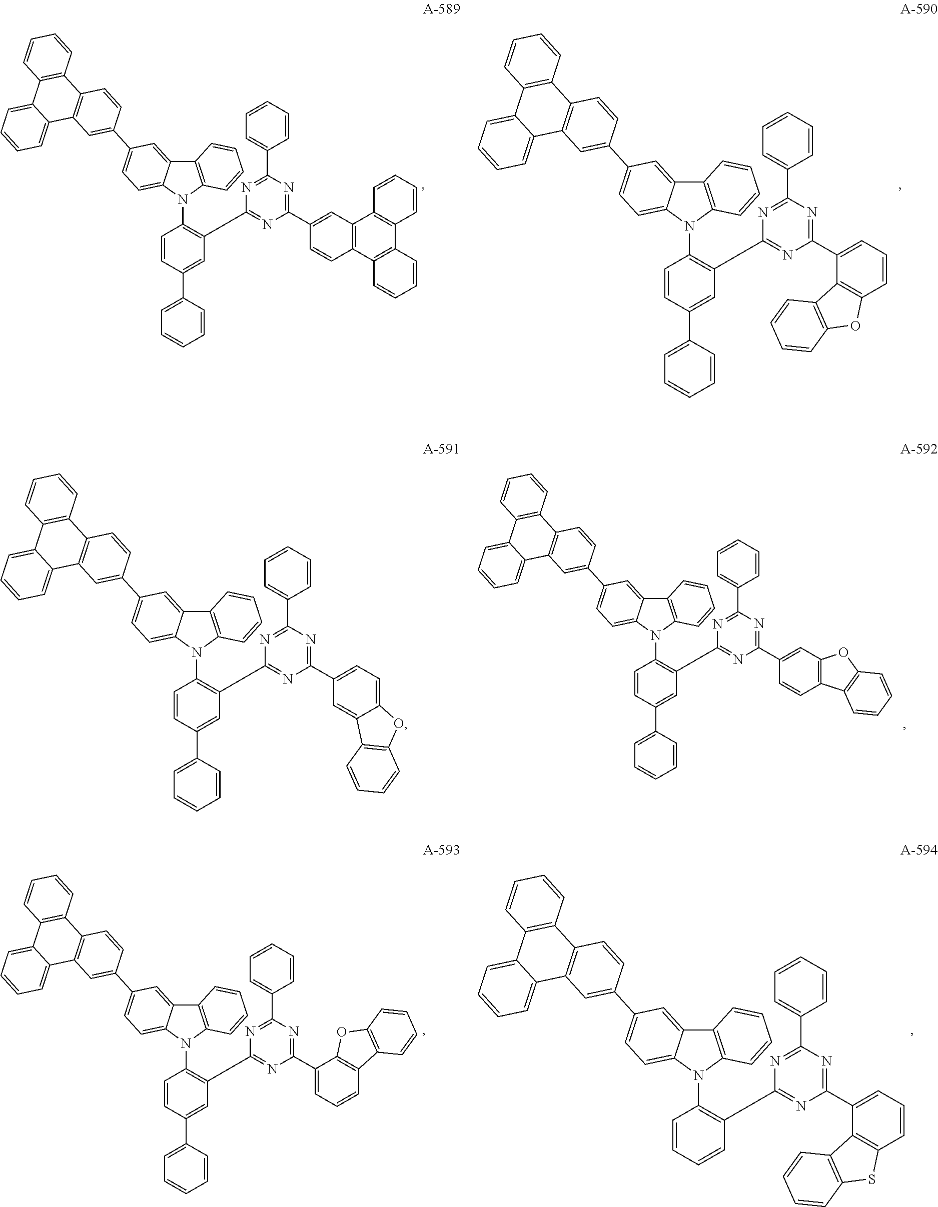 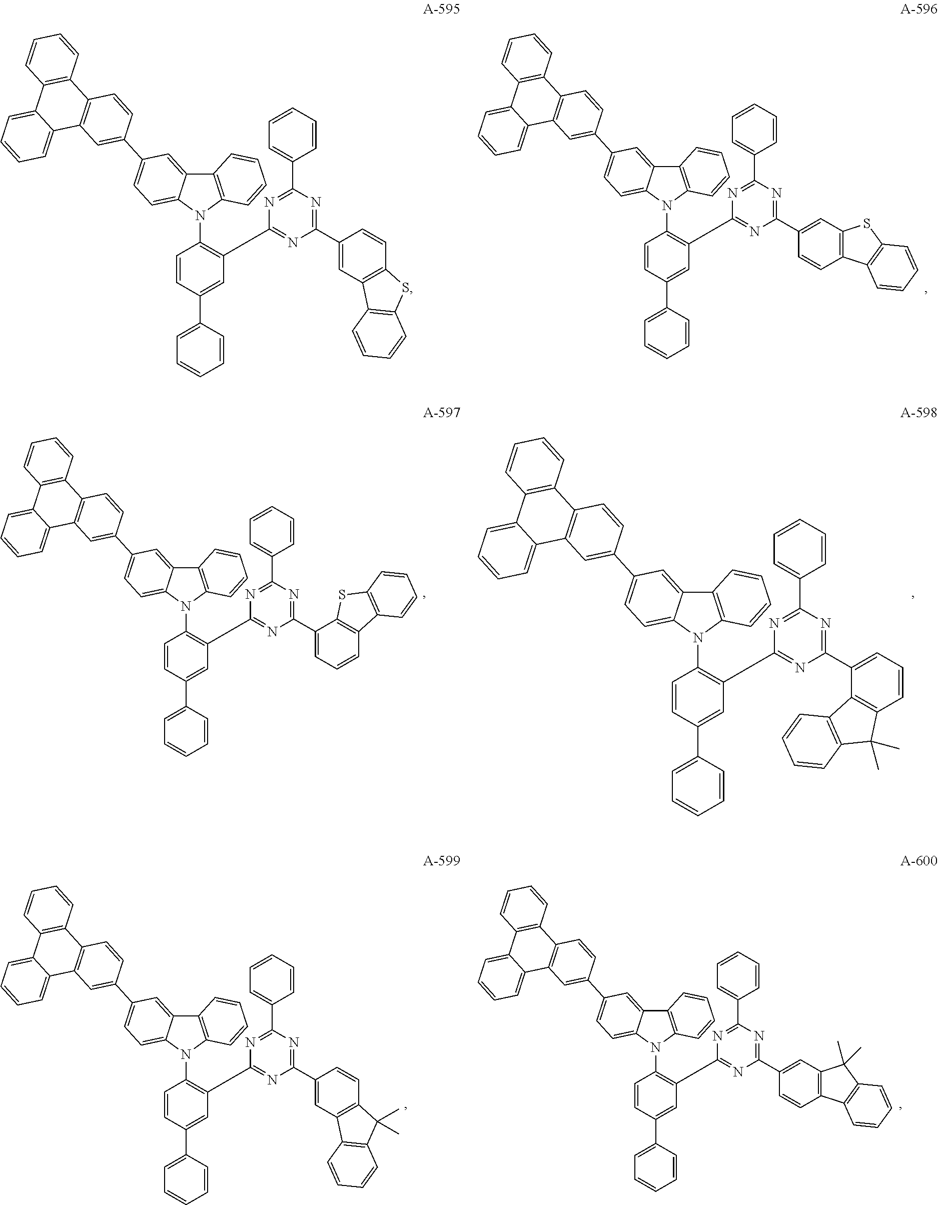 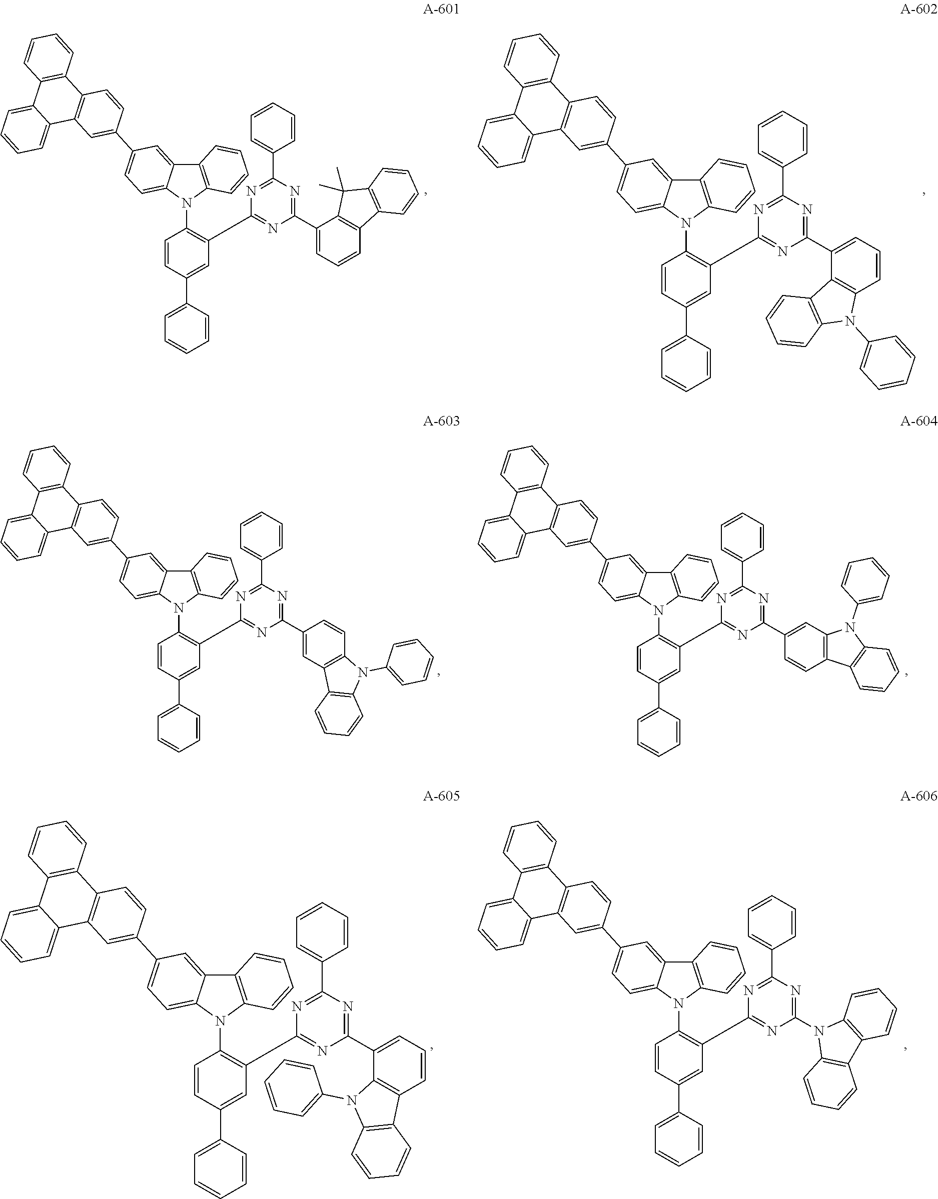 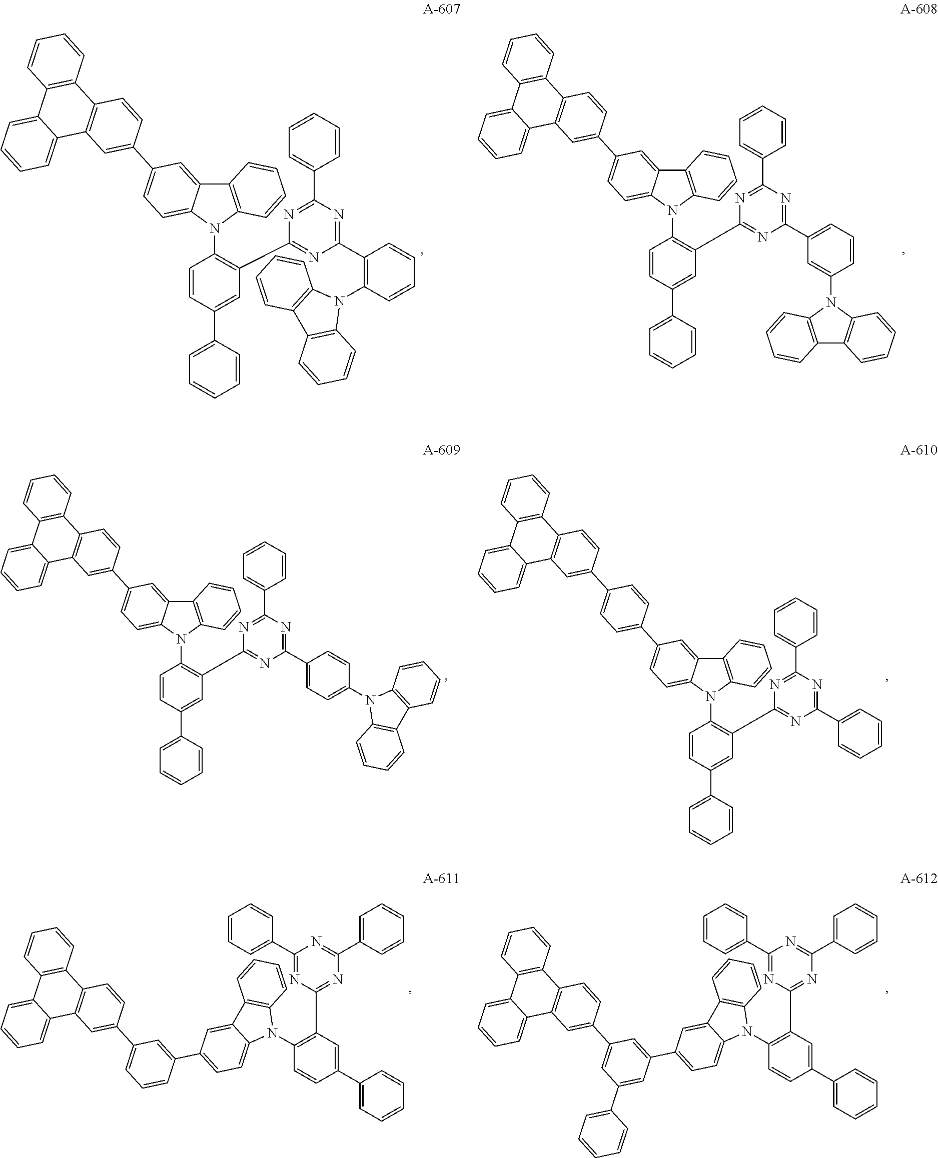 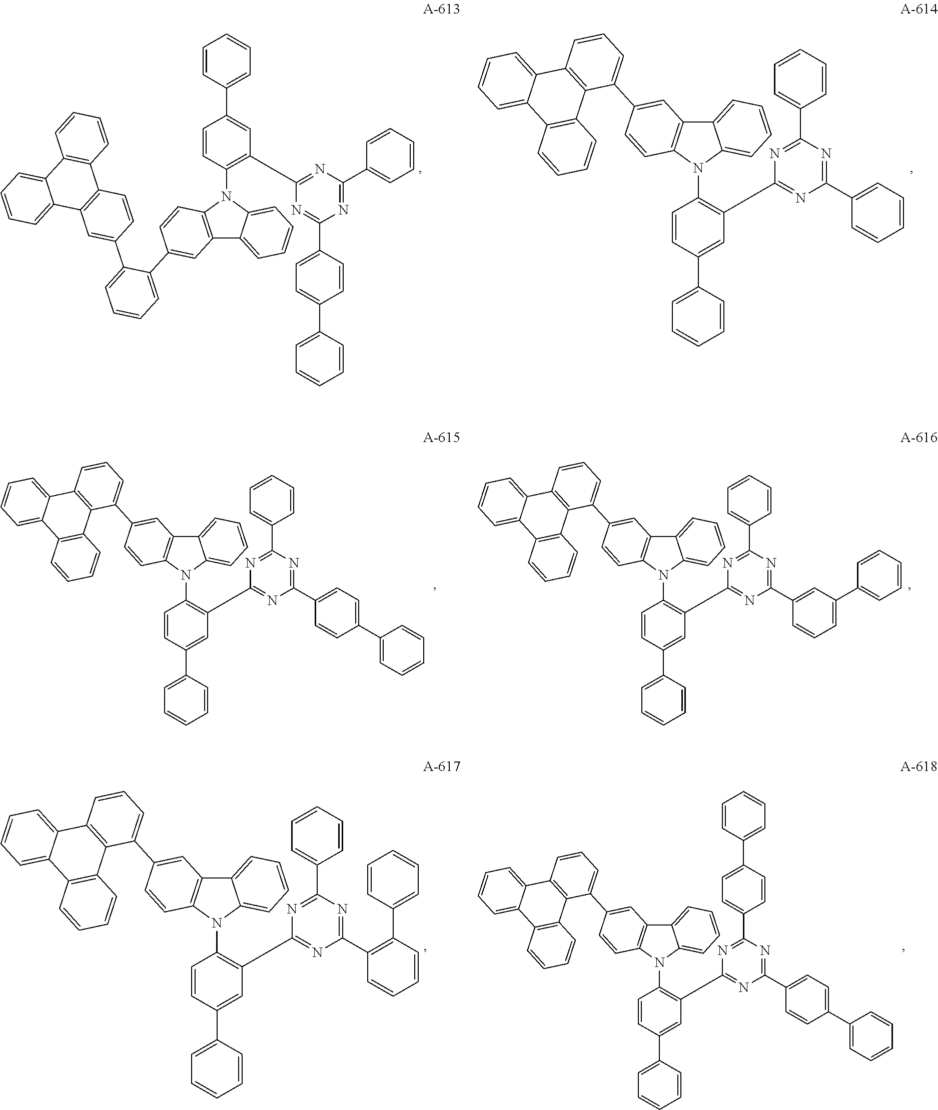 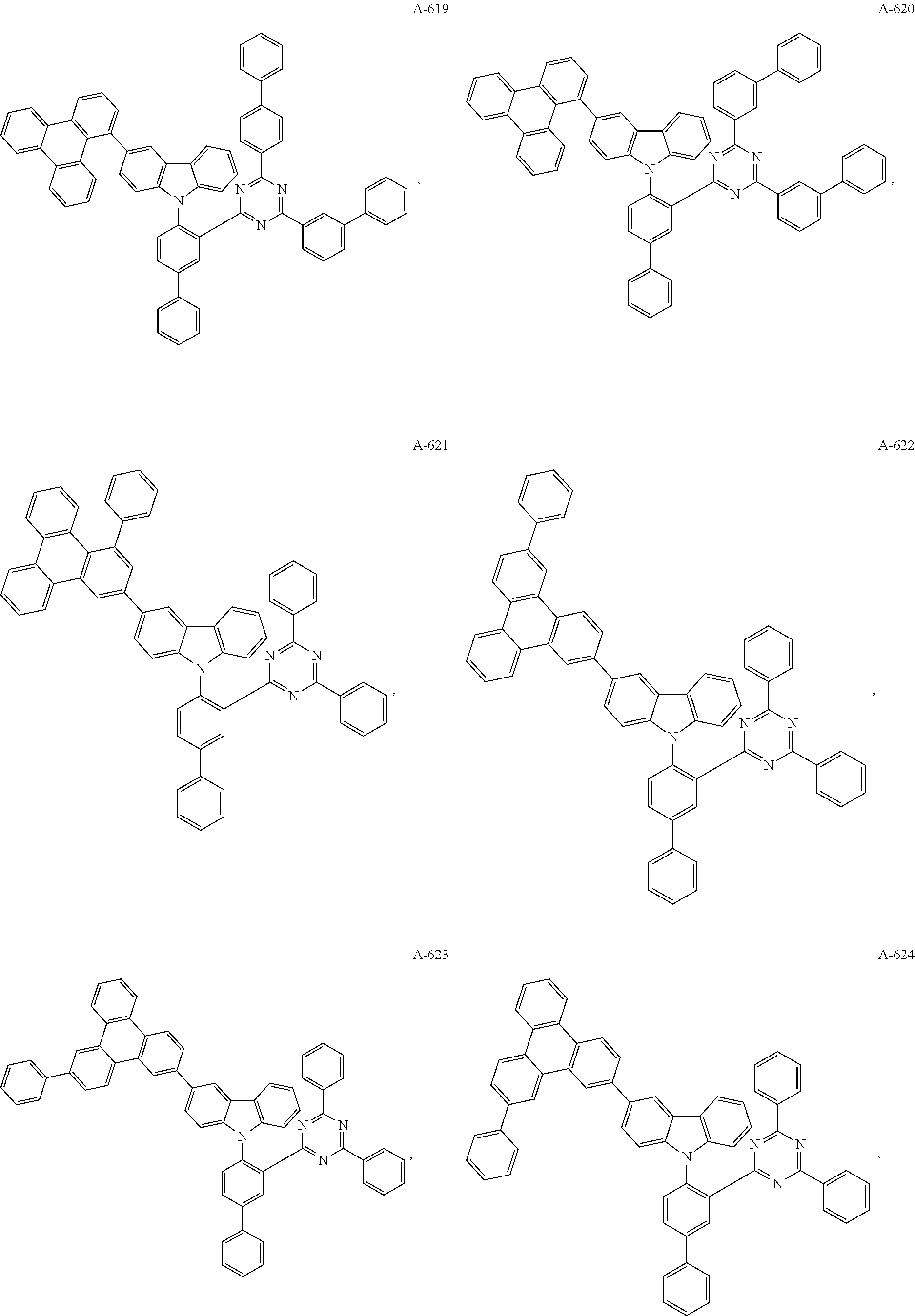 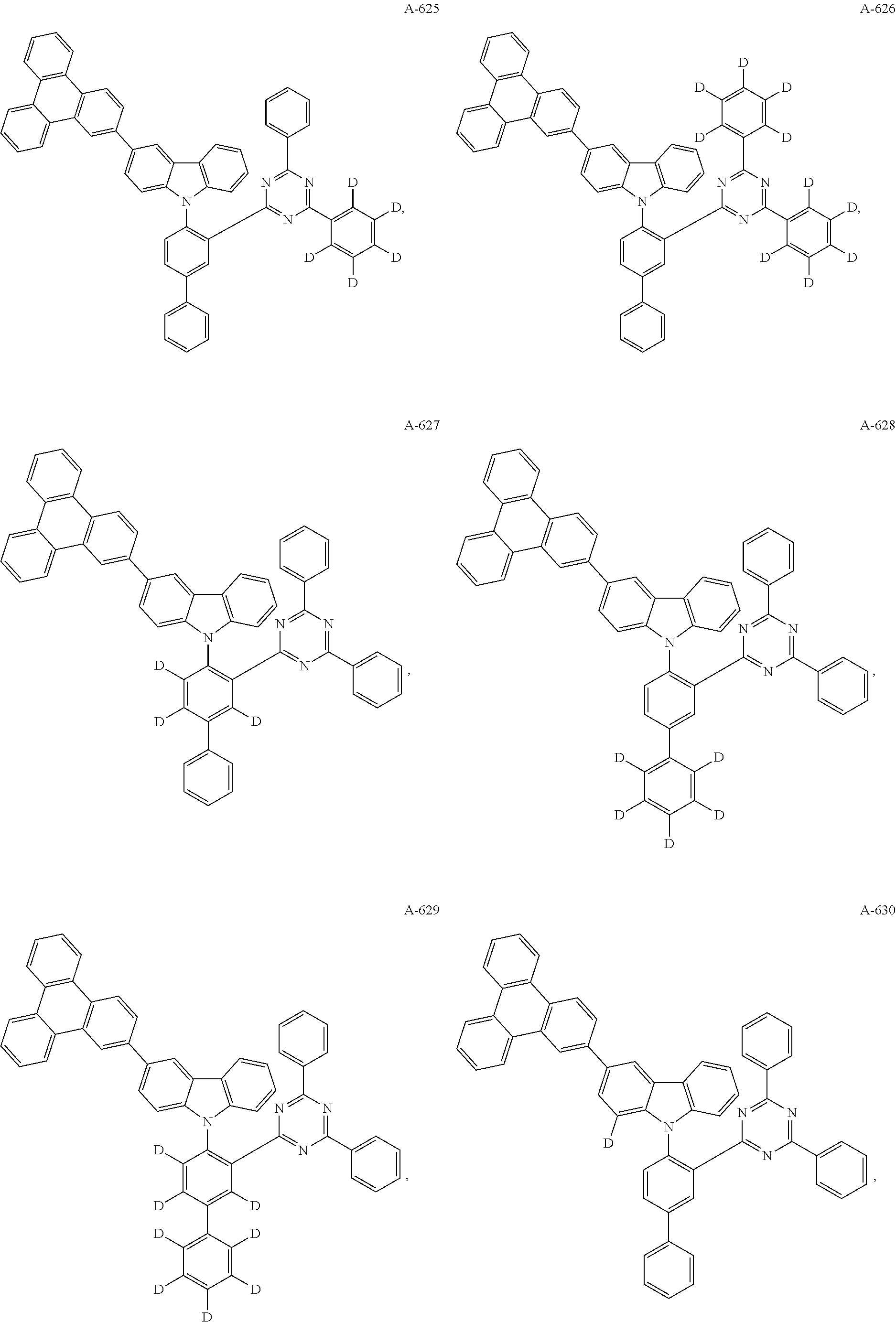 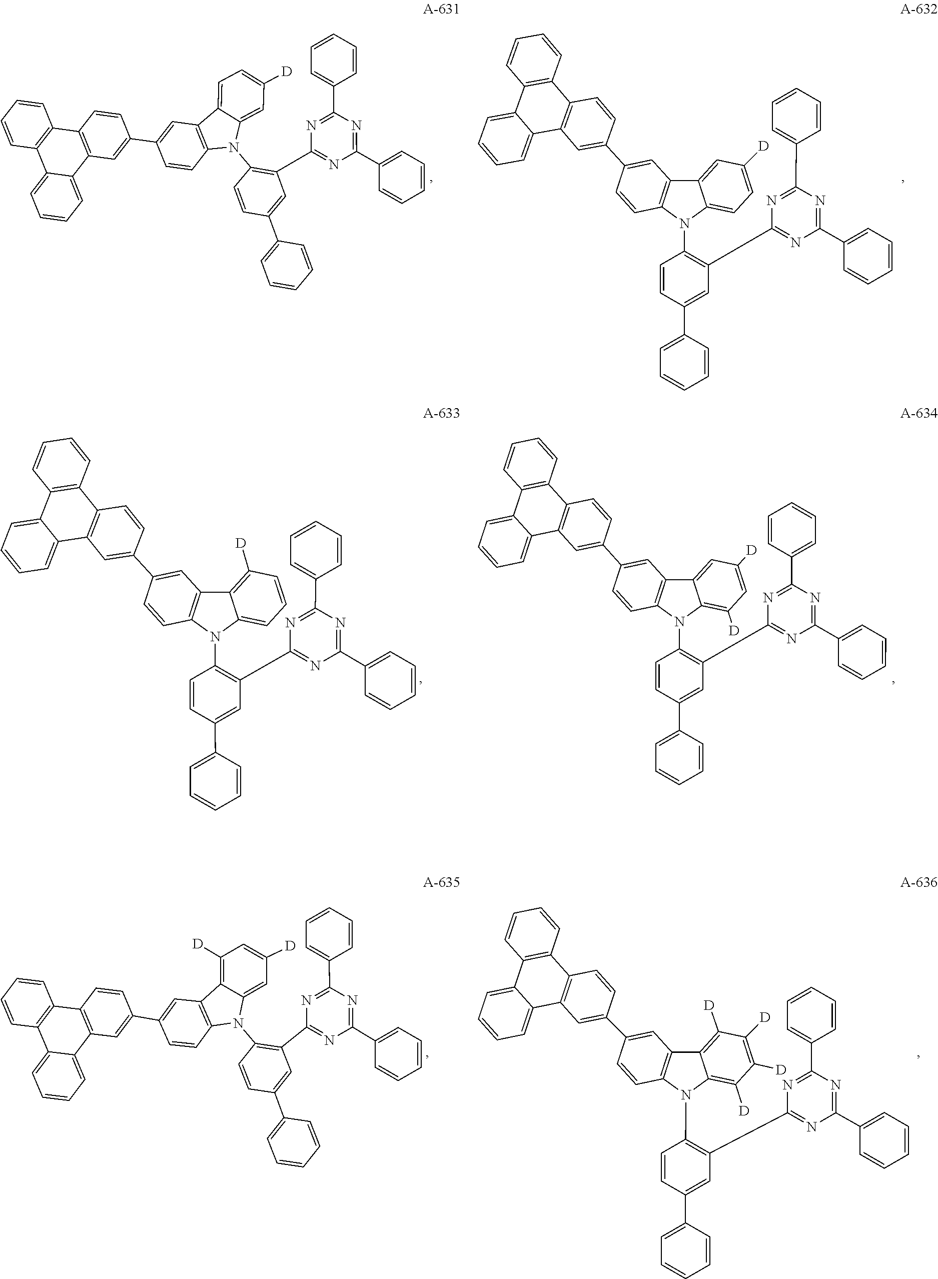 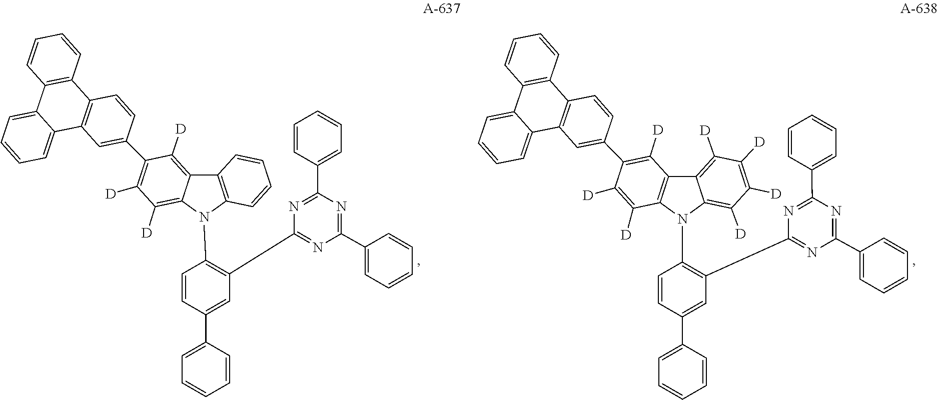 [SMILES: [2H]c1c(-c2ccc3c4ccccc4c4ccccc4c3c2)c([2H])c2c3ccccc3n(-c3ccc(-c4ccccc4)cc3-c3nc(-c4ccccc4)nc(-c4ccccc4)n3)c2c1[2H] ; [2H]c1c([2H])c([2H])c2c(c1[2H])c1c([2H])c(-c3ccc4c5ccccc5c5ccccc5c4c3)c([2H])c([2H])c1n2-c1ccc(-c2ccccc2)cc1-c1nc(-c2ccccc2)nc(-c2ccccc2)n1] 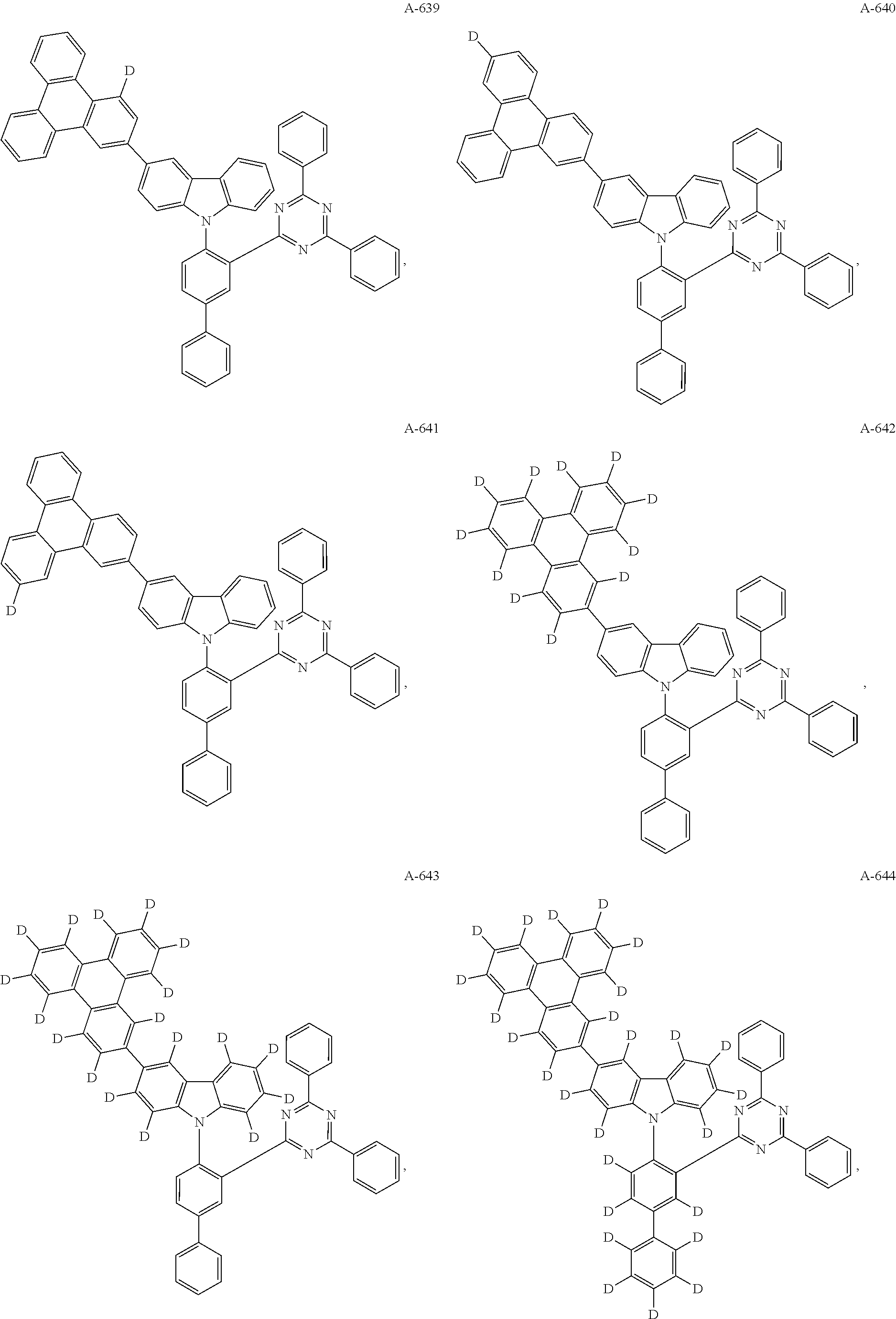 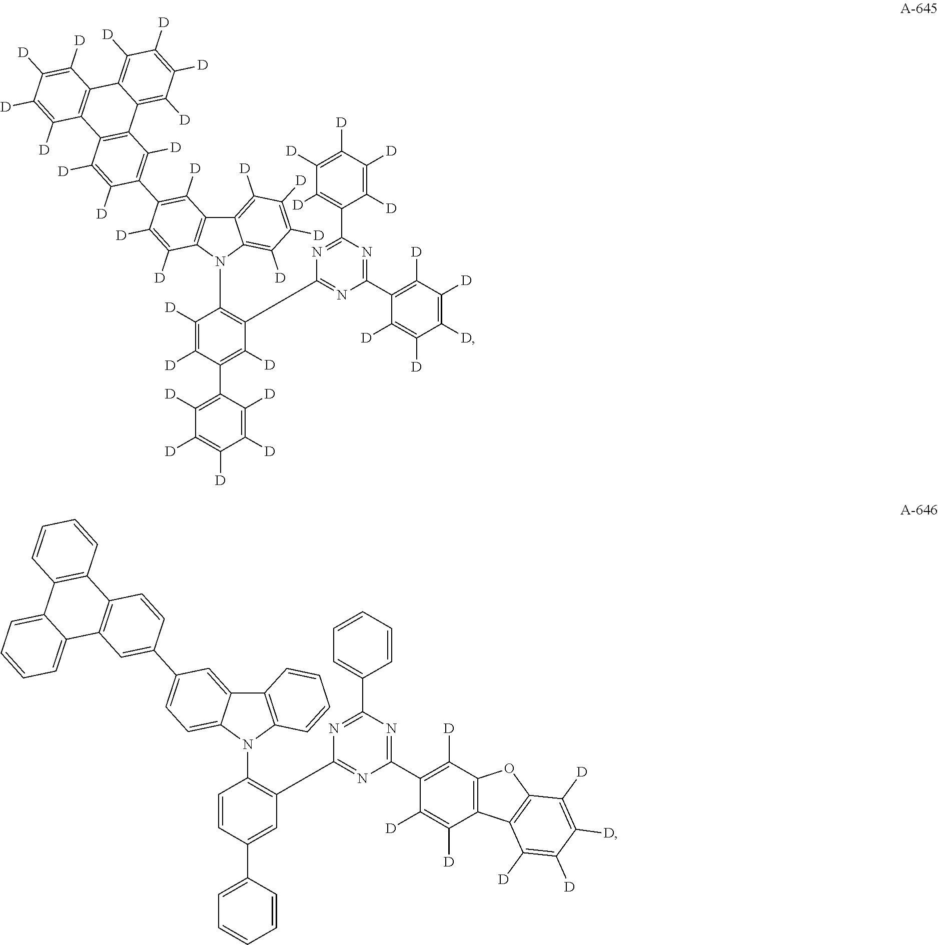 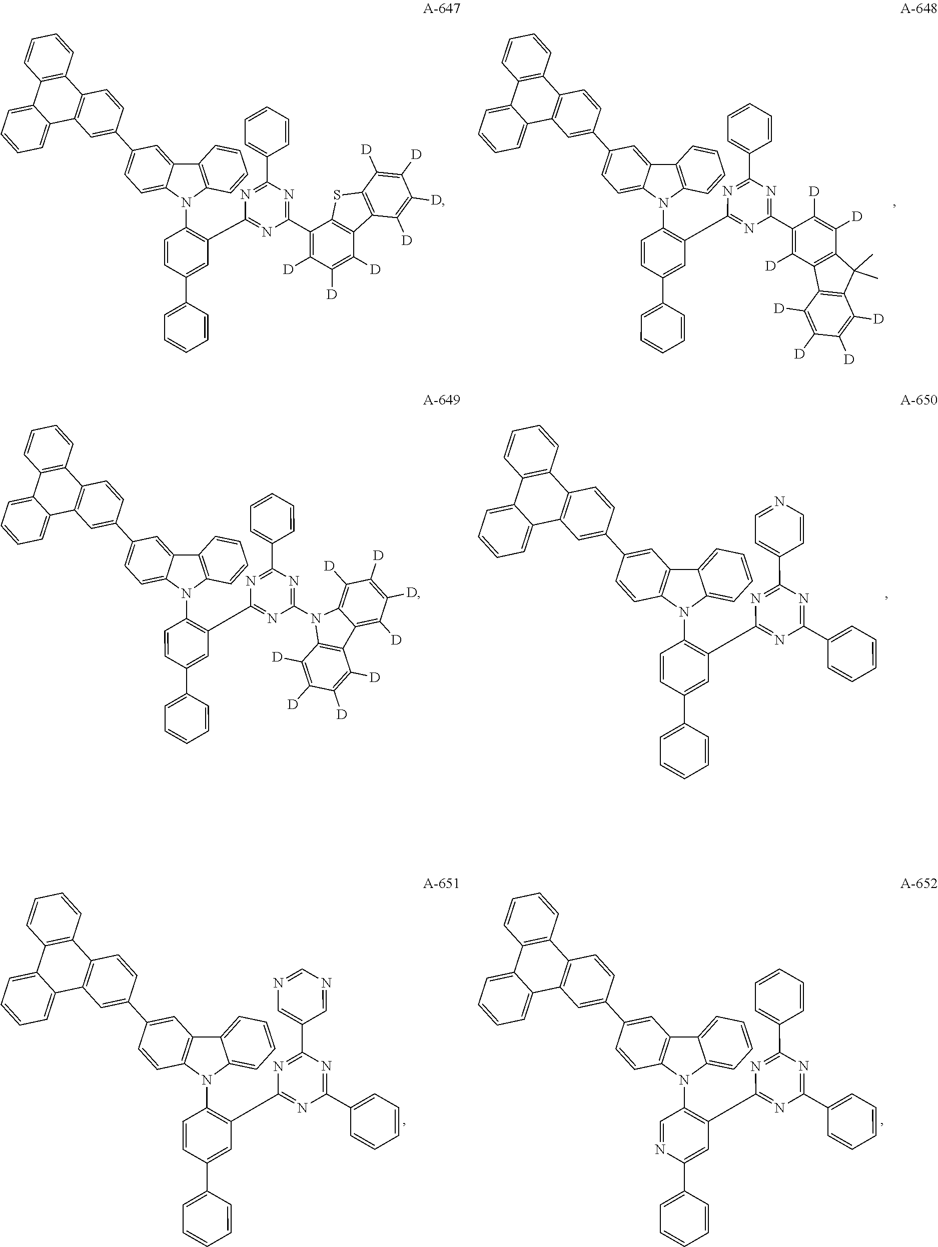 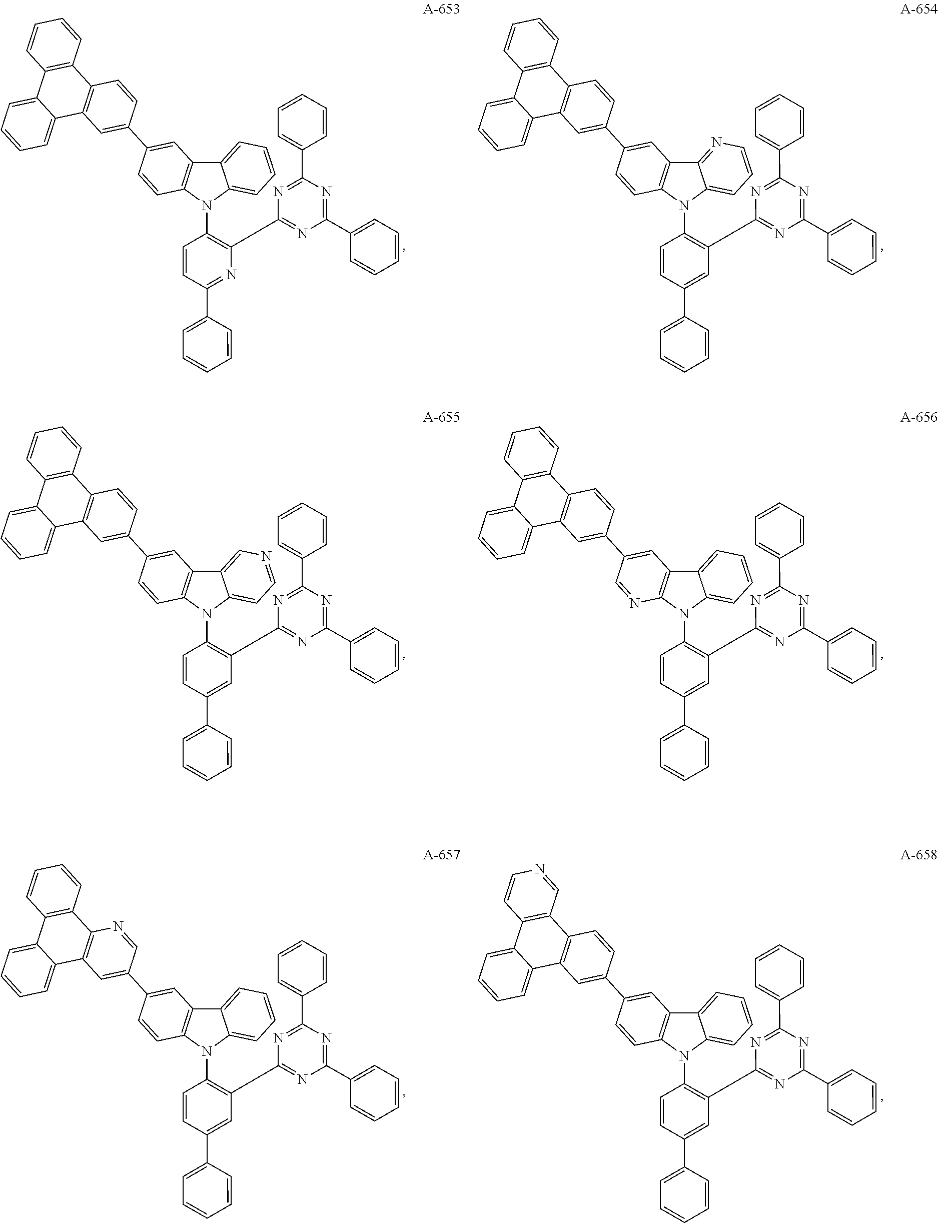 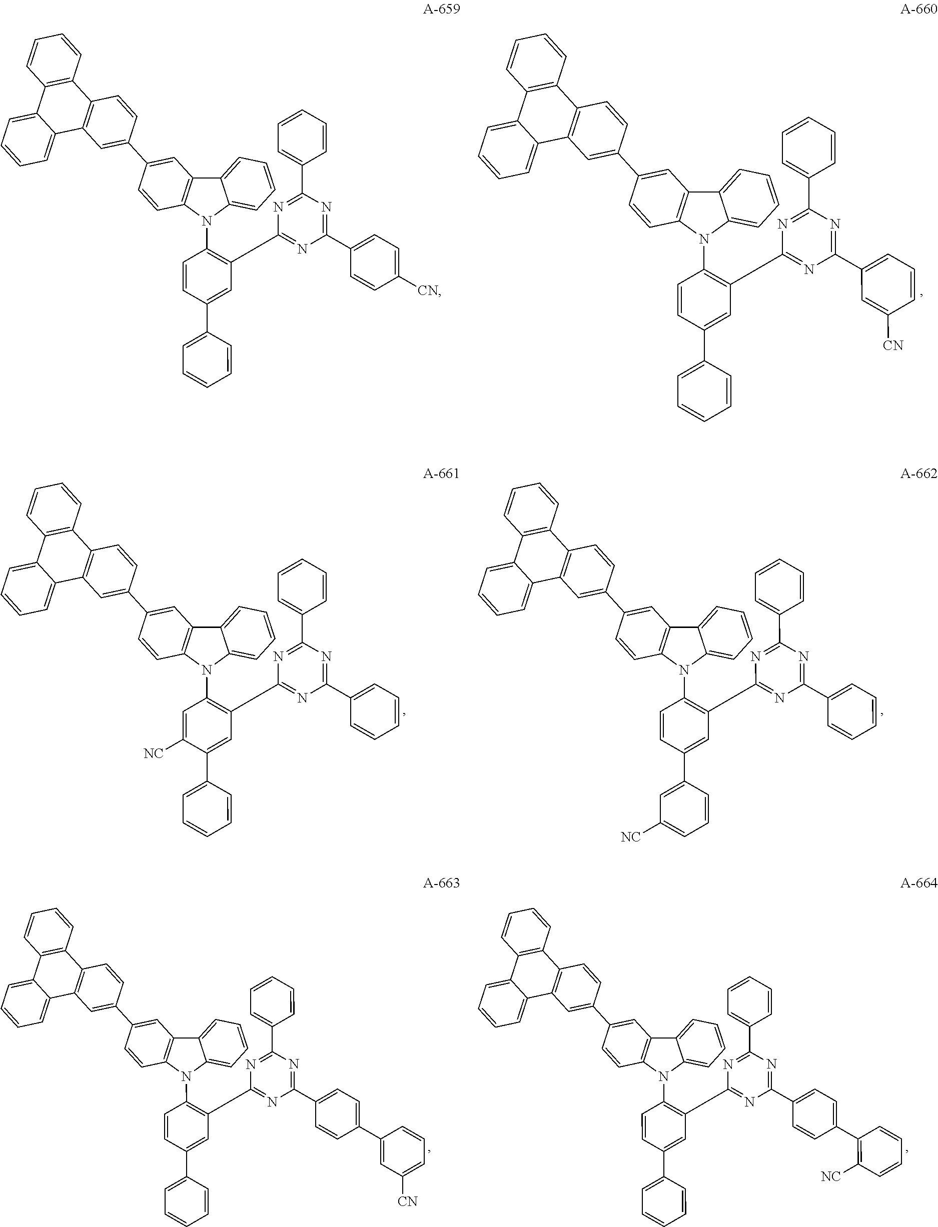 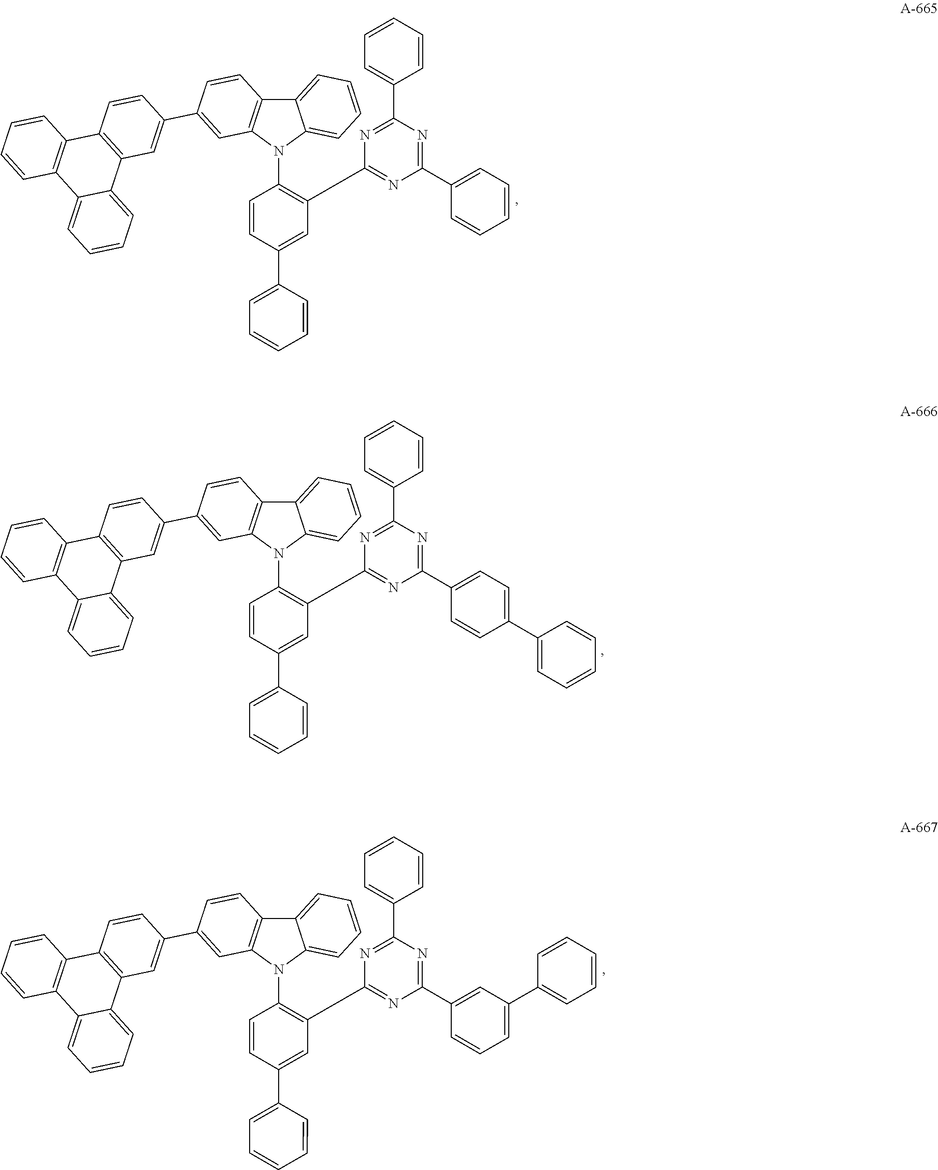 [SMILES: c1ccc(-c2ccc(-n3c4ccccc4c4ccc(-c5ccc6c7ccccc7c7ccccc7c6c5)cc43)c(-c3nc(-c4ccccc4)nc(-c4ccccc4)n3)c2)cc1 ; c1ccc(-c2ccc(-c3nc(-c4ccccc4)nc(-c4cc(-c5ccccc5)ccc4-n4c5ccccc5c5ccc(-c6ccc7c8ccccc8c8ccccc8c7c6)cc54)n3)cc2)cc1 ; c1ccc(-c2cccc(-c3nc(-c4ccccc4)nc(-c4cc(-c5ccccc5)ccc4-n4c5ccccc5c5ccc(-c6ccc7c8ccccc8c8ccccc8c7c6)cc54)n3)c2)cc1] 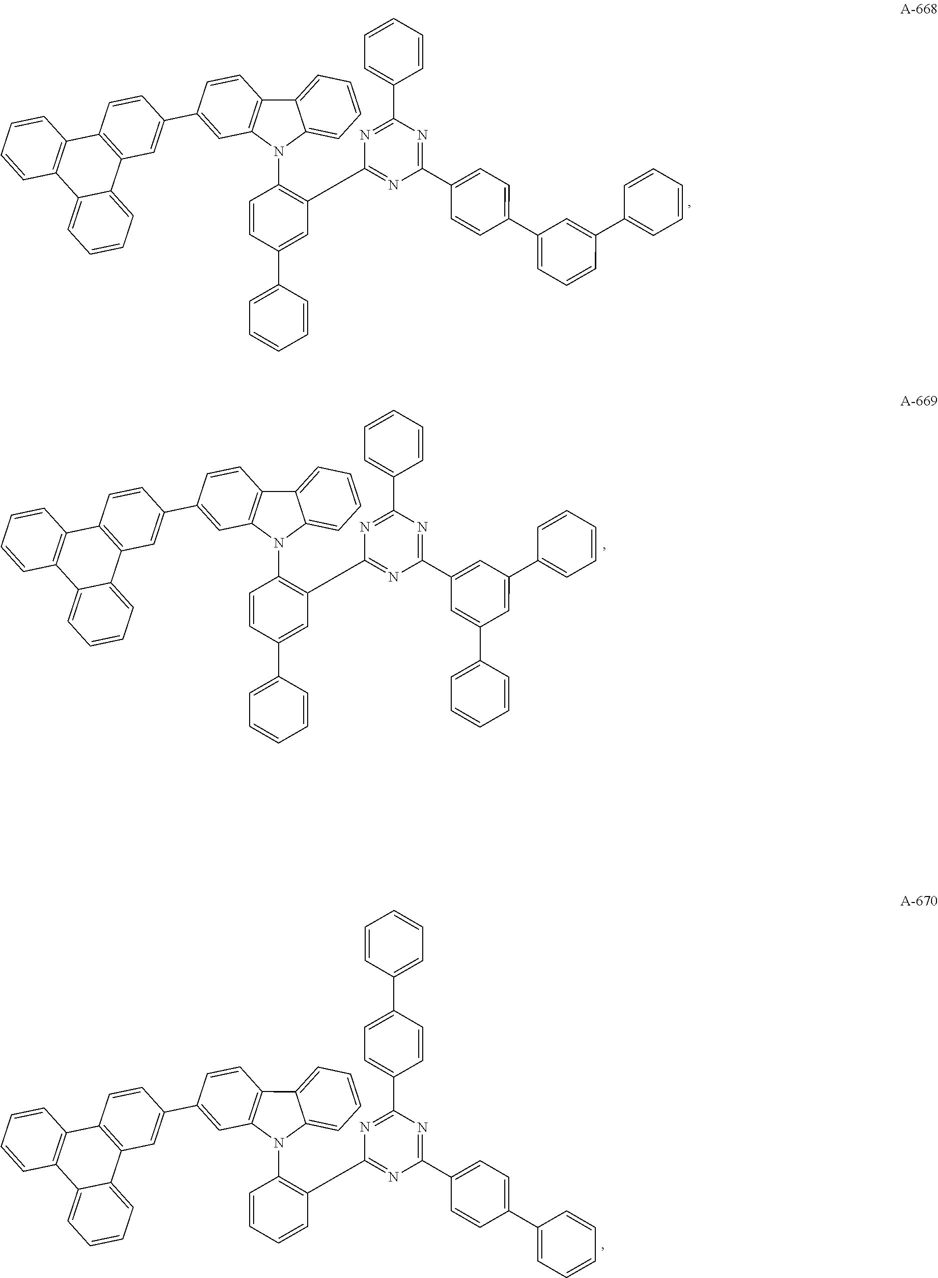 [SMILES: c1ccc(-c2cccc(-c3ccc(-c4nc(-c5ccccc5)nc(-c5cc(-c6ccccc6)ccc5-n5c6ccccc6c6ccc(-c7ccc8c9ccccc9c9ccccc9c8c7)cc65)n4)cc3)c2)cc1 ; c1ccc(-c2cc(-c3ccccc3)cc(-c3nc(-c4ccccc4)nc(-c4cc(-c5ccccc5)ccc4-n4c5ccccc5c5ccc(-c6ccc7c8ccccc8c8ccccc8c7c6)cc54)n3)c2)cc1 ; c1ccc(-c2ccc(-c3nc(-c4ccc(-c5ccccc5)cc4)nc(-c4ccccc4-n4c5ccccc5c5ccc(-c6ccc7c8ccccc8c8ccccc8c7c6)cc54)n3)cc2)cc1] 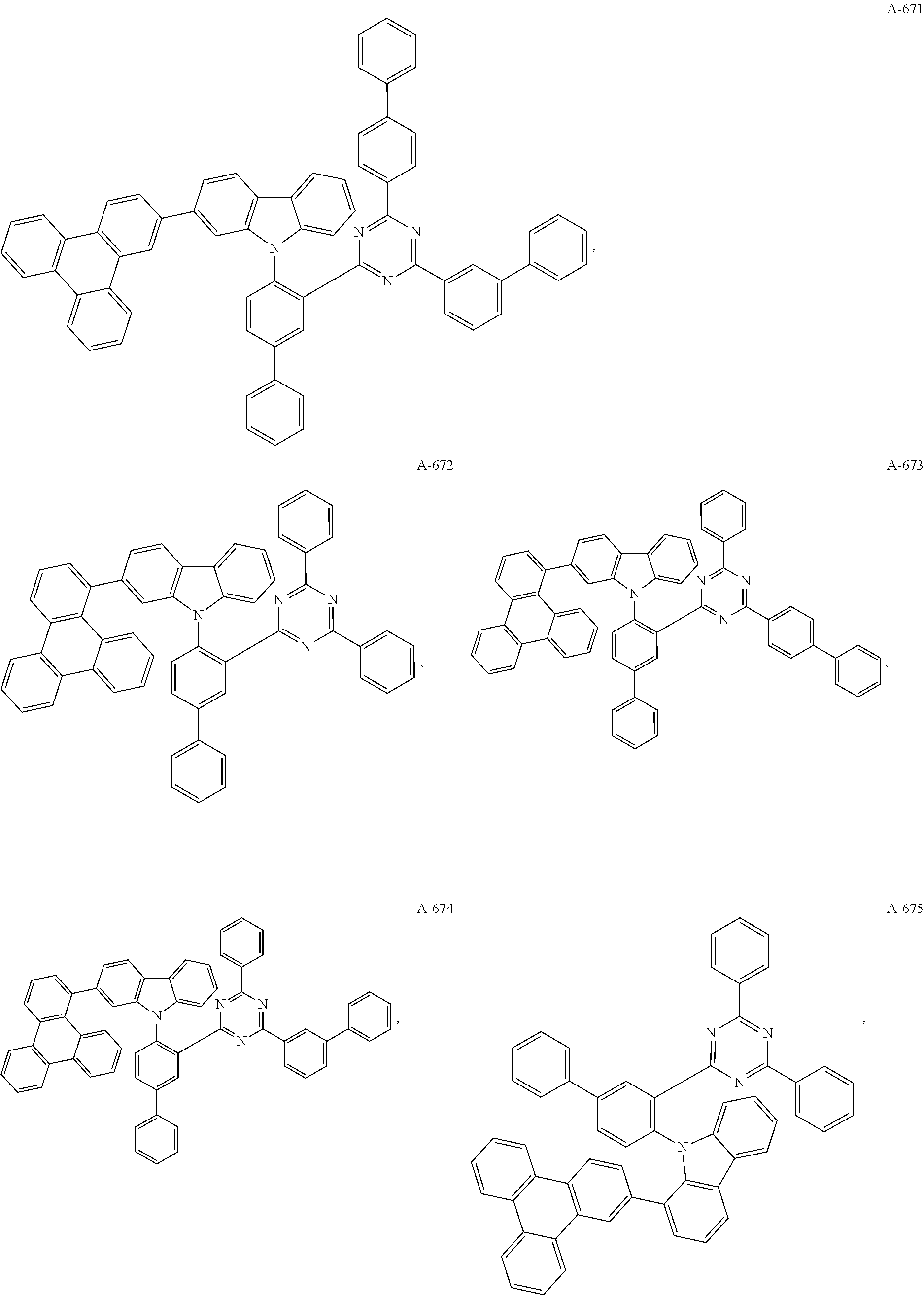 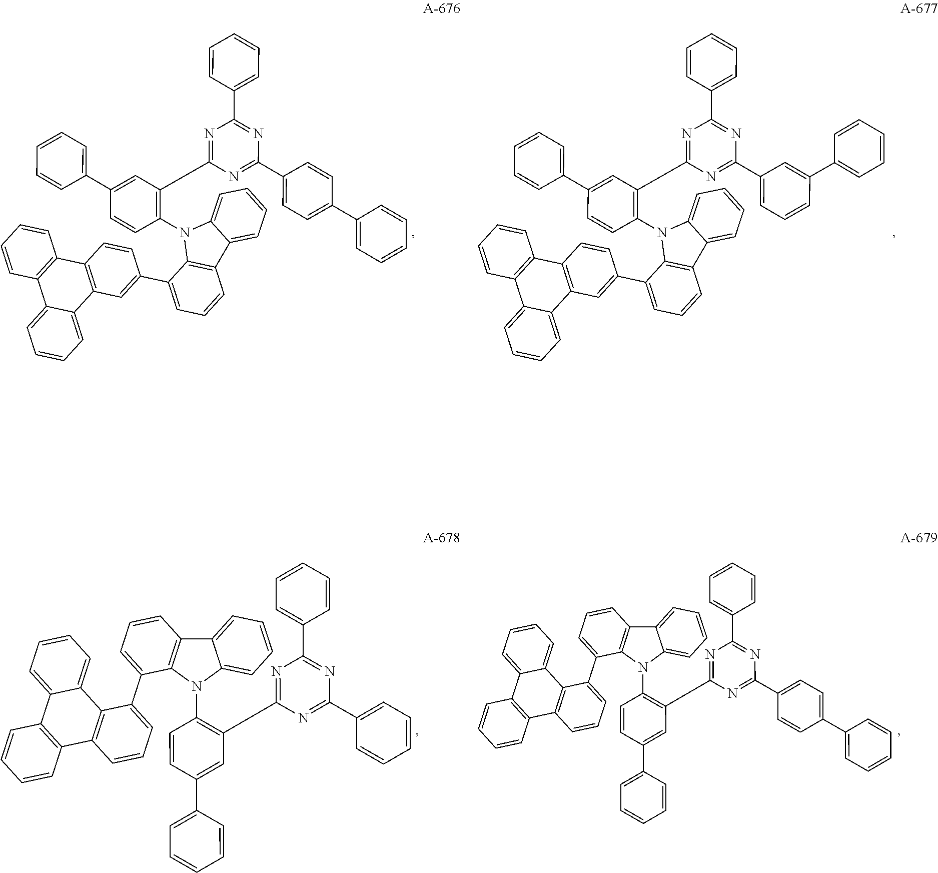 [SMILES: c1ccc(-c2ccc(-c3nc(-c4ccccc4)nc(-c4cc(-c5ccccc5)ccc4-n4c5ccccc5c5cccc(-c6ccc7c8ccccc8c8ccccc8c7c6)c54)n3)cc2)cc1 ; c1ccc(-c2cccc(-c3nc(-c4ccccc4)nc(-c4cc(-c5ccccc5)ccc4-n4c5ccccc5c5cccc(-c6ccc7c8ccccc8c8ccccc8c7c6)c54)n3)c2)cc1 ; c1ccc(-c2ccc(-n3c4ccccc4c4cccc(-c5cccc6c7ccccc7c7ccccc7c56)c43)c(-c3nc(-c4ccccc4)nc(-c4ccccc4)n3)c2)cc1 ; c1ccc(-c2ccc(-c3nc(-c4ccccc4)nc(-c4cc(-c5ccccc5)ccc4-n4c5ccccc5c5cccc(-c6cccc7c8ccccc8c8ccccc8c67)c54)n3)cc2)cc1] 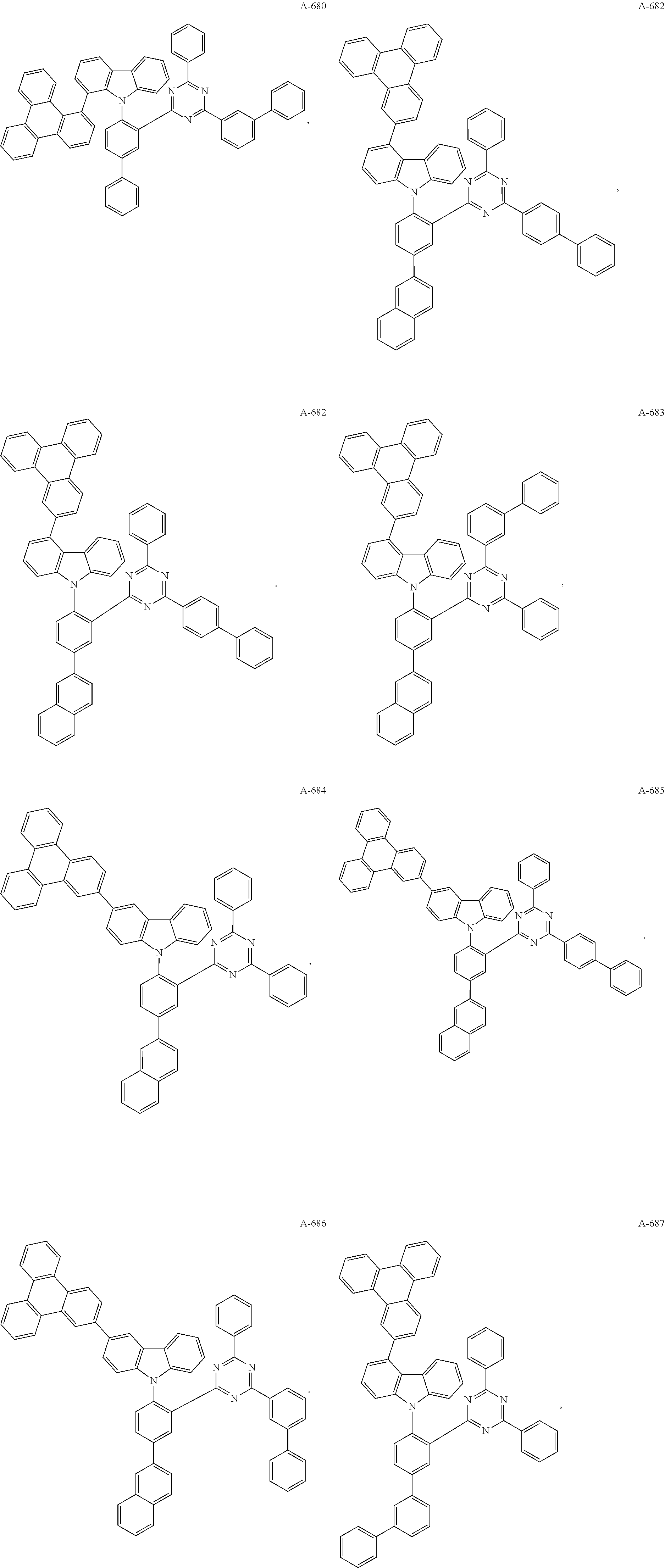 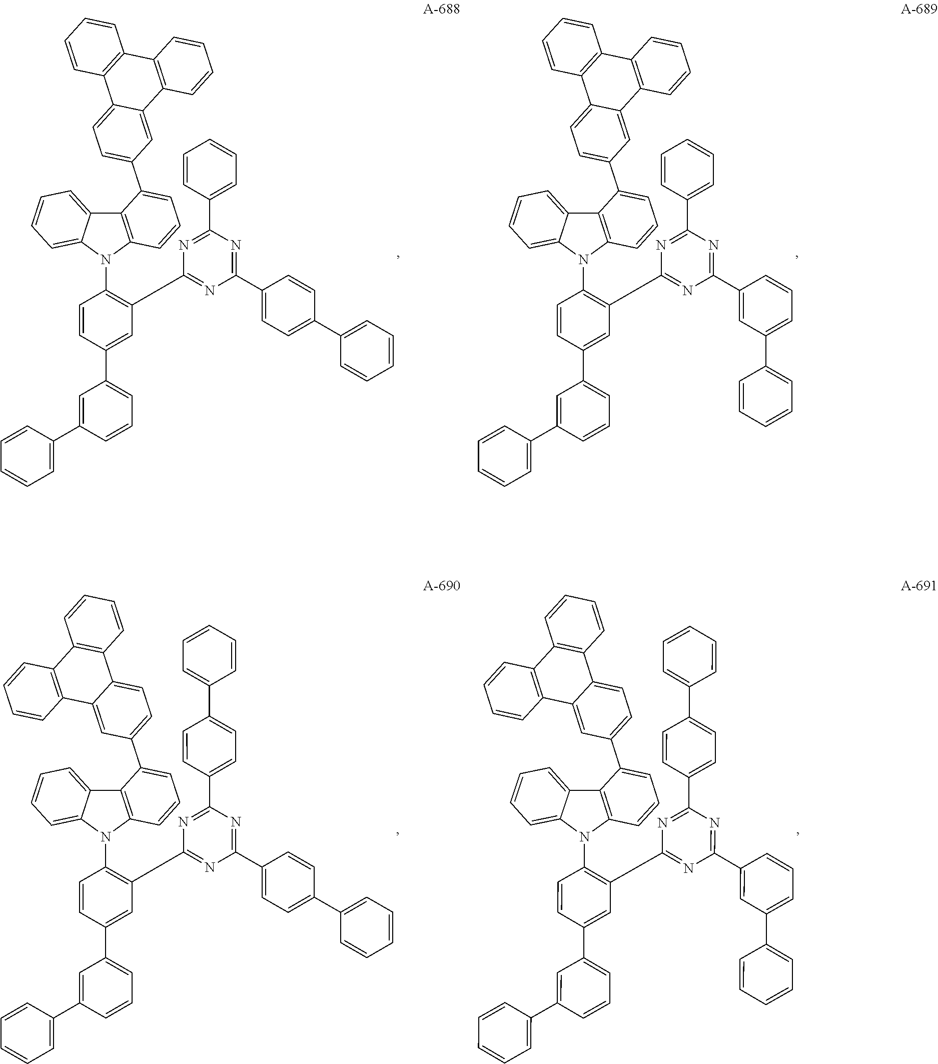 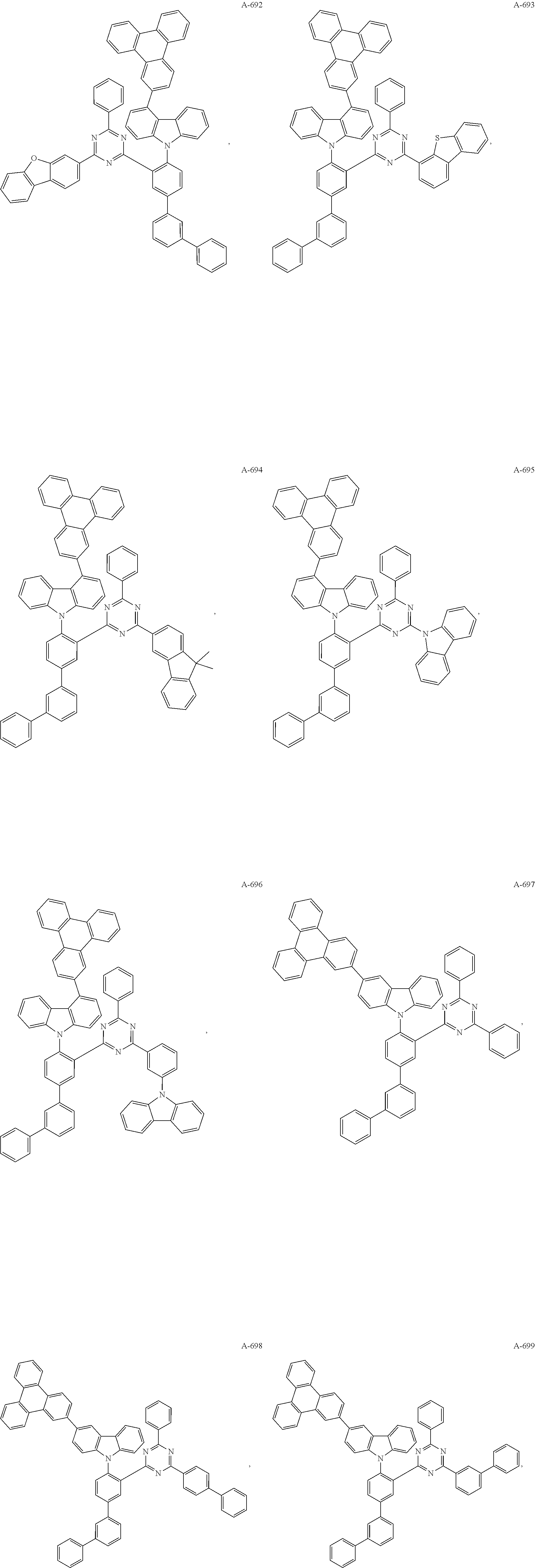 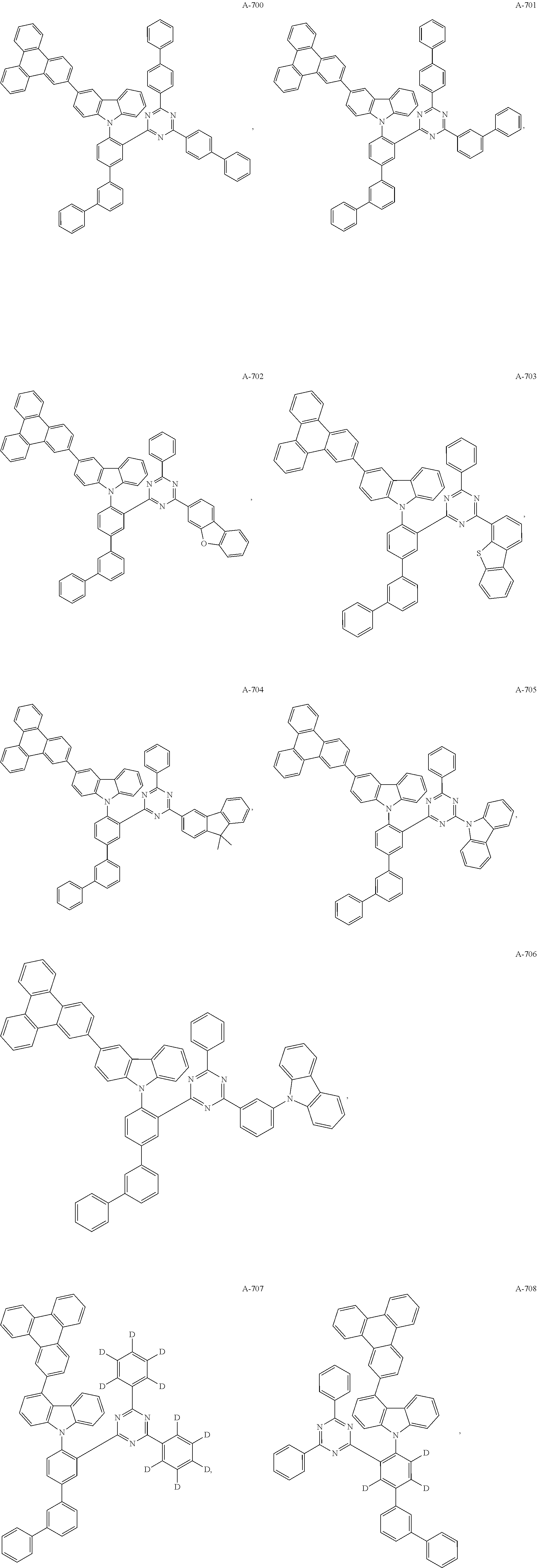 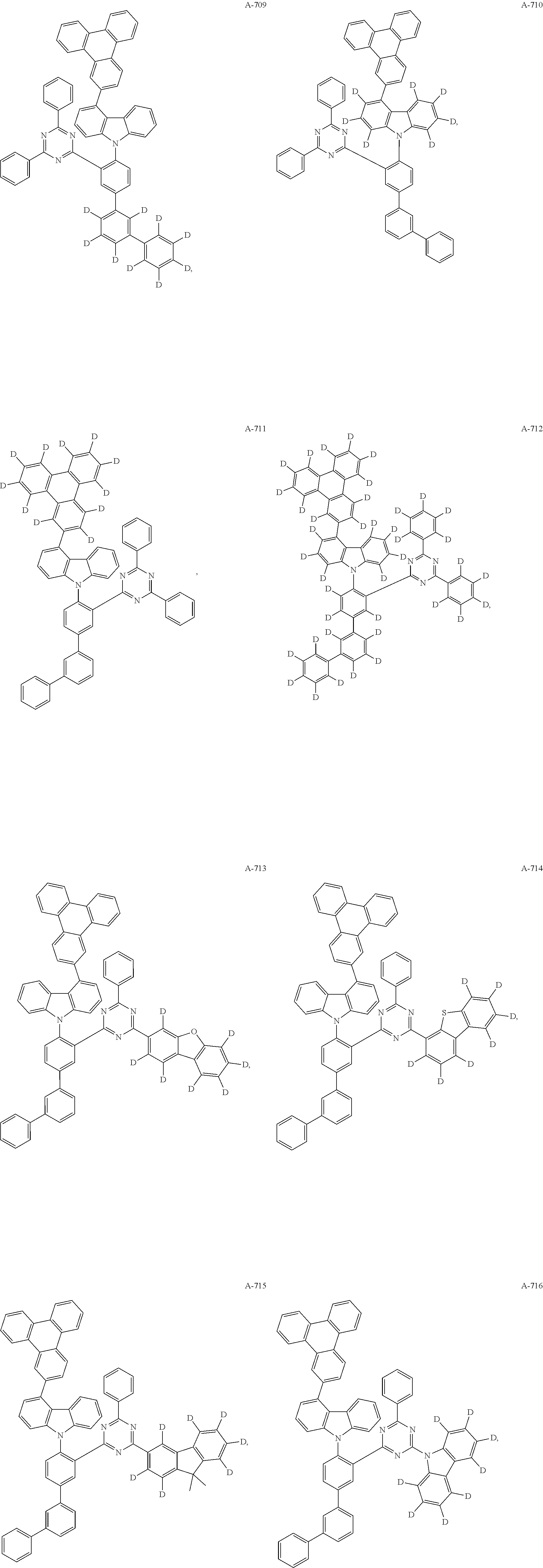 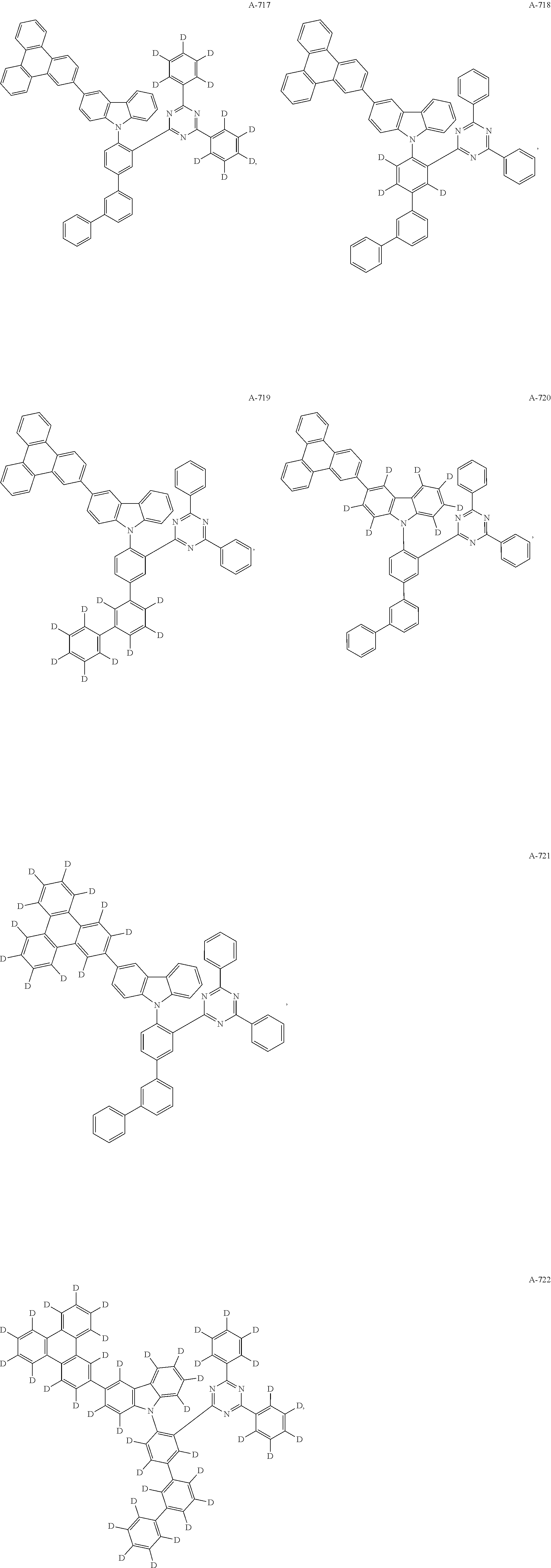 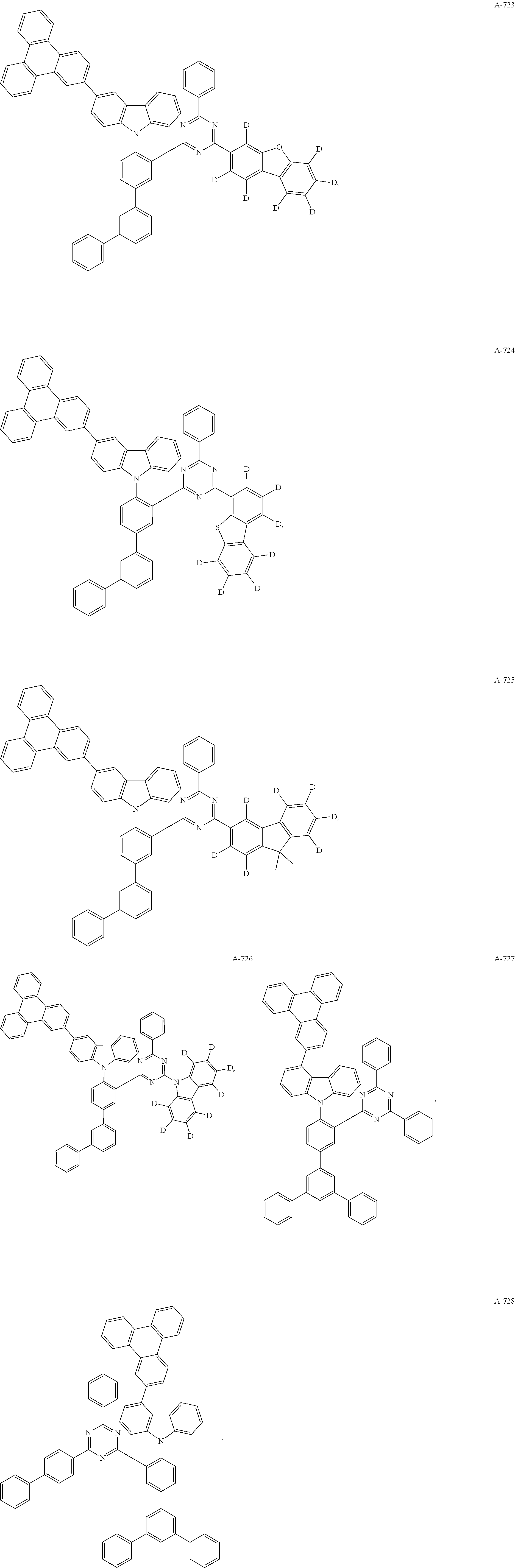 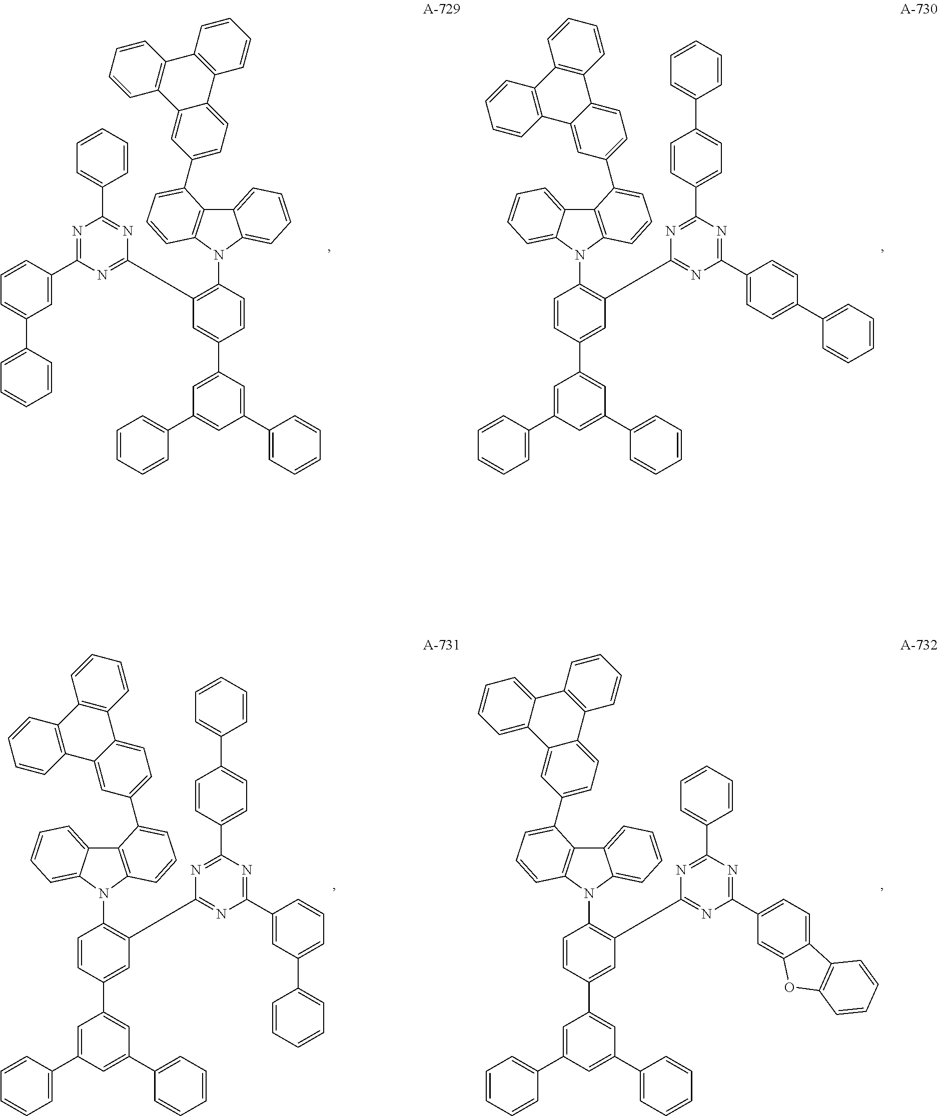 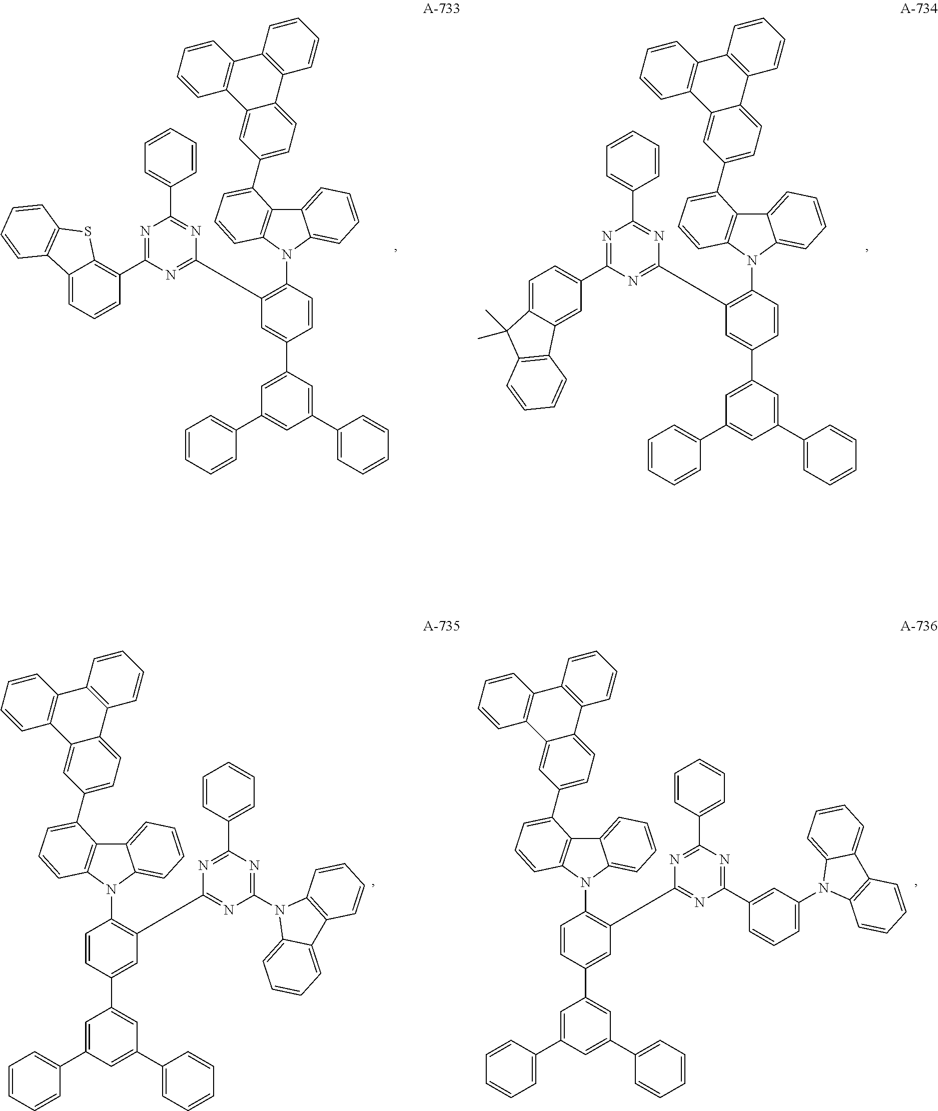 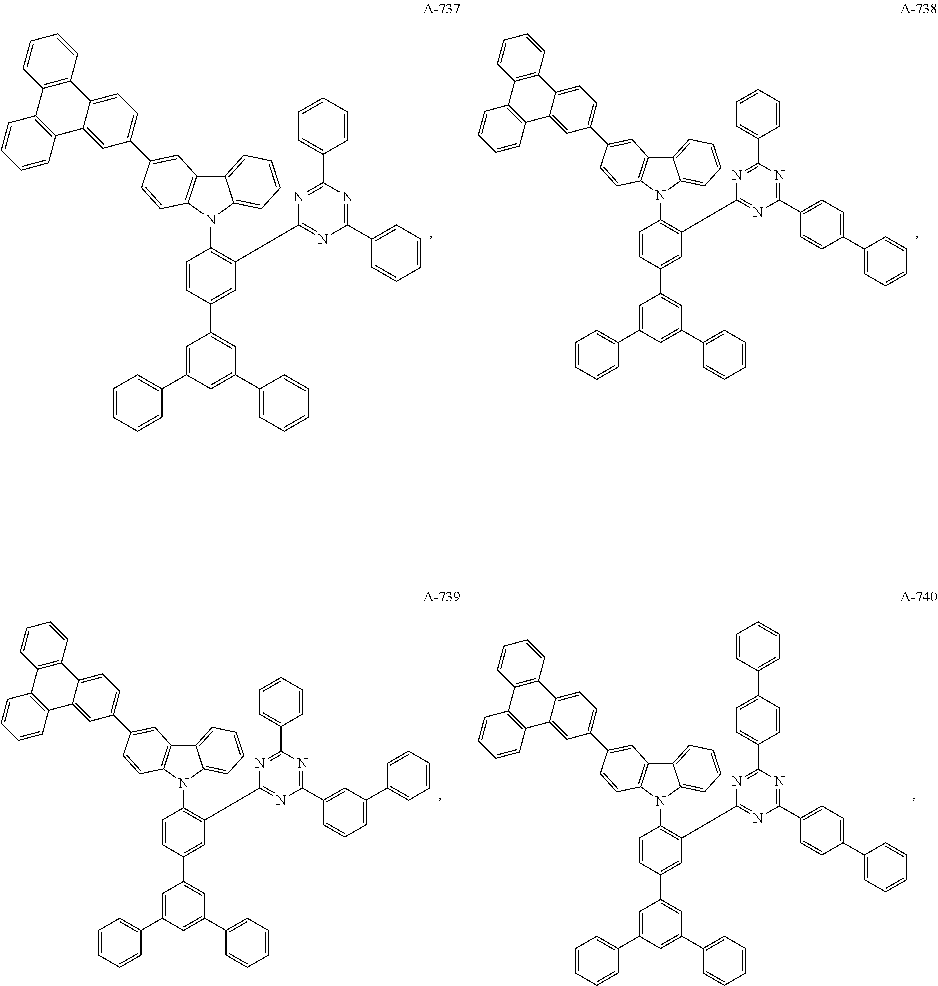 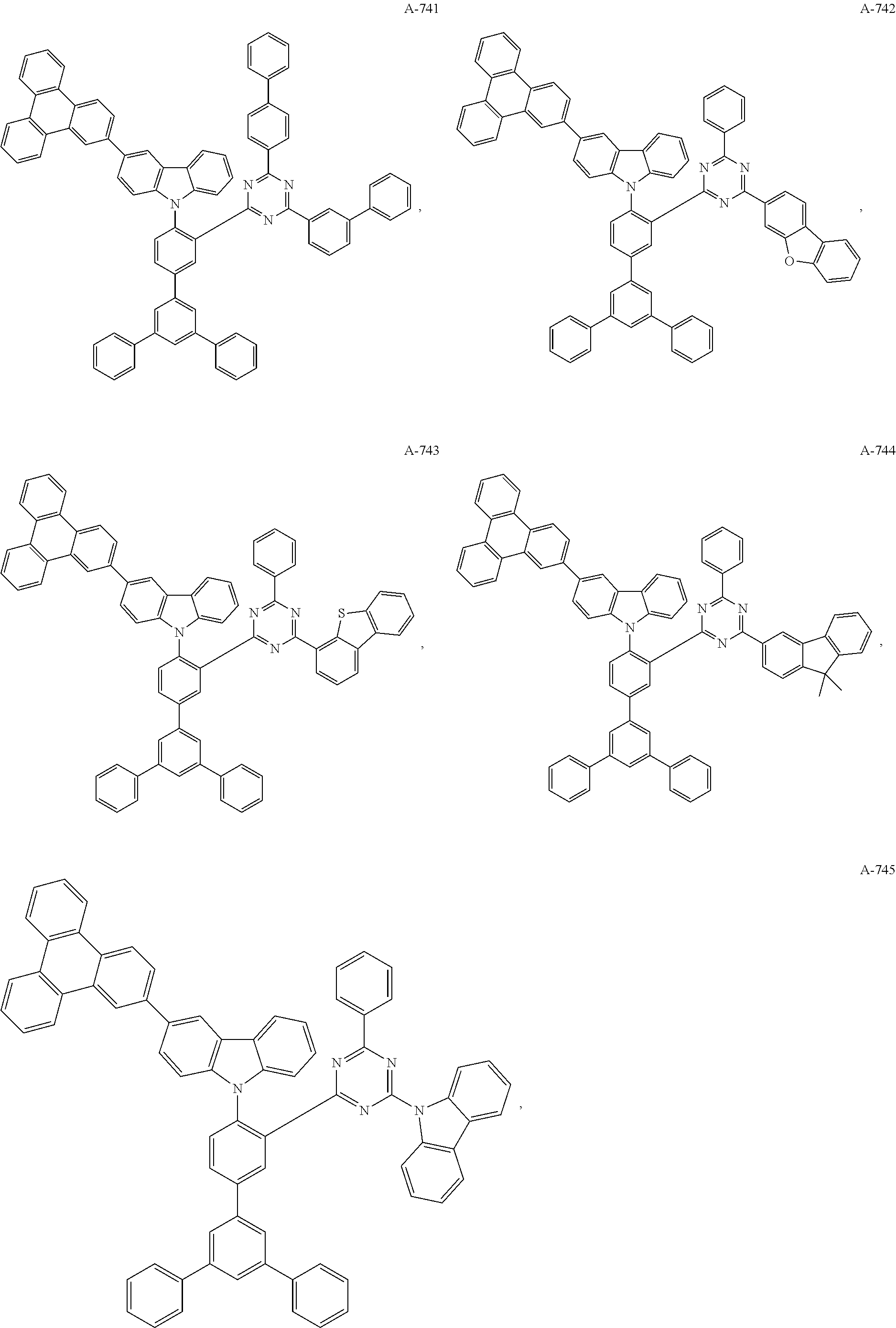 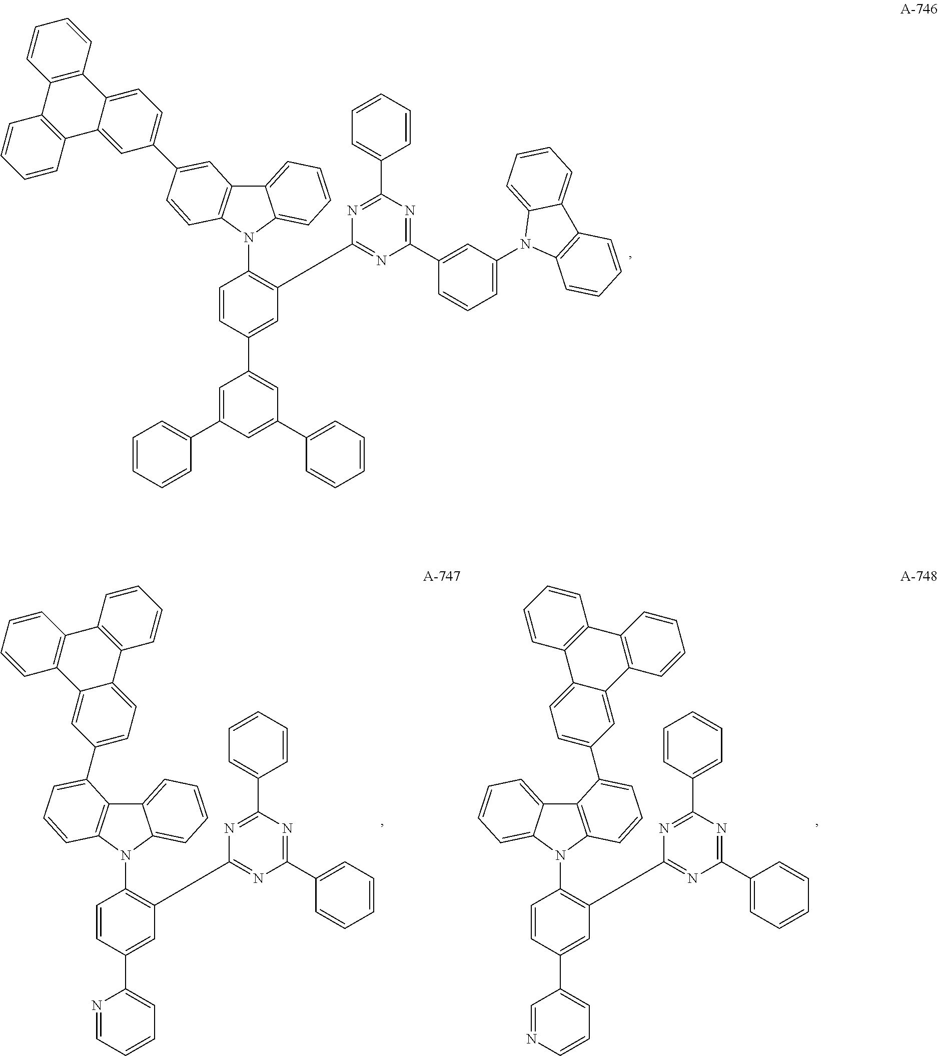 [SMILES: c1ccc(-c2cc(-c3ccccc3)cc(-c3ccc(-n4c5ccccc5c5cc(-c6ccc7c8ccccc8c8ccccc8c7c6)ccc54)c(-c4nc(-c5ccccc5)nc(-c5cccc(-n6c7ccccc7c7ccccc76)c5)n4)c3)c2)cc1 ; c1ccc(-c2nc(-c3ccccc3)nc(-c3cc(-c4ccccn4)ccc3-n3c4ccccc4c4c(-c5ccc6c7ccccc7c7ccccc7c6c5)cccc43)n2)cc1 ; c1ccc(-c2nc(-c3ccccc3)nc(-c3cc(-c4cccnc4)ccc3-n3c4ccccc4c4c(-c5ccc6c7ccccc7c7ccccc7c6c5)cccc43)n2)cc1] 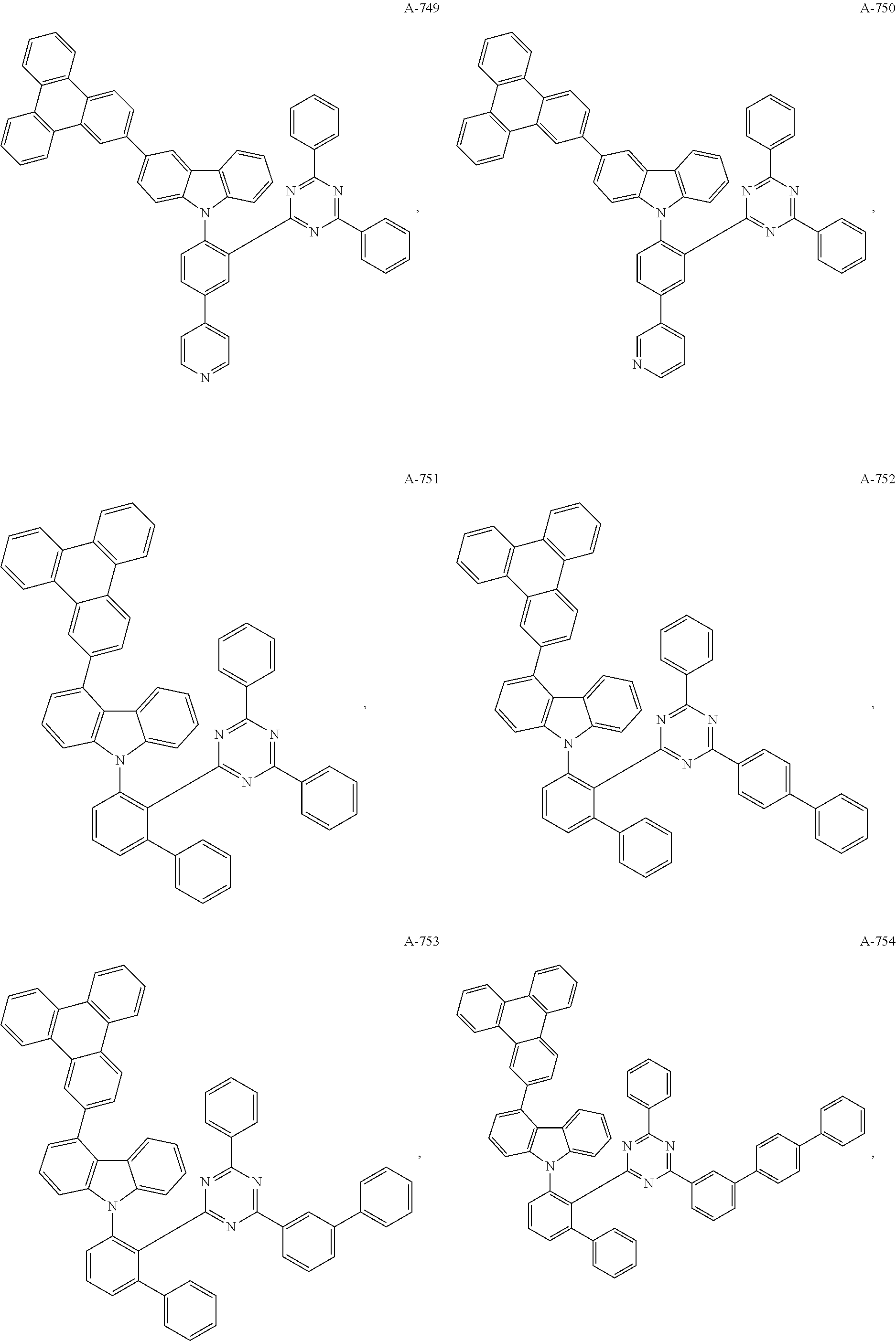 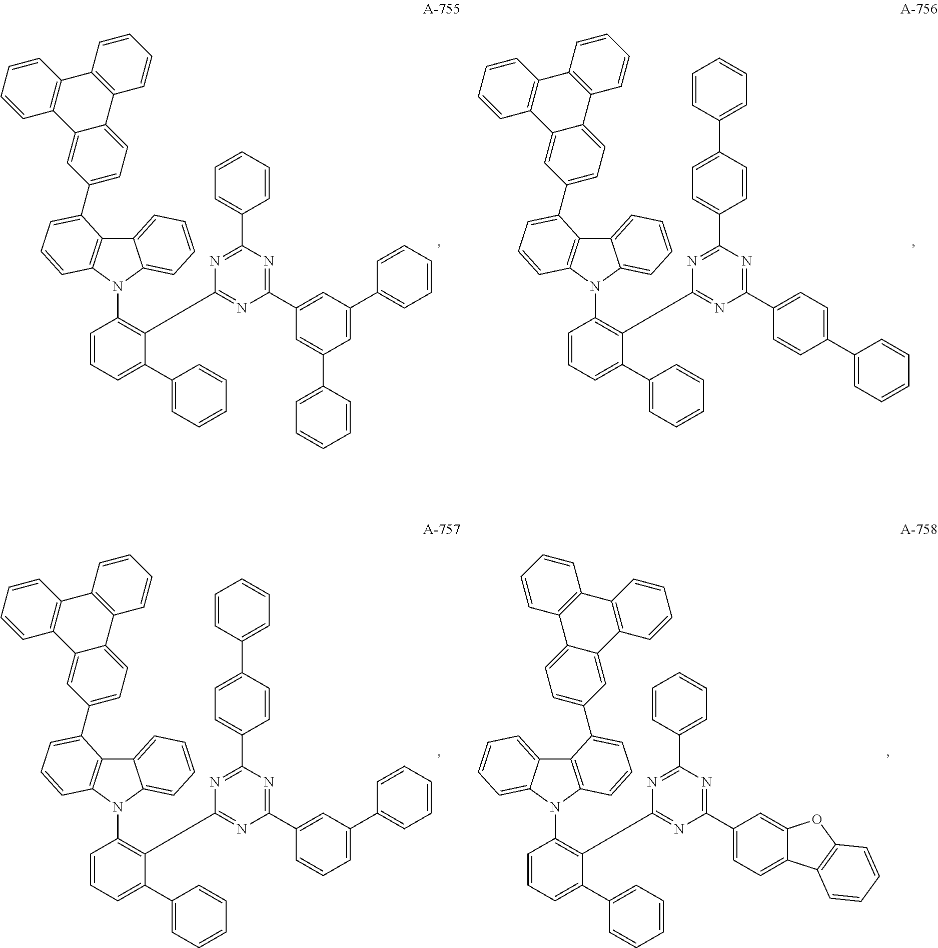 [SMILES: c1ccc(-c2cc(-c3ccccc3)cc(-c3nc(-c4ccccc4)nc(-c4c(-c5ccccc5)cccc4-n4c5ccccc5c5c(-c6ccc7c8ccccc8c8ccccc8c7c6)cccc54)n3)c2)cc1 ; c1ccc(-c2ccc(-c3nc(-c4ccc(-c5ccccc5)cc4)nc(-c4c(-c5ccccc5)cccc4-n4c5ccccc5c5c(-c6ccc7c8ccccc8c8ccccc8c7c6)cccc54)n3)cc2)cc1 ; c1ccc(-c2ccc(-c3nc(-c4cccc(-c5ccccc5)c4)nc(-c4c(-c5ccccc5)cccc4-n4c5ccccc5c5c(-c6ccc7c8ccccc8c8ccccc8c7c6)cccc54)n3)cc2)cc1 ; c1ccc(-c2nc(-c3ccc4c(c3)oc3ccccc34)nc(-c3c(-c4ccccc4)cccc3-n3c4ccccc4c4c(-c5ccc6c7ccccc7c7ccccc7c6c5)cccc43)n2)cc1] 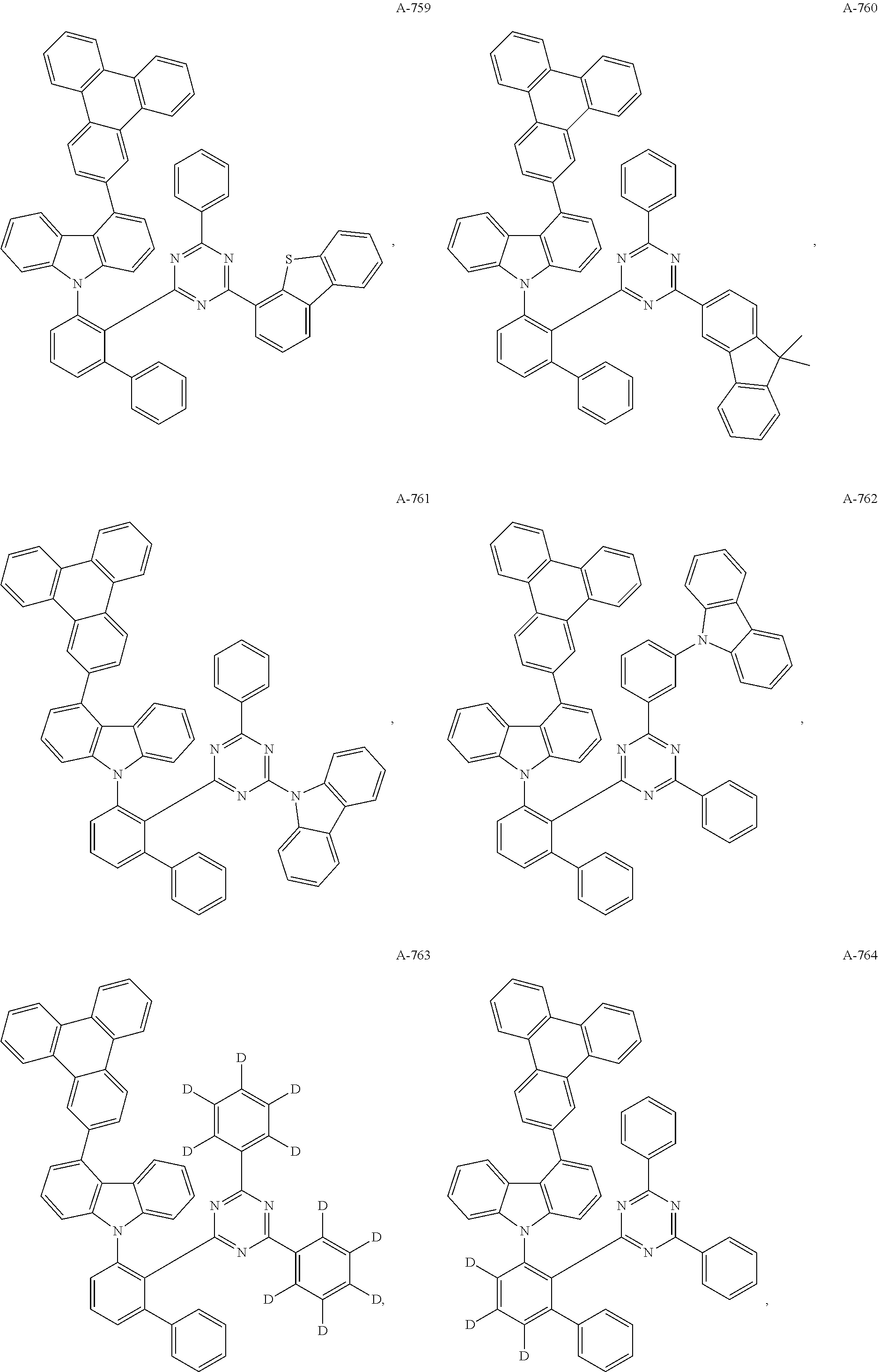 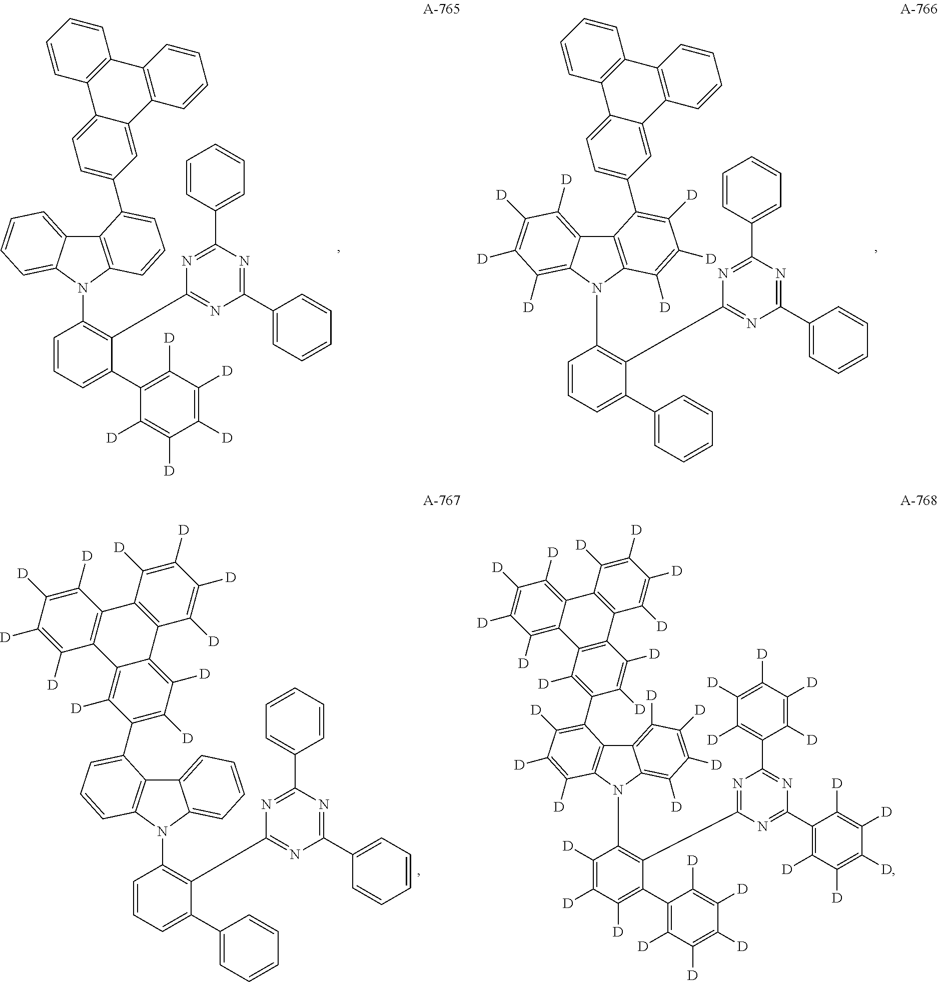 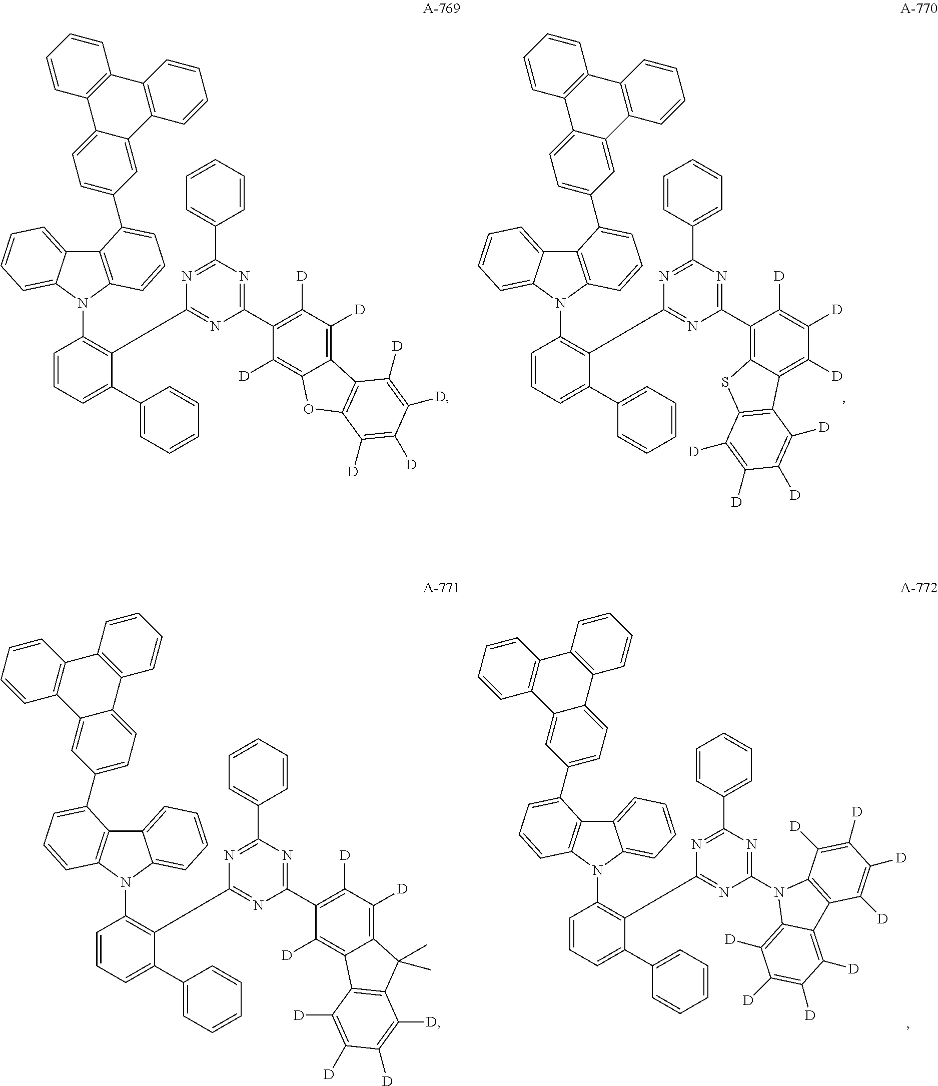 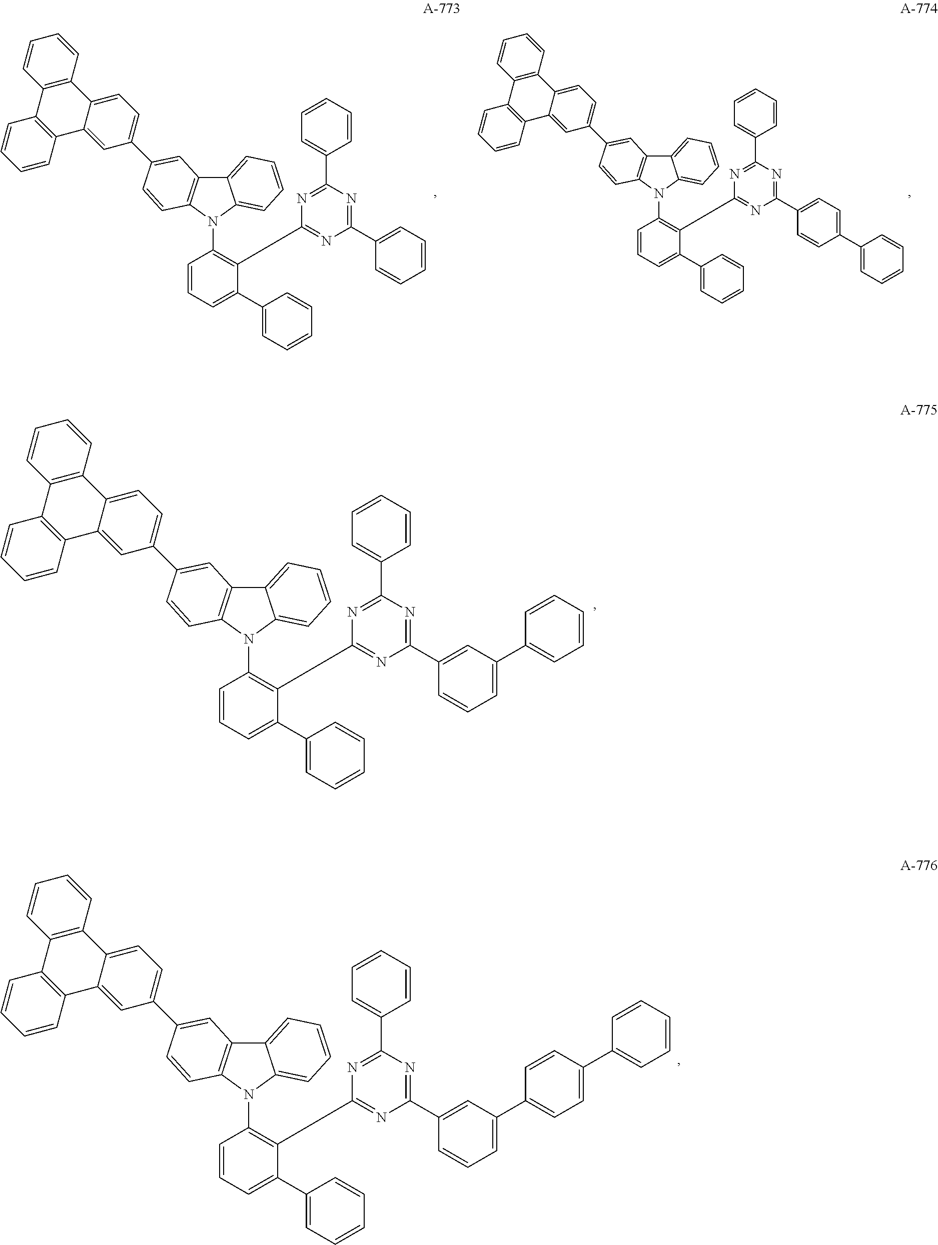 [SMILES: c1ccc(-c2nc(-c3ccccc3)nc(-c3c(-c4ccccc4)cccc3-n3c4ccccc4c4cc(-c5ccc6c7ccccc7c7ccccc7c6c5)ccc43)n2)cc1 ; c1ccc(-c2ccc(-c3nc(-c4ccccc4)nc(-c4c(-c5ccccc5)cccc4-n4c5ccccc5c5cc(-c6ccc7c8ccccc8c8ccccc8c7c6)ccc54)n3)cc2)cc1 ; c1ccc(-c2cccc(-c3nc(-c4ccccc4)nc(-c4c(-c5ccccc5)cccc4-n4c5ccccc5c5cc(-c6ccc7c8ccccc8c8ccccc8c7c6)ccc54)n3)c2)cc1 ; c1ccc(-c2ccc(-c3cccc(-c4nc(-c5ccccc5)nc(-c5c(-c6ccccc6)cccc5-n5c6ccccc6c6cc(-c7ccc8c9ccccc9c9ccccc9c8c7)ccc65)n4)c3)cc2)cc1] 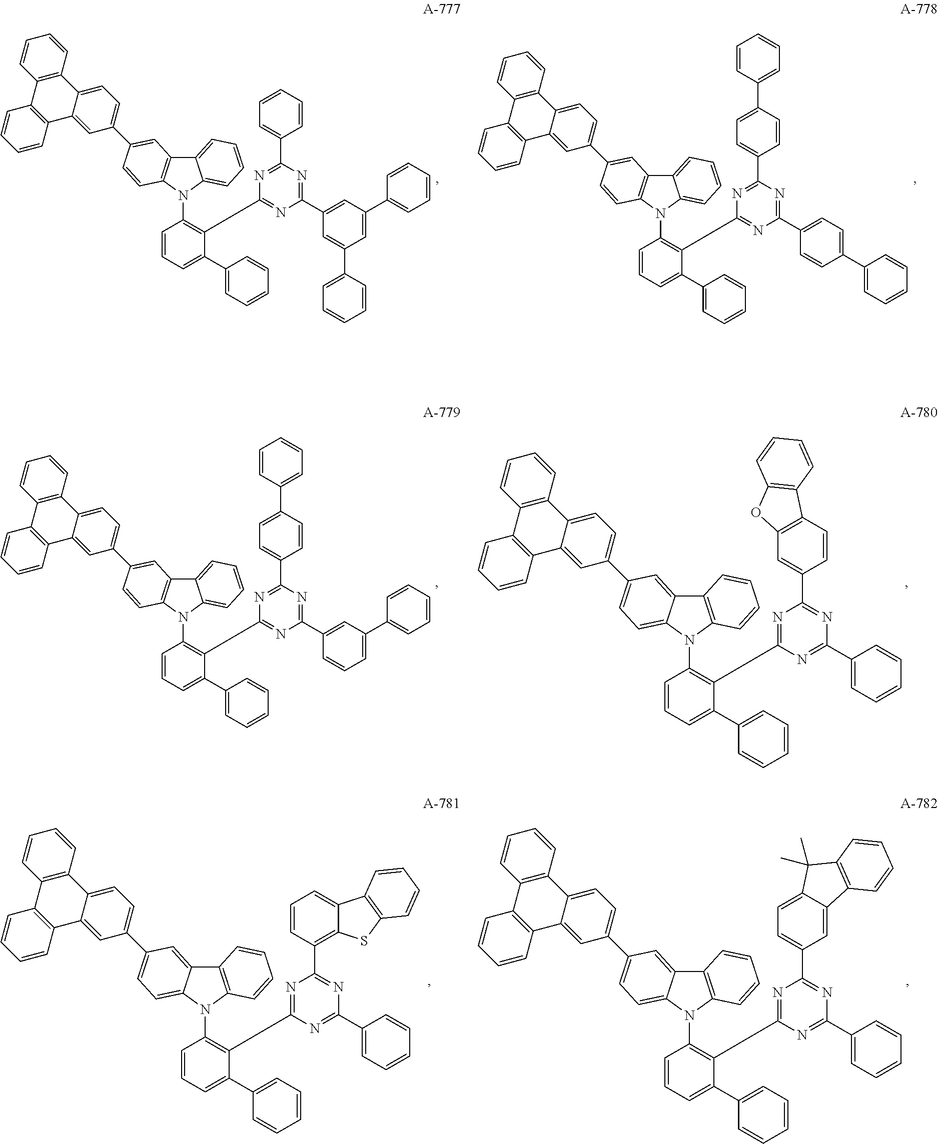 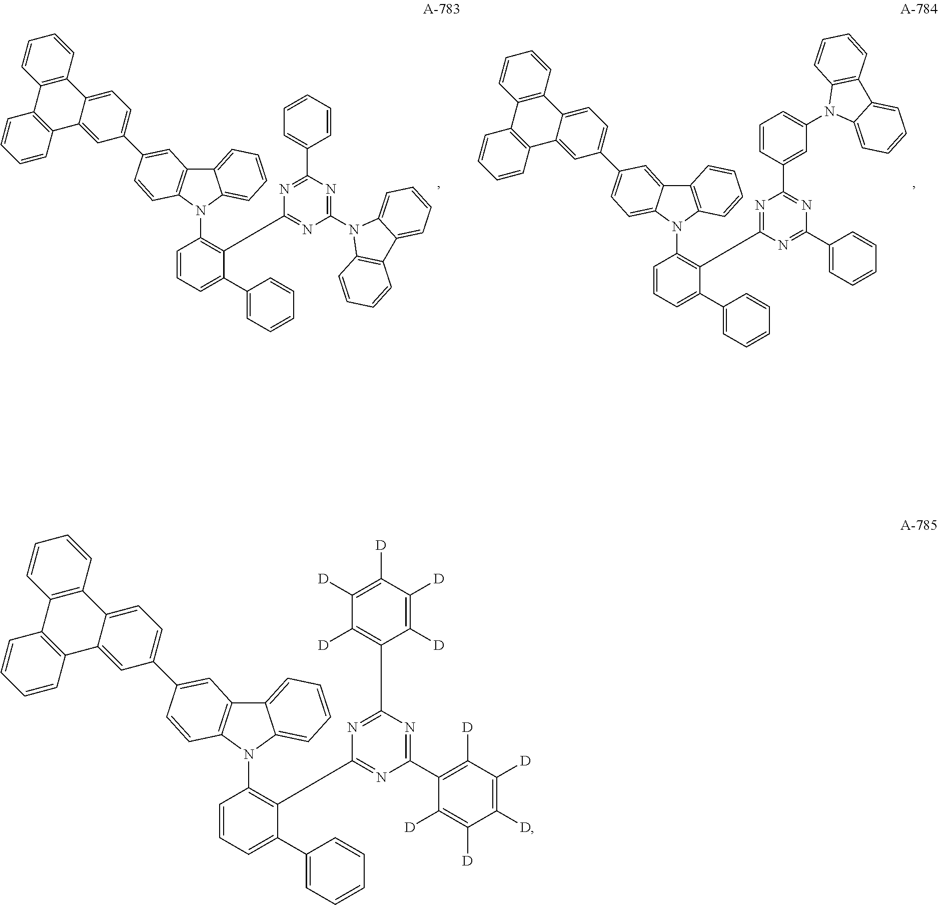 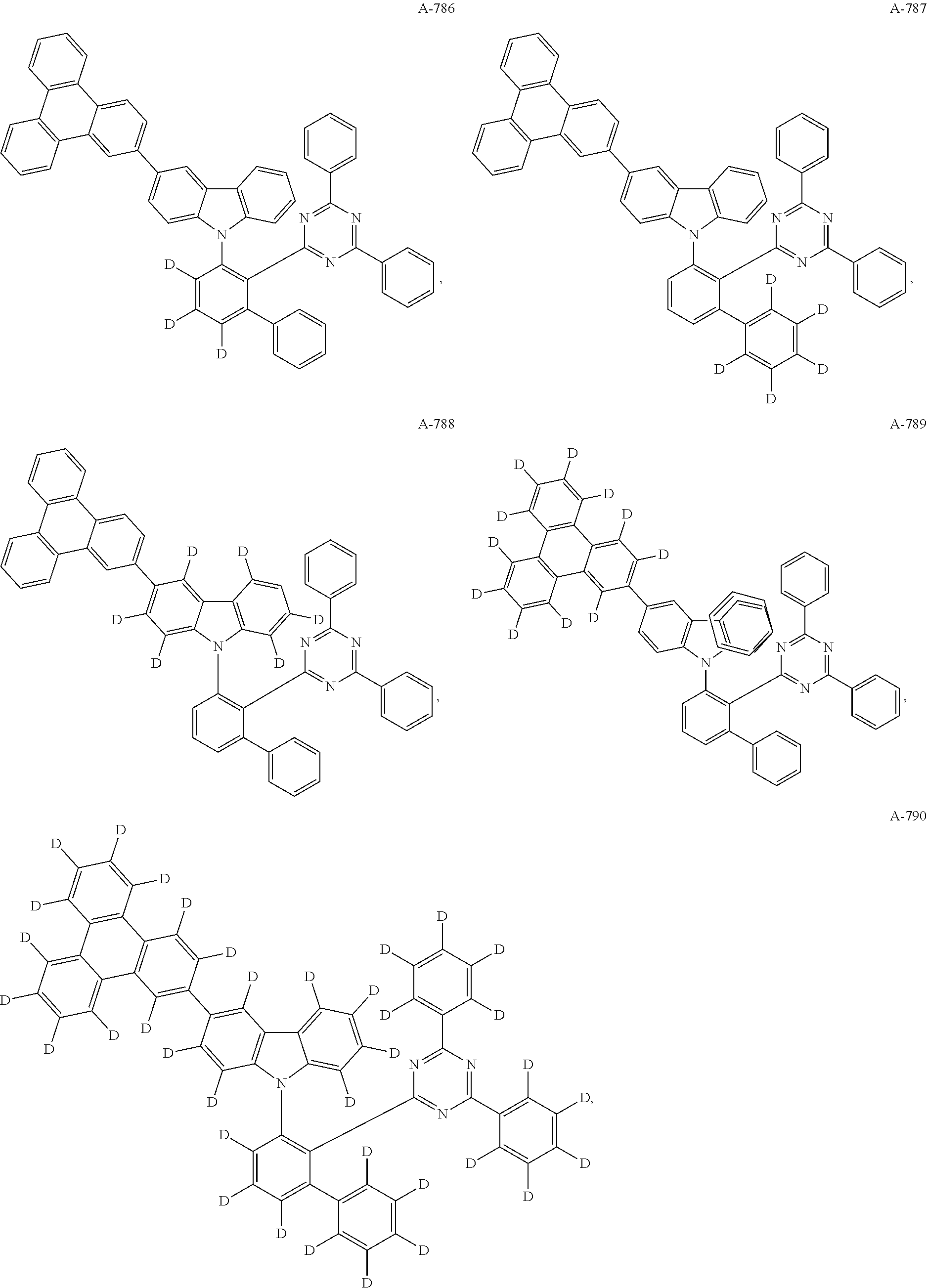 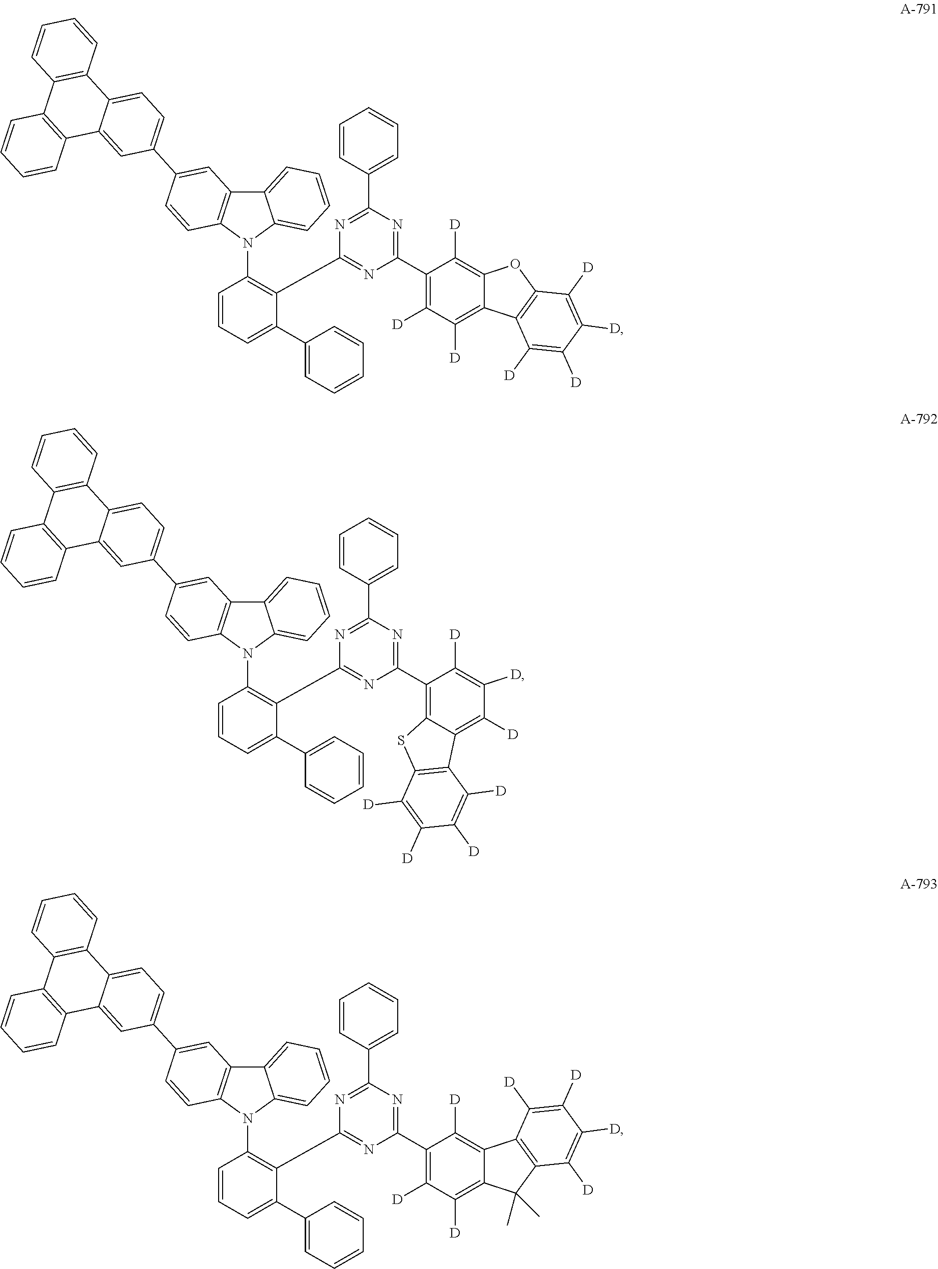 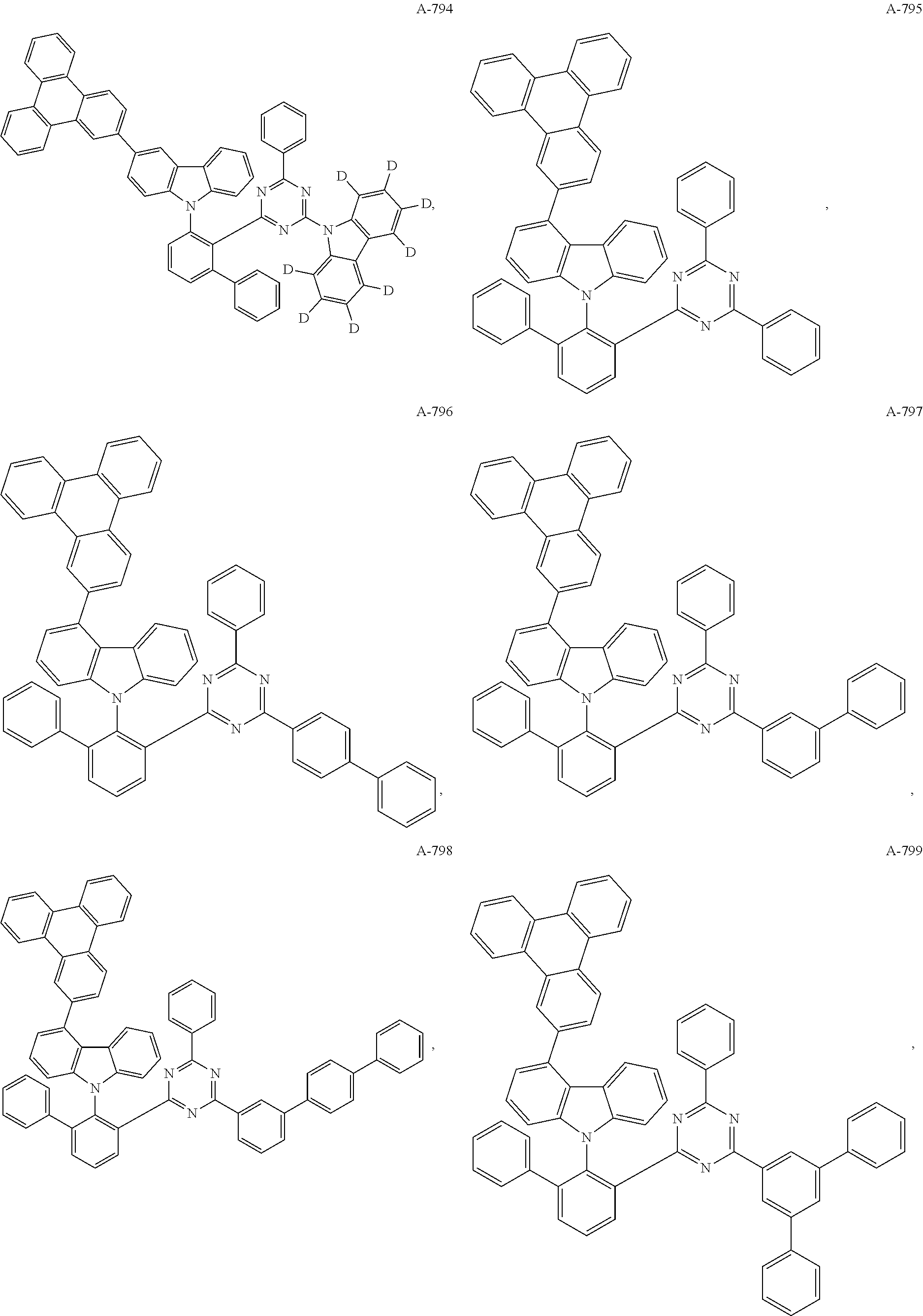 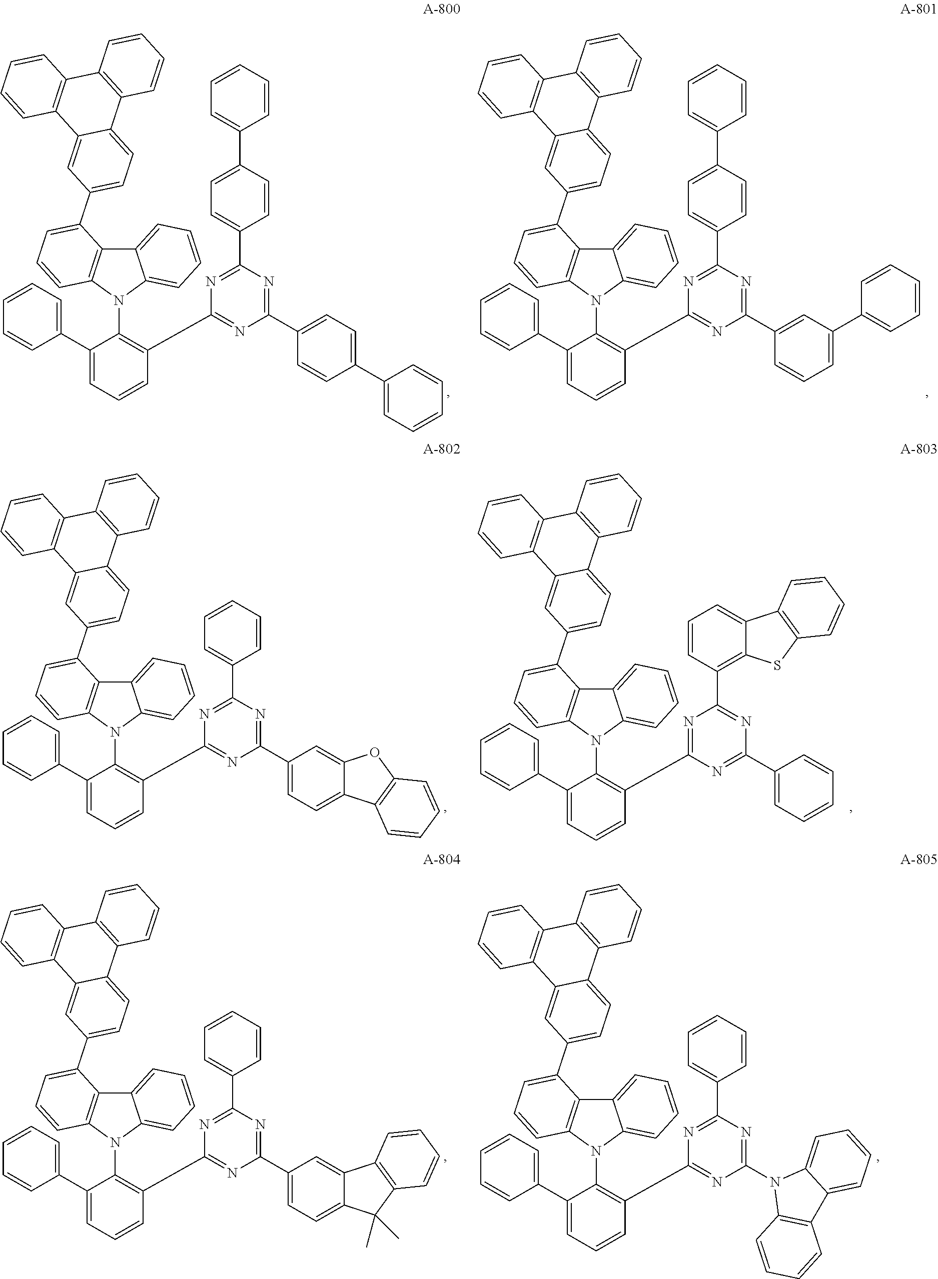 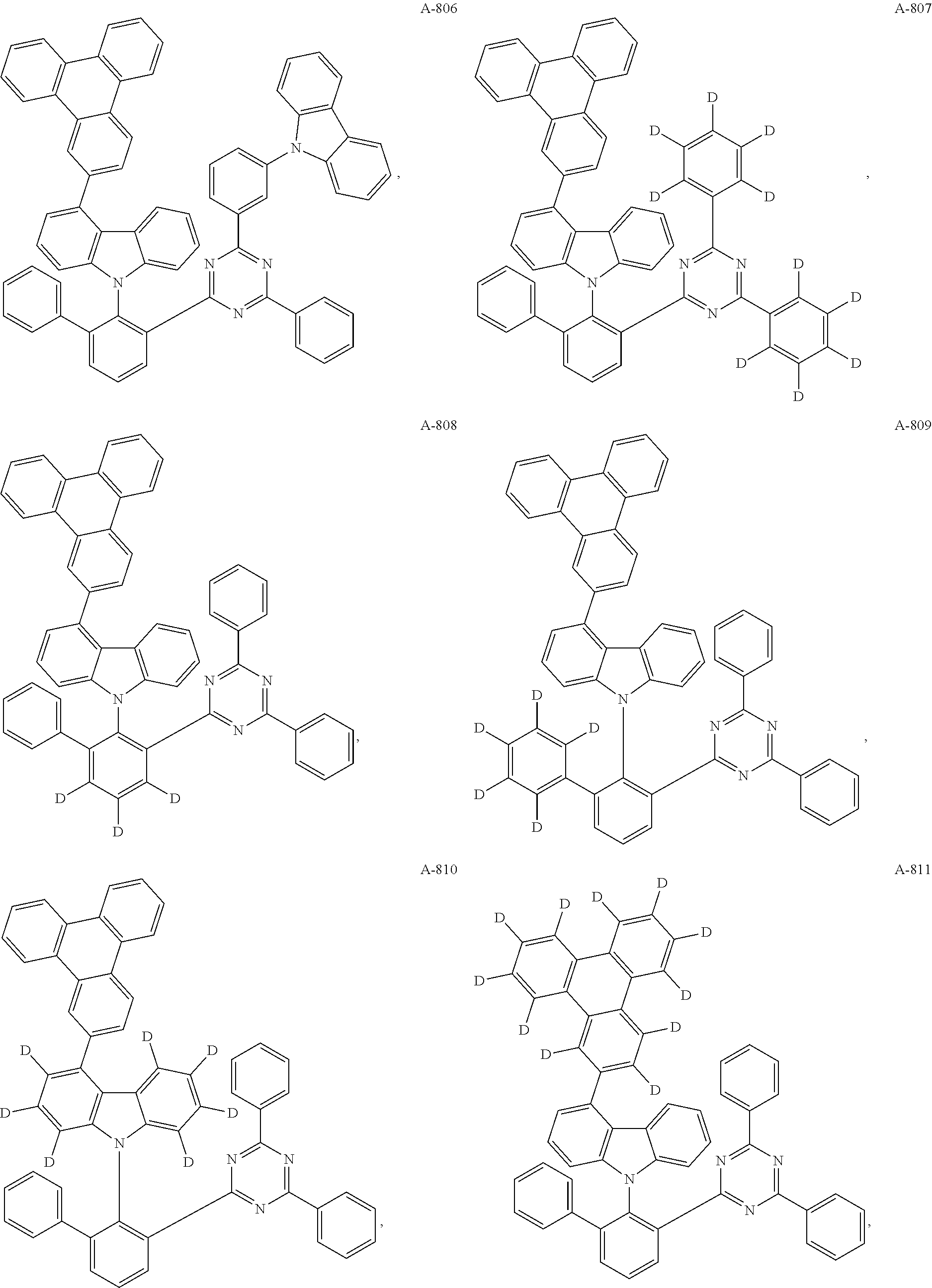 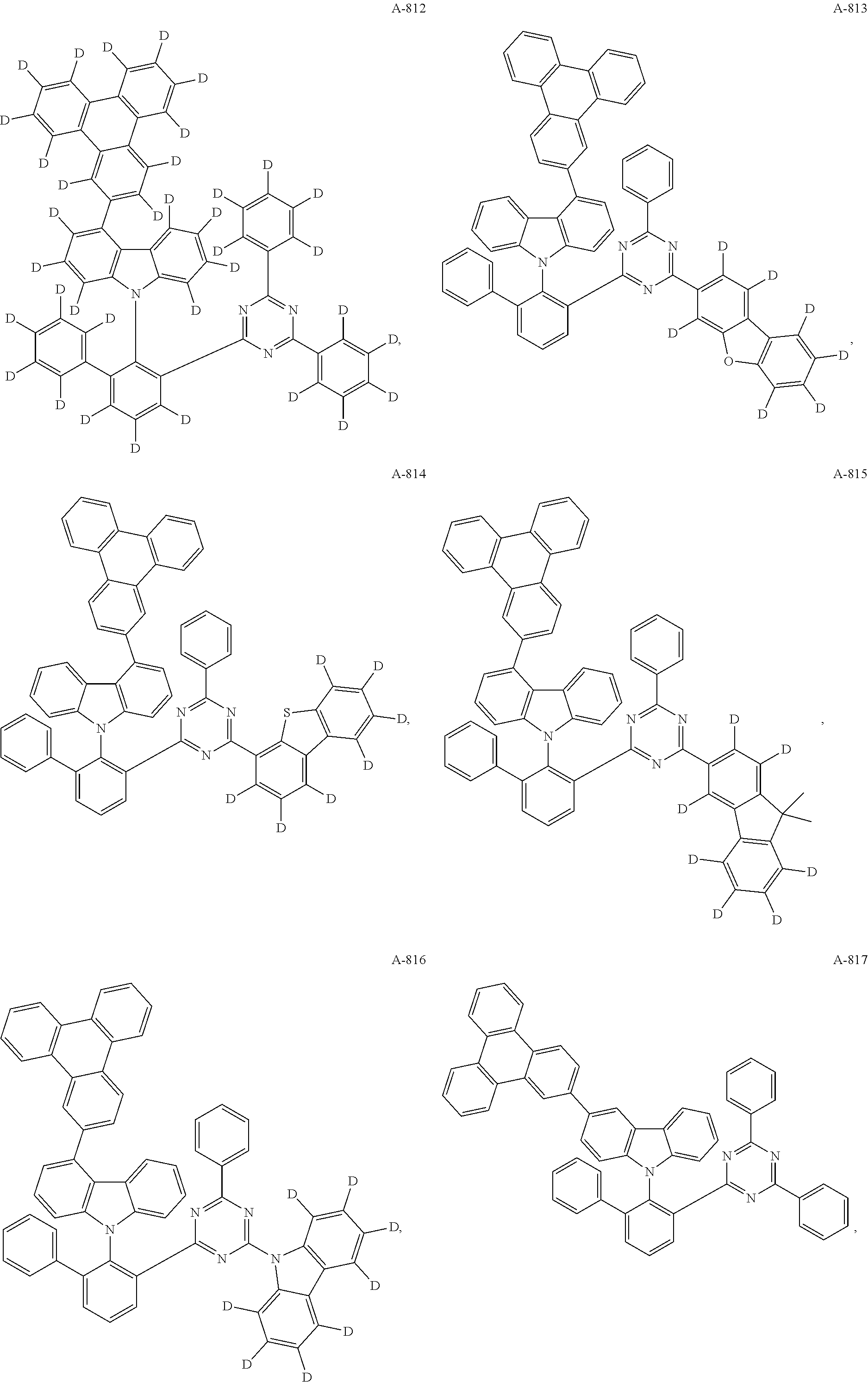 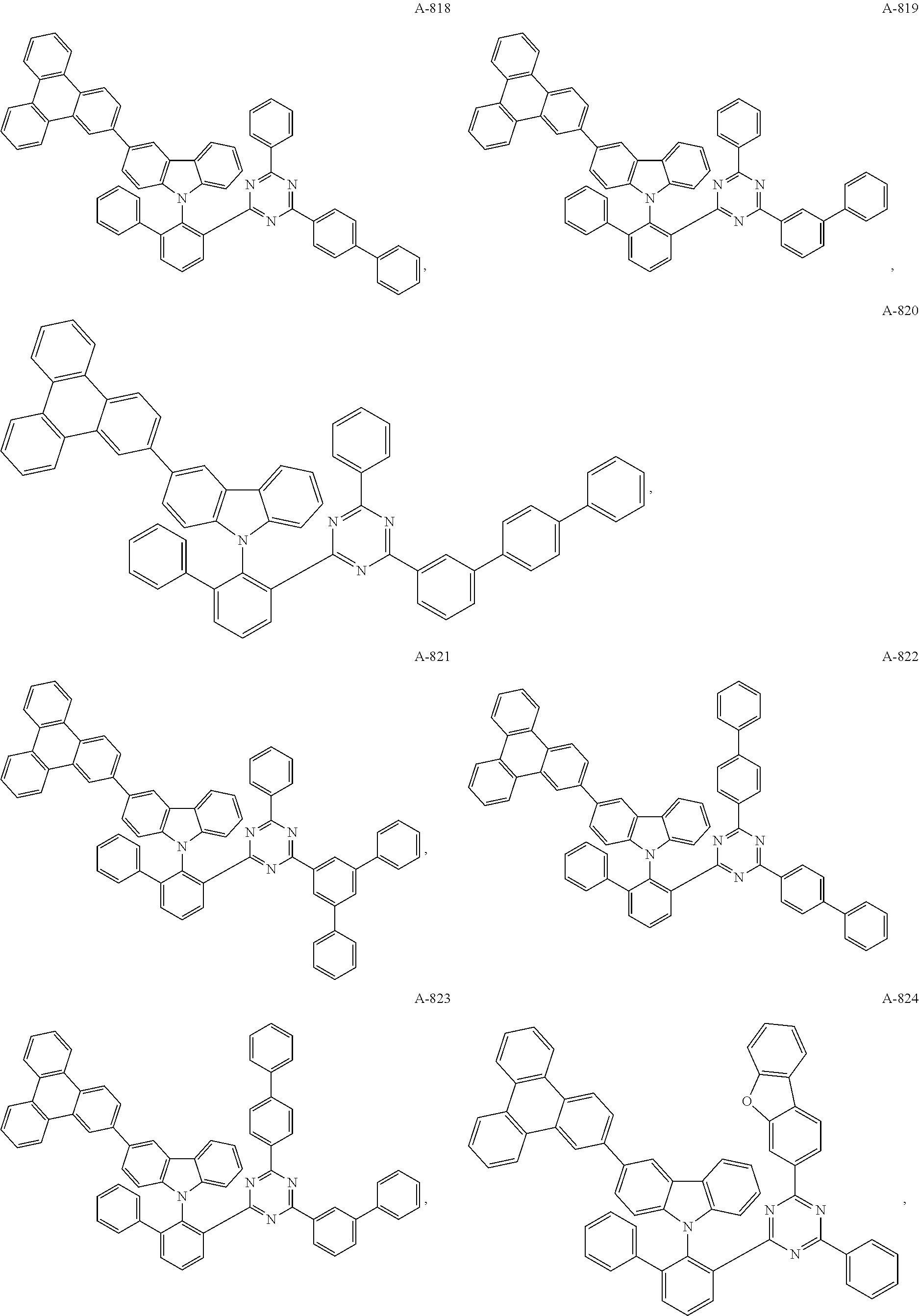 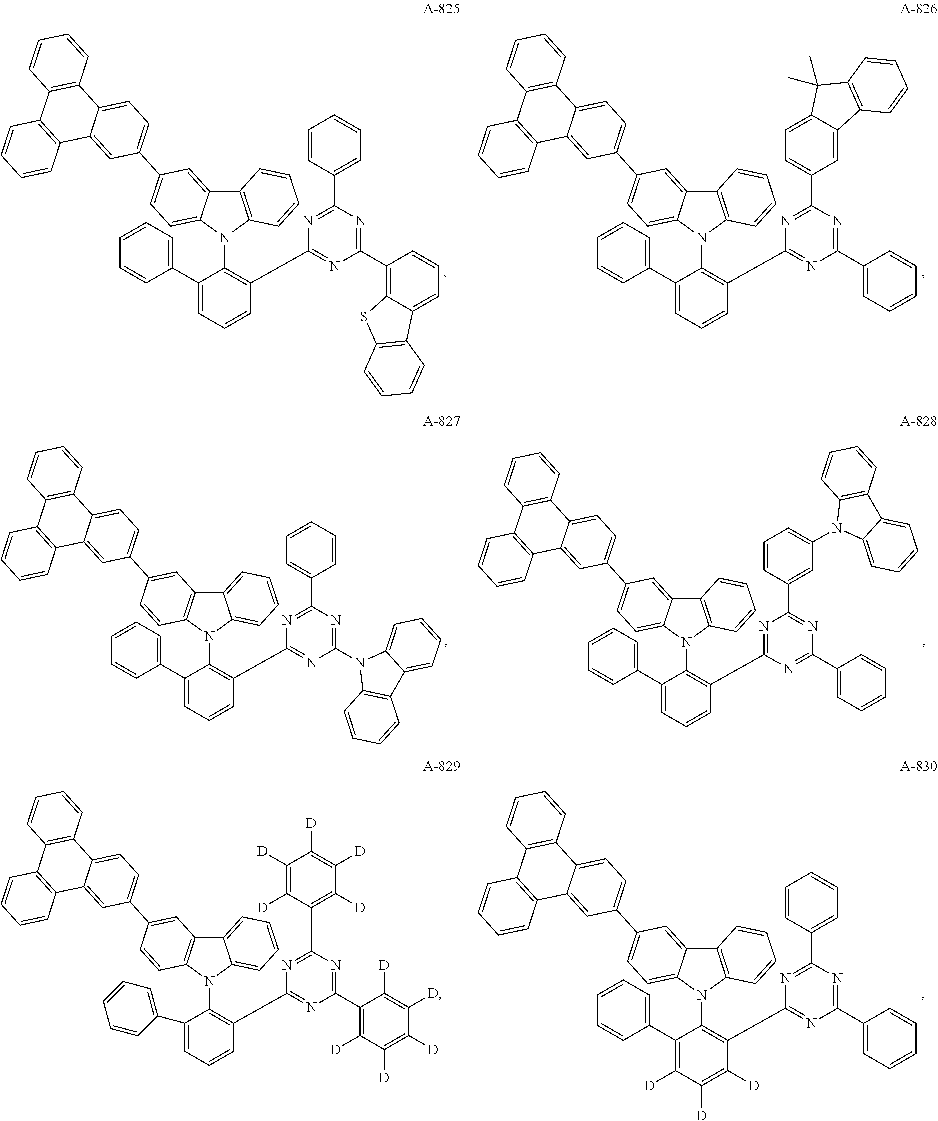 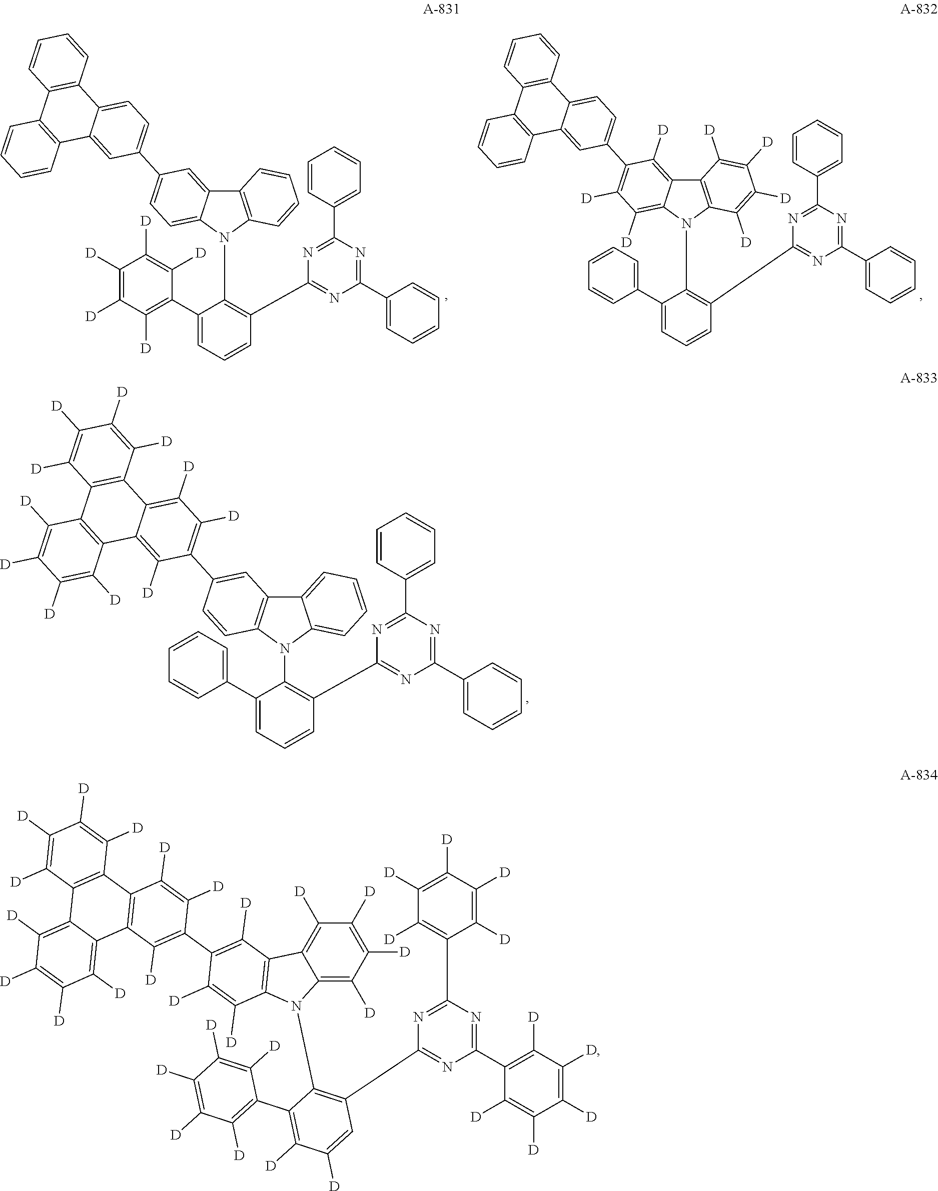 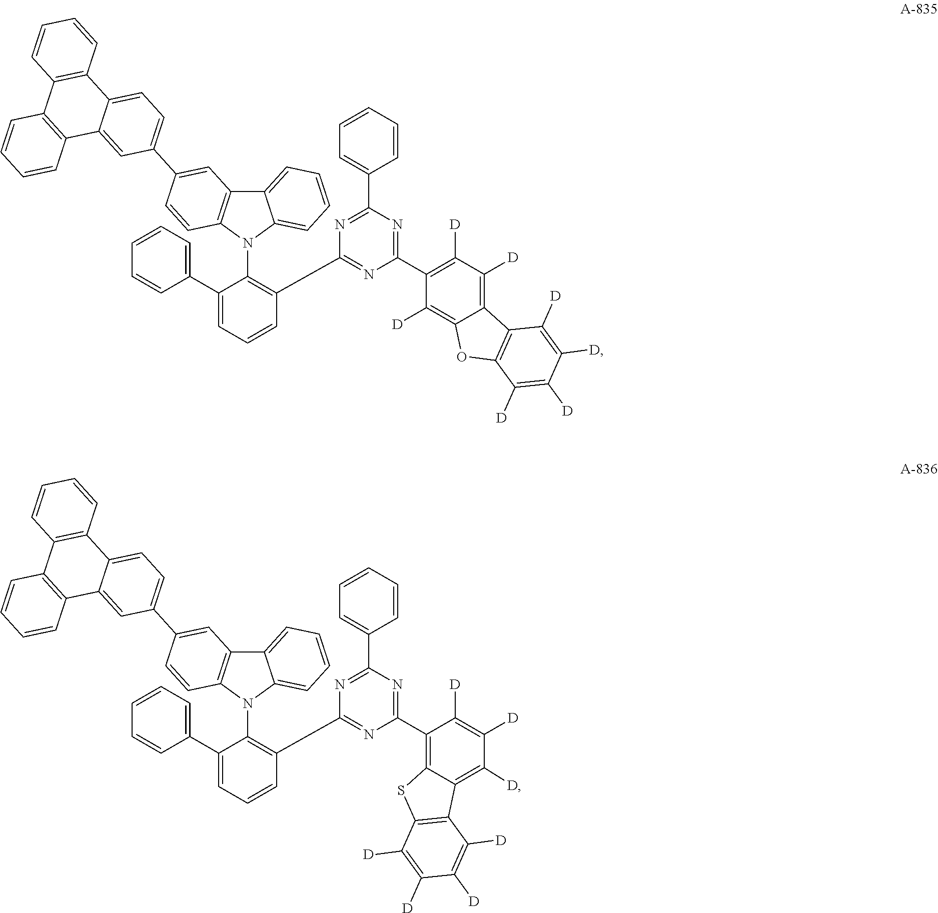 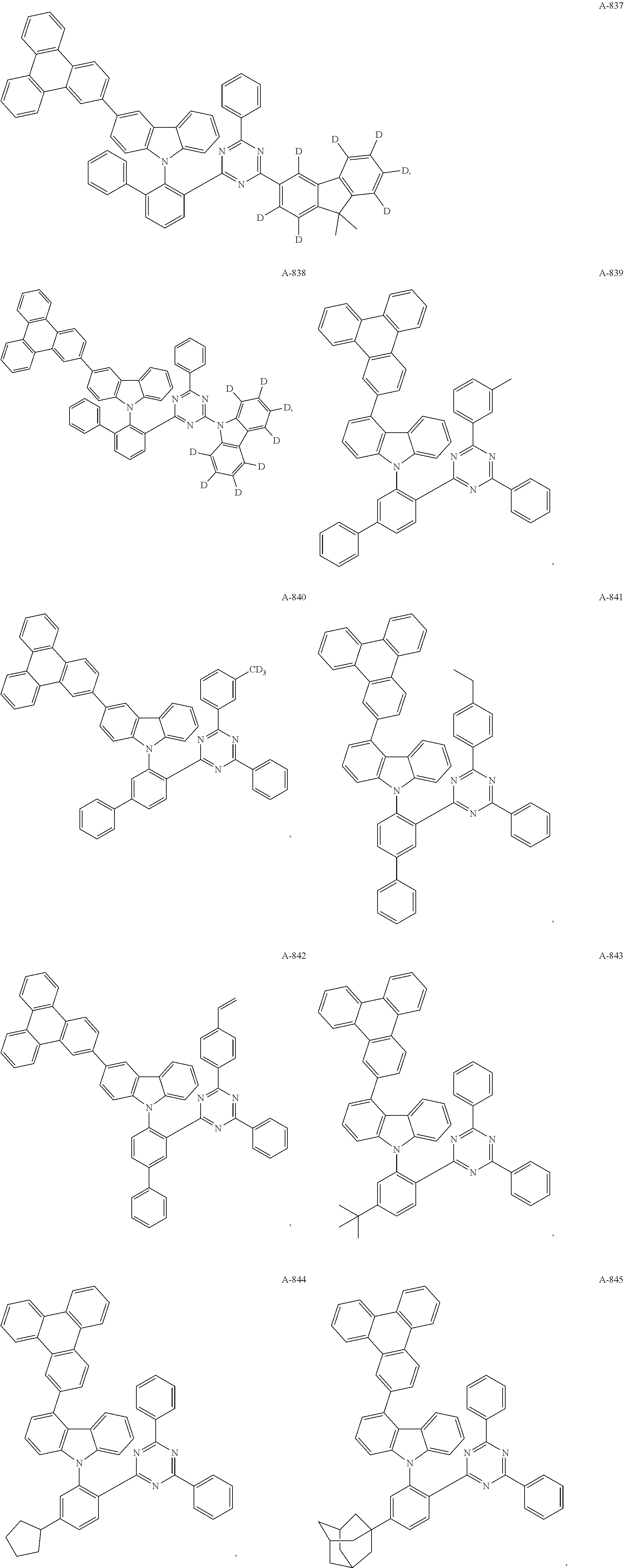 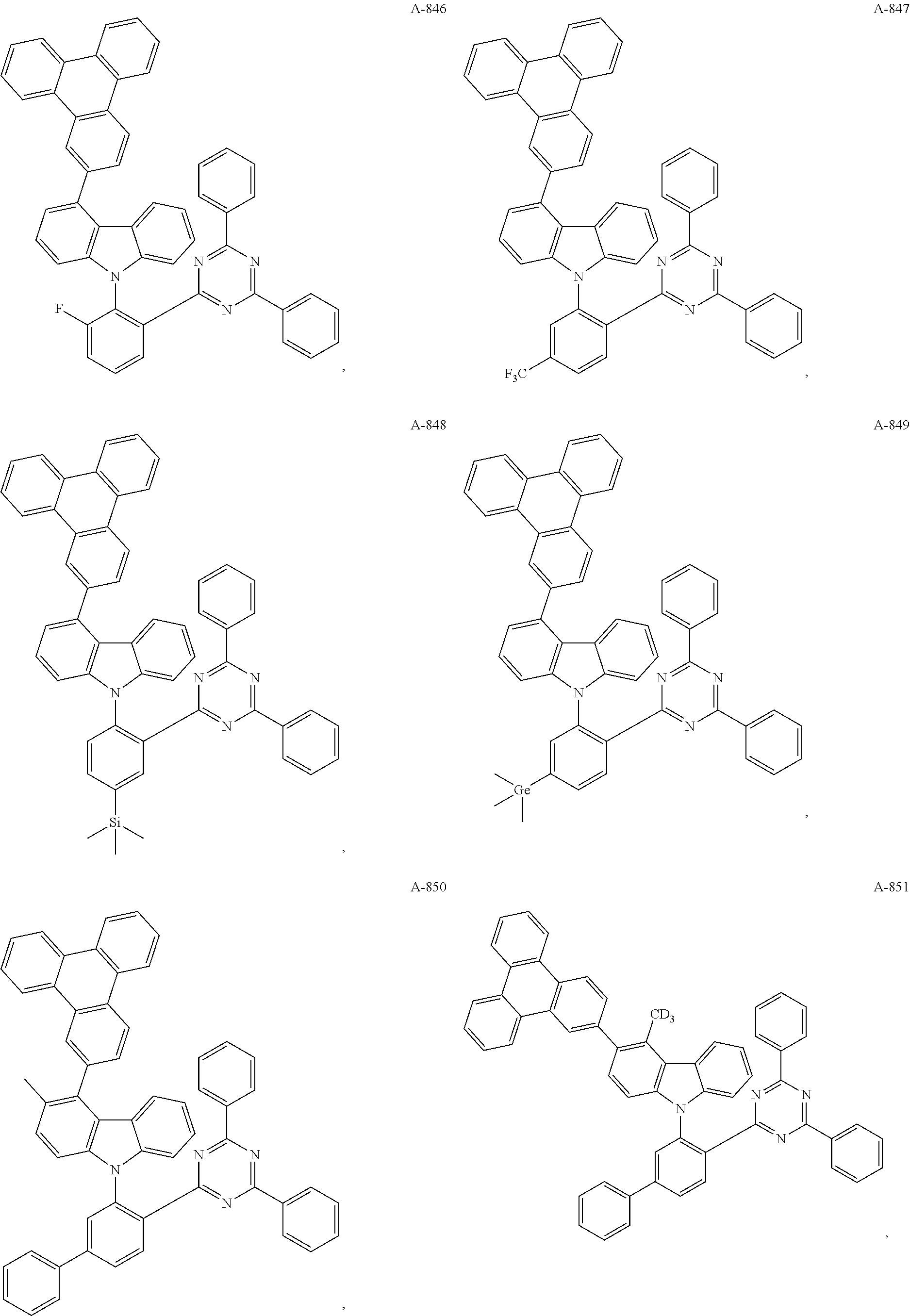 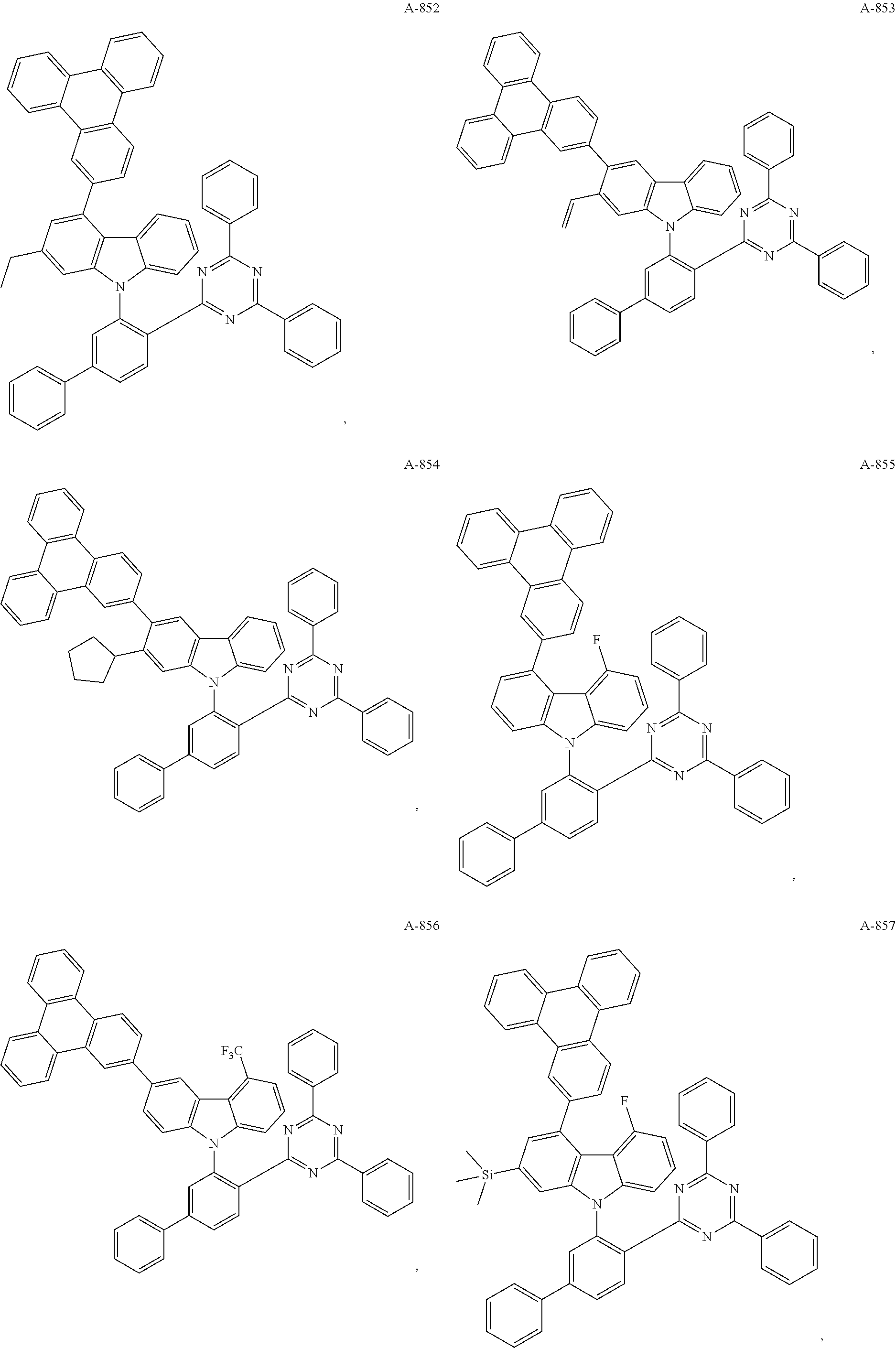 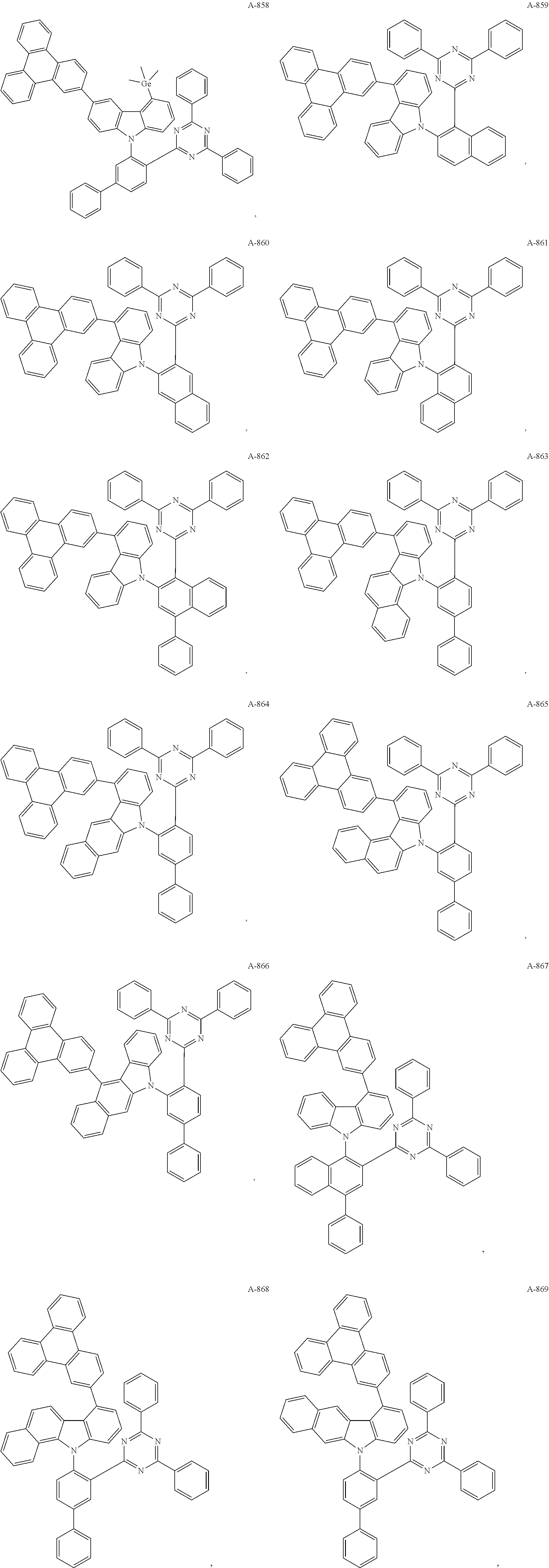 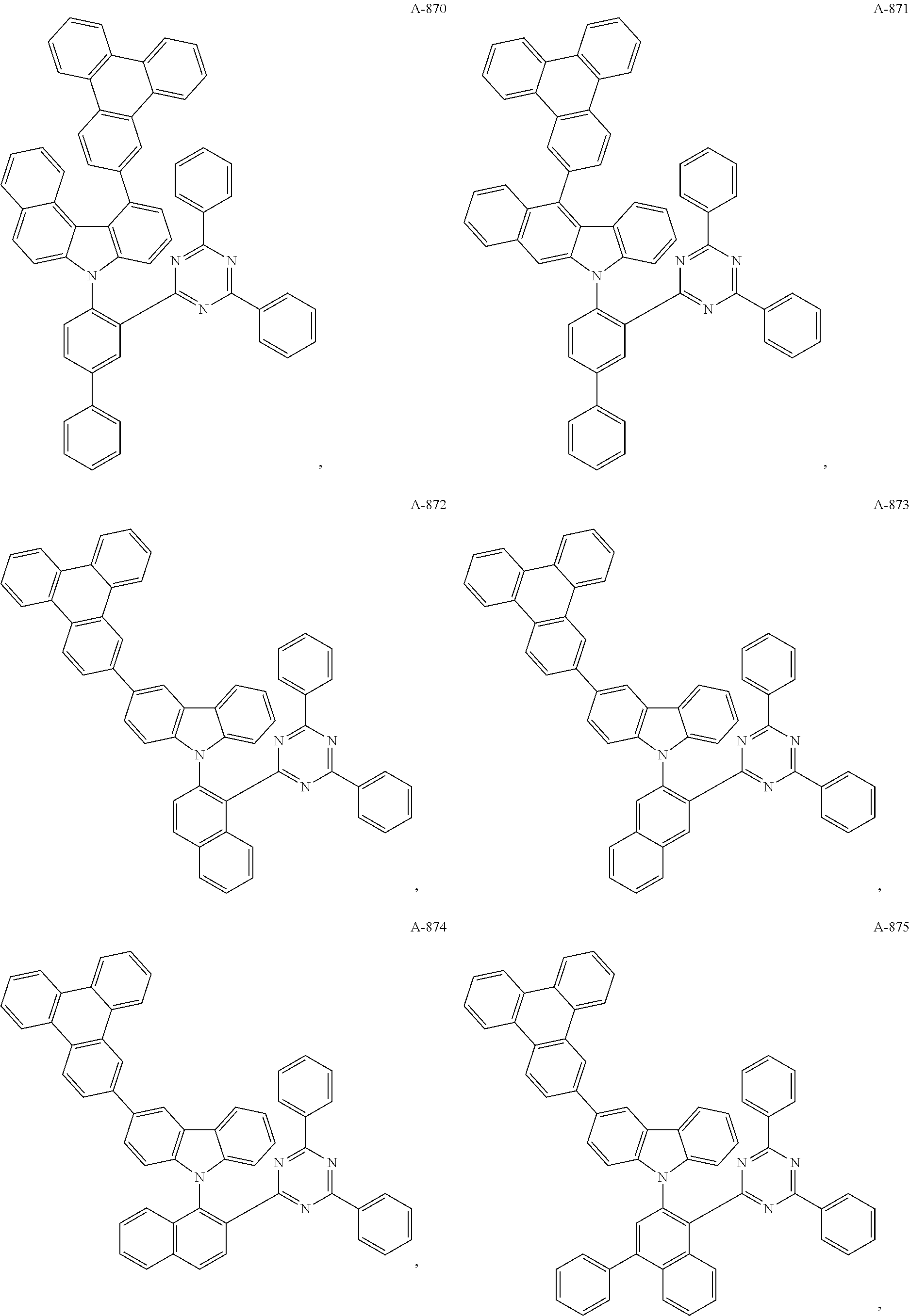 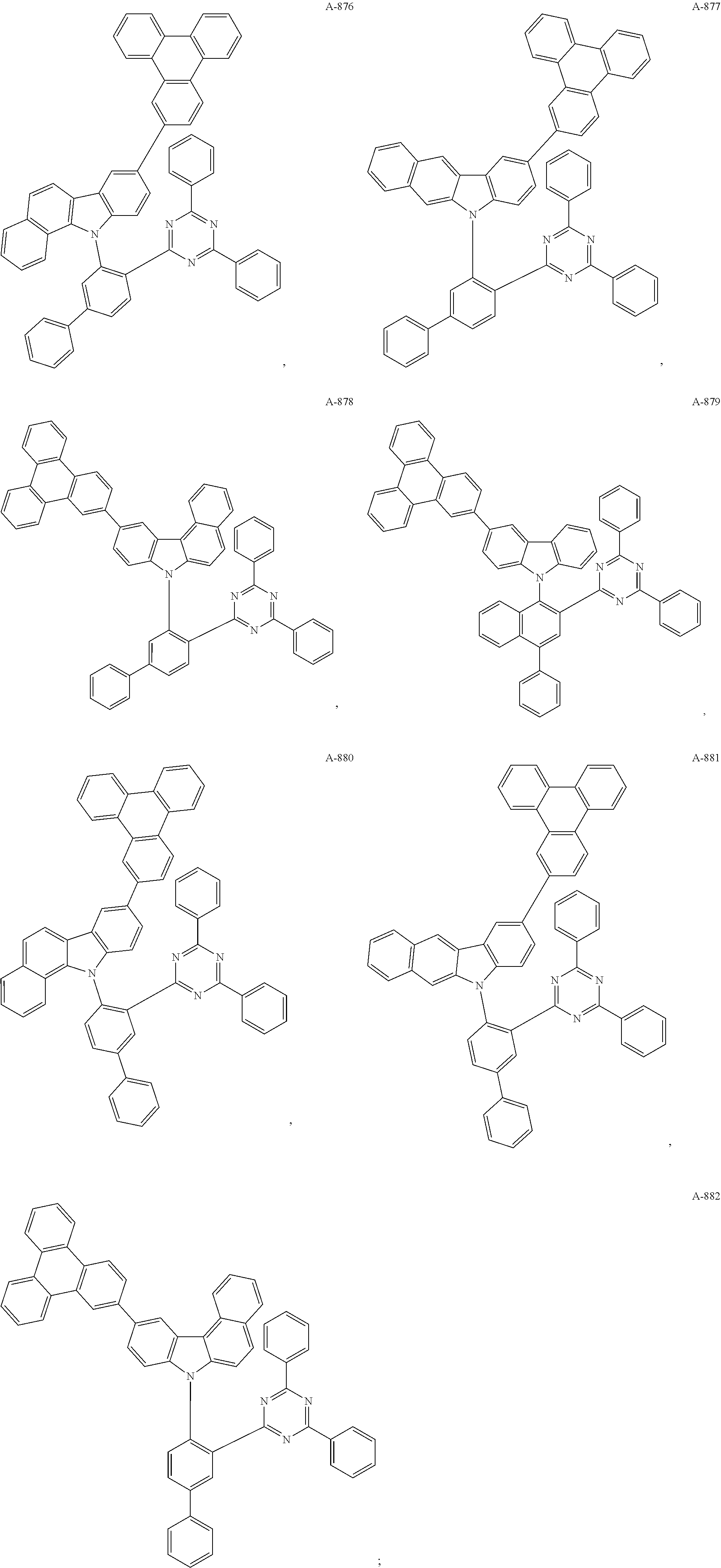
wherein optionally, hydrogens in Compound A-1 to Compound A-882 can be partially or fully substituted with deuterium.

10. An organic electroluminescent device, comprising:
an anode,
a cathode, and
an organic layer disposed between the anode and the cathode, wherein the organic layer comprises the compound of claim 1.

11. The organic electroluminescent device of claim 10, wherein the organic layer is an emissive layer, and the compound is a host compound; or the organic layer is an electron transporting layer, and the compound is an electron transporting compound; or the organic layer is a hole blocking layer, and the compound is a hole blocking compound.

12. The organic electroluminescent device of claim 11, wherein the emissive layer further comprises a second host compound, and the second host compound comprises at least one chemical group selected from the group consisting of: benzene, pyridine, pyrimidine, triazine, carbazole, azacarbazole, indolocarbazole, dibenzothiophene, aza-dibenzothiophene, dibenzofuran, azadibenzofuran, dibenzoselenophene, triphenylene, azatriphenylene, fluorene, silafluorene, naphthalene, quinoline, isoquinoline, quinazoline, quinoxaline, phenanthrene, azaphenanthrene, and combinations thereof;
preferably, the second host compound comprises at least one chemical group selected from the group consisting of: benzene, carbazole, indolocarbazole, dibenzofuran, dibenzothiophene, fluorene, and combinations thereof.

13. The organic electroluminescent device of claim 12, wherein the second host compound has a structure represented by Formula 2 or Formula 3: 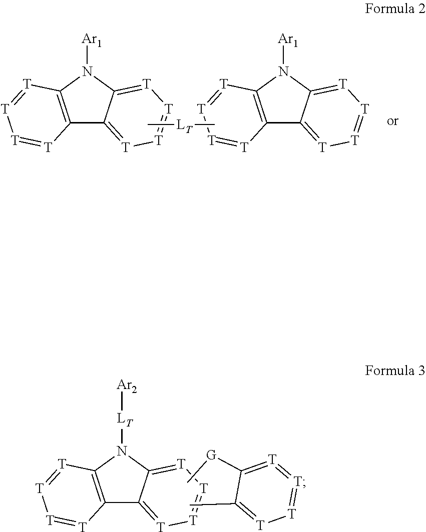
wherein
T is, at each occurrence identically or differently, selected from C, CRₜ, or N;
G is, at each occurrence identically or differently, selected from C(R_{g})₂, NR_{g}, O, or S; when multiple R_{g} are present at the same time, the multiple R_{g} are the same or different;
L_{T} is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms, or a combination thereof;
Rₜ and R_{g} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
Ar₁ and Ar₂ are, at each occurrence identically or differently, selected from substituted or unsubstituted aryl having 6 to 30 carbon atoms, substituted or unsubstituted heteroaryl having 3 to 30 carbon atoms, or a combination thereof; and
adjacent substituents Rₜ, R_{g} can be optionally joined to form a ring.

14. The organic electroluminescent device of claim 10, wherein the organic layer is an emissive layer, the compound is a host compound, the emissive layer further comprises at least one metal complex, and the at least one metal complex has a general formula of M(Lₐ)ₘ(L_{b})ₙ(L_{c})_{q};
the metal M is selected from a metal with a relative atomic mass greater than 40;
Lₐ, L_{b}, and L, are a first ligand, a second ligand, and a third ligand coordinated to the metal M, respectively; Lₐ, L_{b}, and Le may be the same or different;
Lₐ, L_{b}, and L_{c} can be optionally joined to form a multidentate ligand;
m is 1, 2, or 3, n is 0, 1, or 2, q is 0, 1, or 2, and m+n+q is equal to the oxidation state of the metal M; when m is greater than or equal to 2, multiple Lₐ may be the same or different; when n is 2, two L_{b} may be the same or different; when q is 2, two Le may be the same or different;
the ligand Lₐ has a structure represented by Formula 4: 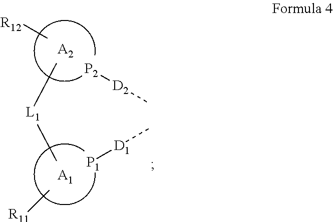
the ring A₁ and the ring A₂ are, at each occurrence identically or differently, selected from a substituted or unsubstituted aromatic ring having 5 to 30 ring atoms, a substituted or unsubstituted heteroaromatic ring having 5 to 30 ring atoms, or a combination thereof;
P₁ and P₂ are, at each occurrence identically or differently, selected from C or N;
D₁ and D₂ are, at each occurrence identically or differently, selected from a single bond, O, or S;
L₁ is selected from the group consisting of: a single bond, BR′, CR′R′, NR′, O, SiR′R′, PR′, S, GeR′R′, Se, substituted or unsubstituted vinylene, ethynylene, substituted or unsubstituted arylene having 6 to 30 carbon atoms, substituted or unsubstituted heteroarylene having 5 to 30 carbon atoms, and combinations thereof; when two R′ are present at the same time, the two R′ are the same or different;
R₁₁ and R₁₂ represent, at each occurrence identically or differently, mono-substitution, multiple substitutions, or non-substitution;
R₁₁, R₁₂, and R′ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof;
adjacent substituents R₁₁, R₁₂, R′ can be optionally joined to form a ring;
the ligands L_{b} and L_{c} are, at each occurrence identically or differently, selected from a monoanionic bidentate ligand;
preferably, the ligands L_{b} and L_{c} are, at each occurrence identically or differently, selected from the group consisting of the following structures: 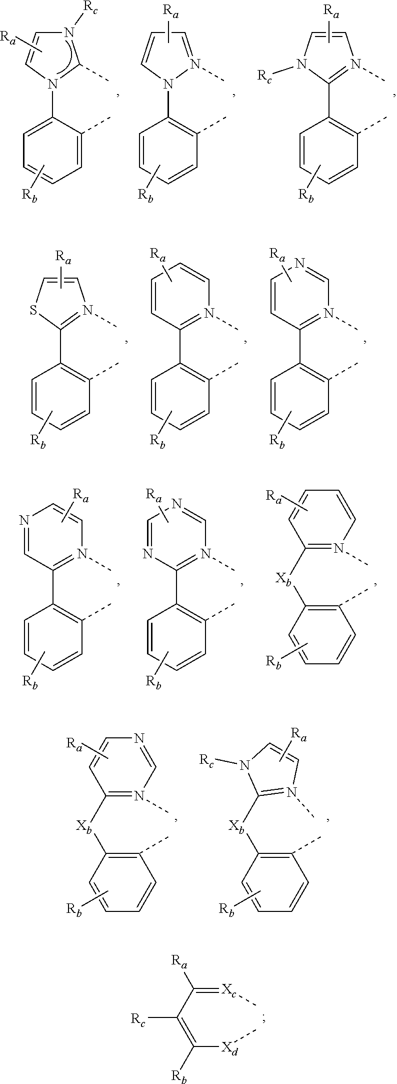
wherein
Rₐ and R_{b} represent, at each occurrence identically or differently, mono-substitution, multiple substitutions, or non-substitution;
X_{b} is, at each occurrence identically or differently, selected from the group consisting of: O, S, Se, NR_{N1}, and CR_{C1}R_{C2};
X_{b} and X_{d} are, at each occurrence identically or differently, selected from the group consisting of: O, S, Se, and NR_{N2};
Rₐ, R_{b}, R_{c}, R_{N1}, R_{N2}, R_{C1}, and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof; and
adjacent substituents Rₐ, R_{b}, R_{c}, R_{N1}, R_{N2}, R_{C1}, and R_{C2} can be optionally joined to form a ring.

15. A compound composition, comprising the compound of claim 1.
